(19) United States
(12) Patent Application Publication
(10) Pub. No.: US 2022/0145350 A1
(43) Pub. Date: May 12, 2022

(54) LIPID BIOSYNTHESIS AND ABIOTIC STRESS RESILIENCE IN PHOTOSYNTHETIC ORGANISMS

(71) Applicant: Board of Trustees of Michigan State University, East Lansing, MI (US)

(72) Inventors: Gregory Bonito, East Lansing, MI (US); Zhi-Yan Du, East Lansing, MI (US)

(21) Appl. No.: 17/435,530

(22) PCT Filed: Feb. 28, 2020
(86) PCT No.: PCT/US2020/020412

§ 371 Date: Sep. 1, 2021

Related U.S. Application Data
(60) Provisional application No. 62/812,722, filed on Mar. 1, 2019.

(51) Int. Cl. C12P 39/00; C12N 1/12; C12N 1/14

(52) U.S. Cl. CPC C12P 39/00; C12N 1/14; C12N 1/12

(57) ABSTRACT

This application describes methods of using fungi to harvest algae. As illustrated herein the algae stick onto and are captured directly by the hyphae of the fungi. The fungi, the algae, or both can be modified to express heterologous proteins or other products. The methods facilitate harvesting of useful strains of algae and the products made by such algae.

### CROSS-REFERENCE TO RELATED APPLICATIONS

This application claims benefit of priority to the filing date of U.S. Provisional Application Ser. No. 62/812,722, filed Mar. 1, 2019, the contents of which application is specifically incorporated herein by reference in its entirety.

This application is related to U.S. Provisional Application Ser. No. 62/458,236, filed Feb. 13, 2017, to U.S. Ser. No. 15/894,457 filed Feb. 12, 2018, and to U.S. Ser. No. 16/058,632 filed Aug. 8, 2018.

### GOVERNMENT FUNDING

This invention was made with government support under 1737898 awarded by the National Science Foundation. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Feb. 25, 2020, is named 2015443.txt and is 376,832 bytes in size.

### BACKGROUND OF THE INVENTION

Microbes have been used for many manufacturing purposes, including for energy production and the production of useful materials. For example, market prices for energy and fuels have been comparatively low but easily accessible petroleum and natural gas deposits have been depleted. In addition, emerging economies are growing, and environmental concerns are also growing. Significant restructuring or replacement of a portion of fossil fuels may be needed, for example, by renewable energy technologies such as biofuels. Currently, the largest volume of biofuels today is in the form of bioethanol for spark-ignition engines, with a smaller amount in the form of biodiesel for compression-ignition engines. Both bioethanol and biodiesel are produced primarily from terrestrial plant material. However, it is not optimal in the long term to produce fuels using food crops since food crops require premium land, abundant water, and large inputs of energy in the form of agricultural machinery and fertilizer. Thus, it would be advantageous to produce biofuels from alternative sources.

Plant and algal oils are some of the most energy-dense naturally occurring compounds that can be used as feedstocks for biofuel products. Microalgae are promising sustainable feedstocks for supplanting fossil fuels because they provide high oil yield, have short generation times, have low agricultural land requirements, have low freshwater needs, and exhibit reduced greenhouse gas emissions during algal cultivation.

In spite of these apparent advantages, the high cost of microalgal-based fuel production prevents its application in the market. The major barriers for the cost-effective production of microalgal biofuels include: (1) high cost for harvesting microalgae; (2) low oil content and suboptimal composition, (3) high cost of lipid extraction; and (4) impasses in sustainable nutrient supply. Among these barriers harvesting microalgae is particularly challenging because of the small cell size (typically 2-20 μm) and low density (0.3-5 g/L) of microalgae, which can account for up to 50% of the total cost of biofuel products. Traditional harvesting methods include chemical flocculation using multivalent cations such as metal salts and cationic polymers to neutralize the negative charge on the surface of microalgal cell walls, filtration for relatively large algae (>70 μm), sedimentation/floatation for species that either fall out of suspension or float without sufficient mixing, thermal drying, and centrifugation, which has a high cost and energy consumption.

### SUMMARY

To overcome the major challenges in algal biofuel production, including the high costs of harvesting, lipid extraction, and the nutrient supply, as well as low oil content in algae, the inventors have developed methods for harvesting algae by using fungi as a filtration system. As illustrated herein the mycelial network of fungi (e.g., Mortierella sp.) is efficient at capturing algae, forming large bio-aggregates that readily flocculate out of solution, so that the bio-aggregates can be easily harvested. The algae, the fungi, or both can be modified to express heterologous products.

Described herein are methods for filtration of algae from culture using fungal mycelia. The methods can include making filtration systems from living fungal mycelia. Algae cultures can be contacted with the fungal filtration systems. The algae stick to the fungal mycelia to form fungal-algal aggregates that can readily be removed from culture. For example, the algae readily stick onto and are directly captured by fungal hyphae (rather than in pores). The fungal filters do not clog, even when saturated with algae. Products made by the fungi or the algae can be isolated from the fungal-algal aggregates. Alternatively, the algae can be isolated from the fungi and components from the algae or the fungi can be isolated. Such methods facilitate manufacturing of useful products made by algae and/or fungi.

Also described herein are aggregates formed by fungi and algae. In some cases, the algae can be incorporated into the fungi to form consortia, which are robust. The fungi and algae can supply each other with nutrients. For example, the photosynthetic apparatus of algae can supply both the algae and the fungus with useful carbon-based nutrients. As illustrated herein, methods of making such fungal/algal consortia are simple and efficient. Hence, the costs of making, growing, and maintaining fungal/algal consortia are low. Such fungal/algal consortia are therefore useful for making a variety of compounds and materials, including oils, biofuels, and biomass.

One aspect of the invention is incubating Mortierella within a culture medium in a container, on a solid surface, or on a solid surface within a container to form a fungal-filter and contacting a culture of algae with the fungal-filter. Prior to forming the consortia described herein, the fungi were heterologous to the algae, meaning that fungi and the algae had not previously formed consortia.

Another aspect is a method that involves contacting a fungal-filter having fungal mycelia with a culture of algae to generate an aggregate of algae bound to the fungal-filter hyphae to thereby capture the algae from the culture. The fungal-filter can be in a container, on a solid surface, or on a solid surface within the container. The fungal-filter can, for example, be pre-made and stored as a dry or moist filter. In some cases the fungal mycelia or fungal cells are in solution and the fungal-filter is formed in situ after the fungal mycelia or fungal cells are contacted with the algae. The container or the solid surface can be a petri dish, a silicon membrane, a mesh, or a large pored fabric membrane.

The algae can be removed from solution by contacting the algae with the fungal-filter to form a flocculate that is readily removed by centrifugation or simply decanting the liquid medium from the flocculate. However, in some cases the culture of the algae can be passed through the fungal-filter.

In some cases, the fungal mycelia include Mortierella mycelia. For example, the Mortierella can be Mortierella elongata or Mortierella alpina.

A variety of algae types can be flocculated and collected by contacting the algae with the fungal-filter. For example, the algae can be microalgae, green algae, or blue-green algae.

The method can also include harvesting an aggregate of algae bound to the fungal-filter hyphae. In some cases, the methods can include separating the algae from the fungal-filter hyphae. Separation from the fungal-filter hyphae can be, for example, by one or more of digestion of the fungal-filter, addition of salt, addition of detergent, vortexing, re-suspension of the algae, or a combination thereof.

The method can further include harvesting the aggregate of algae bound to the fungal-filter hyphae and extracting oil, protein, or carbohydrate therefrom.

In some cases, the algae are modified to express a selected product, the fungal filter have fungal cells modified to express a product, or the algae and the fungal cells are separately modified to express one or more products.

Hence, the algae and/or the fungal filter can produce products such as one or more enzymes that can contribute to synthesizing one or more oils, carbohydrates, vitamins, proteins, or polymers.

Another method described herein in a method that involves inoculating fungal cells into a dish comprising culture medium, and incubating the fungal cells in the culture medium, for a time and under conditions sufficient to form a fungal filter.

### DESCRIPTION OF THE FIGURES

FIG. 1A-1E illustrate interaction between the soil fungus Mortierella elongata and the marine alga Nannochloropsis oceanica. FIG. 1A illustrates co-cultivation of M. elongata AG77 and N. oceanica (Noc) in flasks for 6 days. Tissues indicated by the arrow head are aggregates formed by AG77 mycelia and attached Noc cells. FIG. 1B shows differential interference contrast micrographs of the tissues shown in FIG. 1A. As shown in FIG. 1B, a large number of Noc cells were captured by AG77 mycelia. FIGS. 1C to 1E show images of alga-fungus aggregates by scanning electron microscopy. FIG. 1C illustrates that Noc cells stick to the fungal mycelia after 6-day co-culture. FIG. 1D shows a Noc cell adhering tightly to a hypha by the outer extensions of cell wall as indicated with red arrows. FIG. 1E illustrates irregular tube-like extensions of Noc cell wall attached to the surface of fungal cell wall.

FIGS. 2A-2H illustrate carbon exchange between N. oceanica and M. elongata AG77. FIG. 2A includes FIGS. 2A-1 and 2A-2, which illustrate carbon (C) transfer from [¹⁴C]sodium bicarbonate (NaHCO₃)-labeled N. oceanica (Noc) cells to M. elongata AG77 (FIG. 2A-1) or from [¹⁴C]glucose-labeled AG77 to Noc cells (FIG. 2A-2) after 7-day co-culture in flasks with physical contact between the N. oceanica and M. elongata AG77. Radioactivity of ¹⁴C was measured with a scintillation counter (dpm, radioactive disintegrations per minute) and then normalized to the dry weight of samples (dpm/mg biomass). Free Noc refers to unbound Noc cells in supernatant. Attached refers to Noc cells separated from AG77-Noc aggregates. FAAs refers to free amino acids. The “soluble compounds” refers to compounds in the supernatant after acetone precipitation of proteins extracted by SDS buffer. Data are presented in the average of three biological repeats with standard deviation (Means±SD, n=3). FIG. 2B includes FIGS. 2B-1 and 2B-2, which illustrate radioactive ¹⁴C transfer between Noc and AG77 without physical contact. Algae and fungi were incubated in cell-culture plates with filter-bottom inserts (pore size of 0.4 μm) which separate Noc cells and AG77 mycelia from each other but allow metabolic exchange during co-culture. Error bars indicate SD (n=3). Radioactive carbon (C) transfer was measured from [¹⁴C]sodium bicarbonate (NaHCO₃)-labeled N. oceanica (Noc) cells to M. elongata AG77 (FIG. 2B-1) or from [¹⁴C]glucose-labeled AG77 to Noc cells (FIG. 2B-2). FIG. 2C graphically illustrates the relative abundance of ¹⁴C radioactivity in AG77 recipient cells compared to ¹⁴C-labeled Noc donor cells after 7-day co-culture (total AG77 dpm/total ¹⁴C-Noc dpm). FIG. 2D illustrates the relative abundance of ¹⁴C radioactivity in Noc recipient cells compared to ¹⁴C-labeled AG77 donor cells after 7-day co-culture (total Noc dpm/total ¹⁴C-AG77 dpm). Physical contact refers to living ¹⁴C-labeled cells added to unlabeled cells for co-cultivation in flasks. No contact refers to samples grown separately in plates with inserts. Heat-killed ¹⁴C-cells, heat-killed ¹⁴C-labeled Noc or heat-killed AG77 were killed by heat treatment at 65° C. for 15 min before the addition to unlabeled cells in flasks. Free refers to unbound Noc cells in supernatant. Att refers to Noc cells attached to AG77. Total refers to Noc cells grown separately with AG77 in plates and inserts. Error bars indicate SD (n=3). FIGS. 2E-2H further illustrate ¹⁴C exchange between N. oceanica and M. elongata AG77 without physical contact. FIG. 2E illustrates the beginning of co-culture of N. oceanica (Noc) and M. elongata AG77 in 6-well plates with filter-bottom inserts (i.e., without physical contact). FIG. 2F illustrates co-culture of N. oceanica (Noc) and M. elongata AG77 in 6-well plates with filter-bottom inserts (i.e., without physical contact), and after 7-day co-culture, the inserts were moved to the adjacent empty wells (bottom) for harvesting samples. There is no cross contamination observed between Noc and AG77 samples as indicated by the images. FIG. 2G shows a side-view schematic diagram of alga-fungus co-culture (e.g., as illustrated in FIG. 2E) and sample harvesting (e.g., as illustrated in FIG. 2F) with an insert and plate. The hydrophilic polytetrafluoroethylene filter (pore size of 0.4 μm) at the bottom of the inserts separates Noc and AG77 during co-culture but allows metabolic exchange between the plate well and insert. [¹⁴C]sodium bicarbonate (NaHCO₃)-labeled Noc cells were grown in the plate well or insert while recipient AG77 was grown in the insert or plate well, respectively. Similar incubation conditions were used for [₁₄C]glucose- or [¹⁴C]sodium acetate-labeled AG77 and recipient Noc. FIG. 2H graphically illustrates ¹⁴C transfer from [¹⁴C]sodium acetate-labeled AG77 to recipient Noc. ¹⁴C radioactivity (dpm, radioactive disintegrations per minute) was normalized to the dry weight (dpm/mg). FAAs, free amino acids; soluble compounds, supernatant after acetone precipitation of SDS-protein extraction. Error bars indicate SD (n=3).

FIGS. 3A-3J illustrate that N. oceanica benefits from co-culture with M. elongata. FIG. 3A illustrates nitrogen (N) exchange between N. oceanica (Noc) and M. elongata AG77 as examined by ¹⁵N-labeling experiments. [¹⁵N]potassium nitrate-labeled Noc cells or [¹⁵N]ammonium chloride-labeled AG77 were added to unlabeled AG77 or Noc cells, respectively, for 7-days co-culture in flasks (physical contact) or for 7-days cell culture in plates with inserts (no physical contact). Algae and fungi were separated and weighed (dry biomass) after the co-culture, and their isotopic composition (δ¹⁵N, ratio of stable isotopes ¹⁵N/¹⁴N) and N content (% N) were determined using an elemental analyzer interfaced to an Elementar Isoprime mass spectrometer following standard protocols. The N uptake rate of ¹⁵N-Noc-derived N (¹⁵N) by AG77 from and that of ¹⁵N-AG77-derived N by Noc cells (¹⁵N) were calculated based on the Atom % ¹⁵N [¹⁵N/(¹⁵N+¹⁴N)100%], % N and biomass. C, chloroplast; N, nucleus; Nu, nucleolus; M, mitochondrion; V, vacuole; L, lipid droplet. Values are the average of three biological repeats. FIGS. 3B-3D illustrate viabilities of the N. oceanica (Noc) and M. elongata AG77 under various culture conditions. FIG. 3B shows images illustrating viability assays of Noc cells under nitrogen deprivation (—N). FIG. 3C shows images illustrating viability assays of Noc co-cultured with AG77 under nitrogen deprivation (—N). For FIGS. 3B and 3C, dead Noc cells were detected by SYTOX Green staining (green fluorescence), while red colors indicate Noc chlorophyll fluorescence in the original. FIG. 3D graphically illustrates that the viability of nutrient-deprived Noc cells increased when co-cultured with M. elongata AG77 or M. elongata NVP64. The abbreviation —C indicates carbon deprivation. The abbreviation —N indicates nitrogen deprivation. Results were calculated from 1,000 to 5,000 cells of five biological repeats with ImageJ software. Asterisks indicate significant differences compared to the Noc control by Student's t test (*P≤0.05, **P≤0.01; Means±SD, n=5). FIG. 3E illustrates the total organic carbon (C) measured in the buffer of 18-day fungal cultures of M. elongata AG77 and NVP64 compared to the f/2 medium control (f/2 con). FIG. 3F graphically illustrates the dissolved nitrogen (N) measured in the buffer of 18-day fungal cultures of M. elongata AG77 and NVP64 compared to the f/2 medium control (f/2 con). Fungal cells were removed by 0.22 micron filters. Means±SD, n=4. *P≤0.05, **P≤0.01. FIG. 3G-3H further illustrate nitrogen (N) exchange between N. oceanica and M. elongata AG77 as examined by ¹⁵N-labeling experiments. FIG. 3G graphically illustrates nitrogen uptake by M. elongata AG77 cells after [¹⁵N]potassium nitrate-labeled Noc cells were added to unlabeled AG77 cells. FIG. 3H graphically illustrates nitrogen uptake by N. oceanica cells after [¹⁵N]ammonium chloride-labeled AG77 (2.7%, Atom % ¹⁵N) were added to unlabeled Noc cells. The results in FIG. 2G were generated by addition of [¹⁵N]potassium nitrate-labeled Noc cells [7.1%, Atom % ¹⁵N, ¹⁵N/(¹⁵N+¹⁴N)100%] to unlabeled AG77 for 7-day co-culture in flasks (physical contact, top) or cell-culture plates with inserts (no physical contact, bottom). Similarly, the results in FIG. 3H were generated by addition of [¹⁵N]ammonium chloride-labeled AG77 (2.7%, Atom % ¹⁵N) to unlabeled Noc cells for 7-day co-culture in flasks (physical contact, top) or cell-culture plates with inserts (no physical contact, bottom). Algae and fungi were separated and weighed (dry biomass) after the co-culture, and their isotopic composition (δ¹⁵N, ratio of stable isotopes ¹⁵N/¹⁴N) and N content (% N) were determined using an elemental analyzer interfaced to an Elementar Isoprime mass spectrometer following standard protocols. For FIG. 3G, the nitrogen uptake rates (μmol N/mg biomass/d) of Noc from the media (medium-N, isotope dilution) and that of AG77 from ¹⁵N-Noc-derived N (¹⁵N) were calculated based on the Atom % ¹⁵N, % N and biomass. Error bars indicate SD (n=3). Similar analyses were carried out to obtain the results in FIG. 3H where [¹⁵N]ammonium chloride-labeled AG77 (2.7%, Atom % ¹⁵N) and unlabeled Noc cells were incubated to calculate the uptake rate of medium-N by AG77 and that of ¹⁵N-AG77-derived N (¹⁵N) by Noc cells. Error bars indicate SD (n=3). FIGS. 3I-3J illustrate that various fungi from diverse clades exhibit intensive interaction with N. oceanica. FIG. 3I schematically illustrates the phylogeny of plant root-associated fungal isolates that were used for co-culture bioassay experiments. A phylogenetically diverse panel of basidiomycete, ascomycete and zygomycete fungi were tested. FIG. 3M illustrates co-culture of N. oceanica cells with different fungi and Saccharomyces cerevisiae in flasks containing f/2 media for 6 days. N. oceanica, algal culture control: the others, N. oceanica incubated with respective fungi or S. cerevisiae.

FIGS. 4A-4I (where FIG. 4I includes FIG. 4I-1 to 4I-4) illustrate intracellular localization of long-term co-cultured N. oceanica within M. elongata AG77 hyphae. FIGS. 4A-4C are transmission electron microscope (TEM) images of increasing magnification showing a cross section of AG77 mycelium containing a cluster of dividing Noc cells. AG77 and Noc were co-cultured for ˜one month. Arrow heads indicate same position. M, mycelium; Mw, Mortierella cell wall; Nw, Noc cell wall; C, chloroplast; Cy, cytoplasm; V, vacuole. FIG. 4A shows an image of N. oceanica within M. elongata AG77 hyphae. FIG. 4B shows an enlarged imaged of the boxed area shown in FIG. 4A. FIG. 4C shows a further enlargement of a portion of the image shown in FIG. 4B. FIGS. 4D-4H show differential interference contrast (DIC) images of AG77 “green hyphae” with N. oceanica (Noc) cells inside. Arrow heads indicate putative dividing Noc cells. FIG. 4D shows N. oceanica (Noc) cells inside M. elongata AG77 hyphae after co-culture for about one month. FIG. 4E also shows Noc cells inside M. elongata AG77 hyphae after co-culture for about one month. FIG. 4F shows Noc cells inside M. elongata AG77 hyphae after co-culture for about two months. FIG. 4G also shows Noc cells inside M. elongata AG77 hyphae after co-culture for about two months. FIG. 4H also shows Noc cells inside M. elongata AG77 hyphae after co-culture for about two months. FIG. 4I-1 to 4I-4 illustrate the origin of endosymbiosis of N. oceanica within M. elongata AG77. FIG. 4I-1 shows a differential interference contrast (DIC) micrograph of co-cultured N. oceanica (Noc) and M. elongata AG77 using a Leica DMi8 DIC microscope. After 35-day co-culture in flasks, AG77-Noc aggregates were transferred to 35 mm-microwell dish (glass top and bottom, MatTek) containing soft solid media (f/2 media supplemented with 0.25% low gelling temperature agarose and 10% PDB) to investigate the establishment of the Noc endosymbiosis in AG77. The red arrow head indicates a hypha coated by Noc cells around the hyphal tip. FIG. 4I-2 to 4I-4 show a differential interference contrast (DIC) micrograph of co-cultured Noc and M. elongata AG77 after three days of incubation in soft solid media, the same group of Noc and AG77 cells formed a “green hypha” (with Noc cells inside) as indicated by the red arrow head. Noc cells surrounding the hypha kept growing and dividing and formed a lollipop-like structure because of the solid media, which is not observed in liquid alga-fungus co-culture. In the enlargement of the lollipop region, the cyan arrow head points to Noc cells inside the fungal hypha. FIG. 4I-2 shows a field of N. oceanica (Noc) and M. elongata AG77. FIG. 4I-3 shows an enlargement of a portion of the image shown in FIG. 4I-4. FIG. 4I-4 shows an enlargement of a portion of the image shown in FIG. 4I-2.

FIG. 5A-5H illustrates physical interaction between algal N. oceanica and fungal M. elongata cells led to the degradation of the outer layer of N. oceanica algal cell wall. FIG. 5A shows lower magnification images of N. oceanica (Noc) cells incubated alone in f/2 medium (bar=1 micron). FIG. 5B shows somewhat higher magnification images of Noc cells incubated alone in f/2 medium (bar=1 micron). FIG. 5C shows even higher magnification images of Noc cells incubated alone in f/2 medium (bar=1 micron). FIG. 5D shows an image of an Noc cell wall after incubation of the Noc cell alone in f/2 medium (bar=100 nm). As illustrated, the Noc cells shown in FIG. 5A-5D have a smooth surface. FIG. 5E shows an image of Noc cells attached to M. elongata AG77 (AG77) hyphae in a co-culture (bar=10 microns), illustrating that the outer layer of the Noc algal cell walls is not as intact as that of the Noc controls shown in FIG. 5A-5D. FIG. 5F shows an expanded image of Noc cells attached to M. elongata AG77 (AG77) hyphae in a co-culture (bar=1 micron), illustrating that the outer layer of the Noc algal cell walls is not as intact as that of the Noc controls shown in FIG. 5A-5D. FIG. 5G further illustrates the structure of N. oceanica (Noc) cells without physical interaction with M. elongata AG77 (AG77) (bar=1 micron) when using a 6-well culture plate and membrane insert (pore size of 0.4 μm) that separates the Noc and AG77 cells but allows metabolic exchange between the partners. FIG. 5H shows an expanded view of one N. oceanica (Noc) (bar=1 micron) cell incubated without physical interaction with M. elongata AG77 (AG77) by using a 6-well culture plate and membrane insert (pore size of 0.4 μm) that separates the Noc and AG77 cells but allows metabolic exchange between the partners. As shown in FIG. 5G-5H, the Noc algal cells have intact cell walls, for example in their outer layer, where in contrast, the outer layer is defective when the Noc-algal cells form a consortium with the M. elongata AG77 (AG77) hyphae (compare FIGS. 5E-5F with FIGS. 5G-5H).

FIG. 6A-6D illustrate incubation of N. oceanica cells in the environmental photobioreactor (ePBR). FIG. 6A shows N. oceanica cells when inoculated in f/2 medium containing NH₄Cl. FIG. 6B shows N. oceanica cells that were incubated in the ePBR to stationary phase (day 1, referred to as S1). FIG. 6C shows N. oceanica cells that were incubated in the ePBR after growth for 8 days (referred to as S8). Cultures were incubated under fluctuating light at 23° C. and were sparged with air enriched to 5% CO₂ at 0.37 L min⁻¹ for 2 min per hour. FIG. 6D graphically illustrates light conditions for the cultures in the ePBR: fluctuating lights (0 to 2,000 μmol photons m⁻² s⁻¹) under diurnal 14/10 h light/dark cycle.

FIG. 7A-7F illustrate harvesting Nannochloropsis oceanica by bio-flocculation with Mortierella fungi. FIG. 7A shows and image of a co-culture of N. oceanica (Noc) with M. elongata AG77. The arrow indicates green aggregates formed by AG77 mycelium and attached Noc cells. FIG. 7B shows an image of co-culture of N. oceanica (Noc) with Morchella americana 3668S. For FIGS. 7A-7B, fungal mycelium was added to the Noc culture and the mixture was incubated for 6 days. FIG. 7C shows an image of Noc cells attached to AG77 mycelium as visualized by differential interference contrast (DIC) microscopy. FIG. 7D shows that there was no obvious attachment of Noc cells on the Morchella americana 3668S mycelium. FIG. 7E graphically illustrates bio-flocculation efficiency for harvesting Noc cells by cocultivation with Mortierella elongata AG77, Mortierella elongata NVP64, and Mortierella gamsii GBAus22. The bioflocculation efficiency was determined by the cell density of uncaptured cells compared to that of a no-fungus Noc culture control. A Morchella 3668S culture was used as a negative control. The results are the average of five biological replicates and error bars indicate standard deviation. Asterisks indicate significant differences relative to the 2 hr co-cultures by paired-sample Student's t-test (*P≤0.05; **P≤0.01). FIG. 7F graphically illustrates Noc cell size (diameter) in the Noc culture and in various alga-fungus co-cultures.

FIG. 8A-8C illustrate interaction between Nannochloropsis oceanica and Mortierella mycelium. FIG. 8A shows scanning electron microscopy images illustrating the interaction between N. oceanica (Noc) cells and Mortierella elongata AG77. FIG. 8B shows scanning electron microscopy images illustrating the interaction between N. oceanica (Noc) cells and M. elongata NVP64. Noc cells are attached to the fungal mycelium as shown in the top panels of FIGS. 8A-8B. Higher magnification micrographs shown in the lower panels illustrate that Noc cells have a highly structured cell wall with protrusions, with which they attach to the rough surface of the fungal cell wall. The red arrowheads in the lower panels of FIGS. 8A-8B indicate that tube-like structures connect the algal and fungal cell walls. FIG. 8C shows images of Morchella americana 3668S mycelium collected from Noc-3668S culture after 6-day co-cultivation, where the Morchella americana 3668S mycelium does not aggregate with N. oceanica cells.

FIG. 9A-9I illustrate that Mortierella fungi have more oil droplets than Nannochloropsis oceanica in f/2 medium. FIG. 9A shows confocal micrographs of N. oceanica-M. elongata AG77 after six days of co-culture in PDB medium, illustrating the lipid droplets within the fungal mycelium. Green fluorescence indicates lipid droplets stained with BODIPY. FIG. 9B shows confocal micrographs of N. oceanica-M. elongata NVP64 after six days of co-culture in PDB medium, illustrating the lipid droplets within the fungal mycelium. Green fluorescence indicates lipid droplets stained with BODIPY. FIG. 9C shows confocal micrographs of N. oceanica-Mortierella gamsii GBAus22 after six days of co-culture in PDB medium, illustrating the lipid droplets within the fungal mycelium. Green fluorescence indicates lipid droplets stained with BODIPY. FIG. 9D shows confocal micrographs of N. oceanica-Morchella americana 3668S after six days of co-culture in PDB medium, illustrating the lipid droplets within the fungal mycelium. Green fluorescence indicates lipid droplets stained with BODIPY. FIG. 9E shows images of lipid droplets in N. oceanica (Noc) cells. The red color is from autofluorescence of Noc chloroplast. FIG. 9F shows lipid droplets in the N. oceanica-M. elongata AG77 cells after six days of co-cultivation of the algal and fungal cells in f/2 medium. FIG. 9G shows lipid droplets in the N. oceanica-M. elongata NVP64 cells after six days of co-cultivation of the algal and fungal cells in f/2 medium. FIG. 9H shows lipid droplets in the N. oceanica-Mortierella gamsii GBAus22 cells after six days of co-cultivation of the algal and fungal cells in f/2 medium. FIG. 9I shows lipid droplets in the N. oceanica-Morchella americana 3668S cells after six days of co-cultivation of the algal and fungal cells in f/2 medium.

FIG. 10A-10C graphically illustrate fatty acid profiling of triacylglycerol (TAG) and total lipid in Mortierella fungi, Nannochloropsis oceanica, and algae-fungi aggregates after co-cultivation. FIG. 10A graphically illustrates the amounts of various fatty acids in triacylglycerol and total lipid detected in assays of N. oceanica grown in shaker flasks containing f/2 medium. Fatty acids are indicated with number of carbons:number of double bonds. Results are the average of five biological replicates with error bars indicating standard deviations (n=5). FIG. 10B graphically illustrates the amounts of various fatty acids in triacylglycerol and total lipid detected in assays of M. elongata AG77 incubated in f/2 medium. n=5. FIG. 10C graphically illustrates the amounts of various fatty acids in triacylglycerol and total lipid detected in assays of the algae-fungi aggregates after 6-d co-cultivation. n=5.

FIG. 11A-11B graphically illustrate the triacylglycerol content in Nannochloropsis oceanica cells. FIG. 11A graphically illustrates the mole ratio of triacylglycerol (TAG) compared to total lipid. Cells were grown in shaker flasks. N0-120, Nitrogen deprivation (f/2 medium lacking nitrogen for 0-120 hours; R24-72, nitrogen resupply (f/2) medium for 24-72 hours. The average of three biological replicates and standard deviation are shown (n=3). FIG. 11B graphically illustrates the TAG and total lipid content per gram of whole cell dry weight. n=3.

FIG. 12A-12D illustrate cell growth and biomass in the environmental photobioreactor (ePBR). FIG. 12A graphically illustrates cell counts of N. oceanica (Noc) cells were inoculated to ˜1×10⁶ mL⁻¹ and incubated in the environmental photobioreactor containing modified f/2 media with NH4Cl, KNO3, or urea as nitrogen source. The average of three biological replicates and standard deviation are shown (n=3). FIG. 12B graphically illustrates the dry weight per liter of cells grown in different f/2 media. n=3. FIG. 12C graphically illustrates the cell growth during S1-8 in f/2-NH4Cl. n=3. FIG. 12D graphically illustrates the cell dry weight during S1-8 in f/2-NH4Cl. n=3. L1-6, days 1-6 of log phase; S1 and 2, day 1 and 2 of stationary phase.

FIG. 13A-13B illustrates that chlorophyll as proxy of triacylglycerol accumulation. FIG. 13A illustrates analysis of triacylglycerol (TAG) by thin layer chromatography (TLC). Arrowheads indicate the TAG bands. S1 to S8, day 1 to 8 after the cells reached stationary phase; control, TAG standard. FIG. 13B graphically illustrates a correlation between chlorophyll content and TAG-to-total-lipid ratio following prolonged incubation in the environmental photobioreactor (ePBR) containing f/2-NH4Cl medium. TAG and total lipid were subjected to transesterification reaction and the resulting fatty acid methyl esters were quantified by gas chromatography and flame ionization detection (GC-FID). r2, correlation coefficient; n=4.

FIG. 14A-14B illustrate triacylglycerol accumulation during prolonged incubation in f/2-NH₄Cl medium supplemented with or without sodium bicarbonate. N. oceanica cells were inoculated and incubated in f/2-NH₄Cl medium (with or without NaHCO₃) in ePBRs and sparged with air enriched to 5% CO₂ at 0.37 L min⁻¹ for 2 min per hour. S1 to 8, day 1 to 8 after the cultures reached stationary phase. FIG. 14A illustrates the pH of the culture from S5 to S8. FIG. 14B graphically illustrates TAG content during prolonged incubation. The results are the average of three biological replicates and error bars indicate standard deviation. Asterisks indicate significant difference between CO₂, and CO₂ & NaHCO₃. **, P<0.01; *, P<0.05; n=3.

FIG. 15A-15C illustrate increasing triacylglycerol (TAG) content in Nannochloropsis oceanica using limited ammonium as nitrogen source. FIG. 15A shows images of N. oceanica (Noc) cells, illustrating production of large lipid droplets in N. oceanica (Noc) cells during prolonged incubation in the environmental photobioreactor (ePBR) containing f/2-NH4Cl medium. Noc cells grow fast in f/2-NH4Cl medium and suffer from nutrient limitation after being for 8 days in the stationary phase, when the confocal micrographs were taken. Green fluorescence indicates lipid droplets stained with BODIPY, while red fluorescence represents autofluorescence of Noc chloroplasts. FIG. 15B shows lipid droplet staining of M. elongata AG77 and Noc cells after 6-days co-cultivation. FIG. 15C graphically illustrates fatty acid (FA) analyses of triacylglycerol and total lipid in the alga-fungus aggregate as shown in (FIG. 15B), where the inset shows biomass ratio of TAG, while the larger graph shows total FA relative to the total cell dry weight (DW). n=5.

FIG. 16A-16D shows a schematic diagram illustrating predicted fatty acid/lipid pathways in M. elongata AG77. Proteins likely involved in the synthesis of fatty acids (FA), polyunsaturated fatty acids (PUFA), and triacylglycerol (TAG) are identified in the sequenced genome of M. elongata AG77 at the JGI fungal genome portal MycoCosm (Table 3). FIG. 16A illustrates the fatty acid (FA) synthetic pathway. ACP, acyl carrier protein; AT, acetyltransferase; MPT, malonyl/palmitoyl transferase; ACSL, acyl-CoA synthetase; KS, β-ketoacyl synthase; ER, β-enoyl reductase; DH, dehydratase; KR, β-ketoacyl reductase. FIG. 16B shows the linear domain organization of fatty acid synthase (FASN) of M. elongata AG77. PPT, phosphopantetheine transferase. FIG. 16C illustrates PUFA synthetic pathways. ELOVL, fatty acid elongase; FAD, fatty acid desaturase. Fatty acids are designated by the number of total carbon:the number of double bonds. The position of specific double bonds is indicated either from the carboxyl end (Δ) or from the methyl end (ω). FIG. 16D illustrates TAG synthetic pathways. ALDH, aldehyde dehydrogenase; ADH, alcohol dehydrogenase; GK, glycerol kinase; GPDH, glycerol-3-phosphate dehydrogenase; GPAT, glycero-3-phosphate acyltransferase; PlsC, 1-acyl-sn-glycerol-3-phosphate acyltransferase; LPIN, phosphatidate phosphatase LPIN; PAP, phosphatidate phosphatase 2; Dgk, diacylglycerol kinase; DGAT, diacylglycerol acyltransferase; PDAT, phospholipid diacylglycerol acyltransferase.

FIG. 17A-17B illustrate expression vectors for lipid synthesizing enzymes. FIG. 17A shows a schematic map of a control vector that does not include the DG75 nucleic acid segment, and that is referred to as a pnoc ox cerulean hyg vector control. FIG. 17B shows a schematic map of an expression vector for generating N. oceanica DGTT5-overexpressing strains where the vector is referred to as a pnoc ox DGTT5 cerulean hyg vector.

FIG. 18A-18B illustrate that several species of cyanobacteria (genus Anabaena) form large bio-aggregates when incubated with Mortierella elongata membranes. FIG. 18A shows cultures of Anabaena variabilis, Anabaena cylindrica, and Anabaena sp. PCC 7120 without Mortierella elongata membranes. FIG. 18B shows Anabaena variabilis, Anabaena cylindrica, and Anabaena sp. PCC after co-culture with Mortierella elongata membranes. As illustrated, in the presence of Mortierella elongata membranes these Anabaena species flocculate into clumps that are readily harvested.

FIG. 19 illustrates that Chlorella sorokiniana algae can flocculate with Mortierella alpina.

FIG. 20A-20D illustrate that other species of Mortierella can flocculate with different types of algae. FIG. 20A shows that Chlamydomonas reinhardtii algae clump up or flocculate with Mortierella alpina. As shown on the left, when cultured alone, Chlamydomonas reinhardtii algae form a uniform suspension in culture, but as shown in the right, when Mortierella alpina is co-cultured with Chlamydomonas reinhardtii algae, flocculates form that facilitate harvesting of the Chlamydomonas reinhardtii algae with the Mortierella alpina fungi. FIG. 20B shows that Chlamydomonas reinhardtii algae clump up or flocculate with different strains of Mortierella alpina. FIG. 20C graphically illustrates the flocculation efficiency of different strains of Mortierella alpina. FIG. 20D graphically illustrates that various Mortierella alpina strains are enriched in poly-unsaturated fatty acids such as ARA, EPA, and DHA. Hence, co-cultures of algae with Mortierella alpina form commercially useful sources of such oils.

### DETAILED DESCRIPTION

As described herein, oil-producing fungi are very efficient at harvesting various types of algae. For example, various types of Mortierella fungi can flocculate green algae, blue-green algae (cyanobacteria), microalgae, and the like. Hence, fungi can act as filters for collection of algae.

Microalgae are unicellular photosynthetic organisms that live in a wide range of habitats from fresh, blackish, and saltwater ecosystems to soil environments. Compared to land-based crops, microalgae grow very fast and they are enriched in nutrients such as polyunsaturated fatty acids, neutral lipids, proteins, pigments and anti-oxidants.

Cyanobacteria, also called blue-green algae, are microscopic organisms found naturally in all types of water. Cyanobacteria are single-celled organisms that can live in fresh, brackish (combined salt and fresh water), and marine water. Because cyanobacteria use sunlight to make their own food their nutritional requirements can be small. Cyanobacteria are a popular microorganism for making a variety of useful products.

Green algae and other types of algae are useful for making a variety of products such as oils, carbohydrates, proteins, polymers, biofuels, food supplements (e.g., carrageenan, algin, omega-3 oils, and whole algae), and fertilizers.

The demand for algae products continues to grow in the world market. Although algae are easy to incubate in large scale bioreactors and open ponds, they are very difficult to harvest because of the small size. For example, microalgae, green algae, and cyanobacteria (e.g. Anabaena) are typically 2-20 microns in size. Harvesting such algae cost can account for up to 50% of the total cost of product production using currently available methods (see, e.g., Sun et al., 2011; Du et al., 2018). To efficiently harvest algae at much lower cost, the inventors have developed a high-efficient fungal-filter system, whereby fungal mycelium of the industrial fungus Mortierella is used as a biological filter to capture the algae. Mortierella species are widespread soil fungi and they are usually safe to plants or animals and humans.

Many Mortierella species are used for human nutraceuticals such as arachidonic acid (C20:4, ARA), an omega-6 polyunsaturated fatty acid that are good for heart health and systemic inflammation (Roberts et al., 2007: Chowdhury et al., 2014). Mortierella grow very fast and they can be cultured under simple conditions, including on food and sewage wastes. As illustrated herein the mycelial network of Mortierella is efficient at capturing algae, forming large bio-aggregates that flocculate out of solution, and can be easily harvest with mesh or simple filtration (Du et al., 2018). Based on these findings an algae filtration system was developed that involves growing Mortierella mycelium into a novel fungal-filter, which can significantly reduce the cost of harvesting microalgae compared to the traditional methods such as chemical flocculation, thermal drying, and centrifugation. The algae stick onto and are captured directly by the hyphae, rather than in pores, thus, these fungal-filters do not clog, even when saturated. The algae-based nutraceutical and food industry can benefit from the methods described herein.

Bio-flocculates of algae and Mortierella fungi are highly enriched in protein and omega-3 and omega-6 fatty acids such as EPA (eicosapentaenoic acid) and ARA, and the global omega-3 and omega-6 ingredient market records a revenue of $0.43 billion in 2016 and is expected to grow at an annual rate of 11.5% during 2018-2023 (Mordor Intelligence, 2018a). Algae-based animal feed and ingredient market is also a billion-dollar market, with more than 8% annual growth rate expected during the period of 2018-2022 (Business Wire, 2018).

The algae-fungi aggregates are therefore promising feedstocks for high-value products for nutraceutical, food and animal feed markets. As illustrated herein oleaginous fungi can flocculate algae such as N. oceanica CCMP1779 (a marine alga with the ability to produce high levels of TAG), as well as Chlorella sorokiniana (freshwater green microalga), Chlamydomonas reinhardtii (single-cell green alga), Anabaena variabilis (filamentous cyanobacterium), Anabaena cylindrica (filamentous cyanobacterium), and Anabaena sp. PCC 7120 (filamentous, freshwater cyanobacterium). Results provided herein also illustrate that the various Mortierella species can be used to efficiently harvest N. oceanica, Chlorella sorokiniana cells. Methods are provided herein for increasing TAG content in N. oceanica by optimizing growth conditions and by using genetic engineering approaches in combination with bio-flocculation to harvest algal cells.

Described herein are viable fungi having viable algae within their fungi hyphae. In other words, the fungi with internalized algae form can form a consortium where, for example, the internalized algae may depend on the host fungus for nitrogen and other nutrients, while the algae can provide carbon-based nutrients and other metabolites that can be generated by algal photosynthesis. Compositions of such consortia of fungi with viable algae within the fungi hyphae, as well as methods of making and using such consortia and compositions are also described herein.

The algae employed can include a wide variety of algae. Examples include diatoms (bacillariophytes), green algae (chlorophytes), blue-green algae (cyanophytes), and golden-brown algae (chrysophytes). In addition, a fifth group known as haptophytes may be used. Specific non-limiting examples of bacillariophytes capable of lipid production include the genera Amphipleura, Amphora, Anabaena, Chaetoceros, Cyclotella, Cymbella, Fragilaria, Hantzschia, Navicula, Nitzschia, Phaeodactylum, and Thalassiosira. Specific non-limiting examples of chlorophytes capable of lipid production include Ankistrodesmus, Botryococcus, Chlorella, Chlorococcum, Dunaliella, Monoraphidium, Oocystis, Scenedesmus, and Tetraselmis. In one aspect, the chlorophytes can be Chlorella or Dunaliella. Specific non-limiting examples of cyanophytes capable of lipid production include Oscillatoria and Synechococcus. A specific example of chrysophytes capable of lipid production includes Boekelovia. Specific non-limiting examples of haptophytes include Isochrysis and Pleurochrysis. In some cases, an alkenone-producing alga, for example, a species of the Isochrysis family which includes, but not limited to, Isochrysis galbana, Isochrysis sp. T-Iso, and Isochrysis sp. C-Iso can be employed. Other examples of alkenone-producing algae include Emiliania huxleyi and Gephyrocapsa oceanica. In some cases, the algae is not Nostoc punctiforme.

Examples of algae can be species of Amphipleura, Amphora, Anabaena, Aquamortierella, Chaetoceros, Charophyceae, Chlorodendrophyceae, Chlorella, Chlorokybophyceae, Chlorophyceae, Chlamydomonas, Coleochaetophyceae, Cyclotella, Cymbella, Dissophora, Embryophytes, Endogaceae, Fragilaria, Gamsiella, Hantzschia, Klebsormidiophyceae, Lobosporangium, Mamiellophyceae, Mesostigmatophyceae, Modicella, Mortierella, Mucor, Navicula, Nephroselmidophyceae, Nitzschia, Palmophyllales, Prasinococcales, Prasinophytes, Pedinophyceae, Phaeodactylum, Pyramimonadales, Pycnoccaceae, Pythium, Phytophthora, Phytopythium, Rhizopus, Thalassiosira, Trebouxiophyceae, Ulvophyceae, Zygnematophyceae, or a combination thereof.

In some cases, the algae is a photosynthetic algae. Examples illustrated in the experimental work shown herein include strains of Chlamydomonas, Chlorella, and Nannochloropsis. In some cases the algae type employed can be a strain of Nannochloropsis oceanica, for example Nannochloropsis oceanica CCMP1779.

A variety of fungi can be employed in the formation of consortia with algae. In some cases, the fungus can be a basidiomycete, ascomycete, or zygomycete. For example, one or more fungi can be a member of a genus such as: Aspergillus, Blakeslea, Botrytis, Candida, Cercospora, Cryptococcus, Cunninghamella, Fusarium (Gibberella), Kluyveromyces, Lipomyces, Morchella, Mortierella, Mucor, Neurospora, Penicillium, Phycomyces, Pichia (Hansenula), Puccinia, Pythium, Rhodosporidium, Rhodotorula, Saccharomyces, Sclerotium, Trichoderma, Trichosporon, Xanthophyllomyces (Phqffia), or Yarrowia. For example, the fungus can be a species such as: Aspergillus terreus, Aspergillus nidulans, Aspergillus niger, Atractiella PMI152, Blakeslea trispora, Botrytis cinerea, Candida japonica, Candida pulcherrima, Candida revkaufi, Candida tropicalis, Candida utilis, Cercospora nicotianae, Clavulina PMI390, Cryptococcus curvatus, Cunninghamella echinulata, Cunninghamella elegans, Flagelloscypha PMI526, Fusarium fujikuroi (Gibberella zeae), Grifola frondosa GMNB41, Kluyveromyces lactis, Lecythophora PMI1546, Leptodontidium PMI413, Lachnum PMI1789, Lipomyces starkeyi, Lipomyces lipoferus, Mortierella alpina, Mortierella elongata AG77, Mortierella gamsii GBAus22, Mortierella ramanniana, Mortierella isabellina, Mortierella vinacea, Mucor circinelloides, Neurospora crassa, Phycomyces blakesleanus, Pichia pastoris, Puccinia distincta, Pythium irregulare, Rhodosporidium toruloides, Rhodotorula glutinis, Rhodotorula graminis, Rhodotorula mucilaginosa, Rhodotorula pinicola, Rhodotorula gracilis, Saccharomyces cerevisiae, Sclerotium rolfsii, Trichoderma reesei, Trichosporon cutaneum, Trichosporon pullans, Umbelopsis PMI120, Xanthophyllomyces dendrorhous (Phqffia rhodozyma), Yarrowia lipolytica, or a combination thereof. In some cases, the fungus is not Geosiphon pyriformis.

In some cases, the fungus employed is a multi-celled fungi. For example, the fungus employed can have tissues and/or structures such as hyphae. Many fungi is made up of fine, branching, usually colorless threads called hyphae. Each fungus can have vast numbers of these hyphae, all intertwining to make up a tangled web called the mycelium. The mycelium is generally too fine to be seen by the naked eye, except where the hyphae are very closely packed together.

As illustrated herein, algae can reside and grow within fungal hyphae. The algae can also undergo photosynthesis within the fungi hyphae. In some cases the location of the algae is not within a fungal “bladder” and does not form a multinucleate bladder within the fungi, or a multinucleate bladder within fungal hyphae.

However, in some cases the fungus need not be a multi-celled fungus. For example, the fungus can be a one-celled organism such as a yeast.

In some cases, the fungus can be one or more of Mortierella elongata, Mortierella elongata AG77, Mortierella gamsii, Mortierella gamsii GBAus22, Umbelopsis sp., Umbelopsis PMI120, Lecythophora sp., Lecythophora PMI546, Leptodontidium sp., Leptodontidium PMI413, Lachnum sp., Lachnum PMI789, Morchella sp., Saccharomyces cerevisiae, Atractiella sp., Atractiella PMI152, Clavulina, Clavulina PMI390, Grifola frondosa, Grifola frondosa GMNB41, Flagelloscypha sp., Flagelloscypha PMI526, and combinations thereof.

### Culture Media

Media for forming fungal/algal consortia can be a simple medium, especially when photosynthetic algae are employed because the algae can supply the fungi as well as the algae cells with carbon-based nutrients. Complex carbon nutrients may therefore not be needed, especially when the fungal/algal consortia are formed and the consortia are exposed to light. However, when initially preparing a consortium between one or more fungal species and one or more algae species, the fungi and algae can be cultured in a culture medium that contains some carbohydrate, such as some sugar. The sugar can be any convenient sugar or a combination of sugars. Examples include dextrose, sucrose, glucose, fructose or a combination thereof. The amount of sugar can be included in amounts of about 1 g/liter to about 20 g/liter, or of about 3 g/liter to about 18 g/liter, or of about 5 g/liter to about 15 g/liter.

Fungi can be grown in PDB media (12 g/L potato dextrose broth, 5 g/L yeast extract, pH 5.3). In some cases the fungi and algae can initially be cultured together to form fungal/algae consortia in the presence of a simple medium that can contain small amounts of PDB media. For example, to form fungal/algae consortia a simple medium such as f/2 medium can be used that is supplemented with small amounts of PDB media.

f/2 Medium

| | | |
|---|---|---|
| NaNO₃ (75.0 g/L dH₂O) | 1.0 | mL |
| Na₂SiO₃•9H₂O (30.0 g/L dH₂O) | 1.0 | mL |
| f/2 Trace Metal Solution | 1.0 | mL |
| f/2 Vitamin Solution | 0.5 | mL |
| Filtered seawater to | 1.0 | L |

Further information on the f/2 medium is available at a website describing the composition of f/2 media (algaeresearchsupply.com/pages/f-2-media).

In some cases, the fungal/algae consortia can be grown and maintained in a media that does not supply a nitrogen source (e.g., without nitrate or ammonium salts, or without other nitrogen-containing salts). For example, the fungus that is part of the fungal/algae consortia can supply a nitrogen source to the algae as well as providing for its own nitrogen needs.

Algae cells and fungal/algae consortia can, for example, be grown or maintained in minimal media such as f/2 media, or even in water (e.g., sea water) with little or no added nutrients, especially when the algae cells and fungal/algae consortia are exposed to light. For example, algae and fungal/algae consortia can be grown or maintained in continuous light (for example, at about 20 μmol photons/m²/s to about 120 μmol photons/m²/s, or at about 40 μmol photons/m²/s to about 100 μmol photons/m²/s, or at about 80 μmol photons/m²/s).

Algae, fungi, and consortia of algae and fungi can be grown or maintained at a convenient moderate temperature. For example, algae, fungi, and consortia of algae and fungi can be grown or maintained at about 15° C. to 37° C., or about 18° C. to 32° C., or at about 20° C. to 30° C., or at about room temperature.

Growing rather than non-growing cells and/or tissues can be used to generate consortia of algae and fungi. For example, log-phase cultures of algae can be used. Fungal tissues employed can include fungal mycelia and/or fungal mycelium. Fungal tissues can be chopped or cut up. For example, fungal tissues can be briefly blended or chopped into small pieces (0.1 to 4 cm, or 0.3 to 3 cm, or 0.5 to 2 cm) before combining the fungal tissues with algae.

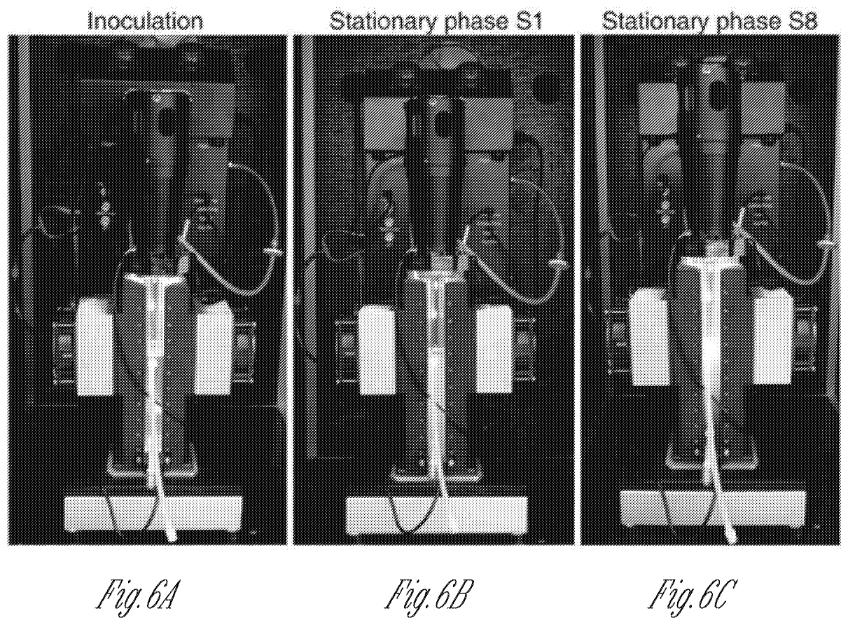

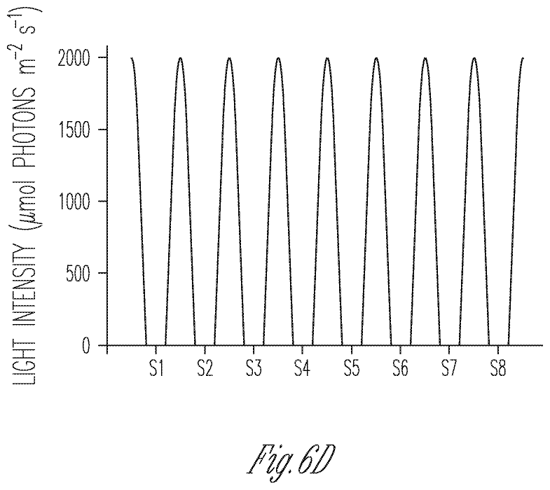

As described herein, culturing consortia in media with limited nitrogen can induce production of increased triacylglycerol (TAG). A limited nitrogen supply culturing method was developed as described herein for large-volume cultures to induce TAG accumulation largely without compromising growth and biomass yields. To mimic natural cultivation conditions for N. oceanica, such as an open-pond system, environmental photobioreactors (ePBRs) were used to grow the alga under varying light (0 to 2,000 μmol photons m⁻² s⁻¹) under long-day (14/10 h light/dark) cycles, and 5% CO₂ was sparged at 0.37 L min⁻¹ for 2 minutes per hour at 23° C. (similar to FIG. 6). Illumination in the ePBR was provided by a high power white LED light on top of a conical culture vessel (total height of 27 cm) containing 330 mL of algal culture (20 cm in depth), which was designed to simulate pond depths from 5 to 25 cm (Lucker et al. Algal research 2014, 6:242-249 (2014)). Several nitrogen sources were tested in f/2 medium for the incubation of N. oceanica including set amounts of ammonium, nitrate, or urea.

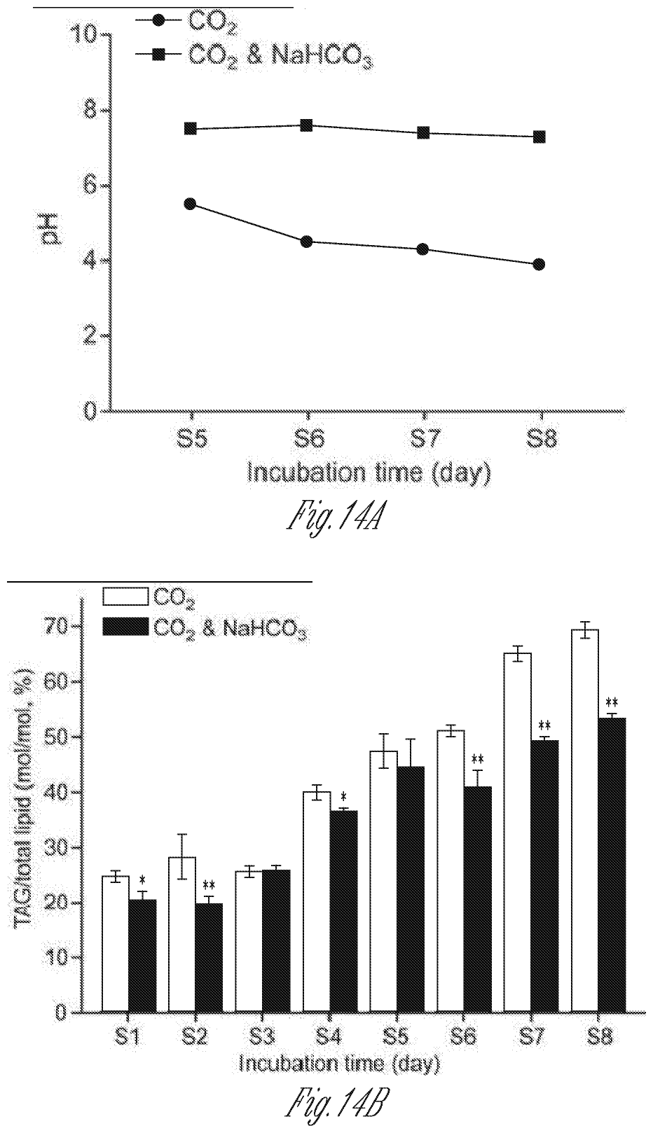

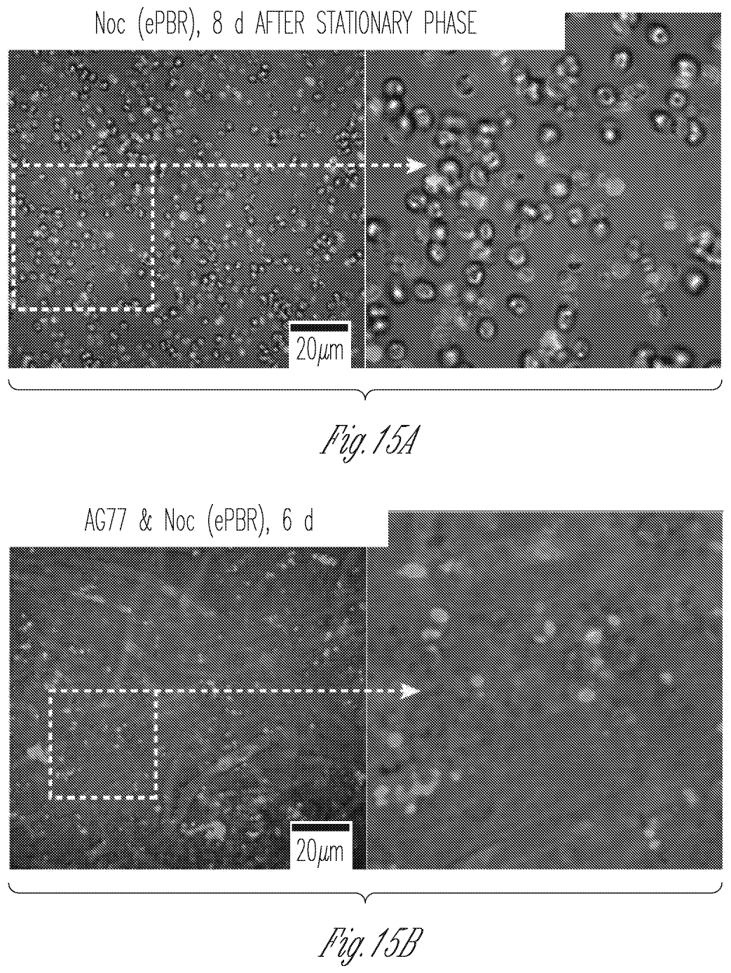

Compared to nitrate and urea, N. oceanica grew faster in the f/2-NH₄Cl medium (FIG. 12A). The dry weight (DW) of N. oceanica cells per liter was also higher in the f/2-NH₄Cl culture after 7-day incubation in the ePBR (FIG. 12B). Hence, use of ammonium salts rather than nitrates or urea can improve TAG production by N. oceanica and consortia containing N. oceanica.

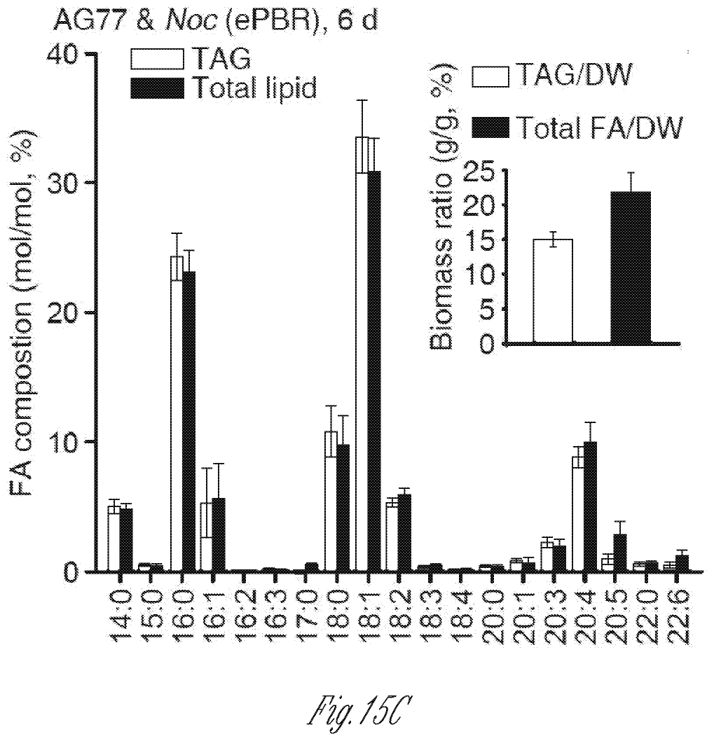

Lipid analysis by TLC (FIG. 13A) and GC-FID (FIG. 13B) demonstrated that TAGs had accumulated during days 2 to 8 after the culture reached stationary phase (incubation time S2 to S8), which is correlated with chlorophyll degradation, while cell density and dry weight remained at similar levels during this period (FIG. 12C-12D). Previously, to prevent carbon limitation, NaHCO₃ was added N. oceanica cultures in shaker flasks (Vieler et al., Plant Physiology 158(4):1562-1569 (2012)). Addition of NaHCO₃ prevented acidification in cultures, which were sparged with 5% CO₂(FIG. 14A). However, N. oceanica cells accumulated more TAG upon acidification in the culture medium without NaHCO₃ supply, especially from S6 to S8, compared to the NaHCO₃ culture (FIG. 12C-12D).

### Generating Fungal/Algal Consortia

To form consortia, the algal cells and fungal cells (or fungal tissues) can be mixed together in a selected culture media and incubated together for one or more days, one or more weeks, one or months, one or more years, or indefinitely. The culture media or growth conditions can be changed or modulated as desired to form and maintain the fungal/algal consortia.

To form the fungal/algal consortia, the fungal tissues/cells and the algal cells can be incubated in sufficient cell/tissue density so that the fungal tissues/cells and the algal cells come into contact. For example, algae can be added to fungal cells/tissues at a density of about 1×10⁴ algae cells/mL to 1×10⁹ algae cells/mL, or at a density of about 1×10⁵ algae cells/mL to 1×10⁸ algae cells/mL, or at a density of about 1×10⁶ algae cells/mL to 1×10⁸ algae, or at a density of about 1-3×10⁷ cells/mL. The ratio of fungal tissues to algae cells can vary. In some cases, it may be useful to use more fungal tissue (by mass) than algal cell mass. For example, the ratio can vary from about 10:1 by mass fungal tissue to algal cells, to about 1:1 by mass fungal tissue to algal cells. In some cases, the ratio can vary from about 5:1 by mass fungal tissue to algal cells, to about 1:1 by mass fungal tissue to algal cells. For example, the ratio can be about 3:1 by mass fungal tissue to algal cells.

In some cases it may be useful to use more algae cell mass than fungal tissue mass. For example, the ratio can vary from about 10:1 by mass algal cells to fungal tissue mass, to about 1:1 by mass algal cells to fungal tissue mass. In some cases, the ratio can vary from about 5:1 by mass algal cells to fungal tissue mass to about 1:1 by mass algal cells to fungal tissue mass.

As indicated in the foregoing section, when initially preparing a consortium between one or more fungal species and one or more algae species, the fungi and algae can be cultured in a culture medium that contains some carbohydrate, such as some sugar. The sugar can be any convenient sugar or a combination of sugars. Examples include dextrose, sucrose, glucose, fructose or a combination thereof. The amount of sugar can be included in amounts of about 1 g/liter to about 20 g/liter, or of about 3 g/liter to about 18 g/liter, or of about 5 g/liter to about 15 g/liter.

The consortium between one or more fungal species and one or more algae species can be formed in a liquid media, in a semi-solid media, or on a solid media.

Consortia of algal cells within fungal tissues can include fungal hyphae with different numbers of algae cells within them. For example, fungal tissues can include 1 to 2000 algae cells per fungal hyphae, or 2 to 1700 algae cells per fungal hyphae, or 5 to 1500 algae cells per fungal hyphae, or 10 to 1000 algae cells per fungal hyphae, or 15 to 500 algae cells per fungal hyphae, or 5 to 100 algae cells per fungal hyphae. Fungal hyphae can typically have any number of algae cells within them, up to about 5000 algae cells.

### Consortia Benefits

The fungal/algae consortia are easier to harvest than algae cells.

The fungal/algae consortia described herein can be more robust than separate cultures of algae or separate fungi. For example, the algae can provide it fungal partner with useful carbon-based nutrients while the fungus can provide its algae partner with useful nitrogen-based nutrients, or vice versa. Hence, the fungal/algae consortia described herein can be more tolerant of environmental stresses such as nutrient-poor conditions.

In addition, a fungal partner can protect its algae cells from environmental stresses such as salt imbalances (too much salt or too little) that would otherwise adversely affect the growth or health of the algae.

Algae are useful for production of useful compounds and materials such as oils, biofuels, nutrients (sugars, vitamins, proteins, etc.), and biomass. The protection and support provided by a fungal partner can help foster the growth and production of algae. Similarly, the algae can support and foster the growth of its fungal partner. Hence, the fungal/algae consortia described herein can be used to produce useful products under low cost conditions that do not require expensive monitoring and maintenance.

For example, fungal/algae consortia described herein can be used to produce various types of oils or biofuels. In certain aspects, the fungal-algae consortium can have lipid content greater than about 20%, and preferably greater than about 30% by weight of the consortium weight. Currently known algae species may contain a practical maximum lipid content of about 40% by weight, although levels as high as 60% have been reported. Such species can be algae partners for formation of fungal/algae consortia. In some embodiments, the lipid-producing consortium can comprise lipid content greater than 40%, 50%, 60%, 70%, 80%, or 90% by weight of the consortium. In a specific embodiment, the subject methods involve selection of consortium which produce high levels of simple and/or complex lipids.

For example, the content of lipids provided by cultures and methods described herein can be at least 20%, 30%, 40%, 50%, 60%, 70%, 80%, or 90° % by weight of the consortium.

Transgenic Algae and/or Fungi

A method is described herein that includes manufacturing a fungus or algae cell by introducing into the cell at least one exogenous nucleic acid encoding a lipid synthetic enzyme. The lipid synthetic enzyme can be a fatty acid, TAG or other lipid synthetic enzyme. Also described herein are modified fungi, algae, and fungal/algae consortia that have at least one exogenous nucleic acid encoding a lipid synthetic enzyme. The modified fungi, algae, and fungal/algae consortia can express at least one exogenous lipid synthetic enzyme. Such modified fungi, algae, and fungal/algae consortia can produce increased amounts of lipid compared to unmodified fungi, algae, and fungal/algae of the same species.

In order to engineer fungi and/or algae to have increased oil content, one of skill in the art can introduce exogenous nucleic acids (expression cassettes or expression vectors) that increase the expression and/or translation of lipid synthetic enzyme to promote the production of oils. The lipid synthetic enzymes can include one or more acetyl-CoA carboxylase, malonyl-CoA decarboxylase, acyl carrier protein, fatty acid synthase, malonyl-CoA:ACP malonyltransferase, 3-oxoacyl-ACP synthase, KASI/II, 3-hydroxydecanoyl-ACP dehydratase, 3-hydroxydecanoyl-ACP dehydratase, 3-ketoacyl-ACP reductase, acyl-CoA elongase, fatty acid desaturase, acyl-CoA thioesterase, acyl-CoA synthetase, aldehyde dehydrogenase, alcohol dehydrogenase, glycerol kinase, glycerol-3-phosphate dehydrogenase, glycero-3-phosphate acyltransferase, 1-sn-acyl-glycero-3-phosphate acyltransferase, phosphatidic acid phosphatase, lipin-like phosphatidate phosphatase, diacylglycerol kinase, diacylglycerol acyltransferase, phospholipid diacylglycerol acyltransferase, or any combination thereof. Examples of such enzymes and enzyme sequences are provided in Examples 9 and 10.

One of skill in the art can generate genetically-modified algae and/or fungi that contain one or more nucleic acids encoding lipid synthetic enzyme(s). Such genetic modification can be accomplished by a variety of procedures. For example, one of skill in the art can prepare an expression cassette or expression vector that can express one or more lipid synthetic enzyme. Algae and/or fungi cells can be transformed by the expression cassette or expression vector, the cells that were successfully transformed with the lipid synthetic enzyme nucleic can be expanded. Selected algae and fungi can be combined to provide the consortia described herein. Some procedures for making such genetically modified algae and/or fungi are described below.

Promoters: The lipid synthetic enzyme nucleic acids can be operably linked to a promoter, which provides for expression of RNA encoding the lipid synthetic enzyme(s). The promoter is typically a promoter functional in algae and/or fungi, and can be a promoter functional growth and development of a fungal/algae consortium. The promoter can be a heterologous promoter. As used herein, “heterologous” when used in reference to a gene or nucleic acid refers to a gene or nucleic acid that has been manipulated in some way. For example, a heterologous promoter is a promoter that contains sequences that are not naturally linked to an associated coding region.

A lipid synthetic enzyme nucleic acid is operably linked to the promoter when it is located downstream from the promoter, to thereby form an expression cassette. One lipid synthetic enzyme encoding nucleic acid can be separately regulated from another lipid synthetic enzyme encoding nucleic acid by use of separate promoters and/or separate expression cassettes.

Promoter regions are typically found in the flanking DNA upstream from the coding sequence in both prokaryotic and eukaryotic cells. A promoter sequence provides for regulation of transcription of the downstream gene sequence and typically includes from about 50 to about 2,000 nucleotide base pairs. Promoter sequences also contain regulatory sequences such as enhancer sequences that can influence the level of gene expression. Some isolated promoter sequences can provide for gene expression of heterologous DNAs, that is a DNA different from the native or homologous DNA.

Promoter sequences are also known to be strong or weak, or inducible. A strong promoter provides for a high level of gene expression, whereas a weak promoter provides a very low level of gene expression. An inducible promoter is a promoter that provides for the turning on and off of gene expression in response to an exogenously added agent, or to an environmental or developmental stimulus. For example, a bacterial promoter such as the P_{tac} promoter can be induced to vary levels of gene expression depending on the level of isothiopropylgalactoside added to the transformed cells. Promoters can also provide for tissue specific or developmental regulation. An isolated promoter sequence that is a strong promoter for heterologous DNAs is advantageous because it provides for a sufficient level of gene expression for easy detection and selection of transformed cells and provides for a high level of gene expression when desired. In some embodiments, the promoter is an inducible promoter and/or a tissue-specific promoter.

Examples of promoters that can be used include, but are not limited to, the CaMV 35S promoter (Odell et al., Nature. 313:810-812 (1985)), or others such as CaMV 19S (Lawton et al., Plant Molecular Biology. 9:315-324 (1987)), nos (Ebert et al., Proc. Natl. Acad. Sci. USA. 84:5745-5749 (1987)), Adh1 (Walker et al., Proc. Natl. Acad. Sci. USA. 84:6624-6628 (1987)), sucrose synthase (Yang et al., Proc. Natl. Acad. Sci. USA. 87:4144-4148 (1990)), α-tubulin, ubiquitin, actin (Wang et al., Mol. Cell. Biol. 12:3399 (1992)), cab (Sullivan et al., Mol. Gen. Genet. 215:431 (1989)), PEPCase (Hudspeth et al., Plant Molecular Biology. 12:579-589 (1989)), the CCR (cinnamoyl CoA:NADP oxidoreductase, EC 1.2.1.44) promoter sequence isolated from Lollium perenne, (or a perennial ryegrass) and/or those associated with the R gene complex (Chandler et al., The Plant Cell. 1:1175-1183 (1989)). Further suitable promoters include the poplar xylem-specific secondary cell wall specific cellulose synthase 8 promoter, cauliflower mosaic virus promoter, the Z10 promoter from a gene encoding a 10 kD zein protein, a Z27 promoter from a gene encoding a 27 kD zein protein, inducible promoters, such as the light inducible promoter derived from the pea rbcS gene (Coruzzi et al., EMBO J. 3:1671 (1971)) and the actin promoter from rice (McElroy et al., The Plant Cell. 2:163-171 (1990)). Seed specific promoters, such as the phaseolin promoter from beans, may also be used (Sengupta-Gopalan, Proc. Natl. Acad. Sci. USA. 83:3320-3324 (1985). Other promoters useful in the practice of the invention are available to those of skill in the art.

Alternatively, novel promoter sequences may be employed in the practice of the present invention. cDNA clones from a particular species are isolated and those clones which are expressed well in algae and/or fungi are identified, for example, using Northern blotting. Preferably, the gene isolated is not present in a high copy number, but is relatively abundant in the cells. The promoter and control elements of corresponding genomic clones can then be localized using techniques available to those of skill in the art.

For example, the promoter can be an inducible promoter. Such inducible promoters can be activated by agents such as chemicals, hormones, sugars, metabolites, or by the age or developmental stage of the algae or fungus. For example, the promoter can be an ethanol-inducible promoter, a sugar-inducible promoter, a senescence-induced promoter or any promoter activated in algae or fungi. One example of a sugar-inducible promoter is a patatin B33 promoter.

A nucleic acid encoding a lipid synthetic enzyme can be combined with the promoter by a variety methods to yield an expression cassette, for example, as described in Sambrook et al. (MOLECULAR CLONING: A LABORATORY MANUAL. Second Edition (Cold Spring Harbor, N.Y.: Cold Spring Harbor Press (1989); MOLECULAR CLONING: A LABORATORY MANUAL. Third Edition (Cold Spring Harbor, N.Y.: Cold Spring Harbor Press (2000)). Briefly, a plasmid containing a promoter such as the 35S CaMV promoter can be constructed as described in Jefferson (Plant Molecular Biology Reporter 5:387405 (1987)) or obtained from Clontech Lab in Palo Alto, Calif. (e.g., pBI121 or pBI221). Typically, these plasmids are constructed to have multiple cloning sites having specificity for different restriction enzymes downstream from the promoter. The nucleic acids encoding lipid synthetic enzymes can be subcloned downstream from the promoter using restriction enzymes and positioned to ensure that the DNA is inserted in proper orientation with respect to the promoter so that the DNA can be expressed as sense RNA. Once the lipid synthetic enzyme encoding nucleic acid is operably linked to a promoter, the expression cassette so formed can be subcloned into a plasmid or other vector (e.g., an expression vector). Using restriction endonucleases, the lipid synthetic enzyme nucleic acid is subcloned downstream of the promoter in a 5′ to 3′ sense orientation.

In some embodiments, a cDNA or other nucleic acid encoding a selected lipid synthetic enzyme is obtained or isolated from a selected species or is prepared by available methods or as described herein. For example, the nucleic acid encoding a lipid synthetic enzyme can be any nucleic acid that encodes any of SEQ ID NO:7-112.

The lipid synthesizing enzymes encoded by the nucleic acids can have sequences that have less than 100% sequence identity to any of SEQ ID NO:7-112. Typically the lipid synthesizing enzymes have about at least 40% sequence identity, or at least 50% sequence identity, or at least 60% sequence identity, or at least 70% sequence identity, or at least 80% sequence identity, or at least 90% sequence identity, or at least 95% sequence identity, or at least 96% sequence identity, or at least 97% sequence identity, or at least 98% sequence identity, or at least 99% sequence identity, or 60-99% sequence identity, or 70-99% sequence identity, or 80-99% sequence identity, or 90-95% sequence identity, or 90-99% sequence identity, or 95-97% sequence identity, or 97-99% sequence identity, or 100% sequence identity with any of SEQ ID NO:7-112.

In some embodiments, a selectively hybridizing sequence can be employed where the selectively hybridizing sequence encodes a lipid synthesizing enzyme that has at least 40% sequence identity, or at least 50% sequence identity, or at least 60% sequence identity, or at least 70% sequence identity, or at least 80% sequence identity, or at least 90% sequence identity, or at least 95% sequence identity, or at least 96% sequence identity, or at least 97% sequence identity, or at least 98% sequence identity, or at least 99% sequence identity, or 60-99% sequence identity, or 70-99% sequence identity, or 80-99% sequence identity, or 90-95% sequence identity, or 90-99% sequence identity, or 95-97% sequence identity, or 97-99% sequence identity to SEQ ID NO:7-112.

The nucleic acids employed in the expression vectors, transgenes, algae, fungi, and methods described herein can also encode a lipid synthesizing enzyme that has less than 100%, or less than 99.5%, or less than 99% sequence identity (or complementarity) with any of SEQ ID NO:7-112. In other words, the lipid synthesizing enzymes and the nucleic acids encoding them that are employed in the expression vectors, transgenes, algae, fungi, consortia, and methods described herein can also not include a wild type sequence.

In some embodiments, the nucleic acids used in the methods, algae, fungi, and consortia provided herein can encode lipid synthesizing enzymes that are less than full length. For example, the enzymes can include those that have at least one amino acid difference, or at least two amino acid differences, or at least three amino acid differences, or at least four amino acid differences, or at least five amino acid differences, or at least six amino acid differences, or at least seven amino acid differences, or at least eight amino acid differences, or at least nine amino acid differences, or at least ten amino acid differences in any of the SEQ ID NO:7-112 sequences. The identical amino acids can be distributed throughout the polypeptide, and need not be contiguous.

A nucleic acid encoding a lipid synthesizing enzyme can have nucleotide sequence variation. For example, the nucleic acid sequences encoding a lipid synthesizing enzyme can be optimized for expression in a particular algal or fungal species by altering selected codons to encode the same amino acid but use nucleotide codons that are more easily ‘read’ by the transcription/translation machinery of a selected species.

Targeting Sequences: Additionally, expression cassettes can be constructed and employed to target the lipid synthetic enzyme nucleic acids to an intracellular compartment within the algae or fungal cells or to direct an encoded protein to particular intracellular environment. This can generally be achieved by joining a DNA sequence encoding a transit or signal peptide sequence to the coding sequence of the nucleic acid that encodes the lipid synthetic enzyme. The resultant transit, or signal, peptide will transport the protein to a particular intracellular, or extracellular destination, and can then be posttranslational removed. Transit peptides act by facilitating the transport of proteins through intracellular membranes, e.g., vacuole, vesicle, plastid and mitochondrial membranes, whereas signal peptides direct proteins through the extracellular membrane. By facilitating transport of the protein into compartments inside or outside the cell, these sequences can increase the accumulation of a particular gene product in a particular location. For example, see U.S. Pat. No. 5,258,300.

3′ Sequences: When the expression cassette is to be introduced into an algal or fungal cell, the expression cassette can also optionally include 3′ nontranslated regulatory DNA sequences that act as a signal to terminate transcription and allow for the polyadenylation of the resultant mRNA. The 3′ nontranslated regulatory DNA sequence preferably includes from about 300 to 1,000 nucleotide base pairs and contains plant transcriptional and translational termination sequences. For example, 3′ elements that can be used include those derived from the nopaline synthase gene of Agrobacterium tumefaciens (Bevan et al., Nucleic Acid Research. 11:369-385 (1983)), or the terminator sequences for the T7 transcript from the octopine synthase gene of Agrobacterium tumefaciens, and/or the 3′ end of the protease inhibitor I or II genes from potato or tomato. Other 3′ elements known to those of skill in the art can also be employed. These 3′ nontranslated regulatory sequences can be obtained as described in An (Methods in Enzymology. 153:292 (1987)). Many such 3′ nontranslated regulatory sequences are already present in plasmids available from commercial sources such as Clontech, Palo Alto, Calif. The 3′ nontranslated regulatory sequences can be operably linked to the 3′ terminus of the nucleic acids encoding the lipid synthetic enzyme by standard methods.

Selectable and Screenable Marker Sequences: In order to improve identification of transformants, a selectable or screenable marker gene can be employed with the nucleic acids that encode the lipid synthetic enzyme(s). “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can ‘select’ for by chemical means, i.e., through the use of a selective agent (e.g., a herbicide, antibiotic, or the like), or whether it is simply a trait that one can identify through observation or testing, i.e., by ‘screening’ (e.g., the R-locus trait). Of course, many examples of suitable marker genes are available and can be employed in the practice of the invention.

Included within the terms selectable or screenable marker genes are also genes which encode a “secretable marker” whose secretion can be detected as a means of identifying or selecting for transformed cells. Examples include markers which encode a secretable antigen that can be identified by antibody interaction, or secretable enzymes that can be detected by their catalytic activity. Secretable proteins fall into a number of classes, including small, diffusible proteins detectable, e.g., by ELISA; and proteins that are inserted or trapped in the cell wall (e.g., proteins that include a leader sequence such as that found in the expression unit of extensin or tobacco PR-S).

With regard to selectable secretable markers, the use of a gene that encodes a polypeptide that becomes sequestered in the cell wall, where the polypeptide includes a unique epitope may be advantageous. Such a secreted antigen marker can employ an epitope sequence that would provide low background in the interior of the cell, a promoter-leader sequence that imparts efficient expression and targeting across the plasma membrane, and can produce protein that is bound in the cell wall and yet is accessible to antibodies. A normally secreted wall protein modified to include a unique epitope would satisfy such requirements.

Examples of proteins suitable for modification in this manner include extensin or hydroxyproline rich glycoprotein (HPRG). For example, the maize HPRG (Stiefel et al., The Plant Cell. 2:785-793 (1990)) is well characterized in terms of molecular biology, expression, and protein structure and therefore can readily be employed. However, any one of a variety of extensins and/or glycine-rich wall proteins (Keller et al., EMBO J. 8:1309-1314 (1989)) could be modified by the addition of an antigenic site to create a screenable marker.

Possible selectable markers for use include, a neo gene (Potrykus et al., Mol. Gen. Genet. 199:183-188 (1985)) which codes for kanamycin resistance and can be selected for using kanamycin, G418, and the like; a bar gene which codes for bialaphos resistance; a gene which encodes an altered EPSP synthase protein (Hinchee et al., Bio/Technology. 6:915-922 (1988)) thus conferring glyphosate resistance; a nitrilase gene such as bxn from Klebsiella ozaenae which confers resistance to bromoxynil (Stalker et al., Science. 242:419-423 (1988)); a mutant acetolactate synthase gene (ALS) which confers resistance to imidazolinone, sulfonylurea or other ALS-inhibiting chemicals (European Patent Application 154,204 (1985)); a methotrexate-resistant DHFR gene (Thillet et al., J. Biol. Chem. 263:12500-12508 (1988)); a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide, CTP (European Patent Application 0 218 571 (1987)).

An illustrative embodiment of a selectable marker gene capable of being used in systems to select transformants is the gene that encode the enzyme phosphinothricin acetyltransferase, such as the bar gene from Streptomyces hygroscopicus or the pat gene from Streptomyces viridochromogenes (U.S. Pat. No. 5,550,318). The enzyme phosphinothricin acetyl transferase (PAT) inactivates the active ingredient in the herbicide bialaphos, phosphinothricin (PPT). PPT inhibits glutamine synthetase, (Murakami et al., Mol. Gen. Genet. 205:42-50 (1986); Twell et al., Plant Physiol. 91:1270-1274 (1989)) causing rapid accumulation of ammonia and cell death.

Screenable markers that may be employed include, but are not limited to, a β-glucuronidase or uidA gene (GUS) that encodes an enzyme for which various chromogenic substrates are known; an R-locus gene, which encodes a product that regulates the production of anthocyanin pigments (red color) in cells (Dellaporta et al., In: Chromosome Structure and Function: Impact of New Concepts, 18^{th} Stadler Genetics Symposium, J. P. Gustafson and R. Appels, eds. (New York: Plenum Press) pp. 263-282 (1988)); a β-lactamase gene (Sutcliffe, Proc. Natl. Acad. Sci. USA. 75:3737-3741 (1978)), which encodes an enzyme for which various chromogenic substrates are known (e.g., PADAC, a chromogenic cephalosporin); a xylE gene (Zukowsky et al., Proc. Natl. Acad. Sci. USA. 80:1101 (1983)) which encodes a catechol dioxygenase that can convert chromogenic catechols; an α-amylase gene (Ikuta et al., Bio/technology 8:241-242 (1990)); a tyrosinase gene (Katz et al., J. Gen. Microbiol. 129:2703-2714 (1983)) which encodes an enzyme capable of oxidizing tyrosine to DOPA and dopaquinone which in turn condenses to form the easily detectable compound melanin; a β-galactosidase gene, which encodes an enzyme for which there are chromogenic substrates; a luciferase (lux) gene (Ow et al., Science. 234:856-859.1986), which allows for bioluminescence detection; or an aequorin gene (Prasher et al., Biochem. Biophys. Res. Comm. 126:1259-1268 (1985)), which may be employed in calcium-sensitive bioluminescence detection, or a green or yellow fluorescent protein gene (Niedz et al., Plant Cell Reports. 14:403 (1995).

A further screenable marker contemplated for use is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for population screening for bioluminescence, such as on tissue culture plates, or even for whole plant screening.

Numerous other possible selectable and/or screenable marker genes will be apparent to those of skill in the art in addition to the one set forth herein below. Therefore, it will be understood that the discussion provided herein is exemplary rather than exhaustive. In light of the techniques disclosed herein and the general recombinant techniques that are known in the art, the present invention readily allows the introduction of any gene, including marker genes, into a recipient cell to generate a transformed algae or fungal cell.

Other Optional Sequences: An expression cassette of the invention can also further comprise plasmid DNA. Plasmid vectors include additional DNA sequences that provide for easy selection, amplification, and transformation of the expression cassette in prokaryotic and eukaryotic cells, e.g., pUC-derived vectors such as pUC8, pUC9, pUC18, pUC19, pUC23, pUC119, and pUC120, pSK-derived vectors, pGEM-derived vectors, pSP-derived vectors, or pBS-derived vectors. The additional DNA sequences include origins of replication to provide for autonomous replication of the vector, additional selectable marker genes, such as antibiotic or herbicide resistance, unique multiple cloning sites providing for multiple sites to insert DNA sequences, and/or sequences that enhance transformation of prokaryotic and eukaryotic cells.

Another vector that is useful for expression in both plant and prokaryotic cells is the binary Ti plasmid (as disclosed in Schilperoort et al., U.S. Pat. No. 4,940,838) as exemplified by vector pGA582. This binary Ti plasmid vector has been previously characterized by An (Methods in Enzymology. 153:292 (1987)). This binary Ti vector can be replicated in prokaryotic bacteria such as E. coli and Agrobacterium. The Agrobacterium plasmid vectors can be used to transfer the expression cassette to algae or fungal cells. The binary Ti vectors preferably include the nopaline T DNA right and left borders to provide for efficient plant cell transformation, a selectable marker gene, unique multiple cloning sites in the T border regions, the colE1 replication of origin and a wide host range replicon. The binary Ti vectors carrying an expression cassette of the invention can be used to transform both prokaryotic and eukaryotic cells.

In Vitro Screening of Expression Cassettes: Once the expression cassette is constructed and subcloned into a suitable plasmid, it can be screened for the ability to express the encoded lipid synthetic enzyme. For example, for expression of one or more lipid synthetic enzymes, the encoding nucleic acid can be subcloned into a selected expression cassette or vector (e.g., a SP6/T7 containing plasmid, which is supplied by ProMega Corp.). The expression of the lipid synthetic enzyme RNA can be detected by Northern analysis, PCR analysis, or other hybridization methods. The lipid synthetic enzyme protein can be detected by antibody staining methods. As a control, a nonsense nucleic acid is expressed from an expression cassette that is introduced into algae or fungal cells. The phenotypes of the control and test cells (e.g., lipid content) can also be assessed.

DNA Delivery of the DNA Molecules into Host Cells: The present invention generally includes steps directed to introducing at least one nucleic acid encoding a lipid synthetic enzyme into a recipient cell to create a transformed cell. The frequency of occurrence of cells taking up exogenous (foreign) DNA may be low. Moreover, it is most likely that not all recipient cells receiving DNA segments or sequences will result in a transformed cell wherein the DNA is stably integrated into the algae and/or fungal genome and/or expressed. Some may show only initial and transient gene expression. However, certain cells from virtually any species may be stably transformed, and these cells regenerated into transgenic algae, fungi, or algae/fungal consortia, through the application of the techniques disclosed herein.

Another aspect of the invention is an algae or fungal species, or a fungal/algae consortium with increased oil content, wherein the algae cells, fungal cells, or a fungal/algae consortia has the introduced nucleic acid that encodes the lipid synthetic enzyme(s). The algae or fungal species can, for example, be any species described herein. The cell(s) may be in a suspension cell culture or may be in a consortium.

Transformation of the cells can be conducted by any one of a number of methods known to those of skill in the art. Examples are: Transformation by direct DNA transfer into cells by electroporation (U.S. Pat. Nos. 5,384,253 and 5,472,869, Dekeyser et al., The Plant Cell. 2:591-602 (1990)); direct DNA transfer to plant cells by PEG precipitation (Hayashimoto et al., Plant Physiol. 93:857-863 (1990)); direct DNA transfer by microprojectile bombardment (McCabe et al., Bio/Technology. 6:923-926 (1988); Gordon-Kamm et al., The Plant Cell. 2:603-618 (1990); U.S. Pat. Nos. 5,489,520; 5,538,877; and 5,538,880) and DNA transfer to cells via infection with Agrobacterium. Methods such as microprojectile bombardment or electroporation can be carried out with “naked” DNA where the expression cassette may be simply carried on any E. coli-derived plasmid cloning vector. In the case of viral vectors, it is desirable that the system retain replication functions, but lack functions for disease induction.

The transformation is carried out under conditions acceptable to the algae and/or fungal cells. The cells are exposed to the DNA or RNA carrying the nucleic acid(s) encoding the lipid synthetic enzyme(s) for an effective period of time. This may range from a less than one second pulse of electricity for electroporation to a 2-3 day co-cultivation in the presence of plasmid-bearing cells. Buffers and media used will also vary with the algae/fungal cells and transformation protocol employed.

Electroporation: Where one wishes to introduce DNA by means of electroporation, it is contemplated that the method of Krzyzek et al. (U.S. Pat. No. 5,384,253) may be advantageous. In this method, certain cell wall-degrading enzymes, such as pectin-degrading enzymes, can be employed to render the target recipient cells more susceptible to transformation by electroporation than untreated cells. Alternatively, recipient cells can be made more susceptible to transformation, by mechanical wounding.

To effect transformation by electroporation, one may employ a suspension cell cultures, or friable fungal tissues, or other organized tissues directly. The cell walls of the preselected cells or organs can be partially degraded by exposing them to degrading enzymes (pectinases, pectolyases, polygalacturonases, pectinmethyl esterases, hemicellulose degrading enzymes such as endoxylanases and xyloglucan endoglucanases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

Microprojectile Bombardment: A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

It is contemplated that in some instances DNA precipitation onto metal particles would not be necessary for DNA delivery to a recipient cell using microprojectile bombardment. A low level of transient expression of the nucleic acid encoding the lipid synthetic enzyme(s) may be observed 24-48 hours following DNA delivery. In addition, stable transformants containing the lipid synthetic enzyme nucleic acids can be recovered following bombardment. It is contemplated that particles may contain DNA rather than be coated with DNA. Hence particles may increase the level of DNA delivery but are not, in and of themselves, necessary to introduce DNA into algae or fungal cells.

An advantage of microprojectile bombardment is that the isolation of protoplasts (Christou et al., PNAS. 84:3%2-3966 (1987)), and the formation of partially degraded cells, or the susceptibility to Agrobacterium infection is not required.

For bombardment, cells in suspension can be concentrated on filters or solid culture medium. The cells to be bombarded are positioned at an appropriate distance below the macroprojectile stopping plate. If desired, one or more screens are also positioned between the acceleration device and the cells to be bombarded. Through the use of techniques set forth here-in one may obtain up to 1000 or more foci of cells transiently expressing a marker gene. The number of cells in a focus which express the exogenous gene product 48 hours post-bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA/microprojectile precipitate or those that affect the path and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. Execution of such routine adjustments will be known to those of skill in the art.

Selection: An exemplary embodiment of methods for identifying transformed cells involves exposing the bombarded cultures to a selective agent, such as a metabolic inhibitor, an antibiotic, herbicide or the like. Cells which have been transformed and have stably integrated a marker gene conferring resistance to the selective agent used, will grow and divide in culture. Sensitive cells will not be amenable to further culturing.

For example, to use the bar-bialaphos or the EPSPS-glyphosate selective system, bombarded tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate may be useful. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

The enzyme luciferase, or fluorescent proteins (e.g., green fluorescent protein, GFP) are also useful as screenable markers. In the presence of the substrate luciferin, cells expressing luciferase emit light which can be detected on photographic or X-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

Determination of Stably Transformed Algae or Fungi: To confirm the presence of the nucleic acid encoding the lipid synthesizing enzymes in the algae and/or fungi, a variety of assays may be performed. Such assays include, for example, molecular biological assays available to those of skill in the art, such as Southern and Northern blotting and PCR: biochemical assays, such as detecting the presence of a protein product, e.g., by immunological means (ELISAs and Western blots) or by enzymatic function; and also, by analyzing the phenotype of the algae, fungi, or consortia. In some embodiments, the amount of oil in algae, fungi, or consortia is quantified. Such a quantified oil content can be compared to a control, for example, a control algae, fungi, or consortia of the same species that has not be modified to express the nucleic acid(s) that encode the lipid synthesizing enzymes.

Whereas DNA analysis techniques may be conducted using DNA isolated from any part of a plant, RNA may only be expressed in particular cells or tissue types and so RNA for analysis can be obtained from those tissues. PCR techniques may also be used for detection and quantification of RNA produced from the introduced lipid synthesizing enzyme nucleic acid(s). RT-PCR also be used to reverse transcribe expressed RNA into DNA, using enzymes such as reverse transcriptase, and then this DNA can be amplified through the use of conventional PCR techniques. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and also demonstrate the presence or absence of an RNA species.

Southern blotting, northern blotting and PCR may be used to detect the inhibitory nucleic acid(s) encoding the lipid synthesizing enzymes in question. Expression may also be evaluated by specifically identifying the presence or absence of protein products of the introduced lipid synthesizing enzyme nucleic acids, by assessing the level of enzyme expressed, or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to confirm the identity of the lipid synthesizing enzyme(s) expressed such as evaluation by nucleic acid or amino acid sequencing following purification. Other procedures may be additionally used.

The expression of a nucleic acid or gene product can also be determined by evaluating the phenotypic results of its expression. These assays also may take many forms including but not limited to analyzing changes in the chemical composition, morphology, or physiological properties of the algae, fungus or consortium. For example, the lipid composition of algae, fungus or consortium can be evaluated and/or quantified.

The following non-limiting Examples illustrate how aspects of the invention have been developed and can be made and used.

### Example 1: Materials and Methods

This Example describes some of the materials and methods that were used in the development of the invention.

### Strains and Growth Conditions

Marine alga Nannochloropsis oceanica CCMP1779 was obtained from Provasoli-Guillard National Center for Culture of Marine Phytoplankton and incubated as described by Vieler et al. (PLoS Genet. 8, e1003064 (2012)). In brief, N. oceanica cells were grown in flasks containing f/2 media under continuous light (˜80 μmol/m²/s) at 22° C. with agitation (100 rpm). Log-phase algal culture (1˜3×10⁷ cells/mL) was used for co-culture with fungi. Cell size and density of algal culture were determined using a Z2 Coulter Counter (Beckman). Mortierella elongata AG77 and NVP64 were isolated from soil samples collected at North Carolina, USA (AG77) and Michigan, USA (NVP64). M. elongata AG77 and NVP64 hosting bacterial endosymbiont had been cured of their endobacteria by a series of antibiotic treatments as described by Partida-Martinez et al. (Chembiochem. 8, 41-45 (2007)), and the resultant clean strains were used in this study. Other fungal isolates obtained from healthy surface sterilized Populus roots were obtained from the Plant-Microbial Interfaces (PMI) project (Bonito et al., Fungal Ecol. 22, 35-42 (2016)) (new strains). Fungi were incubated in flasks containing PDB media (12 g/L potato dextrose broth, 5 g/L yeast extract, pH 5.3) at room temperature (RT, ˜22° C.).

For the co-culture of algae and fungi, fungal mycelia were briefly blended into small pieces (0.5 to 2 cm) using a sterilized blender (speed, 30 s). After 24-h recover in PDB medium, fungal tissues were collected by centrifugation (3,000 g for 3 min), washed twice with 172 medium and resuspended in ˜15 mL f/2 medium. A portion of fungal tissues (3-4 mL) were used for the calculation of dry biomass: 1 mL of fungal tissues were transferred with cut-off pipette tip and filtrated through pre-dried and pre-weighed Whatman GF/C filters and dried overnight at 80° C. Similar method was used for the measurement of alga biomass. Fungal tissues about 3 times of alga biomass were added into N. oceanica culture for co-cultivation on a shaker (˜60 rpm) under continuous light (˜80 μmol/m²/s) at RT. After 18-days of co-culture, the shaker was turned off for free settling of algae and fungi overnight. Supernatant was removed with Pasteur pipettes and the same volume of fresh f/2 medium containing 10% PDB was added to the culture. After that, the alga-fungus co-culture was biweekly refreshed with f/2 medium supplemented with 10% PDB.

Nutrient deprivation of the co-culture was performed according to a published protocol for N. oceanica (Vieler et al., PLoS Genet. 8, e1003064 (2012)). Mid-log-phase N. oceanica cells (˜1×10⁷ cells/mL) grown in f/2 media (25 mL) were harvested by centrifugation and washed twice with nutrient-deficient f/2 media [without carbon (—C), nitrogen (—N) or phosphorus (—P)] and resuspended in 25 mL nutrient-deficient f/2 media, respectively. AG77 mycelia grown in PDB medium were washed twice with the nutrient-deficient f/2 and added into respective N. oceanica cultures for co-cultivation. To block carbon dioxide from air, the flasks of —C cultures were carefully sealed with Parafilm M® over aluminum foil wrap. Cell viabilities were analyzed by confocal microscopy after 10-d co-culture of —N and 20 d of —C and —P.

### Light Microscopy

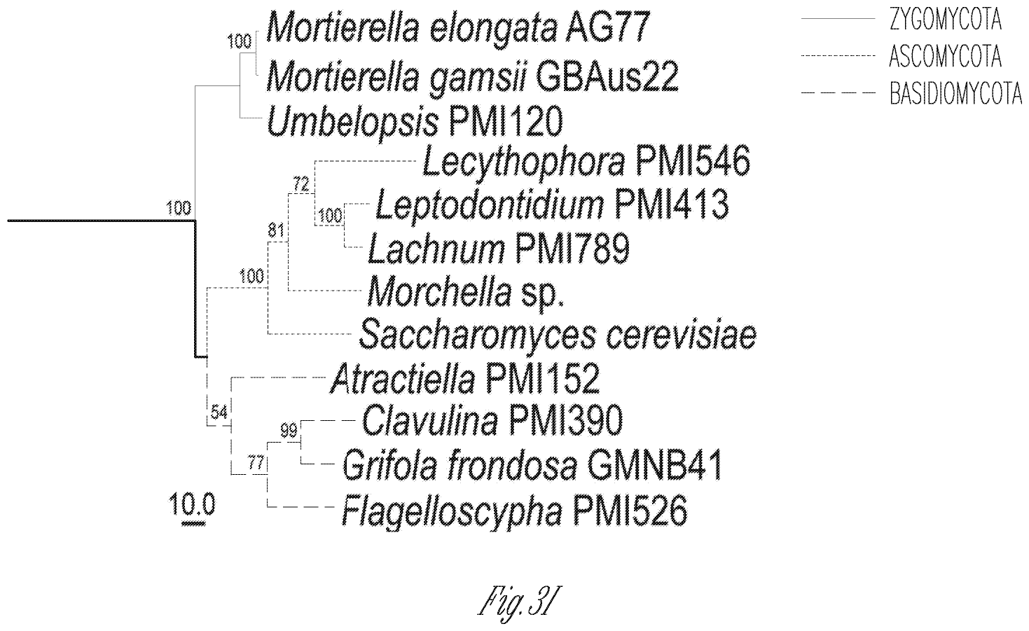

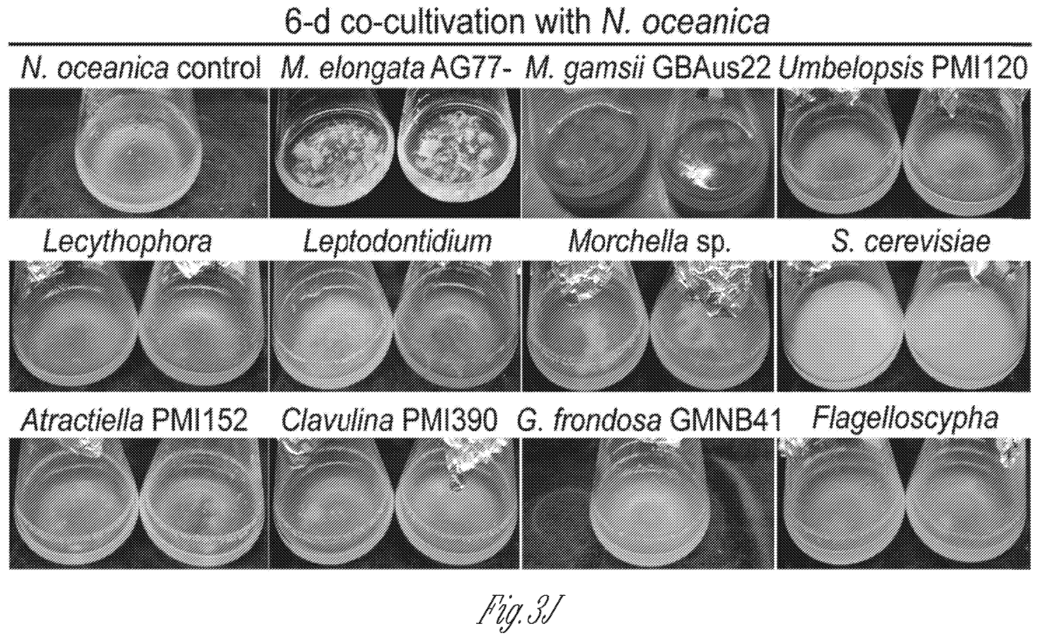

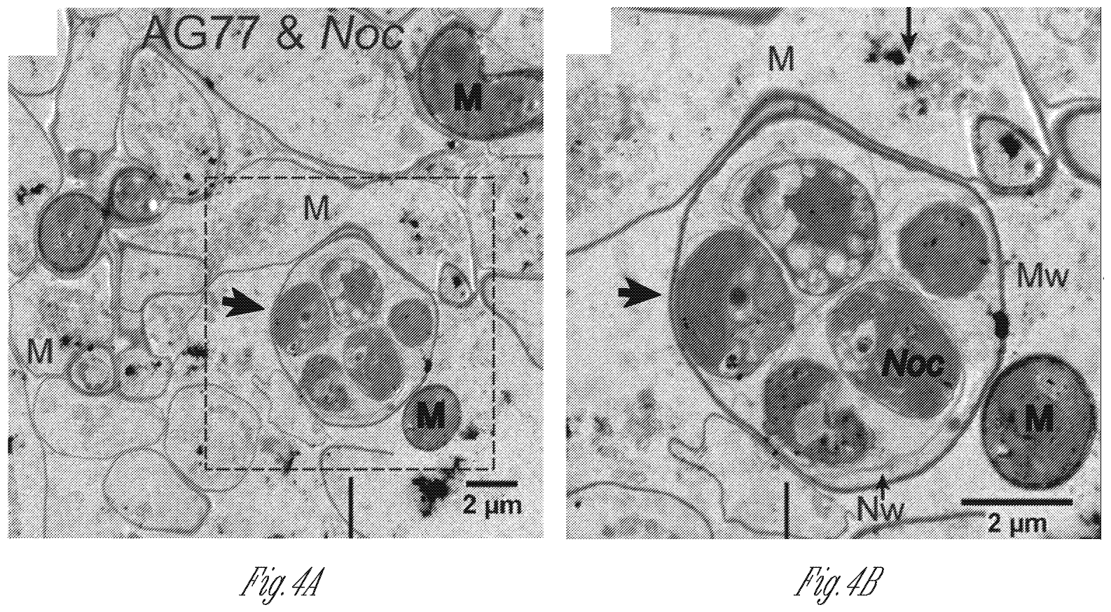

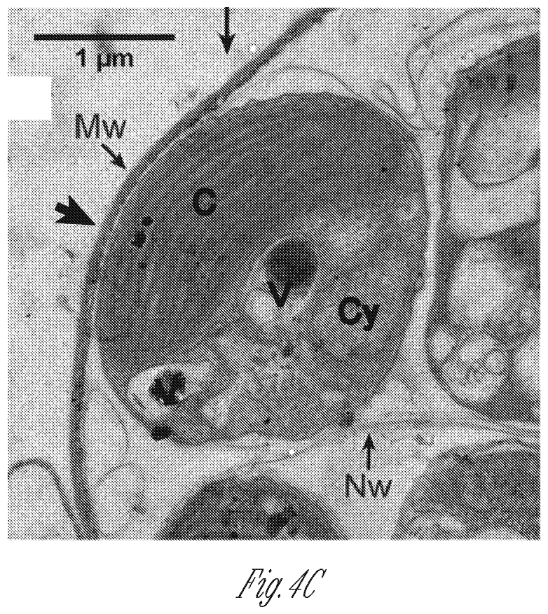

Interaction and symbiosis between algae and fungi were examined with an inverted microscope with differential interference contrast (DIC) and time-lapse modules (DMi8, Leica). DIC images were taken from the alga-fungus aggregates after short-term (6 days) and long-term (over one month) co-cultivation. To characterize the algal endosymbiosis in fungi, differential interference contrast (DIC) and time-lapse photography were performed using different period of long-term co-culture of algae and fungi (from 1 to 6 months). Alga-fungus aggregates grown in flasks were transferred to 35 mm-microwell dish (glass top and bottom, MatTek) and embedded in a thin layer of soft-solid f/2 medium supplemented with 10% PDB and 0.25% low gelling temperature agarose (Sigma-Aldrich) that immobilized cells for microscopy. Morphology of different age green hyphae (AG77 hyphae containing intracellular N. oceanica cells) was recorded in DIC micrographs (FIG. 4A to 4E), as well as real-time videos that showed four groups of green hyphae with manually adjusted focus. Videos were put side by side in a movie (data not shown) using video-editing software VideoStudio X9 (Corel). To investigate the establishment of algal endosymbiosis in fungi, randomly selected alga-fungus aggregates from 35-d co-culture were incubated and observed in 35 mm-microwell dish containing soft-solid f/2 medium with 10% PDB and 0.25% agarose up to two weeks. Time-lapse photographs were combined together to create another movie (data not shown) with VideoStudio.

### Scanning Electron Microscopy

SEM was performed to investigate the physical interaction between N. oceanica and M. elongata at the Center for Advanced Microscopy of Michigan State University (CAM, MSU). Alga-fungus aggregates from 6-d co-culture of N. oceanica and M. elongata (AG77 or NVP64) were fixed in 4% (v/v) glutaraldehyde solution and dried in critical point dryer (Model 010, Balzers Union). After drying, the samples were mounted on aluminum stub using high vacuum carbon tabs (SPI Supplies) and coated with osmium using a NEOC-AT osmium coater (Meiwafosis). Processed exocarp tissues were examined using a JSM-7500F scanning electron microscope (Japan Electron Optics Laboratories).

### Confocal Microscopy

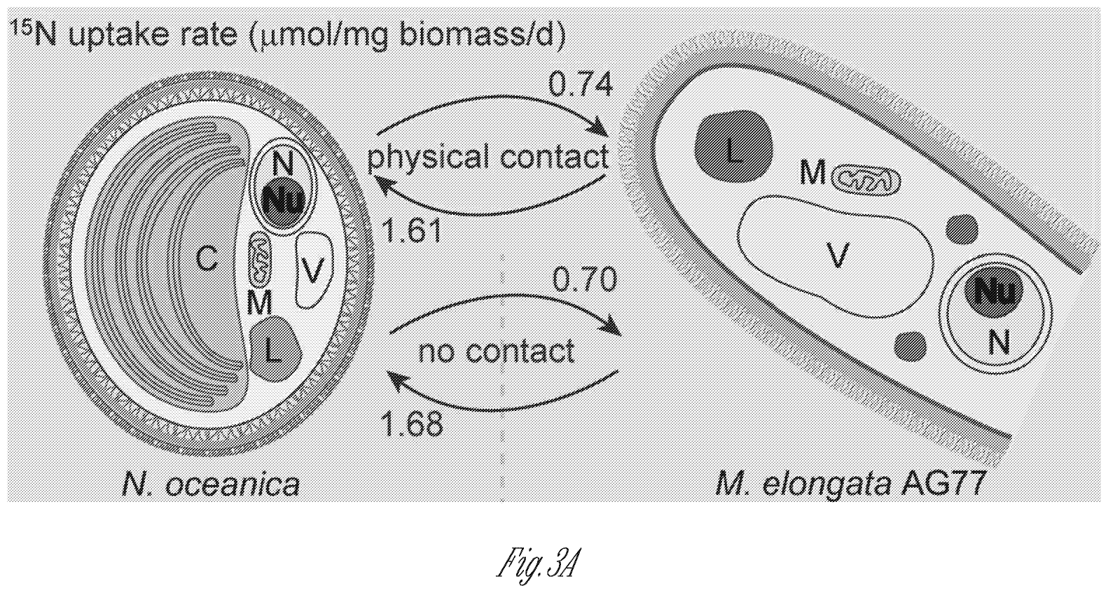

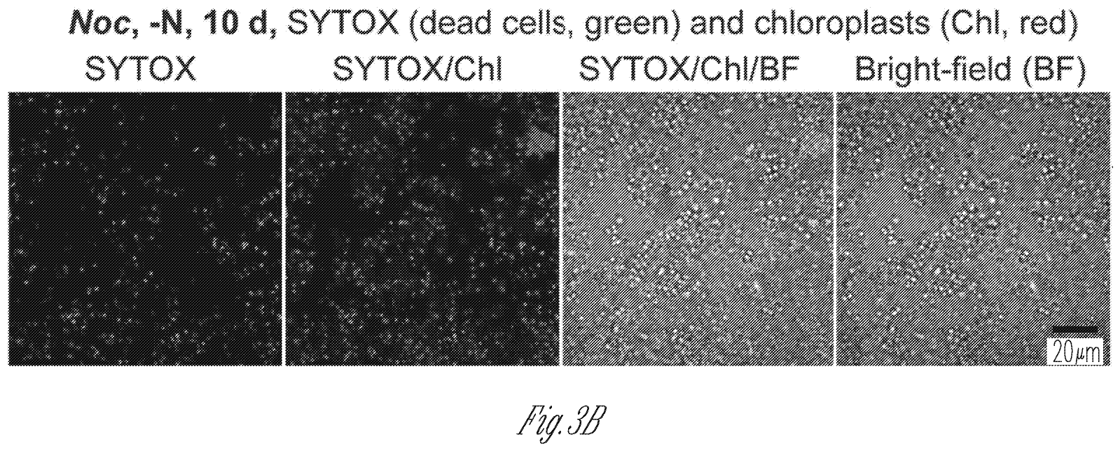

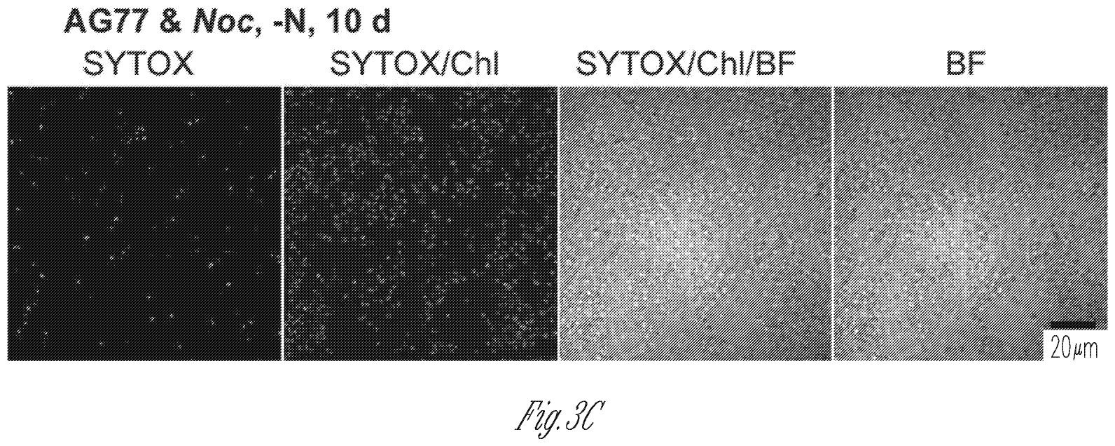

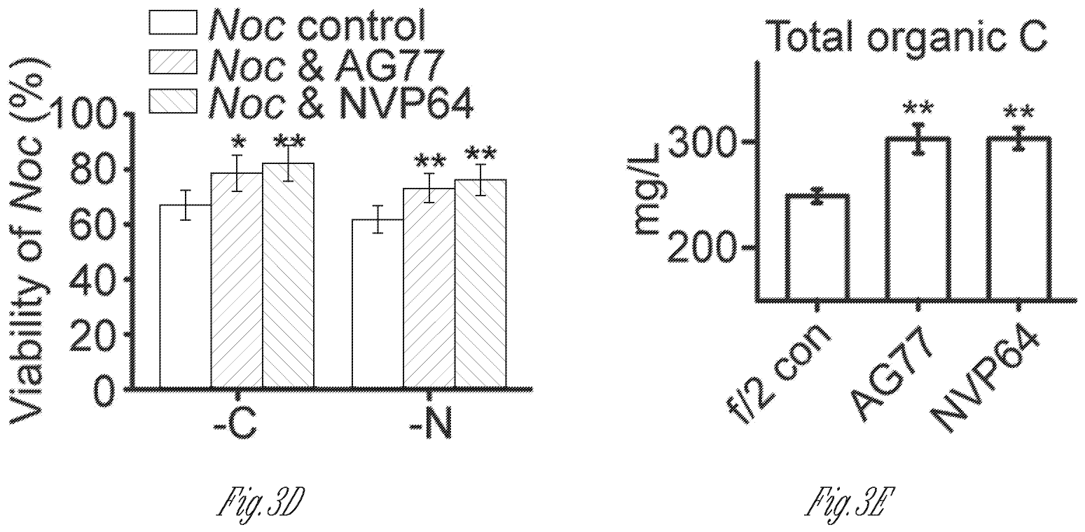

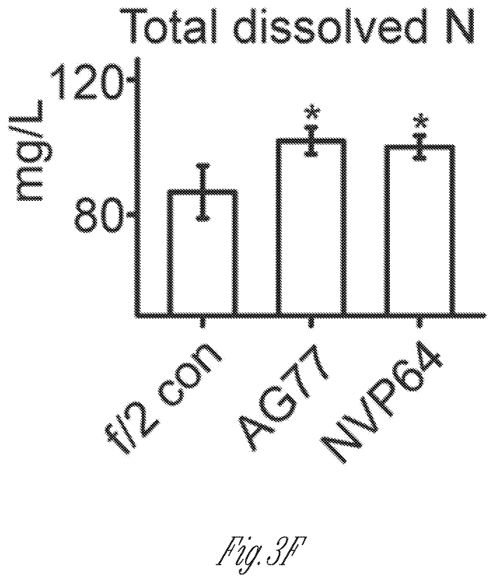

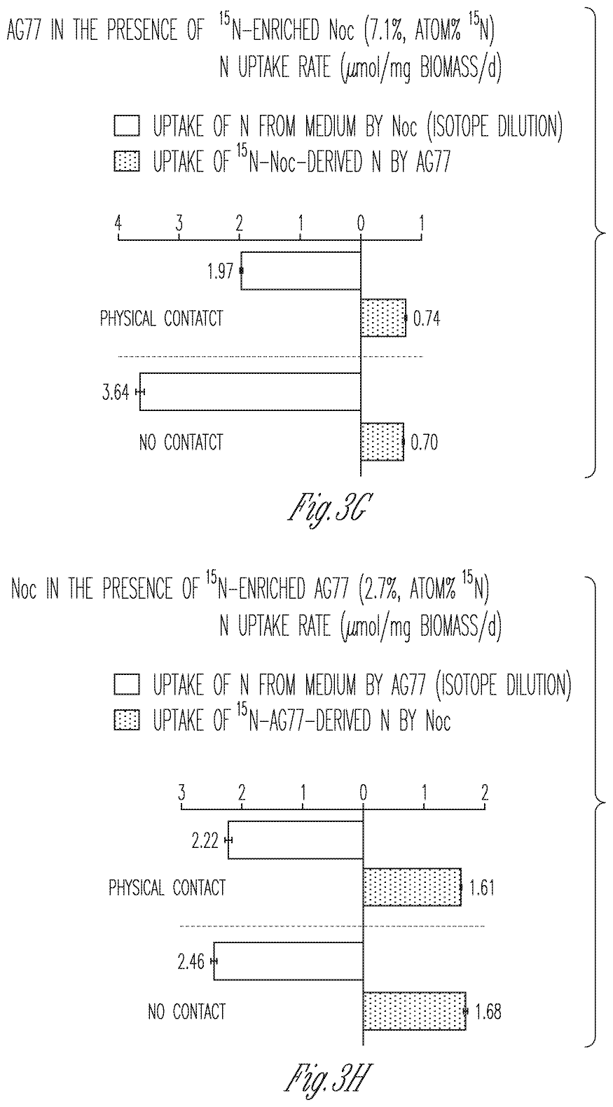

Viability of N. oceanica and M. elongata cells (e.g., during their co-culture) was determined by confocal microscopy using a confocal laser scanning microscope FluoView 1000 (Olympus) at CAM, MSU. SYTOX® Green nucleic acid stain (Molecular Probes, Life Technologies), a green-fluorescent nuclear and chromosome counterstain impermeant to live cells, was used to indicate dead cells of algae and fungi following a protocol described by Tsai et al. (Proc. Natl. Acad. Sci. U.S.A. 111, 15833-15838 (2014)). Briefly, 1 μL of 5 mM SYTOX Green was added to 1 mL of cell culture and incubated for 5 min in the dark at room temperature. Samples were washed twice with f/2 medium before observation (SYTOX Green, 488 nm excitation, 510 to 530 nm emission; chlorophyll, 559 nm excitation, 655 to 755 nm emission). Viability of N. oceanica cells was analyzed using ImageJ software. Cell viability was analyzed during alga-fungus co-culture in flasks containing f/2 medium (1, 4 and 7 days) to investigate whether the cells were living or dead during the 7-day co-culture of ¹⁴C- and ¹⁵N-chasing experiments. Viability of N. oceanica cells co-cultivated with M. elongata AG77 and NVP64 under nutrient deprivations (without a nitrogen source (—N), without a carbon source (—C), and/or without a phosphate source (—P)) was tested to evaluate whether N. oceanica benefits from the co-culture with Mortierella fungi (FIG. 3B-3D). Viability of M. elongata AG77 was analyzed during its 30-day incubation in f/2 medium to check whether the cells were living or dead when the culture media were collected for nutrient analyses (total organic C and dissolved N, FIG. 3F-3G).

Localization of N. oceanica cells in alga-fungus aggregates was investigated by cell-wall staining using Wheat Germ Agglutinin Conjugate Alexa Fluor® 488 (WGA, Molecular Probes) following the manufacturer's instruction. In brief, alga-fungus aggregates were collected by centrifugation and washed once with PBS buffer (pH7.2), followed by addition of 5 μg/mL WGA and incubation at 37° C. for 10 min. Samples were washed twice with f/2 medium and observed under the FluoView 1000 microscope (WGA, 488 nm excitation, 510 to 530 nm emission; chlorophyll, 559 nm excitation, 655 to 755 nm emission).

### Transmission Electron Microscopy

TEM was performed on Nannochloropsis oceanica and Mortierella aggregates co-cultured for about one month. Randomly collected alga-fungus aggregates were fixed overnight at 4° C. in sodium cacodylate buffer (50 mM, pH 7.2) supplemented with 2.5% (v/v) glutaraldehyde. The fixed samples were washed three times with sodium cacodylate buffer, post-fixed in 1% OsO₄ (v/v) for 2 hours at room temperature and then washed three times with sodium cacodylate buffer. After dehydration through a graded series of ethanol and acetone, samples were infiltrated with a series of acetone/resin Epon/Araldite mixtures and finally embedded in resin Epon/Araldite mixture (Electron Microscopy Sciences). Ultrathin sections (70 nm) were cut with an ultramicrotome (RMC Boeckeler) and mounted onto 150 mesh formvar-coated copper grids, followed by staining with uranyl acetate for 30 min at room temperature. The sections were then washed with ultrapure water and stained 10 min with lead citrate and used for observation. Images were taken with a JEOL100 CXII instrument (Japan Electron Optics Laboratories) equipped with SC1000 camera (Model 832, Gatan) and processed with ImageJ (FIG. 4F-4H).

### Example 2: Methods for Evaluating Nutrient Exchange Between Fungi and Algae

Light microscopy and SEM showed tight physical interaction between soil fungus Mortierella elongata and the marine algae Nannochloropsis oceanica. This Example describes experiment procedures for evaluating whether metabolic exchanges occur between N. oceanica and M. elongata.

Isotope labeling and chasing experiments were performed using labeled carbon and nitrogen (¹⁴C and ¹⁵N) nutrients for N. oceanica and M. elongata. For ¹⁴C assays, 20 μL of [¹⁴C]sodium bicarbonate (1 mCi/mL, 56 mCi/mmol, American Radiolabeled Chemicals) was added to 20 mL of early log-phase culture of N. oceanica (˜2×10⁶ cells/mL) and incubated for 5 days when the ¹⁴C incorporation reached ˜40%. The ¹⁴C-labeled N. oceanica cells were harvested by centrifugation (4,000 g for 10 min) and washed three times with f/2 medium. The supernatant of the last wash was analyzed in Bio-Safe II counting cocktail (Research Products International) using a scintillation counter (PerkinElmer 1450 Microbeta Trilux LSC), to confirm that ¹⁴C-labeling medium was washed off. The pellet of ¹⁴C-labeled N. oceanica was resuspended in 20 mL f/2 medium. Subsequently, non-labeled M. elongata AG77 mycelia (˜3 times of algae biomass, intact cells without blending) grown in PDB medium were washed twice with f/2 medium and added to the 20 mL ¹⁴C-labeled algal culture for 7-d co-cultivation. Alga-fungus aggregates were then harvested by PW200-48 mesh (Accu-Mesh) and algal cells in the flow through were collected by centrifugation (4,000 g for 10 min) and kept as the first part of ¹⁴C-labeled alga control. Alga-fungus aggregates were intensively washed in 50 mL conical centrifuge tube containing 40 mL of f/2 medium using a bench vortex mixer (˜1500 rpm, 15 min). Fungal mycelia were collected by NITEX 03-25/14 mesh (mesh opening 25 μm, SEFAR), and algal cells in the flow through were harvested by centrifugation and stored as the second fraction of ¹⁴C-labeled alga control. Mesh-harvested fungal mycelia (with obviously reduced amount of algae attached) were added to 1.5 mL microcentrifuge tube containing 300 μL of PBS buffer (pH 5.0) supplemented with 4% hemicellulase (Sigma-Aldrich) and 2% driselase (Sigma-Aldrich) and incubated overnight at 37° C. This step was performed to digest the algal cell walls (Chen et al. J. Phycol. 44, 768-776 (2008)). After cell-wall digestion, 700 μL of f/2 medium was added and algae were separated from fungi by intensive vortex for 15 min. Fungal mycelia were collected by NITEX 03-25/14 mesh while the flow-through was kept as the last fraction of alga control. Three fractions of ¹⁴C-labeled alga controls were combined together while fungi were washed three times with f/2 medium. Half of the samples were dried and weighed for biomass and the others were used for ¹⁴C measurements. To examine cross contamination after alga-fungus isolation, non-radioactive samples were processed the same way and analyzed by light microscopy and PCR. PCR primers were used that were specific for the N. oceanica gene encoding Aureochrome 4 (AUREO4), a blue light-responsive transcription factor that only conserved in photosynthetic stramenopiles such as N. oceanica: Aureo4pro F+ (5′-AGAGGAGCCATGGTAGGAC-3′; SEQ ID NO:1) and Aureo4 DNAD R− (5′-TCGTTCCACGCGCTGGG-3′; SEQ ID NO:2). Primers specific for M. elongata were also used, including genes encoding translation elongation factor EF1α and RNA polymerase RPB1: EF1αF (5′-CTFGCCACCCTTGCCATCG-3′; SEQ ID NO:3) & EF1αR (5′-AACGTCGTCGTTATCGGACAC-3′; SEQ ID NO:4), RPB1F (5′-TCACGWCCTCCCATGGCGT-3′; SEQ ID NO:5) and RPB1R (5-AAGGAGGGTCGTCTTCGTGG-3′; SEQ ID NO:6).

Isolated algae and fungi were frozen by liquid nitrogen and ground into fine powders by steel beads and TissueLyser II (QIAGEN), followed by lipid extraction in 1.2 mL chloroform:methanol (2:1, v/v) with vortex for 20 min. Double-distilled water (ddH₂O, 100 μL) was added to the samples, briefly mixed by vortex and then centrifuged at 15,000 g for 10 min. Organic phase was collected as total lipids. One mL of 80% methanol (v/v) was added to the water phase and cell lysis to extract free amino acids (FAAs). After centrifugation at 20,000 g for 5 min, supernatant was kept as total FAAs and the pellet was air-dried and used to extract protein with 200 μL of SDS protein extraction buffer at 42° C. for 15 min. After centrifugation at 10,000 g for 10 min, supernatant (˜200 μL) was collected for further protein precipitation (−20° C., 1 h) with the addition of 800 μL pre-cold acetone, while the pellet was kept for carbohydrate analyses. Total proteins (pellet) and soluble compounds (supernatant) were separated by centrifugation at 20,000 g for 15 min after protein precipitation. The pellet of total proteins was resuspended in 200 μL of SDS protein extraction buffer for scintillation counting. The pellet of carbohydrates was air-dried, resuspended in 200 μL ethanol, transferred to glass tube with Teflon-liner screw cap, and then dissolved by 2 to 4 mL of 60% sulfuric acid (v/v) according to described protocols (Velichkov, World J. Microbiol. Biotechnol. 8: 527-528 (1992); Scholz et al., Eukaryot. Cell. 13, 1450-1464 (2014)). Vortex and incubation at 50° C. were performed for the hard ones. Total lipids and soluble compounds were counted in 3 mL of xylene-based 4a20 counting cocktail (Research Products International), whereas total FAAs, proteins and carbohydrates were counted in 3 mL of Bio-Safe II counting cocktail. ¹⁴C radioactivity of the samples (dpm, radioactive disintegrations per minute) was normalized to their dry weight (dpm/mg).

To examine carbon transfer from fungi to algae, 200 μL of 0.1 mCi/mL [¹⁴C]D-glucose (268 mCi/mmol, Moravek Biochemicals) or 100 μL of 1 mCi/mL [¹⁴C]sodium acetate (55 mCi/mmol, American Radiolabeled Chemicals) were added to 20 mL of M. elongata AG77 grown in modified Melin-Norkrans medium [MMN, 2.5 g/L D-glucose, 0.25 g/L (NH₄)₂HPO4, 0.5 g/L KH₂PO4, 0.15 g/L MgSO4, 0.05 g/L CaCl₂)]. After 5-d ¹⁴C-labeling, fungal mycelia were harvested and washed three times with f/2 medium. Supernatant of the last wash was confirmed clean of ¹⁴C with scintillation counting. ¹⁴C-labeled fungi were added to 20 mL of N. oceanica culture for a 7-day co-culture. Alga-fungus aggregates were harvested using PW200-48 (first filtration) and NITEX 03-25/14 (second filtration) meshes. Algae in the flow-through were harvested and washed twice with f/2 medium by centrifugation and kept as free N. oceanica (unbound algal cells). The rest steps of sample preparation and ¹⁴C measurement was performed in the same way as described above.

To test whether physical contact is necessary for the carbon exchange between N. oceanica and M. elongata, ¹⁴C-labeling and chasing experiments were carried out using standard 6-well cell culture plates coupled with cell culture inserts that have a bottom made by hydrophilic polytetrafluoroethylene membrane filters (pore size of 0.4 μm, Millipore) to grow algae and fungi together with metabolic exchange but without physical contact. ¹⁴C-labeling was performed in the same way as described above. For alga-fungus co-culture, ¹⁴C-labeled algae (or fungi) were added in either plate wells or cell culture inserts while respective fungi (or algae) were grown separately in the inserts or plate wells to examine cross contamination. After 7-day co-culture, algae and fungi grown in the insert-plate system were easily separated by moving the insert to adjacent clean well. Samples were then processed following the protocol described above (without the steps of mesh filtration and cell-wall digestion).

Considering that Mortierella fungi are saprotrophic. Experiments were performed that involved ¹⁴C-labeling and chasing experiments using heat-killed ¹⁴C-cells to test whether algae and fungi utilize ¹⁴C from dead cells. Briefly, ¹⁴C-labeled algae or fungi were washed three times with f/2 medium and incubated in a water bath at 65° C. for 15 min, which killed the cells without causing serious cell lyses and addition of chemicals. Heat-killed ¹⁴C-algae (or fungi) were co-cultivated with unlabeled fungi (or algae) for 7 days in flasks. Subsequently, algae and fungi were separated by cell-wall digestion and mesh filtration, and ¹⁴C radioactivity of the samples was measured by scintillation counting as described above.

Nitrogen is another major nutrient for N. oceanica and Mortierella. Nitrogen exchange between N. oceanica and M. elongata was tested by ¹⁵N-labeling and chasing experiments using isotope ratio mass spectrometry. For ¹⁵N labeling of algae and fungi, N. oceanica cells were inoculated and grown in 200 mL of ¹⁵N-f/2 medium containing ˜5% of [¹⁵N]potassium nitrate [¹⁵N/(¹⁵N+¹⁴N), mol/mol], while M. elongata mycelia were inoculated and incubated in 2 L of ¹⁵N-MMN medium containing ˜5% of [¹⁵N]ammonium chloride for two weeks. Algal culture was diluted by the addition of fresh ¹⁵N-f/2 medium to maintain cell density at log phase. ¹⁵N-labeled N. oceanica cells from a 4 liter culture and ¹⁵N-labeled M. elongata mycelia from a 2 liter culture were harvested and a portion of the samples was kept as ¹⁵N-labeled controls. The rest of the sample was added to unlabeled cells in flasks (with physical contact) or to unlabeled cells in 6-well-culture plates with inserts (no physical contact) for a 7-day co-cultivation. Algae and fungi were separated after the co-culture as described above. Samples were then washed three times with ddH₂O. Fungal mycelia were homogenized in TissueLyser II (QIAGEN) using steel beads. Algae and fungi were then acidified with 1.5 to 3 mL of 1 N HCl, dried in beakers at 37° C. and weighed for biomass. Isotopic composition of algae or fungi (δ¹⁵N, ratio of stable isotopes ¹⁵N/¹⁴N) and nitrogen (N) content (% N) were determined using a Eurovector (EuroEA3000) elemental analyzer interfaced to an Elementar Isoprime mass spectrometer following standard protocols (Fry et al., Rapid Commun. Mass Spectrom. (2007)). The N uptake rates (μmol N/mg biomass/day) of ¹⁵N-labeled N. oceanica cells from the media (medium-N, isotope dilution) and that of AG77 from ¹⁵N-labeled N. oceanica-derived N (¹⁵N) were calculated based on the Atom % ¹⁵N [¹⁵N/(¹⁵N+¹⁴N)100%], % N and biomass following a protocol by Ostrom et al. (2016). The N uptake rates of ¹⁵N-AG77 from the media and that of recipient N. oceanica from ¹⁵N-AG77-derived N (¹⁵N) were calculated in the same way.

### Carbon and Nitrogen Measurements

Total organic carbon (TOC) and total dissolved nitrogen (TDN) in the media of Mortierella cultures were measured with a TOC-Vcph carbon analyzer with total nitrogen module (TNM-1) and ASI-V autosampler (Shimadzu) (FIG. 3F-3G). M. elongata AG77 and NVP64 were incubated for 18 days in flasks containing 25 mL of f/2 medium. Fungal tissues were removed by filtration with 0.22 micron filters (Millipore) and the flow-through was subject to TOC and TDN analyses.

### Example 3: Carbon Nutrient Exchange Between Fungi and Algae

To test whether carbon or nitrogen exchange underlies the interaction between the soil fungus Mortierella elongata AG77 and the marine algae Nannochloropsis oceanica, a series of experiments were conducted using reciprocally ¹⁴C- and ¹⁵N-labeled algal and fungal partners. For carbon exchange assays algal cells were labeled with [¹⁴C]-sodium bicarbonate and co-cultivated with non-labeled hyphae in flasks for one week. Conversely, fungal hyphae were grown in either [¹⁴C]-glucose- or [¹⁴C]-acetate-containing medium, then were co-incubated with non-labeled algal cells in flasks that allowed the two organisms to interact physically. Co-cultured algal and fungal cells were separated from each other by mesh filtration and were then analyzed for ¹⁴C exchange.

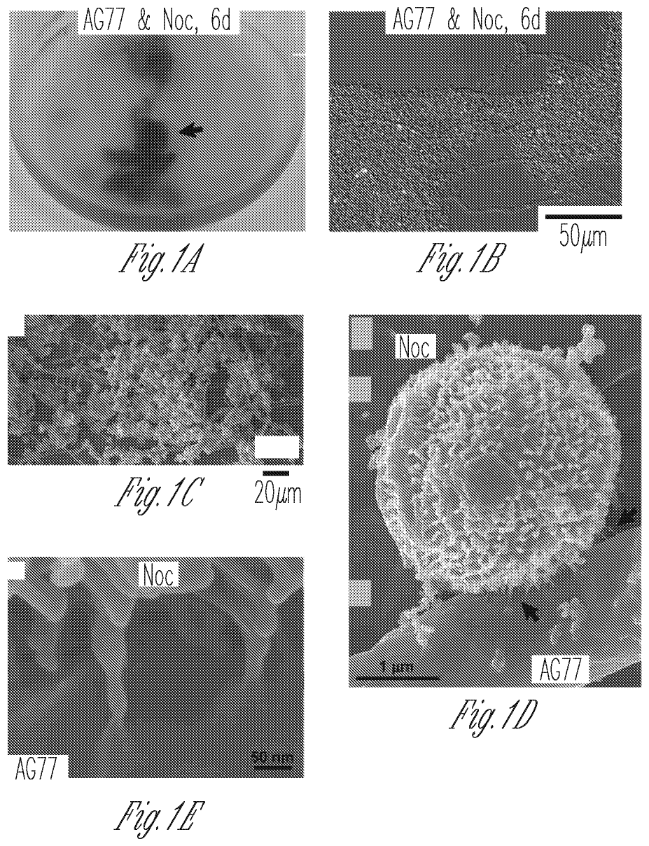

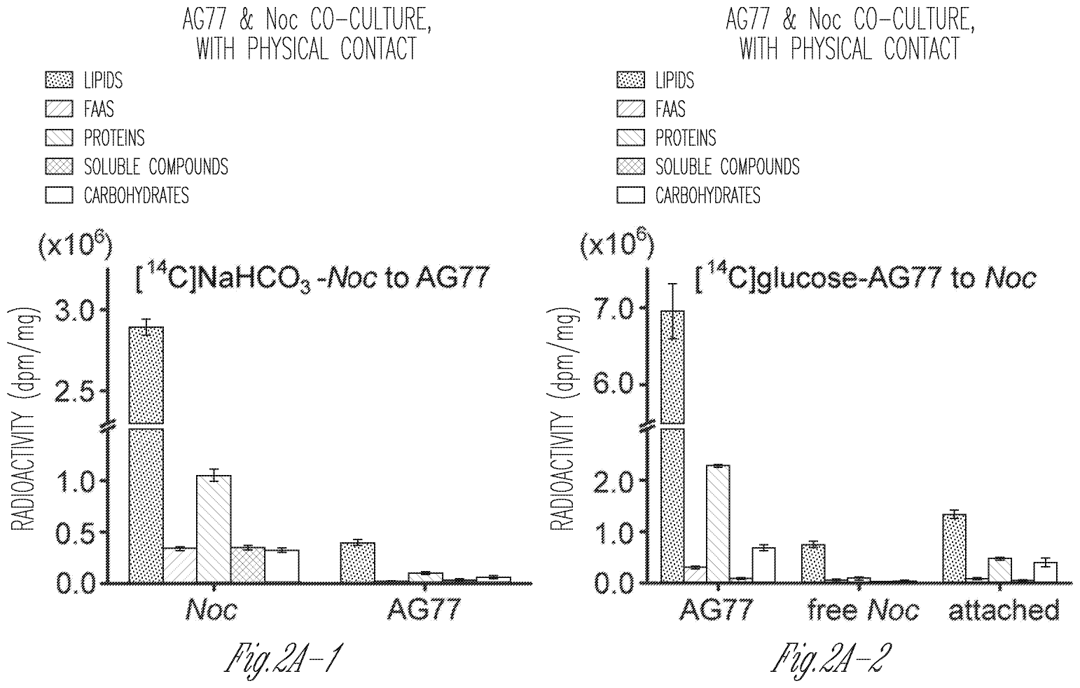

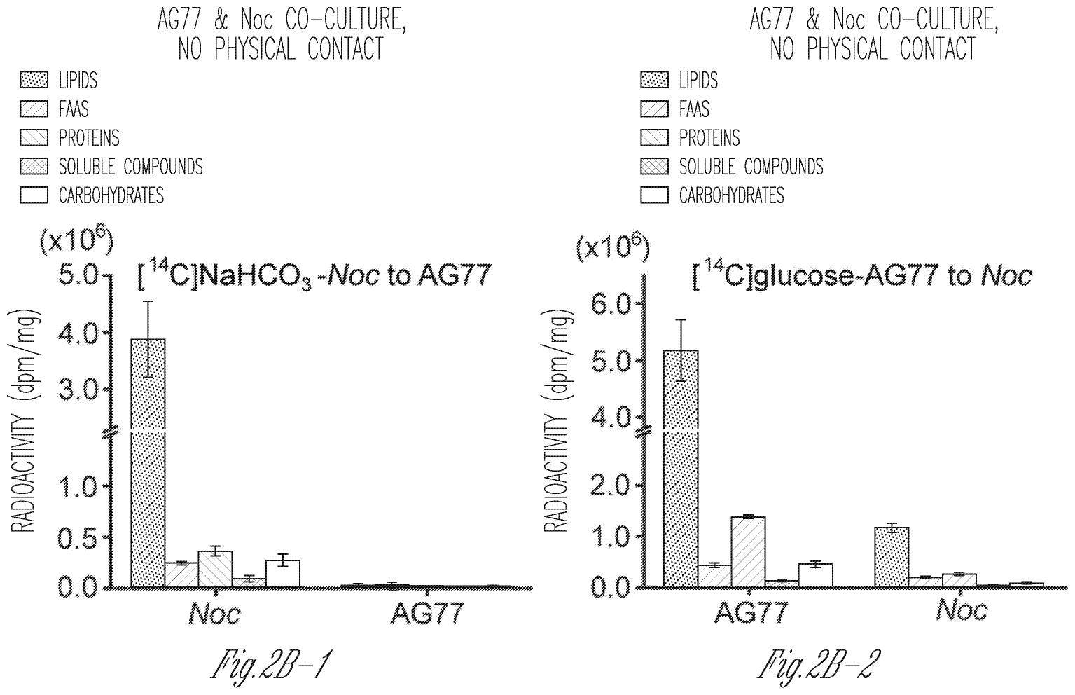

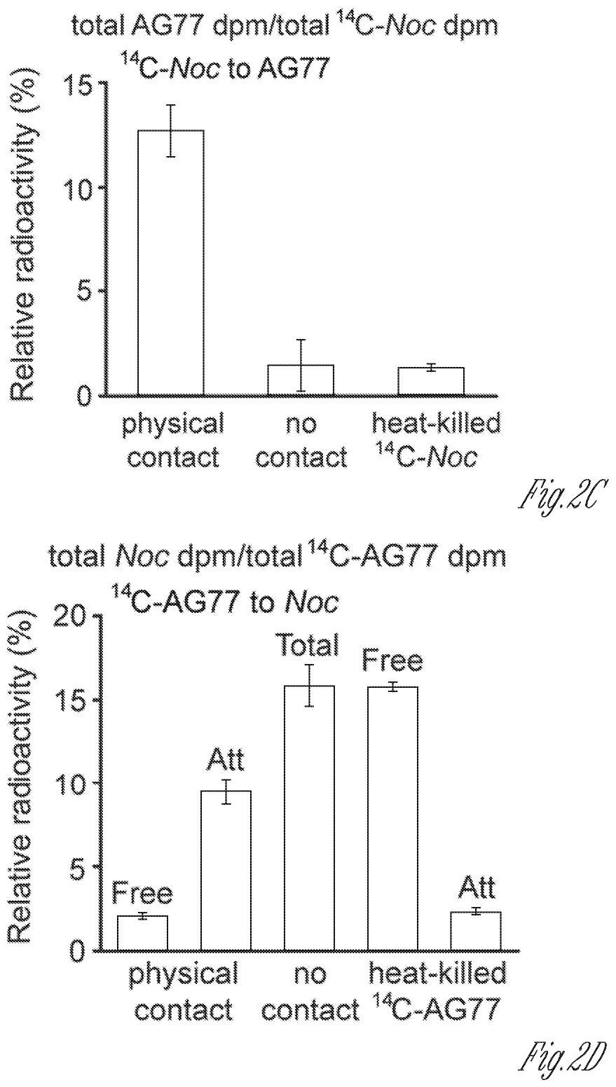

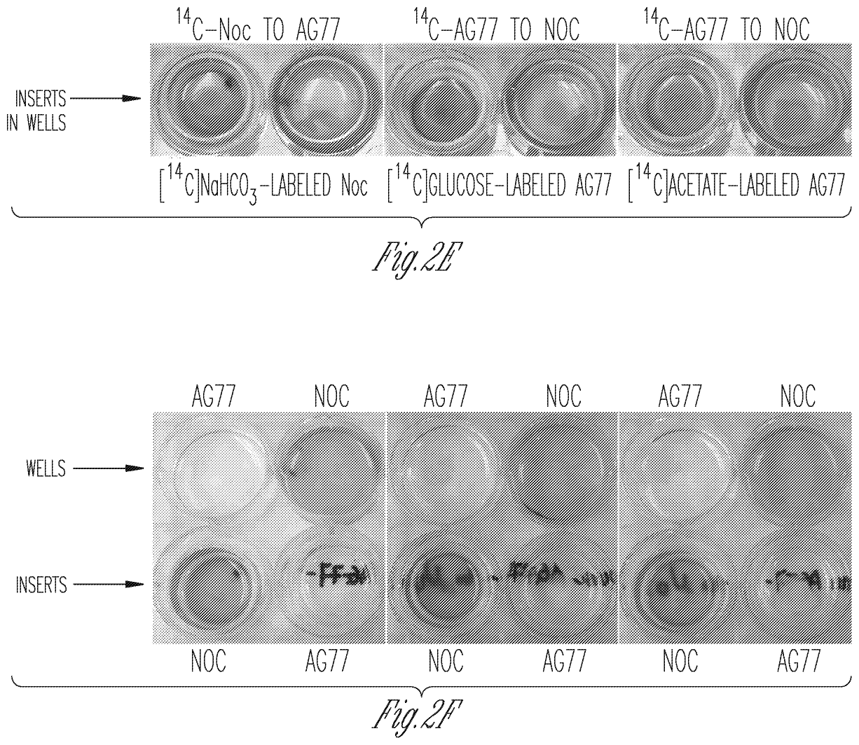

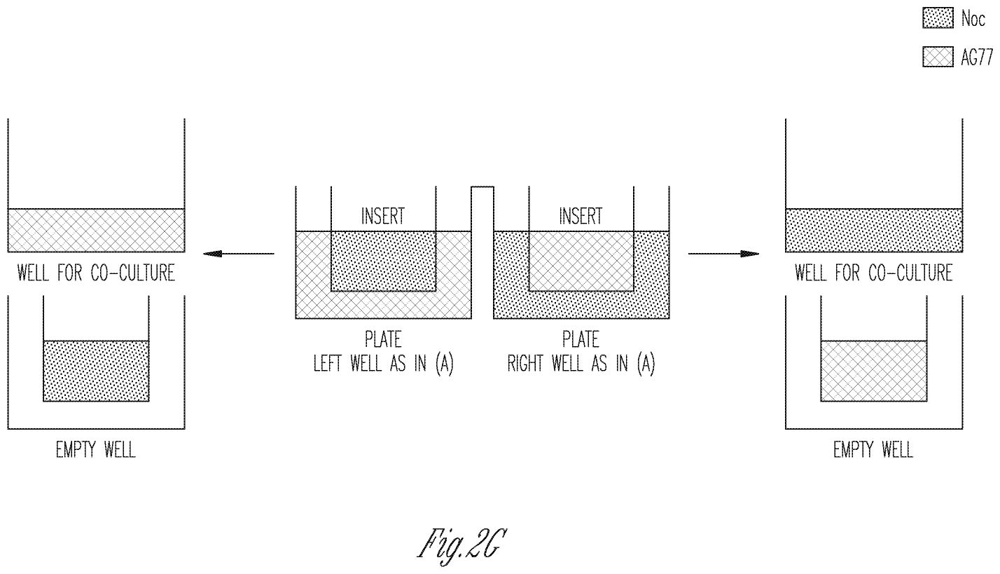

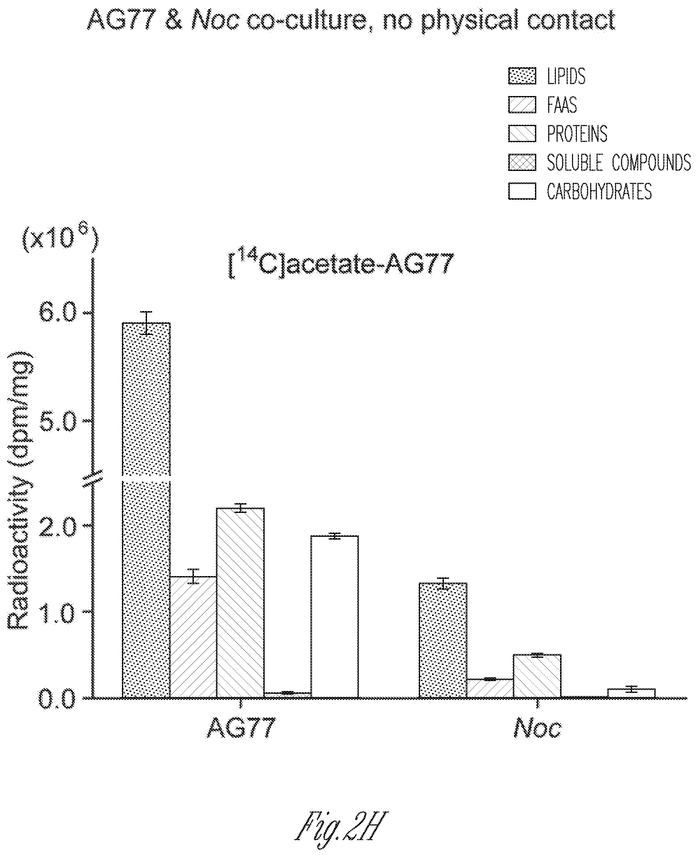

FIG. 2A-1 shows that ¹⁴C-carbon is transferred from the alga (Nannochloropsis oceanica: Noc) to the fungus (Mortierella elongata AG77). Nearly 70% of the transferred ¹⁴C-carbon was incorporated into the fungal lipid pool. Similarly, ¹⁴C-carbon transfer was observed from the labeled fungus (Mortierella elongata AG77) to its algal recipient (Nannochloropsis oceanica: Noc) (FIG. 2A-2). Intriguingly, algal cells attached to the fungal hyphae acquired more ¹⁴C than unattached cells grown in the same flask (FIG. 2A).

To further assess whether a physical interaction is required for carbon exchange between the photosynthetic alga and the putative fungal saprotroph, membrane inserts were used to physically separate reciprocally ¹⁴C-labeled algal and fungal partners (FIG. 2E-2H). These experiments showed that the physical contact between the algae and fungus is essential for ¹⁴C-carbon transfer to the fungus (FIG. 2B-2C), but is not necessary for ¹⁴C-carbon transfer to the algal cells (FIG. 2B, 2D and FIG. 2H).

Mortierella is regarded as a saprotroph that acquires carbon from dead organic matter. Experiments were performed, first, to test whether alga-derived carbon obtained by Mortierella elongata was due to the consumption of algal detritus. The ¹⁴C-labeling experiment described above was repeated using a 65° C. water bath to kill ¹⁴C-labeled cells prior to algal-fungal reciprocal pairings. Mortierella elongata incorporates a small amount (1.3%) of ¹⁴C-carbon from dead algal cells, compared to ¹⁴C-carbon acquired from living algal cells (12.7%) (FIG. 2C). In contrast, the algal cells attached to fungal hyphae (att) and those free in the medium (free) acquired more ¹⁴C-carbon (att, 2.4%; free, 15.8%) from dead fungal cells (FIG. 2D). The total abundance of ¹⁴C-carbon was higher in the free algal cells, because most of the Nannochloropsis oceanica cells were free in the medium.

Second, confocal microscopy and Sytox Green staining was used to assess whether fungal and algal cells remained alive during co-culture. These results confirmed that most algal and fungal cells remain alive throughout the co-cultivation of ¹⁴C-labeling experiment and also demonstrate that the heat treatment was effective in killing algal and fungal cells (data not shown). Together these data indicate that carbon-transfer from the algae to the fungus is dependent upon an intimate physical interaction between living partners. In contrast, algae are able to utilize carbon from the fungus grown in the same culture regardless of whether the hyphae are alive or physically connected.

### Example 4: Nitrogen Exchange Between Fungi and Algae

Nitrogen is a major macronutrient that can limit net primary productivity in terrestrial and aquatic ecosystems, including for microalgae such as N. oceanica. To determine whether nitrogen-exchange occurs between fungi (M. elongata) and algae (N. oceanica), the algae were labeled with [¹⁵N]potassium nitrate and the fungus were labeled with [¹⁵N]ammonium chloride. The labeled fungal and algal cells were separately co-cultivated with unlabeled partners for one week and then the different cultures were then analyzed for ¹⁵N. Nitrogen (¹⁵N) transfer occurred between algal and fungal partners, irrespective of whether they were in physical contact or not (FIG. 3A, 3G-3H). Further, over twice as much ¹⁵N (˜1.6 μmol/mg biomass/d) was transferred from the ¹⁵N-fungus to the algal recipient, than from the ¹⁵N-algae to the fungus (˜0.7 μmol/mg biomass/d—see FIG. 3A, 3G-3H), showing a net nitrogen benefit for the algae when in symbiosis with the fungus.

A nutrient-deficiency test was also performed to assess algae benefits from the nutrient transfer by it fungal partner. Results showed that N. oceanica had significantly increased viability when co-cultivated with M. elongata under nitrogen or carbon deprivation but not under phosphorus deficient conditions (FIG. 3B-3D). These results indicate that a functional Mortierella-Nannochloropsis interaction is established that may be based upon the carbon and nitrogen acquisition and transfer and that is adaptive under nutrient-limited conditions.

Further analysis of the culture supernatant showed an increase in total organic carbon and dissolved nitrogen when the living Mortierella fungi were incubated alone in f/2 medium (FIG. 3E-3F) indicative of extracellular release of nutrients by the fungus, and perhaps explaining why physical contact is not required for the ¹⁴C transfer from the fungus to the algae. It appears that algae benefit from this interaction with Mortierella by acquiring both nitrogen and carbon from its fungal symbiont. On the other hand, through an intimate interaction with living photosynthetic algae, Mortierella is able to grow in nutrient-limited conditions (PBS buffer) by incorporating algal-derived carbon and nitrogen.

Numerous lineages of fungi have evolved to interact with plants and algae, and the question arises whether the observed interaction is unique to Mortierella or alternatively, if it is conserved across diverse lineages of fungi. This was addressed through a series of interaction experiments where N. oceanica was paired with a series of fungi sampled across the fungal phylogeny (FIG. 3I-3J). This diverse panel of 21 isolates included the yeast Saccharomyces cerevisiae, and filamentous ascomycetes, basidiomycetes, and mucoromycetes isolates representing 3 phyla, 9 orders and 13 families of Fungi. Aside from some Mortierella species tested, interactions between these fungi and algae were negative or neutral. Mortierella elongata showed the most obvious phenotype and physical attraction to algae, with the algae clustered tightly around the fungal mycelium (FIG. 3J).

Microbial consortia may persist in a stable state, improving the resilience of each to fluctuating environments and stress (Brenner et al., Trends Biotechnol. 26, 483-489 (2008)). To determine whether the observed interactions between N. oceanica and M. elongata are stable or transient we carried out a series of long-term incubations (from 1 to 6 months) in which the partners were grown together with nutrients refreshed biweekly. After about one month, co-culture confocal microscopy was used to visualize cells inside the thick aggregates that formed between algae and fungus, using the Wheat Germ Agglutinin Conjugate cell wall probe which binds to N-acetylglucosamine, a component in fungal and algal cell walls. From these images some algal cells were within fungal hyphae. Subsequent light and transmission electron microscopies (TEM) were used to provide more details of this interaction and provide evidence for the endosymbiosis of the algae by the fungus. In the algal-fungal aggregates the algae are trapped by the fungus, and some algal cells are indeed intracellular within the hyphae, as shown in TEM micrographs (FIG. 4A-4C). Additional imaging with differential interference contrast (DIC) micrographs and videos demonstrated morphology of the “green hyphae” after different periods of long-term co-culture, further confirming algal endosymbiosis by the fungus and incorporation of intact and functional algal cells intracellularly within the fungal hyphae (FIG. 4D-4H). Both algal and fungal cells remained viable after months of co-culture. This fungal-algae symbiosis may conjure the idea of a lichen, but it differs by the lack of distinct tissue and hyphal structures (i.e. thallus, haustoria) and by the fact that Mortierella fungi actually incorporate algal cells intracellularly while lichens do not. The result of this remarkable incorporation of intact and functional algal cells within living fungal mycelia has the hallmarks of a secondary endosymbiosis event.

While observations on endosymbiosis of living eukaryotic cells by fungi have not been reported previously, the rare fungus Geosiphon pyriformis (a relative of arbuscular mycorrhizae and of Mortierella) is reported to form a unique intracellular association with the cyanobacterium Nostoc punctiforme (Mollenhauer et al., Protoplasma. 193, 3-9 (1996)). In this system, the fungus envelops Nostoc within a specialized swollen multinucleate fungal “bladder” that is morphologically distinct from the rest of the hyphae. Within this bladder, the cyanobacteria are surrounded by a host-derived symbiosome membrane (Brenner et al., Trends Biotechnol. 26, 483-489 (2008)).

Biogenesis of endosymbiosis of N. oceanica by M. elongata was evaluated through DIC and time-lapse microscopy. Endosymbiosis was preceded by dense aggregates of algal cells around the fungal hyphal tip (FIG. 4I-1 to FIG. 4I-4). Further, aggregates of algal cells were observed surrounding fungal hyphal tips early in the endosymbiosis process, for example, by 1-2 months. Dense clusters of algal cells formed at the tip of a hypha were consistently observed when the endosymbiosis of algal cells within fungal hyphae happened in plates. Also, hyphae downstream from these tips are often green, and the amount of algae within the cells increased over time (e.g., over 1-2 months). Given these observations we hypothesize that the hyphal tip is the initial point of entry for the algal cells into the fungal protoplasm, as this also where the fungal cell wall is least developed. Not only do algae enter the fungal mycelium, but once inside the mycelium they remain active, appear healthy and are able to multiple. We suspect that the coenocytic nature of Mortierella, which has few septa within its mycelium, is one attribute of this fungus that facilities its ability to pack cells with photosynthetic algae. TEM and DIC images show that the fungal host's cell membrane remains intact around the internalized algae (FIG. 4A-4I). Removed from their natural environment, internalized algae would become more completely dependent on the host for nitrogen and other nutrients, which could be exchanged for carbon photosynthate and possibly other metabolites.

### Example 5: N. oceanica Cell Wall Degradation Upon Interaction with M. elongata

N. oceanica and M. elongata cells were incubated together as described in the previous Examples. Micrographs were taken using scanning electron microscopy (SEM) to view N. oceanica cell walls, particularly at the outer layer of the N. oceanica cells, after the co-cultivation of N. oceanica and M. elongata fungi AG77.

A previous study on cell wall structure of Nannochloropsis gaditana (Scholz et al., Eukaryot Cell 13(11):1450-64 (2014)) indicates that Nannochloropsis gaditana cells have a layer of extensions in their cell wall when observed using high-resolution quick-freeze deep-etch electron microscopy (QFDE-EM). Those studies suggest that there may be a very thin layer of cell wall outside and connected to an extension layer. The thin outer cell wall observed by Scholz et al. (2014) may be fragile because some cells partially lost the thin outer layer during the QFDE-EM.

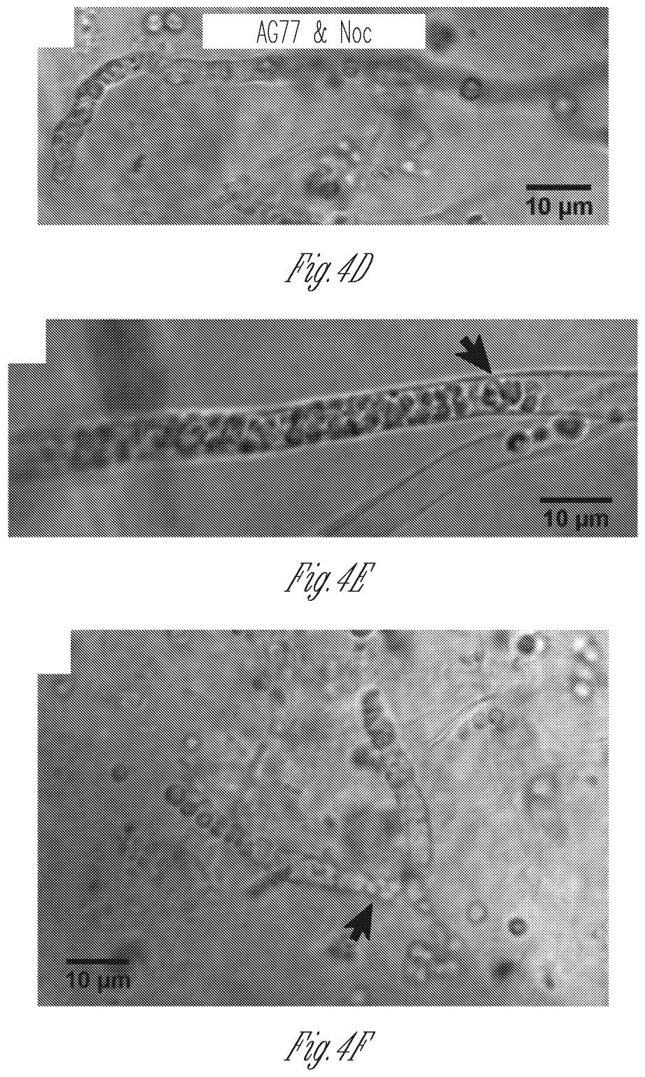

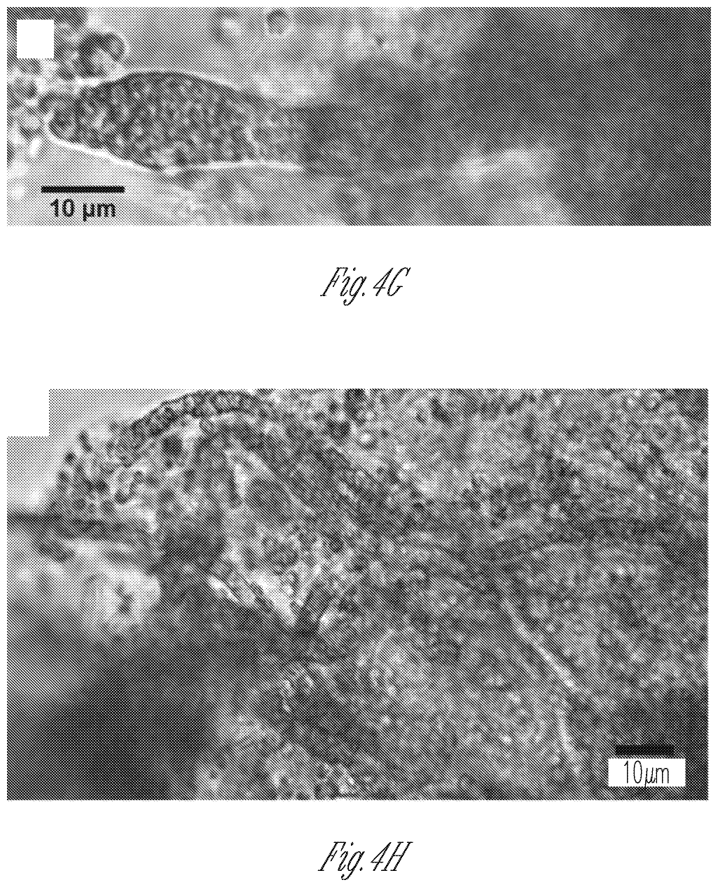

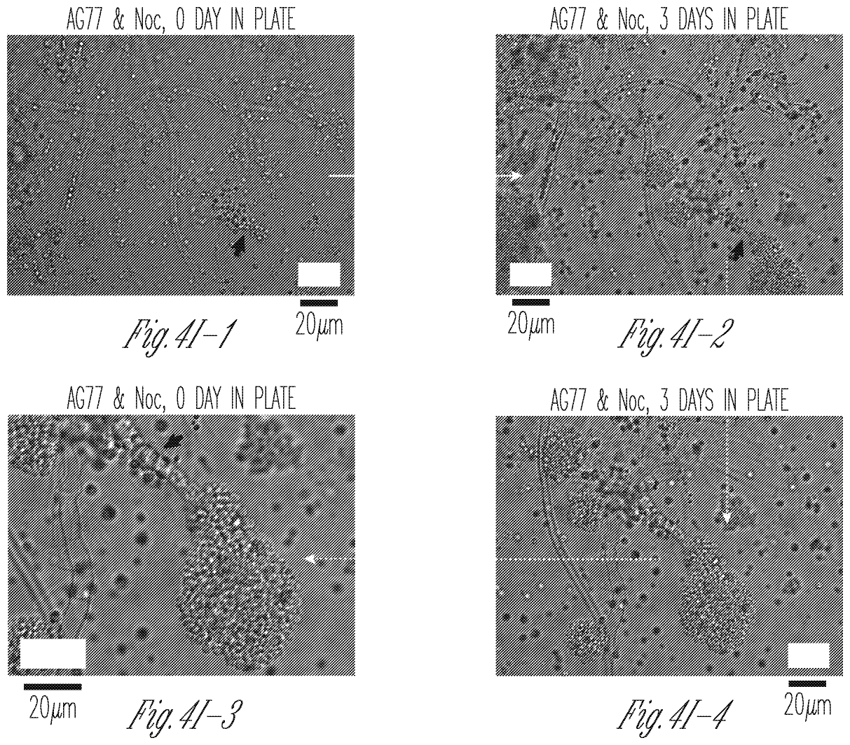

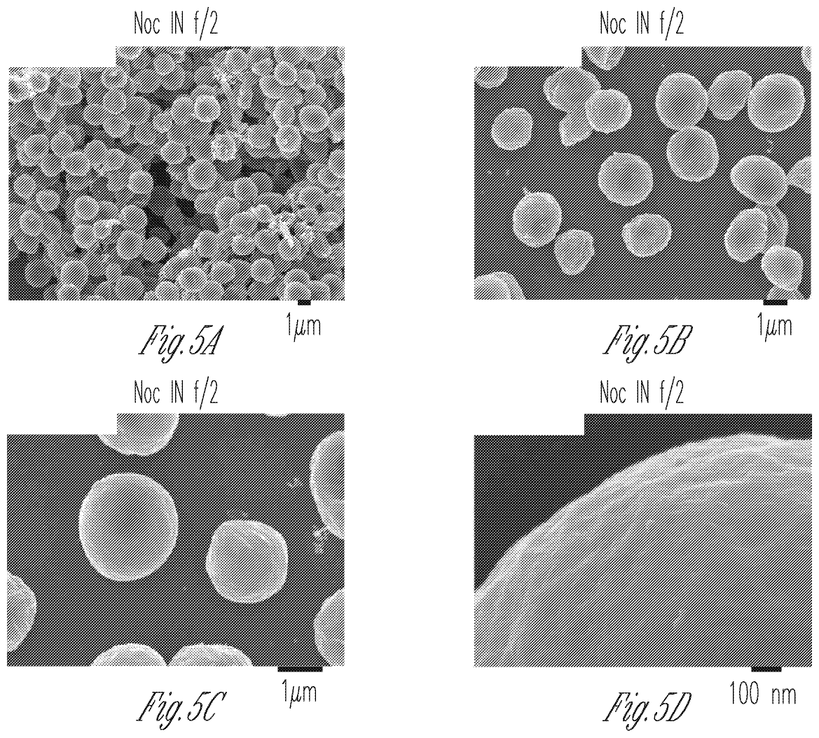

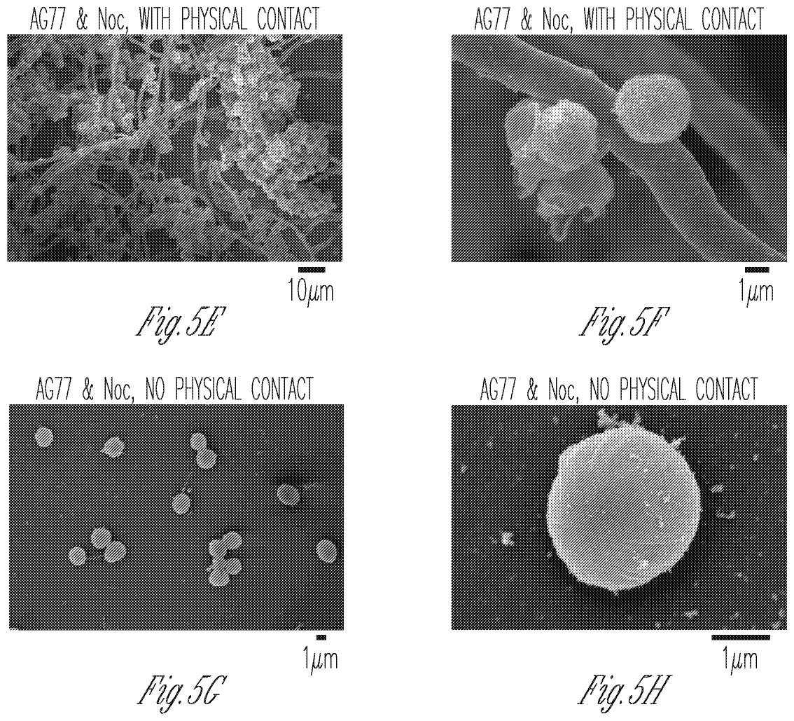

As illustrated in FIG. 5A-5H, physical interaction between N. oceanica and M. elongata fungus AG77 led to degradation of the thin outer layer of the N. oceanica cell wall, which exposed an extension layer attached to the rugged surface of fungal hypha. This algal extension layer formed irregular-tube-like structures. Such degradation of the N. oceanica cell wall was not observed in N. oceanica algal cells co-cultivated with M. elongata AG77 but separated from the M. elongata AG77 fungi by a membrane insert that physically separates the algal and fungal cells but allows metabolic exchange between the two organisms.

These data indicate that physical or intimate interaction is required for the algal cell wall degradation.

### Example 5: Additional Materials and Methods

This Example describes some alternative materials and methods for generating fugal-algal aggregates.

### Materials and Growth Condition

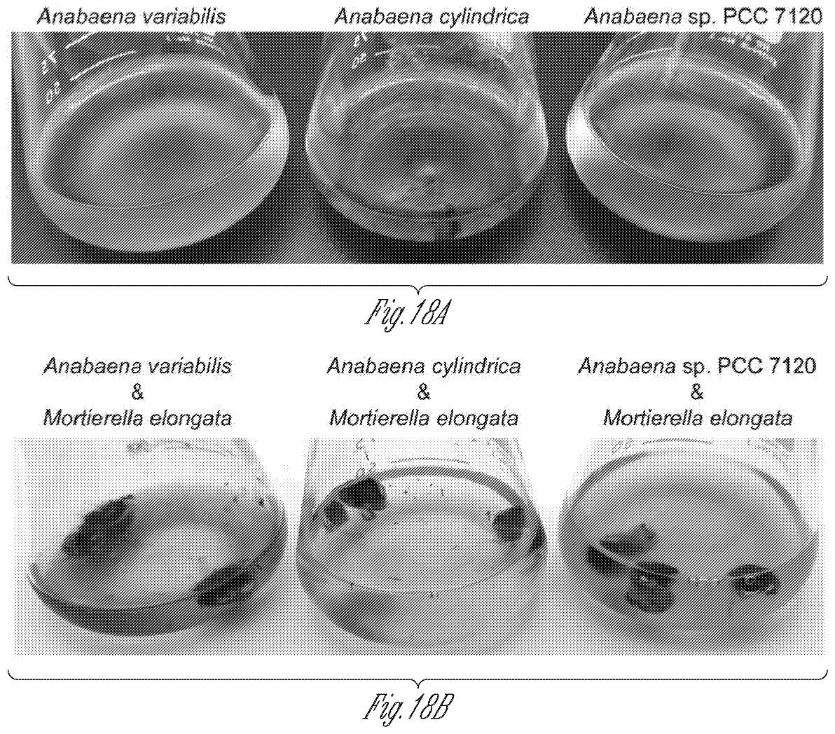

The marine alga Nannochloropsis oceanica CCMP1779 was obtained from the Provasoli-Guillard National Center for Culture of Marine Phytoplankton. N. oceanica DGTT5-overexpressing strains DGTT5ox3 and DGTT5ox6 were generated using the expression vector shown in FIG. 17A-17B. The N. oceanica DGTT5-overexpressing DG7T5ox3 and DG7T5ox6 lines were examined using quantitative RT-PCR methods described by Zienkiewicz et al. (Biotechnology for biofuels 10:8 (2017)). f/2 medium was used to grow the alga that contains f/2 nutrients (Andersen et al., Appendix A. Algal Culturing Techniques. San Diego: Elsevier Academic Press (2005)) and 20 mM sodium bicarbonate and 15 mM Tris buffer (pH 7.6) to prevent carbon limitation (Vieler et al. Plant physiology 158(4):1562-1569 (2012)). The cells were grown in batch cultures in two systems: shaker flask with f/2 medium (under ˜80 μmole photons m⁻² s⁻¹ at 23° C.) or in environmental photobioreactors (ePBRs) (Lucker et al., 2014) with f/2-NH₄Cl (2.5 mM NH₄Cl replacing 2.5 mM NaNO₃) or f/2-urea (2.5 mM urea replacing 2.5 mM NaNO₃) media with varying light as indicated in FIG. 6A-6D (e.g., as shown in FIG. 6, the S2 cells were exposed to 0 to 2,000 μmol photons m⁻² s⁻¹ under diurnal 14/10 h light/dark cycle) at 23° C. and sparged with air enriched to 5% CO₂ at 0.37 L min⁻¹ for 2 min per hour. For prolonged-incubation in the ePBR, N. oceanica cells were inoculated to ˜1×10⁶ mL⁻¹ in f/2-NH₄Cl medium and grown to stationary phase. The cultures were further incubated for 8 days to increase TAG content.

Mortierella fungi M. elongata AG77, M. elongata NVP64, and M. gamsii GBAus22 isolates were isolated from soil samples collected in North Carolina (AG77), Michigan (NVP64), USA, and Australia (GBAus22). Morchella americana 3668S was obtained from the USDA NRRL Agriculture Research Station.

Fungal samples were incubated in PDB medium (12 g/L potato dextrose broth and 1 g/L yeast extract, pH5.3) at 23° C. For the algal-fungal cocultivation, fungal mycelia were briefly blended into small pieces (˜1 cm) with a sterilized blender and were collected by centrifugation (3,000 g for 3 min) after 24-h recovery in PDB medium. The samples were washed twice with f/2 or f/2-NH₄Cl medium and resuspended in 5-10 mL of the respective medium. One third of the samples were used for determining dry biomass: 1 mL culture was transferred and filtered with pre-dried and—weighed Whatman GF/C filters and dried overnight at 80° C. The remaining fungal mycelia were added to the N. oceanica culture (˜3 times to algal biomass) for 6-day co-cultivation on a shaker (˜60 rpm) under continuous light (˜80 μmol photons m⁻² s⁻¹) at 23° C.

Cell size and concentration of N. oceanica cultures were calculated with a Z2 Coulter Counter (Beckman). The bio-flocculation efficiency of N. oceanica cells using fungal mycelium was determined by the cell density of uncaptured algal cells compared to that of an algal culture control, to which no fungus was added.

### Light Microscopy

Interactions between the algal and fungal cells were examined by light microscopy using an inverted microscope with DIC function (DMi8, Leica). DIC images were taken of the algae-fungi aggregates after 6 day co-cultivation.

### Scanning Electron Microscopy

SEM was performed to investigate the physical interaction between N. oceanica and fungi at the Center for Advanced Microscopy of Michigan State University (CAM, MSU). Algae-fungi aggregates were collected after 6-day co-culture of the alga N. oceanica with M. elongata (AG77 and NVP64) or M. americana 3668S and were fixed in 4% (v/v) glutaraldehyde solution, followed by drying in a critical point dryer (Model 010, Balzers Union). The samples were then mounted on aluminum stubs with high vacuum carbon tabs (SPI Supplies), and were coated with osmium using a NEOC-AT osmium coater (Meiwafosis). The samples were observed with a JSM-7500F scanning electron microscope (Japan Electron Optics Laboratories).

### Confocal Microscopy

Confocal microscopy was carried out to visualize and briefly quantify lipid droplets in the alga and fungi. The samples were stained with 10 μg mL⁻¹ BODIPY 493/503 (ThermoFisher Scientific) in PBS buffer for ˜30 min at 23° C. After two washes with PBS buffer, the samples were observed using an Olympus Spectral FV1000 microscope at CAM, MSU. An argon (488 nm) laser and a solid-state laser (556 nm) were used for BODIPY (emission, 510 to 530 nm) and chloroplast (emission, 655 to 755 nm) fluorescence. N. oceanica DGTT5 fused to the cerulean fluorescent protein was overproduced using the EF promotor (Zienkiewicz et al., Biotechnology for biofuels 10:8 (2017)). The presence of the fluorescent protein in the DGTT5ox strains was detected by confocal microscopy (emission 420-440 nm) using a LSM 510 Meta Confocal Laser Scanning Microscope (Zeiss).

### Lipid Extraction and Analysis

For lipid extraction, log phase N. oceanica cells grown in f/2 medium were collected by centrifugation (4,000 g for 5 min). To test lipid content in different media, Mortierella fungi grown in PDB medium were washed twice with different media: PDB medium, pH7.6; f/2 medium with 1% glucose; f/2 medium. The cells were incubated in the respective medium for 48 h and were subsequently collected for lipid extraction by centrifugation (3,000 g for 3 min). For total lipid extraction, algae-fungi aggregates were collected by mesh filtration and frozen in liquid nitrogen prior to grinding with mortar and pestle. The fine powders were transferred to a pre-weighed and -frozen glass tube and total lipids were extracted with methanol-chloroform-88% formic acid (1:2:0.1 by volume) on a multi-tube vortexer (1,500 g for ˜20 min; Benchmark Scientific), followed by addition of 0.5 volume of 1 M KCl and 0.2 M H₃PO₄. After phase separation by centrifugation (2,000 g for 3 min), total lipids were collected for TAG separation and fatty acid analysis. The solids were dried at 80° C. overnight to provide the non-lipid biomass.

TAG was separated by TLC using G60 silica gel TLC plates (Machery-Nagel) developed with petroleum ether-diethyl ether-acetic acid (80:20:1 by volume). An internal standard of 5 μg of tridecanoic acid (C13:0) or pentadecanoic acid (C15:0) was added to each tube containing TAG or total lipid. FAMEs were then prepared with 1 M methanolic HCl at 80° C. for 25 min, and were phase separated with hexane and 0.9% NaCl and nitrogen-dried and resuspended in ˜50 μL of hexane. Gas chromatography and flame ionization detection (Agilent) were used to quantify the FAMEs in TAG and total lipid as described (Liu et al., Bioresource technology 146:310-316 (2013)) [64]. Dry weight of algae-fungi biomass was obtained by summing up non-lipid and total lipid mass.

### Chlorophyll Measurement

N. oceanica cells were collected by centrifugation from 1 mL culture aliquots during prolonged-incubation in the ePBRs. Chlorophyll of the pelleted cells was extracted with 900 μL of acetone:DMSO (3:2, v/v) for 20 min with agitation at 23° C., and measured with an Uvikon 930 spectrophotometer (Kontron) (Du et al., The Plant cell 30(2):447-465 (2018)).

### Prediction of Fatty Acid and TAG Pathways

The sequenced genome of M. elongata AG77 (Uehling et al. Environmental microbiology 19(8):2964-2983 (2017)) was annotated for genes and proteins likely involved in the synthesis of fatty acids, PUFAs, and TAGs using by BLAST searches against KOG and KEGG databases at the JGI fungal genome portal MycoCosm M. elongata AG77 v2.0 and by comparison to previously published annotations of lipid pathways of Mortierella alpina (Wang et al. PloS one 2011, 6(12):e28319.

### Abbreviations

ARA: arachidonic acid; DGTT5: a gene encoding the type II acyl-CoA:diacylglycerol acyltransferase 5; DHA: docosahexaenoic acid; DW: dry weight; EF: elongation factor gene; EPA: eicosapentenoic acid; ePBR: environmental photobioreactor; FAMEs: fatty acid methyl esters; GC-FID: gas chromatography and flame ionization detection; PDAT: phospholipid:diacylglycerol acyltransferase; PDB: potato dextrose broth; PUFAs: polyunsaturated fatty acids; S2 to S8: days 2 to 8 after the culture reached stationary phase; SEM: scanning electron microscopy; TAG: triacylglycerol; TLC: thin layer chromatography.

### Example 6: N. oceanica Cells are Captured by the M. elongata Mycelium

This Example describes experiments illustrating that N. oceanica cells are captured by the M. elongata mycelium.

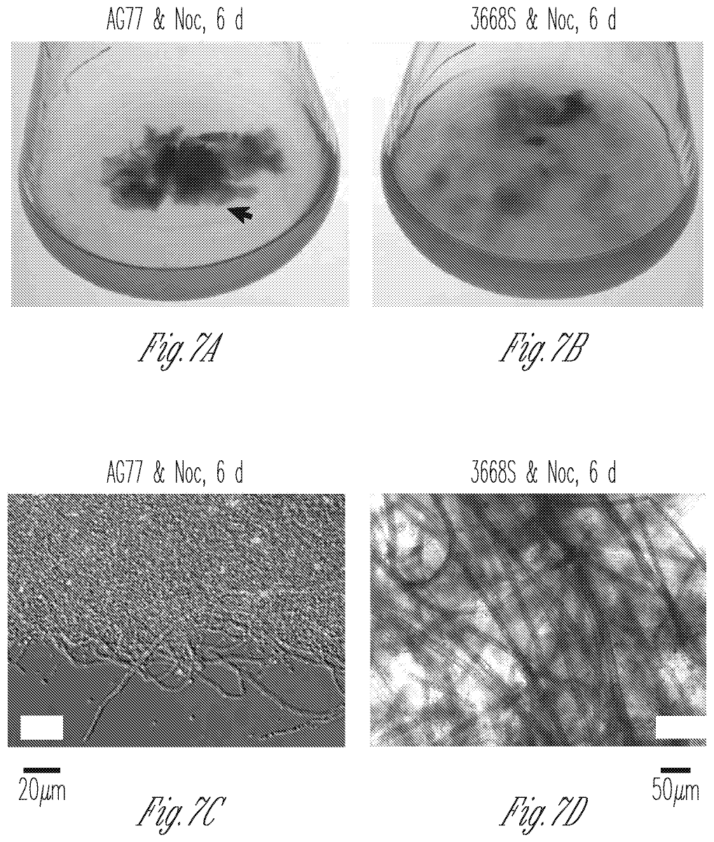

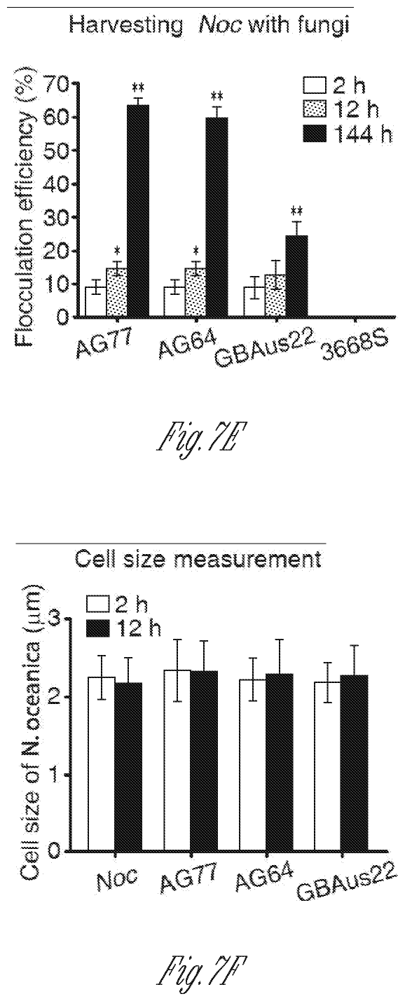

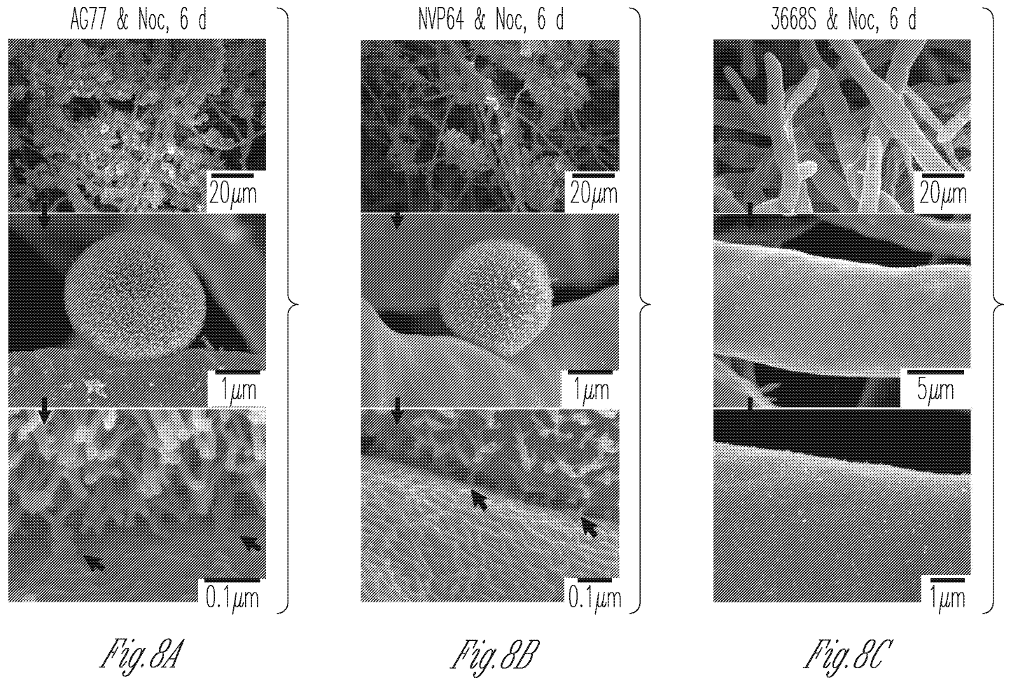

Fungi were incubated in potato dextrose broth (PDB). Fungal mycelium (˜3 times of algal biomass) was added to the N. oceanica culture containing log-phase cells in f/2 medium. After 6-days co-cultivation with M. elongata, N. oceanica cells aggregated in dense green clumps along the mycelium of the fungus (FIG. 7A). The interaction of N. oceanica with filamentous fungi appeared specific to M. elongata, as it was not observed in co-culture with Morchella americana 3668S (FIG. 7). Differential interference contrast (DIC) light microscopy showed dense numbers of N. oceanica cells attached to the M. elongata mycelium (FIG. 7C); in comparison, mycelium of M. americana hardly captured any algal cells (FIG. 7D). Three Mortierella strains, M. elongata AG77, M. elongata NVP64, and M. gamsii GBAus22 were used to test flocculation efficiency for harvesting of N. oceanica with M. americana as a negative control. All three Mortierella isolates aggregated ˜10% of algal cells after 2-hour co-culture and up to ˜15% after 12 h (FIG. 7E). After 6-day cocultivation, M. elongata AG77 and NVP64 captured ˜60% of algal cells M. gamsii GBAus 22 captured ˜25%. The short period of co-cultivation with fungi did not appear to affect the morphology of the algal cells and did not significantly change their diameter (FIG. 7F).

### Example 7: Physical Interaction Between the Cell Walls of N. oceanica and Mortierella Fungi

This Example illustrates physical interaction between N. oceanica and Mortierella elongata.

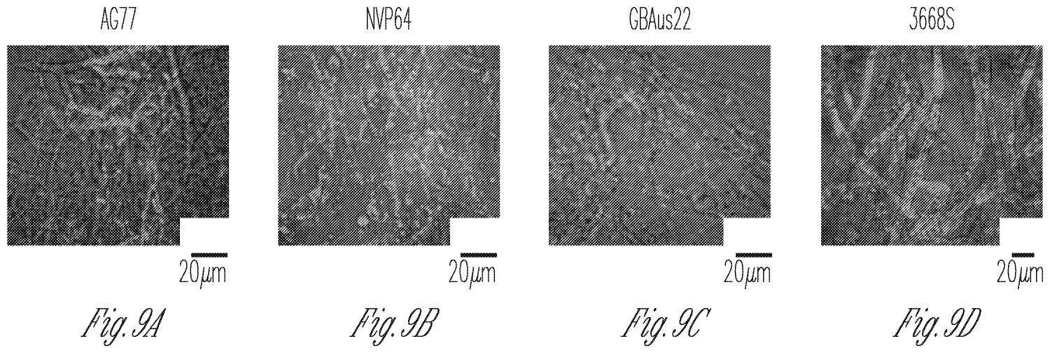

Scanning electron microscopy (SEM) was performed to investigate the physical interaction between N. oceanica and M. elongata strains AG77 (FIG. 8A) and NVP64 (FIG. 8B). Low magnification images (FIG. 8, top panels) showed an aggregation of algal cells around the fungal mycelium as seen in the light micrographs (FIG. 8C). Higher magnification images displayed details of the physical interaction between the alga and fungi (FIG. 8, middle and bottom panels). Similar to the cell wall structure of N. gaditana (Scholz et al. Eukaryotic cell 13(11):1450-1464 (2014)), N. oceanica has extensions on the outer layer of the cell wall, which are attached to the rugged surface of the fungal hyphae; irregular tube-like structures are formed between the algal and fungal cell walls, which very likely contribute to anchoring the algal cells to the mycelium. The M. americana strain 3668S, which has much thicker hyphae (10-20 μm in diameter) than the M. elongata strains AG77 and NVP64 (<2 μm), showed no obvious capture of N. oceanica cells (FIG. 8C) or flocculation.

### Example 8: Flocculation of N. oceanica with Mortierella Fungi Increases the Yield of TAG and PUFAs

This Example illustrates that increased TAG and PUFA yield is obtained when N. oceanica flocculates with Mortierella fungi.

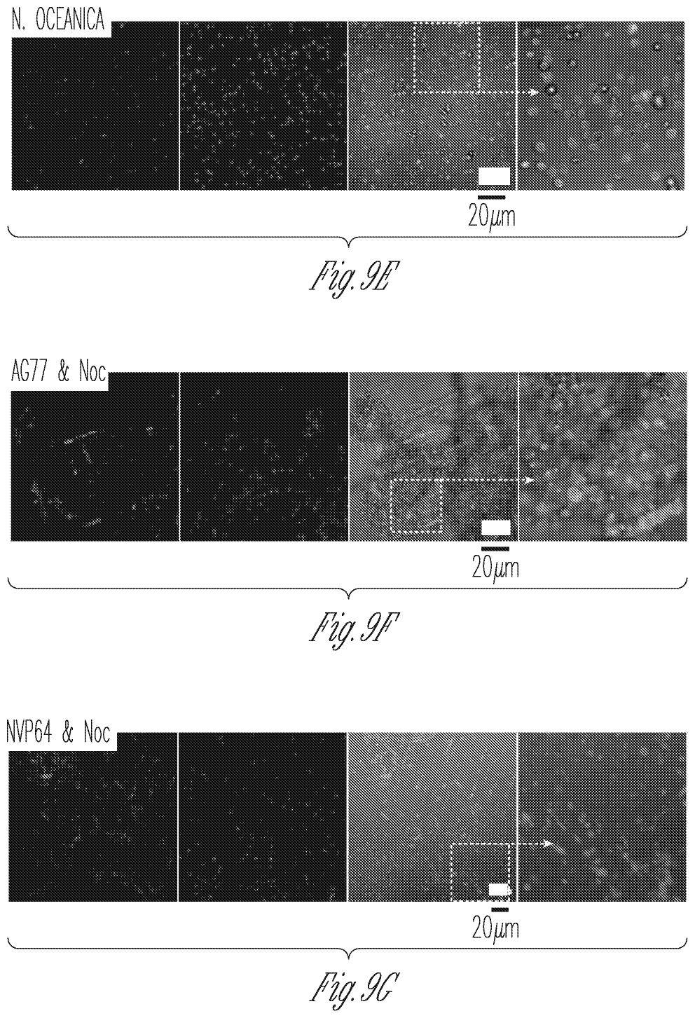

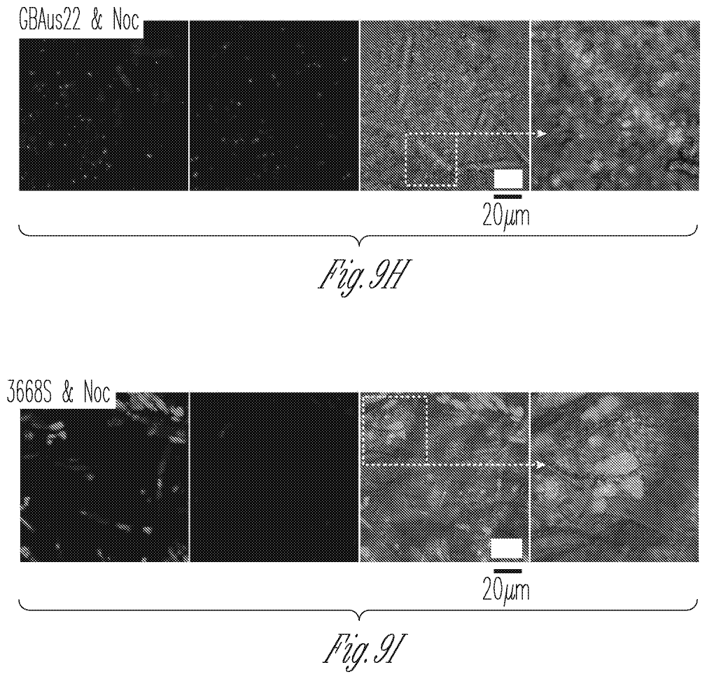

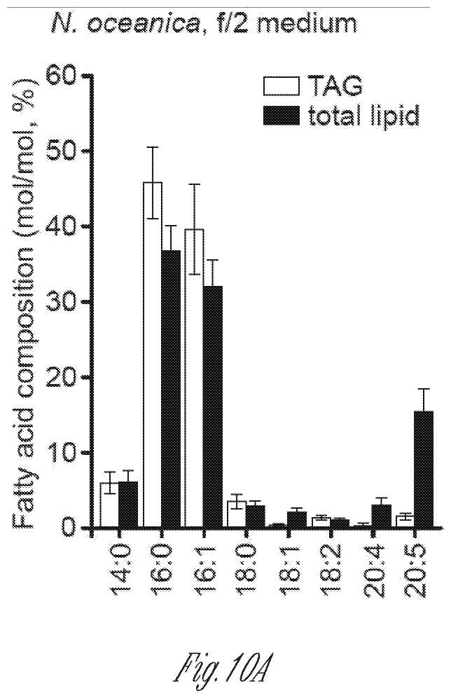

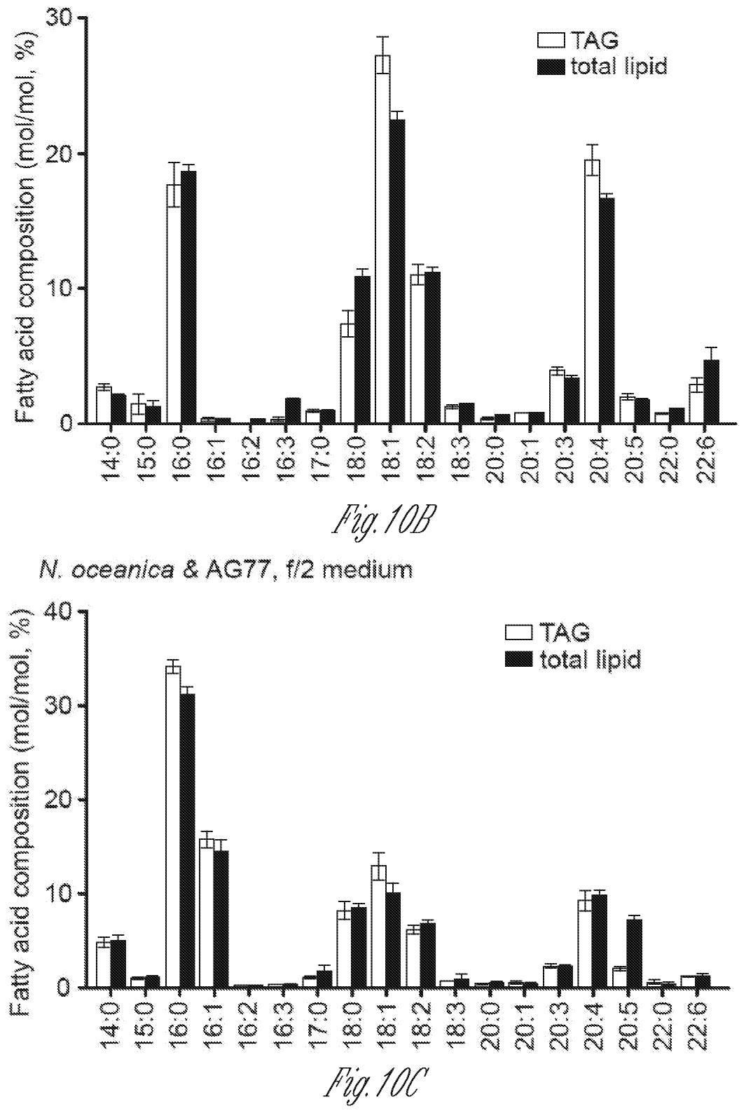

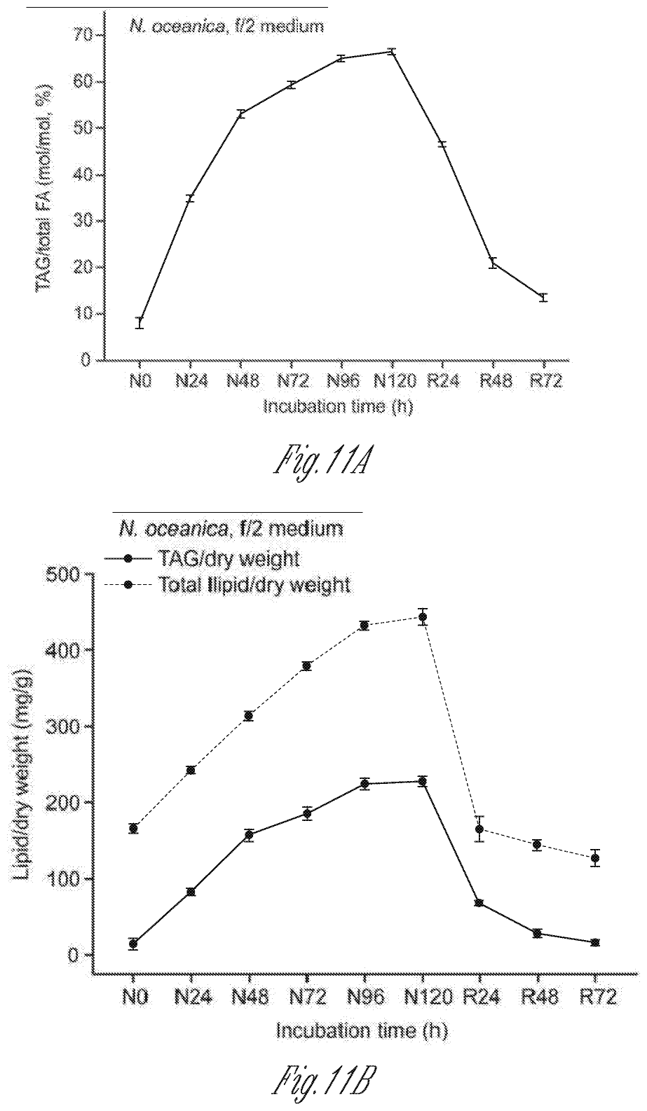

Mortierella fungi can produce TAG and PUFAs including ARA (Sakuradani et al. Applied microbiology and biotechnology 84(1):1-10 (2009); Ji et al., Critical reviews in biotechnology 34(3):197-214 (2014)). Indeed, numerous lipid droplets were observed in both Mortierella and Morchella fungi tested for alga flocculation (FIG. 9A-9D). In contrast, N. oceanica had fewer and smaller lipid droplets when grown in nutrient-sufficient f/2 medium with or without fungi (FIG. 9E-9I).

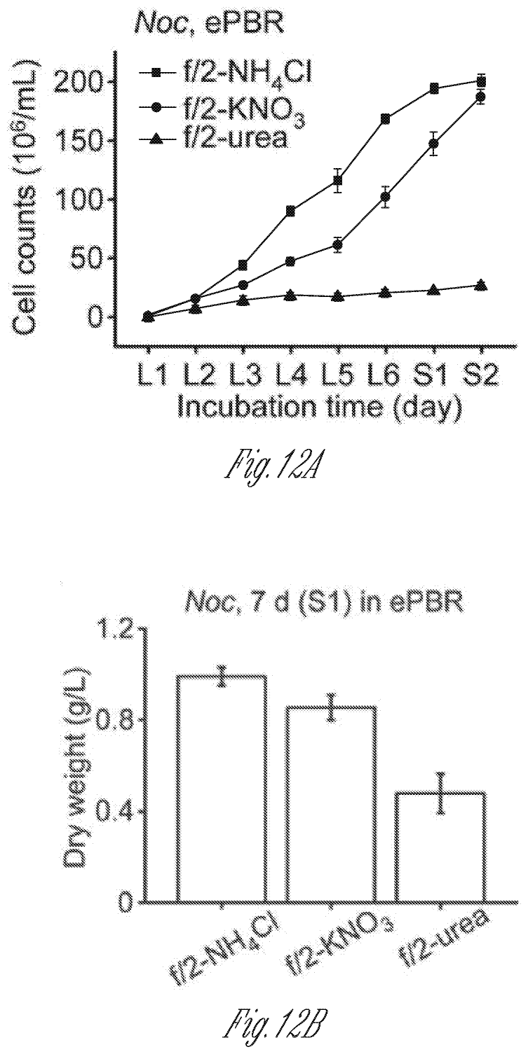

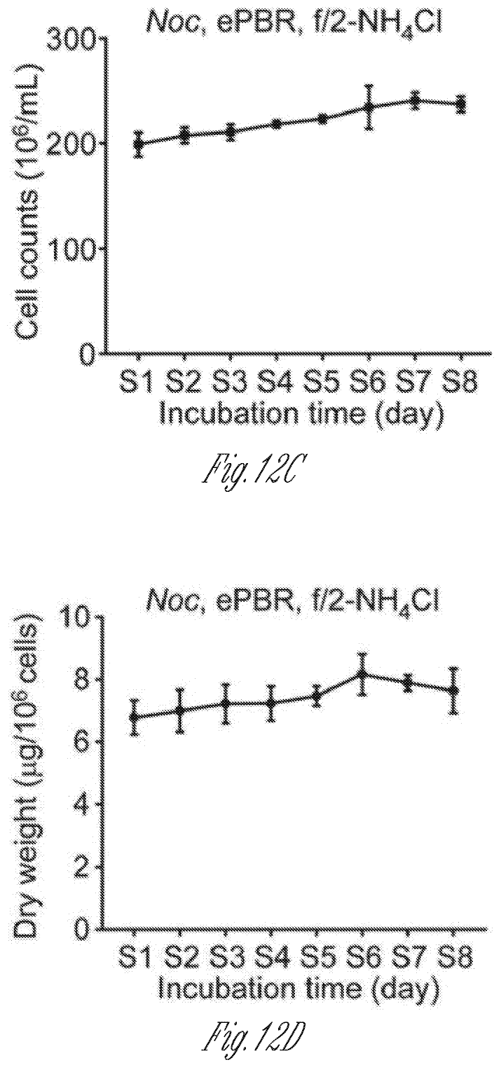

Lipids were extracted and separated by thin-layer chromatography (TLC) and fatty acid methyl esters were quantified by gas chromatography and flame ionization detection (GC-FID) to determine the lipid and fatty acid composition. As shown in Table 1, M. elongata AG77 and M. gamsii GBAus22 had much higher content of TAG, ARA, total PUFAs and total fatty acids but less EPA compared to N. oceanica, which affects the final yield of these compounds in the alga-fungus aggregate. N. oceanica TAG is mainly composed of saturated and monounsaturated fatty acids such as C16:0 and C16:1 (FIG. 10A), whereas Mortierella fungi have more PUFAs, especially ARA (FIG. 10B). N. oceanica has more EPA in total lipid than in TAG (FIG. 10A), and the alga-fungus aggregate contains ˜10% ARA and ˜7% EPA of total lipid (FIG. 10C).

TABLE 1
Lipid contents of different strains grown in f/2 medium (mg g⁻¹ total dry weight).

| Strains | Total fatty acid | TAG | ARA | EPA | Total PUFAs |
|---|---|---|---|---|---|
| N. oceanica | 118.7 ± 18.4 | 15.1 ± 2.3 | 3.1 ± 0.5 | 17.0 ± 2.6 | 21.5 ± 3.3 |
| M. elongata AG77 | 238.8 ± 14.5 | 94.6 ± 4.5 | 42.4 ± 2.3 | 4.3 ± 0.5 | 89.1 ± 4.8 |
| M. gamsii GBAus 22 | 178.0 ± 23.9 | 54.9 ± 3.9 | 29.3 ± 2.1 | 1.7 ± 0.5 | 66.1 ± 2.2 |
| M. elongata AG77 & N. oceanica | 168.5 ± 8.9 | 62.1 ± 3.0 | 16.3 ± 1.1 | 12.0 ± 0.9 | 46.5 ± 3.7 |
| M. gamsii GBAus22 & N. oceanica | 163.3 ± 10.5 | 42.0 ± 9.5 | 17.5 ± 1.7 | 9.0 ± 1.4 | 36.1 ± 6.1 |

Compared to regular PDB medium, f/2 medium has a high salt concentration and an elevated pH (pH=7.6) and lacks sugar (Guillard RRL (ed.): Culture of phytoplankton for feeding marine invertebrates. New York, USA.: Plenum Press 1975)).

M. elongata AG77 and M. gamsii GBAus22 were incubated in different media to test the impact on lipid metabolism of high pH (PDB medium, pH 7.6), high pH and high salinity (f/2+1% sugar), and high pH and high salinity with sugar starvation (f/2 medium). These adverse conditions generally increased the TAG and total lipid content of M. elongata AG77 and M. gamsii GBAus22, especially under high salinity condition (PDB pH7.6 compared to f/2+1% sugar) (Table 2). Compared to M. gamsii GBAus22, M. elongata AG77 showed a significant increase in TAG and total lipid under high pH (PDB, from pH 5.3 to 7.6), and a lower increase in total lipid, and slight decrease in TAG, upon sugar starvation (f/2+1% sugar compared to f/2) (Table 2). These adverse conditions reduced the content of ARA and total PUFAs in M. gamsii GBAus22, while EPA increased upon high pH but decreased under high salinity and sugar starvation (Table 2). In contrast, M. elongata AG77 had increased content of ARA and PUFAs in response to sugar starvation but these fatty acids decreased under high pH and high salinity conditions; EPA of M. elongata AG77 was decreased under all stress conditions compared to regular growth condition (Table 2).

TABLE 2
Lipid and fatty acid contents of Mortierella fungi incubated
in different media in shaker flasks (mg g⁻¹ total dry weight).

| Strains | Total lipid | TAG | ARA | EPA | PUFAs |
|---|---|---|---|---|---|
| M. elongata AG77, PDB, pH 5.3 | 128.2 ± 11.9 | 15.3 ± 1.0 | 27.9 ± 1.3 | 6.14 ± 0.8 | 78.9 ± 1.3 |
| M. elongata AG77, PDB, pH 7.6 | 170.2 ± 17.6 | 31.8 ± 2.0 | 25.2 ± 3.1 | 1.7 ± 1.1 | 48.9 ± 2.9 |
| M. elongata AG77, f/2 + 1% sugar | 233.2 ± 21.8 | 106.1 ± 12.3 | 15.5 ± 0.2 | 3.0 ± 0.1 | 41.5 ± 1.1 |
| M. elongata AG77, f/2 | 238.8 ± 14.5 | 94.6 ± 4.5 | 42.4 ± 2.3 | 4.3 ± 0.5 | 89.1 ± 4.8 |
| M. gamsii GBAus22, PDB, pH 5.3 | 101.2 ± 13.6 | 5.3 ± 1.4 | 33.8 ± 2.4 | 2.09 ± 0.08 | 69.9 ± 0.9 |
| M. gamsii GBAus22, PDB, pH 7.6 | 108.9 ± 12.5 | 11.7 ± 1.4 | 31.7 ± 1.4 | 2.9 ± 0.2 | 58.3 ± 1.8 |
| M. gamsii GBAus22, f/2 + 1% sugar | 139.4 ± 12.5 | 34.7 ± 4.4 | 16.4 ± 1.6 | 2.1 ± 0.2 | 39.0 ± 3.1 |
| M. gamsii GBAus 22, f/2 | 178.0 ± 23.9 | 54.9 ± 3.9 | 29.3 ± 2.1 | 1.7 ± 0.5 | 66.1 ± 2.2 |

TAG, triacylglycerol; ARA, arachidonic acid (20:4); EPA, eicosapentaenoic acid (20:5); PUFAs, polyunsaturated fatty acids; f/2 + 1% sugar, f/2 medium supplemented with 1% glucose, pH 7.6. Results are the average of five biological replicates with error bars indicating standard deviations.

### Example 9: Increasing TAG Content in N. oceanica Cells Using Ammonium as the Nitrogen Source

This Example illustrates that TAG content in N. oceanica cells using ammonium as the nitrogen (N) source.

It has been reported that TAG is the major compound for transitory carbon storage in N. oceanica cells grown under light/dark cycles (Poliner et al. The Plant journal: for cell and molecular biology 83(6):1097-1113 (2015)). However, the TAG content was relatively low when cells were grown under regular conditions (Vieler et al. PLoS genetics 8(11):e1003064 (2012); Jia et al. Algal Research 7:66-77 (2015)). Indeed, N. oceanica cells produced much less and smaller lipid droplets than the fungi apparent in confocal micrographs (FIG. 10).

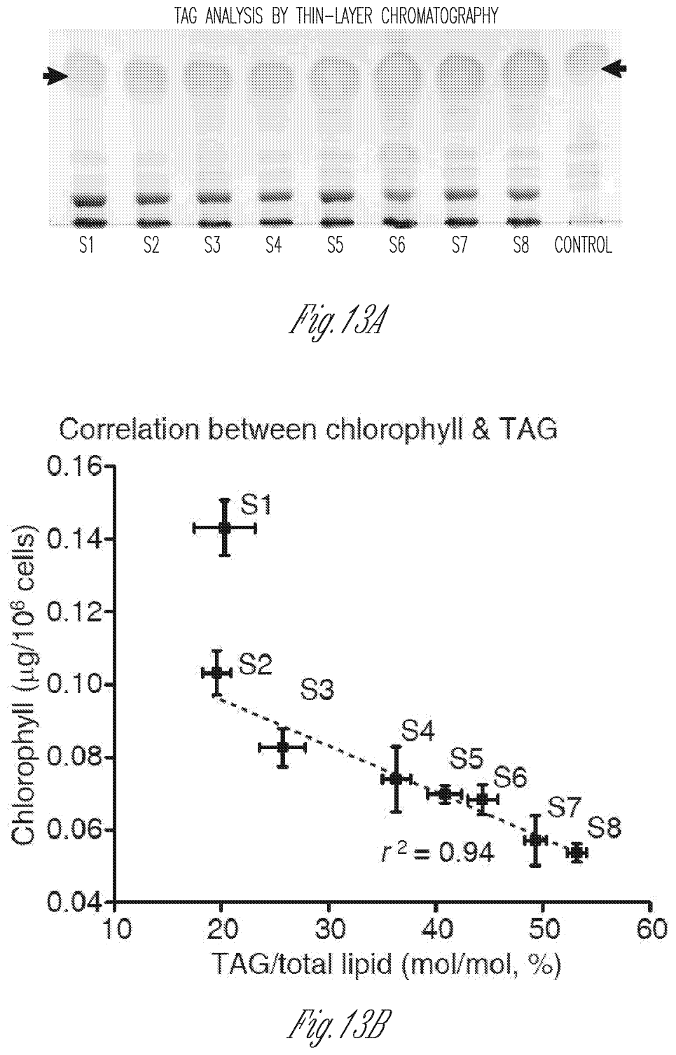

To increase TAG yield in N. oceanica, two approaches were employed: nutrient deprivation and genetic engineering. Nitrogen deprivation is one of the most efficient ways to promote TAG synthesis in microalgae. Following 120-hour nitrogen deprivation in shaker flasks, TAG accumulated in N. oceanica accounted for up to about 70% of the total lipid fraction (FIG. 11A), which is over 20% of DW (FIG. 11B). The content of TAG quickly increased following nitrogen deprivation and decreased following nitrogen resupply, indicating that N. oceanica cells are very sensitive to nitrogen supply (FIG. 11). Under laboratory conditions, nitrogen deprivation of algal cultures can be performed by centrifugation to pellet the algal cells, followed by washes and resuspension in N-deprived medium. However, this approach is not practical during scale up for industrial purposes.

A limited nitrogen supply culturing method was developed for large-volume cultures to induce TAG accumulation largely without compromising growth and biomass yields. To mimic natural cultivation conditions for N. oceanica, such as an open-pond system, environmental photobioreactors (ePBRs) were used to grow the alga under varying light (0 to 2,000 μmol photons m⁻² s⁻¹) under long-day (14/10 h light/dark) cycles, and 5% CO₂ was sparged at 0.37 L min⁻¹ for 2 minutes per hour at 23° C. (similar to FIG. 6). Illumination in the ePBR is provided by a high power white LED light on top of a conical culture vessel (total height of 27 cm) containing 330 mL of algal culture (20 cm in depth), which was designed to simulate pond depths from 5 to 25 cm (Lucker et al. Algal research 2014, 6:242-249 (2014)). Several nitrogen sources were tested in f/2 medium for the incubation of N. oceanica including set amounts of ammonium, nitrate, or urea.

Compared to nitrate and urea, N. oceanica grew faster in the f/2-NH₄Cl medium (FIG. 12A). The dry weight (DW) of N. oceanica cells per liter was also higher in the f/2-NH₄Cl culture after 7-day incubation in the ePBR (FIG. 12B). Intriguingly, the cells grown in f/2-NH₄Cl medium turned from vivid green to yellow following 7 days of incubation once they reached stationary phase, indicative of chlorophyll degradation in the algal cells.

Lipid analysis by TLC (FIG. 13A) and GC-FID (FIG. 13B) demonstrated that TAGs had accumulated during days 2 to 8 after the culture reached stationary phase (incubation time S2 to S8), which is correlated with chlorophyll degradation, while cell density and dry weight remained at similar levels during this period (FIG. 12C-12D). Previously, to prevent carbon limitation, NaHCO₃ was added N. oceanica cultures in shaker flasks (Vieler et al., Plant Physiology 158(4):1562-1569 (2012)). Addition of NaHCO₃ prevented acidification in cultures, which were sparged with 5% CO₂(FIG. 14A). N. oceanica cells accumulated more TAG upon acidification in the culture medium without NaHCO₃ supply, especially from S6 to S8, compared to the NaHCO₃ culture (FIG. 12C-12D).

### Example 10: Fatty Acid and TAG Synthesis Pathways in M. elongata AG77

The genome of N. oceanica CCMP1779 has been sequenced and analyzed for the presence of metabolic pathway genes for PUFA and TAG biosynthesis (Vieler et al., PLoS genetics 8(11):e1003064 (2012)), information used in the genetic engineering for increased EPA content (Poliner et al., Plant biotechnology journal 16(1):298-309 (2018)). For Mortierella fungi, nuclear transformation methods were established (Takeno et al. Journal of bioscience and bioengineering 2005, 100(6):617-622 (2005); Ando et al., Current genetics 55(3):349-356 (2009)), and the M. elongata AG77 genome has been sequenced and annotated (Uehling et al., Environmental microbiology 19(8):2964-2983 (2017)), but lipid metabolic pathways have not yet been reconstructed.

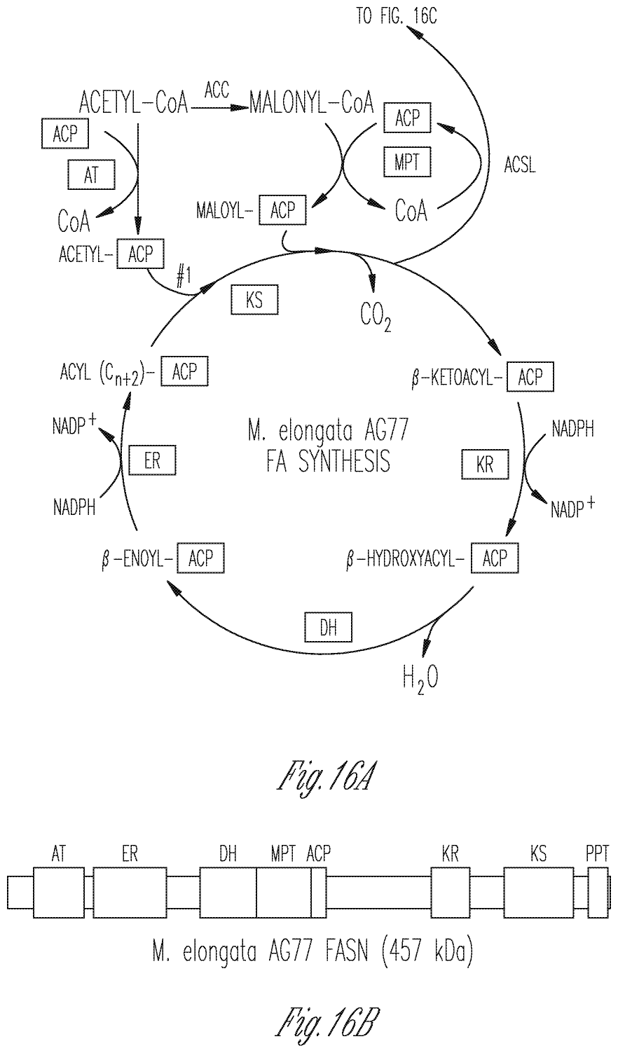

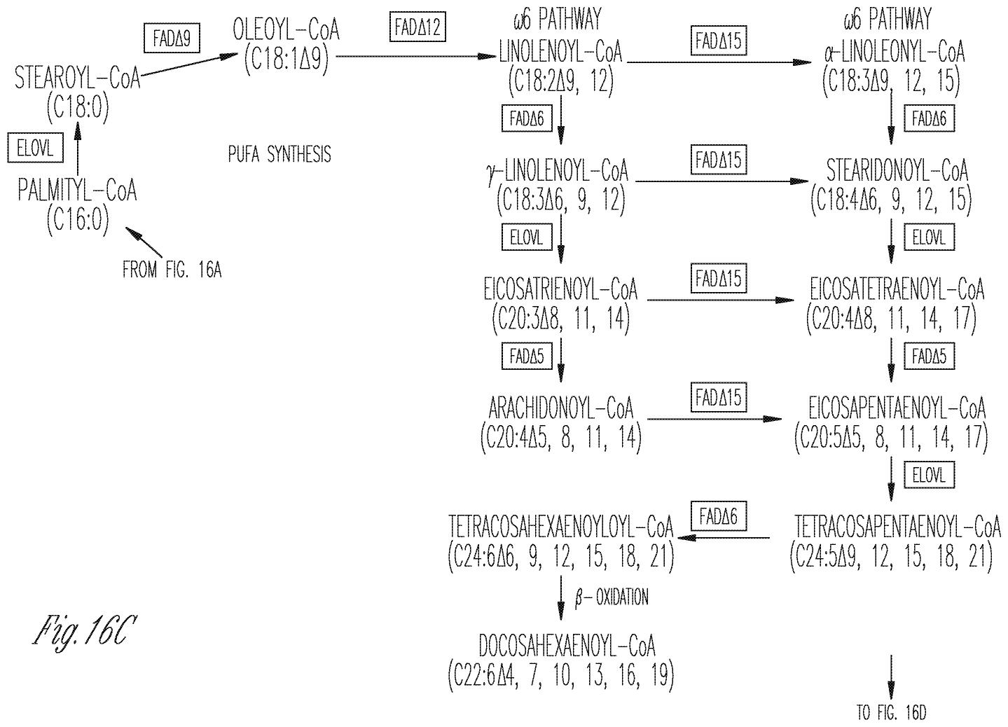

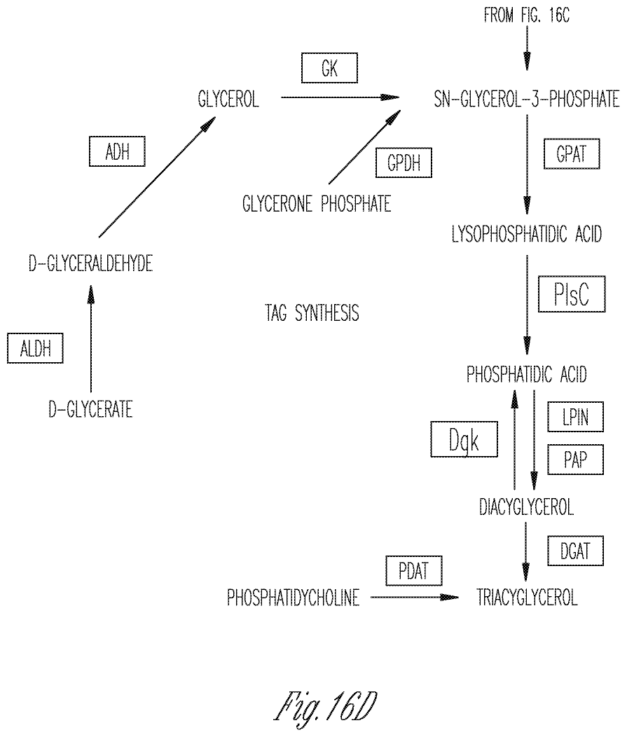

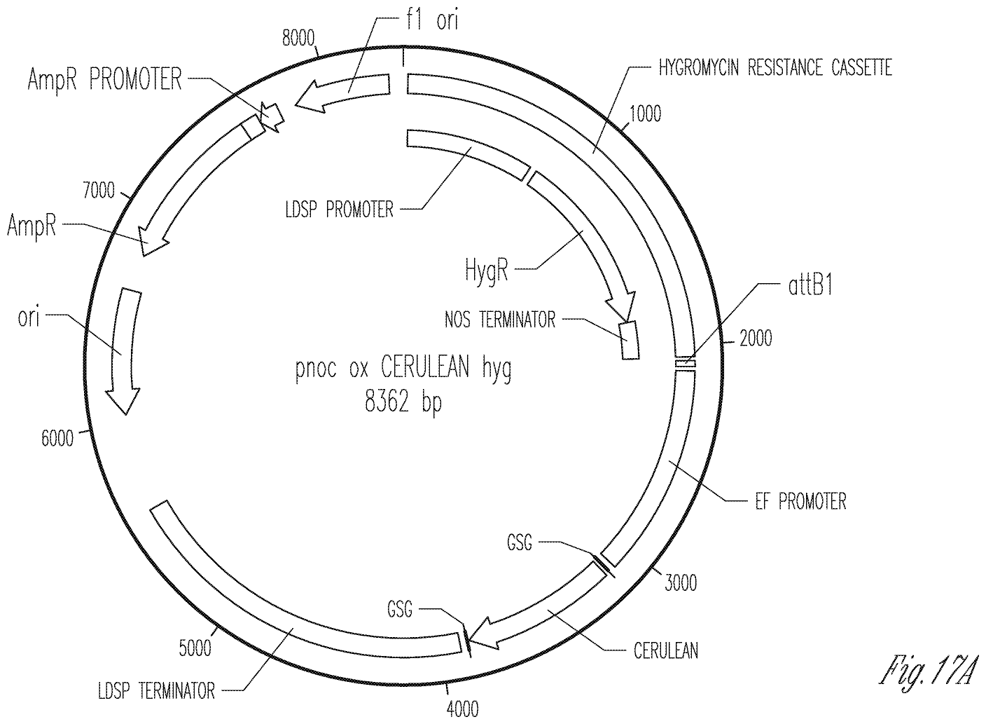

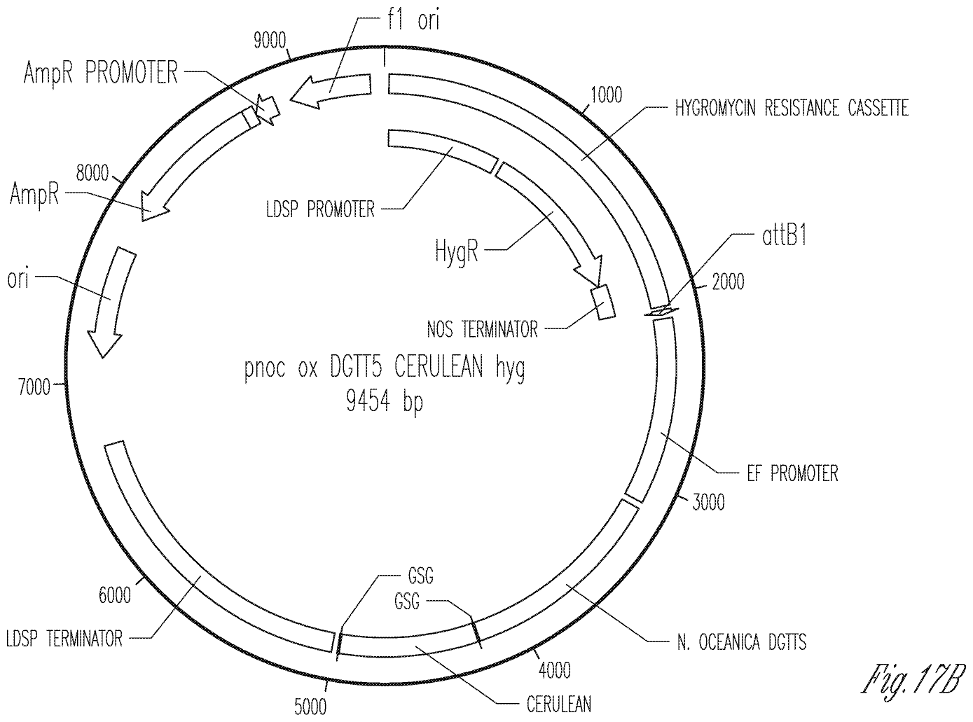

Thus, the inventors applied the genome browser and BLAST tools from the JGI fungal genome portal MycoCosm to predict fatty acid, PUFA, and TAG synthesis pathways for M. elongata AG77. The fatty acid synthesis pathway (FIG. 16A) was predicted according to gene candidates (Table 3).

TABLE 3
Fatty acid and TAG Synthetic Genes and Proteins involved in
fatty acid and glycerolipid synthesis in M. elongata AG77.

| | | | |
|---|---|---|---|
| Description | Name | Transcript | Protein ID |

Fatty Acid Biosynthesis

| | | | | |
|---|---|---|---|---|
| Acetyl-CoA | acetyl-CoA carboxylase | ACC | 134167 | 133928 |
| carboxylase | acetyl-CoA carboxylase, subunit beta | ACC | 67410 | 67171 |
| components | acetyl-CoA carboxylase, subunit beta | ACC | 75685 | 75446 |
| | acetyl-CoA carboxylase, subunit beta | ACC | 75799 | 75560 |
| | malonyl-CoA decarboxylase | MLYCD | 100665 | 100426 |
| | malonyl-CoA decarboxylase | MLYCD | 81573 | 81334 |
| | acyl carrier protein | ACP | 128202 | 127963 |
| | acyl carrier protein | ACP | 139468 | 139229 |
| Type I fatty acid | fatty acid synthase | FAS | 1805138 | 1804883 |
| putative fatty acid | malonyl-CoA:ACP | FabD | 144910 | 144671 |
| synthase components | malonyl-CoA:ACP | FabD | 522882 | 522643 |
| | 3-oxoacyl-ACP synthase, KASI/II | FabB/F | 115244 | 115005 |
| | 3-oxoacyl-ACP synthase, KASI/II | FabB/F | 1878602 | 1878347 |
| | 3-hydroxydecanoyl-ACP dehydratase | FabA | 131674 | 131435 |
| | putative 3-Ketoacyl-ACP reductase | FabG | 1769266 | 1769011 |
| Elongases | acyl-CoA elongase | ELO | 132697 | 132458 |
| | acyl-CoA elongase | ELO | 134272 | 134033 |
| | acyl-CoA elongase | ELO | 140756 | 140517 |
| | acyl-CoA elongase | ELO | 141020 | 140781 |
| | acyl-CoA elongase | ELO | 14820 | 14581 |
| | acyl-CoA elongase | ELO | 147783 | 147544 |
| | acyl-CoA elongase | ELO | 148635 | 148396 |
| | acyl-CoA elongase | ELO | 165821 | 165582 |
| | acyl-CoA elongase | ELO | 1880273 | 1880018 |
| Desaturases | fatty acid Δ9-desaturase | FADS9 | 107360 | 107121 |
| | fatty acid Δ9-desaturase | FADS9 | 108744 | 108505 |
| | fatty acid Δ9-desaturase | FADS9 | 138135 | 137896 |
| | fatty acid Δ9-desaturase | FADS9 | 1816261 | 1816006 |
| | fatty acid Δ6-desaturase | FADS6 | 134789 | 134550 |
| | fatty acid Δ6-desaturase | FADS6 | 158522 | 158283 |
| | fatty acid desaturase | FAD | 140331 | 140092 |
| | fatty acid desaturase | FAD | 1751385 | 1751130 |
| | fatty acid desaturase | FAD | 15652 | 15413 |
| | fatty acid Δ12-desaturase | FADS12 | 17302 | 17063 |
| | fatty acid Δ5-desaturase | FADS5 | 87849 | 87610 |
| | fatty acid Δ15-desaturase | FADS15 | 152410 | 152171 |
| Acyl-CoA thioesterase | acyl-CoA thioesterase | ACOT | 14633 | 14394 |
| and synthetase | acyl-CoA thioesterase | ACOT | 54405 | 54166 |
| | acyl-CoA thioesterase | ACOT | 561278 | 561039 |
| | acyl-CoA thioesterase | ACOT | 33252 | 33013 |
| | acyl-CoA synthetase | ACSL | 123145 | 122906 |
| | acyl-CoA synthetase | ACSL | 134960 | 134721 |
| | acyl-CoA synthetase | ACSL | 143367 | 143128 |
| | acyl-CoA synthetase | ACSL | 75546 | 75307 |
| | acyl-CoA synthetase | ACSL | 131674 | 131435 |
| | acyl-CoA synthetase | ACSL | 150818 | 150579 |
| | acyl-CoA synthetase | ACSL | 72538 | 72299 |
| | acyl-CoA synthetase | ACSL | 74248 | 74009 |
| | acyl-CoA synthetase | ACSL | 81012 | 80773 |
| | acyl-CoA synthetase | ACSL | 94221 | 93982 |
| | acyl-CoA synthetase | ACSL | 126107 | 125868 |
| | acyl-CoA synthetase | ACSL | 73494 | 73255 |

Glycerolipid biosynthesis

| | | | | |
|---|---|---|---|---|
| | aldehyde dehydrogenase | ALDH | 14282 | 14043 |
| | aldehyde dehydrogenase | ALDH | 138532 | 138293 |
| | aldehyde dehydrogenase | ALDH | 138027 | 137788 |
| | aldehyde dehydrogenase | ALDH | 145556 | 145317 |
| | aldehyde dehydrogenase | ALDH | 36004 | 35765 |
| | aldehyde dehydrogenase | ALDH | 34024 | 33785 |
| | alcohol dehydrogenase | ADH | 103662 | 103423 |
| | alcohol dehydrogenase | ADH | 144920 | 144681 |
| | alcohol dehydrogenase | ADH | 157172 | 156933 |
| | alcohol dehydrogenase | ADH | 80690 | 80451 |
| | alcohol dehydrogenase | ADH | 150046 | 149807 |
| | alcohol dehydrogenase | ADH | 36977 | 36738 |
| | alcohol dehydrogenase | ADH | 21055 | 20816 |
| | alcohol dehydrogenase | ADH | 84445 | 84206 |
| | glycerol kinase | GK | 95496 | 95257 |
| | glycerol-3-phosphate dehydrogenase | GPDH | 141744 | 141505 |
| | glycerol-3-phosphate dehydrogenase | GPDH | 133004 | 132765 |
| | glycerol-3-phosphate dehydrogenase | GPDH | 143386 | 143147 |
| | glycero-3-phosphate acyltransferase | GPAT | 132665 | 132426 |
| | glycero-3-phosphate acyltransferase | GPAT | 71699 | 71460 |
| | glycero-3-phosphate acyltransferase | GPAT | 136092 | 135853 |
| | glycero-3-phosphate acyltransferase | GPAT | 426195 | 425956 |
| | glycero-3-phosphate acyltransferase | GPAT | 114545 | 114306 |
| | glycero-3-phosphate acyltransferase | GPAT | 156906 | 156667 |
| | glycero-3-phosphate acyltransferase | GPAT | 142242 | 142003 |
| | glycero-3-phosphate acyltransferase | GPAT | 138636 | 138397 |
| | 1-sn-acyl-glycero-3-phosphate acyltransferase | PlsC | 133934 | 133695 |
| | 1-sn-acyl-glycero-3-phosphate acyltransferase | PlsC | 15247 | 15008 |
| | phosphatidic acid phosphatase | PAP | 72762 | 72523 |
| | phosphatidic acid phosphatase | PAP | 67757 | 67518 |
| | phosphatidic acid phosphatase | PAP | 118493 | 118254 |
| | phosphatidic acid phosphatase | PAP | 143215 | 142976 |
| | phosphatidic acid phosphatase | PAP | 141373 | 141134 |
| | Lipin like/phosphatidate phosphatase | LPIN | 22296 | 22057 |
| | Lipin like/phosphatidate phosphatase | LPIN | 33916 | 33677 |
| | diacylglycerol kinase | Dgk | 32027 | 31788 |
| | diacylglycerol kinase | Dgk | 143293 | 143054 |
| | diacylglycerol kinase | Dgk | 133967 | 133728 |
| | diacylglycerol kinase | Dgk | 111955 | 111716 |
| | diacylglycerol kinase | Dgk | 133379 | 133140 |
| | diacylglycerol kinase | Dgk | 134894 | 134655 |
| TAG synthesis | diacylglycerol acyltransferase | DGAT | 102618 | 102379 |
| | diacylglycerol acyltransferase | DGAT | 14740 | 14501 |
| | diacylglycerol acyltransferase | DGAT | 135508 | 135269 |
| | phospholipid diacylglycerol acyltransferase | PDAT | 872488 | 872249 |

M. elongata AG77 has a type-I fatty acid synthase with a similar domain organization as found in yeast (FIG. 16B). Nine elongases and twelve desaturases were identified within the M. elongata AG77 genome for PUFA synthesis, including a Δ15 fatty acid desaturase (FAD) for EPA synthesis (FIG. 16C, Table 3). Three DGATs and one PDAT (phospholipid:diacylglycerol acyltransferase) were present in the M. elongata AG77 genome, which is similar to what was reported for M. alpina (Wang et al., PloS one 6(12):e28319 (2011)).

### Example 11: Sequences of Some Lipid Synthesizing Enzymes

Amino acid and nucleic acid sequences for lipid synthesizing enzymes are available from various databases including the National Center for Biotechnology Information (see website at ncbi.nlm.nih.gov), and UNIPROT (see website at uniprot.org). Such databases provide both amino acid and nucleic acid sequences for lipid synthesizing enzymes. Some examples of lipid synthesizing enzyme sequences are provided below.

A sequence for Mortierella elongata AG-77 acetyl-CoA carboxylase with protein ID 133928 is shown below as SEQ ID NO:7 (Uniprot A0A197K7T6).

```
                10 20 30 40
MTSNVQSFIG GNALDKAPAG AVHDFVSQHG GHSVITKILI
                50 60 70 80
ANNGIAAVKE IRSVRKWAYE TFGDERAIQF TVMATPEDLK
                90 100 110 120
VNAEYIPMAD QYVEVPGGSN NNNYANVDLI VDIAERTGVH
              130 140 150 160
AVWAGWGRAS ENPKLPESLR DSPQKIIFIG PPGSAMRSLG
              170 180 190 200
DKISSTIVAQ SADVPTMGWS GTGITETEMD PNGFVTVPED
              210 220 230 240
AYQAACVTDA EDGLKKAHAI GFPIMIKASE GGGGKGIRKV
              250 260 270 280
EDPEKFAQAF HQVLGEVPGS PVFIMKLAGN APHLEVQLLA
              290 300 310 320
DQYGHAISLF GRDCSVQPPE QKIIEEAPVT IAKPDTFEAM
              330 340 350 360
EKAAVRLAKL VGYVSAGTVE YLYSHATDTY FFLELNPRLQ
              370 380 390 400
VEHPTTEIVS GVNLPAAQLQ IAMGLPLNRI KDIRVLYGLQ
              410 420 430 440
PSGTSEIDFE FAQQVSFETQ RKPAPKGHVI AVRITAENPD
              450 460 470 480
AGFKPSSGMM HDLNFRSSTN VWGYFSVSSA GGLHEFADSQ
              490 500 510 520
FGHIFAYGQD RGQSRKNMVV ALKELSIRGD FRTTVEYLIR
              530 540 550 560
LLETQEFEEN TINTGWIDSL ISNNLTAERP ETMLAVMCGA
              570 580 590 600
VNRAHTISEN CLKEYKKSLE KGQIPSKDVL RSVNQLDFIY
              610 620 630 640
DGVRYNFTAT RSGPNSYTMY LNGSMISISV RPLTDGGLLV
              650 660 670 680
LLDGKAHTTY SLEEVQATRL MVDGKTCLLE KENDPTQLRS
              690 700 710 770
PSPGKLVRFL VESGDHVKAS QAYAEIEVMK MYMPLIATED
              730 740 750 760
GIVQFIKQPG TTLDAGDIIG ILSLDDPSRV KHAKPFEGQL
              770 780 790 800
PPMGQPTIHG AKPHQPYREL RLILDNAMDG YDNQAIVQPT
              810 82 830 840
LKEIFEVLQT PELPYLEFNE VFAALSGRIP PKLEISLHQE
              850 860 870 880
VDQSMKNHEH FPARTLQALI DAHCRANFSK PADVSSFLAS
              890 900 910 920
VAPLTTIIQE YQTGLKTHSW TFIAHYLTKY HEVESLFDDS
              930 940 950 960
AREEETILAI RDQYKDDVEK VINIALSHSR VTAKNNLVLS
              970 980 990 1000
LLDQIKPTSS GGAIDKFFSP ILKKLAELNG RLTSKVSLKA
            1010 1020 1030 1040
RELLIHVQLP SFEERQAQME KILRSSVTEE IYGGDHEARM
            1050 1060 1070 1080
PNYDNLKELV DTTYTVFDVL PNFFYHESAH VRLAAFEVYC
            1090 1100 1110 1120
RRAYHAYEIL DINYHMEHNP LLITWKFLLN TPNKSSEGGP
            1130 1140 1150 1160
NRVASVSDMS YLINKADPEP VRTGGILAVR DIKELEGRFQ
            1170 1180 1190 1200
SVLDFFPTVK SNKHLAHVQA TSVHNNVINV VLKSESIHPN
            1210 1220 1230 1240
DDDYWLNLLS PIVKGQSEHL RSHGIRRMTF LIFRQGNYPS
            1250 1260 1270 1280
YFTFRERNNY AEDQTIRHIE PAMAYRLELS RLSNFDIKPC
            1290 1300 1310 1320
FIDNRQVHVY YAVGKENVSD CPFFVCALVR PGRLRSSVRT
            1330 1340 1350 1360
ADYLISETDR LLNDILDALE IVGATYKQSD CNHLFINFIP
            1370 1380 1390 1400
TFQIDATEVE SALKGFIDRH GKPLWRLRVT GAEIPFNVQS
            1410 1420 1430 1440
KNDAADPIPL RFIISNVSGY VLNVDTYREI OTDKGAIFKS
            1450 1460 1470 1480
VGPSGPFHLL PVNQPYPTKE WLQPRRYKAH LMGTTYVYDF
            1490 1500 1510 1520
GELFRQAVRA QWNHAVKVNP SLKAPNQVLE MRELVLDEKQ
            1530 1540 1550 1560
QLQQVVREAG SNNCGMVAWI FTLRTPEYPE GRQIIVIAND
            1570 1580 1590 1600
ITYNIGSFGP EEDLVFYKAS ELARKIGIPR VYLSANSGAR
            1610 1620 1630 1640
IGLASEVIGL FNSCWNDASN PSKGFKYIYL TDAGLKQLEA
            1650 1660 1670 1680
QEERSGKKSV LTETVVEDGE TRHKITDVIG AVDGLGVENL
            1690 1700 1710 1720
RGSGLIAGET SRAYDDIFTI TLVTCRSVGI GAYLVRLGQR
            1730 1740 1750 1760
TIQNEGQPII LTGAPALNKL LGRDVYTSNL QLGGTQIMYK
            1770 1780 1790 1800
NGVSHLTAQN DYEGIGKIVN WLSYIPERKN APVPITVSND
            1810 1820 1830 1840
TWDRDIDYLP PKGAVYDPPW LIGGKDAEEE CAAFQTGFED
            1850 1860 1870 1880
KGSFTETLTG WARTVVVGRA RLGGVPMGVI AVETRSVEHI
            1890 1900 1910 1920
IPADPANGDS VEQVLMEAGN VWYPNSAYKT AQAINDFNKG
            1930 1940 1950 1960
EQLPLMIFAN WRGFSGGQRD MYNEILKYGS FIVDALSSYK
            1970 1980 1990 2000
QPVEVYVVPN GELRGGAWVV VDPTINENMM EMYADKRSRA
            2010 2020 2030 2040
GVLEPEGIVE IKFRKAQLLA TMERLDDKYR DLKAQYEKPD
            2050 2060 2070 2080
LAGADREAIK TKLTEREQEL LPVYQQLAIQ FADLHDTAGR
            2090 2100 2110 2120
MKAKGTIRES LDWTNARRYF YWRVRRRIAE EYIRRRMTIA
            2130 2140 2150 2160
SKTQTRDDQT ATLKAWFGRD TVHASEAELT QIWEHEDRVV
            2170 2180 2190 2200
LEWFEGQSRK VDALIQELTA AGTAEEVVRM YTSDRAGVVE
            2210 2220
GFDRILQSLS DQEKQDILAK FATMTV
```

A sequence for Nannochloropsis oculate acetyl-CoA carboxylase is shown below as SEQ ID NO:8 (NCBI AHI17198.1).

```
      1 MATTIPSSNR RAMRAGAALV AVSSILVLLM GPVAEAWRVP
    41 GFGQGRSSGV TKPVHAPGFL GRFSTPSSLG PSSASCPTIS
    81 AVGPLSAATM APPALSPEAQ KKKDAVAAYV KSRGGNLAIR
  121 KVLIANNGMA ATKSILSMRQ WAYMELGDDR AIEFVVMATP
  161 EDLNANAEFI RLADRFVEVP GGSNKNNYAN VDLIVQMAQR
  201 EGVDAVWPGW GHASENPRLP NTLKQLGIKF IGPTGPVMSV
  241 LGDKIAANIL AQTAKVPSIP WSGDGLTAEL TAEGTIPDET
  281 FQKAMVRTSE EALAAANRIG YPVMLKASEG GGGKGIRMSN
  321 NDKELETNFI QVQNEVPGSP MFMMQLCTQA RHIEVQIVGD
  361 EHGNAAALNG RDCSTQRRFQ KIFEEGPPTI VPPEVEKQME
  401 LAAQRLTQSI GYIGAGTVEY LFNAATGKYF FLELNPRLQV
  441 EHPVTEGLSL VNLPATQLQI AMGIPLNRIP DIRRFYGKDD
  481 PYGDSPIDFF NDDYAELPSH VIAARITAEN PDEGFKPTSG
  521 RIERVKFQST ANVWGYFSVG ANGGIHEYAD SQFGHLFAKG
  561 KSREDARKSL VLALKEIEVR GDIRTTVEYL VQLLETEAFK
  601 ENTIDTSWLD GLIREKSVRV ELNPHDVALS AAIARAFARS
  641 VDEERKFVEN LSKGQVSIQG IRSINSFPME ITYKDYKYSF
  681 HCTRVGPDKL RLAINDQILE TKVRQQPDGS LIAEFGGTTH
  721 TIYALEEPLG LRMVLDGVTV LLPTVYDPSE LRTDVTGKIV
  761 RYLQEDGTEI QAGQPYVEVE AMKMIMPLKA TESGTVAHRL
  801 SPGSIITAGD LLANVQLKDP SKVKKITPFK GALELVGSDD
  841 EPGVTGFQAV LKTMNMVLDG YDYEVEFLAQ NLVTSAQDGK
  881 ELLDAATALV TKYLAVEEQF AGKVLDEAMV GLVKANKDSL
  921 PTVLALATAH RELPRRNKMV SALIRQLQAL VERSSNDLSL
  961 DTLIALLDRA SRLPGKEYGE VAISSAQALL ALRAPPFSTR
1001 QDELRTTLLN TKDNDALARS ATLTAGVDLL TAMFTDPDAN
1041 VRKNAIEVYI RRIYRAHRIL SLTVEEVDGV MIANWSFKFA
1081 DTPDEESPLR RGFFTVFPSL EAYTAGSEKF SKVLKTALAG
1121 QEAYSQPINV FHVAVAQLPE SQQPEVIANI EGILAENKDL
1161 LTECRVRMVN VLFVQGAKNP RYFTFTAVKD FKEDPLRRDM
1201 RPTFPQLLEL SRLAANYELQ RLPSIGRNTQ VYLGSERAPV
1241 GTKKRGPGNQ VLFVRGISHS EQTQTPMGAE RVLLMAMDEL
1281 DYALLDERVG GSASSRLFLN LLVPIDSDPK TLAGEWSKIM
1321 DRLLAKYATR LLKLGVDEIE IKVRVAAGSG SAITPVRLMA
1361 SSMTGEFLRT DAFLEYPDPV TGITKQFCSV TSEDQVCLLN
1401 PYPASNSIQT RRASARRIGS TYAYDFLGVM EVSLIQKWDK
1441 HLKELTSVYT SRVDDKMPEQ LFQADELVLE DGVLKPTQRL
1481 VGLNDVGMVA WHATMKTPEY PEGRELVIIA NDVTFQSGSF
1521 GVKEDDFFRA ASEYARVRGL PRIYLSSNSG ARIGLVDDLK
1561 GKFRIAWNDP ANPSLGFKYL YLTPEEYEGL KPGTVNANLV
1601 LSEEGEKRWA LQDIIGQVHG IGVENLRGSG MIAGETSRAY
1641 DETFTLSYVT GRSVGIGAYL VRLGQRTIQM VNGPLILTGY
1681 SALNKLLGRE VYTSQDQLGG PQIMAPNGVS HLVVDNDKEG
1721 ISSIIDWLSF VPKDKFSSVP IIDLPTDSPE RDVEFQPTKT
1761 PYDPRHMLAG TVGPDGAFVP GFFDRGSFIE TLGGWGKSVV
1801 TGRAKLGGIP MGIISVETRL VEQRIPADPA NPESRESLLP
1841 QAGQVWYPDS AFKTAQAIED FNRGENLPLM IFANWRGFSG
1881 GTRDMYGEIL KFGAKIVDAL RTYRHPVFVY IPPNGELRGG
1921 AWVVIDPTIN EEMMEMYADK DSRGGILEPP GICEVKFRAA
1961 DQISAMHRLD PVIQALDGEL QNAKTEADAI KLKQQLKERE
2001 EALLPLYMQV AHEFADLHDR AGRMKAKGVI RDVVTWKRSR
2041 SYFYWRARRR VAEDGLVRAM QKADASLSVQ DGREKLEALA
2081 TSGVYGDDKA FVAWVTESGS KIEEQLVSVK HAAVKASLAS
2121 LLEELSPEER KKVLSGL
```

A sequence for Nannochloropsis gaditana CCMP526 acetyl-CoA carboxylase is shown below as SEQ ID NO:9 (Uniprot I2CQP5).

```
                10 20 30 40
MASFPPSNRR ATPARVMVVI FSSVLILLAG PVGDAWRMPS
                50 60 70 80
IAPGQSTGVA KTSRWAGFLG NFARRSPSIS TSPSLPPSLP
                90 100 110 120
ASSLGPLSAA TMAPPSTLSP AAQKKKDAVA AYVKSRGGNL
              130 140 150 160
GIRKVLIANN GMAATKSILS IRQWAYMELG DDKAIEFVVM
              170 180 190 200
ATPEDLNANA EFIRLADRFV EVPGGSNKNN YANVDLIVQV
              210 220 230 240
AEREGVDAVW PGWGHASENP RLPNTLKEMG IKFIGPTGPV
              250 260 270 280
MSVLGDKIAA NILAQTAKVP SIPWSGDGLT AELTAEGTIP
              290 300 310 320
DETFQKAMVR TAEEALAAAN RIGYPVMLKA SEGGGGKGIR
              330 340 350 360
MSNNDEELKN NFVQVSNEVP GSPMFMMQLC TQARHIEVQI
              370 380 390 400
VGDEHGNAAA LNGRDCSTQR RFQKIFEEGP PTIVPPEVFK
              410 420 430 440
QMELAAQRLT QSIGYIGAGT VEYLFNAATG KYFFLELNPR
              450 460 470 480
LQVEHPVTEG LSLVNLPATQ LQIAMGIPLN RIPDIRRFYG
              490 500 510 520
KEDPYGDSPI EFFEDDYADL ASHVIAARIT AENPDEGFKP
              530 540 550 560
TSGRIERVKF QSTANVWGYF SVGANGGIHE FADSQFGHLF
              570 580 590 600
AKGKTREDAR KSLVLALKEI EVRGDIRTTV EYLVQLLETD
              610 620 630 640
AFKENTIDTS WLDGLIREKS VRVELAPHEV ALSAAIARAF
              650 660 670 680
ARSQEEEKKF VENLGKGQVS IQSIRSINSF PMEITYKDSK
              690 700 710 720
YSFLCSRIGP DKLRLTINGQ VLETKVRQQP DGSLIAEFGG
              730 740 750 760
TTHTIYALEE PLGLRMVLDG VTVLLPTVYD PSELRTDVTG
              770 780 790 800
KVVRYLQDDG AEIQAGQPYV EVEAMKMIMP LKASESGTVT
              810 820 830 840
HRLSPGSIIT AGDLLANIQL KDPSKVKKII PFKDTLELAG
              850 860 870 880
SGEEPGTTEI ESVLKTMNLV LDGFDYEVEF LAQNLVTSVR
              890 900 910 920
DGKELLDAAV ALVSKYLAVE EQFAGKALDE AMVALVKANK
              930 940 950 960
ESLGTVLQLA TAHRELPRRN KMVSALIRQL QALVERPGTS
              970 980 990 1000
ELALGPLIDL LERTSHLPGK EYGEVAISSA QALLALKAPP
            1010 1020 1030 1040
FNIRKDELRA TLMQTQDNDA LARSATLTAG VDLLTAMFTD
            1050 1060 1070 1080
PDVTVRKNAI EVYIRRIYRA HRILSLSVEE VDGVMVARWS
            1090 1100 1110 1120
FKFADTPDEE SPLRYGFFTV FPSLEAYTEG TEKFSKVLKS
            1130 1140 1150 1160
SLGGKEVYSE PTNVFHVAVA QLPESDQPEV IANIEAILAE
            1170 1180 1190 1200
KKELLTECQV RMVNVLFVKG ASNPRYYTFT AAENFKEDPL
            1210 1220 1230 1240
RRDMRPTFPQ LLELSRLAAN YELQRLPSIG RNTQVYLGTE
            1250 1260 1270 1280
RAAAGVKKRG GSQVLFVRGI SHSEQTQTPL GAERVLLMAM
            1290 1300 1310 1320
DELDYALLDP RVGGSASSRL FLNLLVPITT DPEALAGEWN
            1330 1340 1350 1360
QVMDRLLAKY ATRLLKLGVD EIEIKVRVTA DGNTITPVRL
            1370 1380 1390 1400
MATSMTGEFL RTDAFLEYPD PVNGITKQFC SITREDQICL
            1410 1420 1430 1440
LNPYPASNSI QTRRASARRI GSTYAYDFLG VMEVSLIQKW
            1450 1460 1470 1480
DKHLKELSSV YPSRVDDKMP EQLFTAHELV LEDDELQPTQ
            1490 1500 1510 1520
RLVGLNDIGM IAWHATMKTP EYPEGRELVI IANDVTFQSG
            1530 1540 1550 1560
SFGVKEDEFF RAASEYARVR GLPRIYLSSN SGARIGLVDD
            1570 1580 1590 1600
LKGKFRIAWN DPANPSLGFK YLYLPPEEYE ALKPGTVNAN
            1610 1620 1630 1640
LVETEEGEKR WALQDIVGQV HGIGVENLRG SGMIAGETSR
            1650 1660 1670 1680
AYDETFTLSY VTGRSVGIGA YLVRLGQRTI QMVNGPLILT
            1690 1700 1710 1720
GYSALNKLLG REVYTSQDQL GGPQIMAPNG VSHLVVGNDK
            1730 1740 1750 1760
EGVSSIIDWL SFVPKDKFSA PPILDLPTDS PERDVEFLPT
            1770 1780 1790 1800
KTPYDPRHML AGTVGPDGAF VPGFFDRGSF IETLGGWGKS
            1810 1820 1830 1840
VVTGRAKLGG IPMGVISVET RLVEQRVPAD PANPDSRESI
            1850 1860 1870 1880
LPQAGQVWYP DSAFKTAQAM EDFNRGENLP LIIFANWRGF
            1890 1900 1910 1920
SGGTRDMFGE ILKFGAKIVD ALRTYRHPVF VYIPPNGELR
            1930 1940 1950 1960
GGAWVVIDPT INEEMMEMYA DKDSRGGILE PPGICEVKFR
            1970 1980 1990 2000
NADQVSAMHR LDPVIQALDG ELQNAKTEQD AAKLTQQLKE
            2010 2020 2030 2040
REEALLPLYT QVAHEFADLH DRAGRMKAKG VIRDVVTWKR
            2050 2060 2070 2080
SRSYFFWRAR RRIAEDGLIR EMQRVDPTLS VQQGREKVSA
            2090 2100 2110 2120
LASPAVYEDD KAFVAWVEEG GEAIAKELEK IKQAAVKASL
            2130
ASLLEGLSAE ERKQVLAGL
```

A sequence for a Streptococcus salivarius acetyl-CoA carboxylase beta subunit is shown below as SEQ ID NO:10 (NCBI WP_014633943.1).

```
    1 MGLFDRKEKY IRINPNRSVR NGVDHQVPEV PDELFAKCPG
  41 CKQAIYQKDL GQAKICPNCS YTFRISAKER LDLTVDEGSF
  81 QELFTGIKTE NPLNFPGYME KLAATKEKTG LDEAVVTGFA
121 SIKGQKTALA IMDSNFIMAS MGTVVGEKIT KLFEHAIEEK
161 LPVVIFTASG GARMQEGIMS LMQMAKISAA VKRHSNAGLL
201 YLTVLTDPTT GGVTASFAME GDIILAEPQT LIGFAGRRVI
241 ENTVRETLPD DFQKAEFLQE HGFVDAIVKR TELADTIATL
281 LSFHGGVQ
```

A sequence for a Collimonas fungivorans acetyl-CoA carboxylase beta subunit is shown below as SEQ ID NO:11 (NCBI AMO95008.1).

```
    1 MYRTDLESNI HVCPKCDHHM RIRARERLDA LLDAGGRYEI
  41 GQETLPIDTL KFKDSKKYPD RLKAAMDATG ETDALIVLGG
  81 SIMTLPVVVA AFEFEFMGGS MGSVVGERFV RGAQVALEQK
121 VPFICITATG GARMQEGLLS LMQMAKTTSM LTKLSEKKLP
161 FISVLTDPTM GGVSASFAFM GDVVIAEPKA LIGFAGPRVI
201 ENTVREKLPE GFQRAEFLVT KGAVDMIVDR RKMREEIARL
241 LALLQDQPVE SIA
```

A sequence for a Marinobacter sp. acetyl-CoA carboxylase beta subunit is shown below as SEQ ID NO: 12 (Uniprot A0A2G1ZII3).

```
                10 20 30 40
MSNWLDKIMP SKIRSESKQR TGVPEGLWKK CPKCGAFLYK
                50 60 70 80
PELDKNLDVC PKCQHHLRIT ARRRLDVFLD ADGRQEIAAD
                90 100 110 120
LEPWDRLKFK DSKRYKDRLS QNQKTTGEKD ALVAMRGACL
              130 140 150 160
DIPLVAVAFE FNFLGGSMGQ VVGEKFVQAA NVCLEERIPL
              170 180 190 200
VCFSASGGAR MQEAILSLMQ MSKTAAVLER MKQEGIPYIS
              210 220 230 240
VMTDPVFGGV SASLAMLGDL NIAEPYALIG FAGPRVIEQT
              250 260 270 280
VREKLPEGFQ RSEFLLEHGA IDMILHRHQM RERIAAVLAK
              290 300
FTDLDQPATE APIEFEVSER PETDVPAE
```

A sequence for Helicosporidium ex Simulium jonesi acetyl-CoA carboxylase beta subunit (plastid) is shown below as SEQ ID NO: 13 (NCBI ABD33968.1).

```
    1 MTILAWIKDK KNKAILNTPE YSSQSSLSWC FTHKEAASNK
  41 AVSFINLSKR RALWTRCEKC GMIQFMRFFK ENANLCLSCS
  81 YHHIMTSDER IALLVEKGTW YPLNETISPK DPIKFTDTQS
121 YAQRIQSTQE KLGMQDAVQT GTGLINGIPF AIGIMDFRFM
161 GGSMGSVVGE KLTRLIEYAT KQGLFLLIVS ASGGARMQEG
201 IYSLMQMAKI SAALNVYQNE ANLLYISLCT SPTTGGVTAS
241 FAMLGDIIFS EPEAIIGFAG RRVIQQTLQQ ELPEDFQTSE
281 SLLHHGLIDA IVPRCFLVNA ISEVASIFAY APSKYKKLGN
321 ISHYHENTLS WATEEILRRN CINNKKVEYR TIEKIYQTTL
361 YKESFFRLNK LLSKLKSEIN FTNKMKKQNN AFNTSSVYAN
401 YYDVMLCNYN IGTHSLNLLF NEESEFCKYF PFNMDHMKKE
441 NRIKYNFITE NSNDFIRKKT INDFSIMLIG D
```

A sequence for Mortierella elongata AG-77 malonyl-CoA decarboxylase with protein ID 100426 is shown below as SEQ ID NO:14 (Uniprot A0A197JJC1).

```
                10 20 30 40
MSRRLIISHL SKPSSRVWSS SSSSSSFYSP AFSTSTTVRS
                50 60 70 80
PFHIATLQRH RTMASISNGG SNNNNNNSAS SSSNAAGSGT
                90 100 110 120
LQALRANVVE QYWNDIAAHF REPGFSTFDK ERTRRAADRD
              130 140 150 160
PEFMRKLLLA VITDRPGQGD ILPSVIAKSS CDFFSSLDRN
              170 180 190 200
GKTEFLRLLA RDFGVLQEDV VKAAEQYQDY AHKEPESKAL
              210 220 230 240
LRAEQLLRHA IVPGHSKFFD RVSRLPGGLK FLIDMRQDLL
              250 260 270 280
SIIQANKGDV YLSSLNESLK EKLQAWFVGF LDLERLTWQS
              290 300 310 320
PAVLLEKITQ YEAVHKFKDV QDLKRRVGPG RRVFALMNKS
              330 340 350 360
LPAEPLVFVQ VALVERLSDN VQDILNDPSP GHANPAETVK
              370 380 390 400
CAIFYSITTQ QPYLQWLSGI ELGNFLIKRV VRSLKVEFPQ
              410 420 430 440
IETFSTLSPI PGFRKWIGQC QNLGQKLLLP QEESIVSQLG
              450 460 470 480
QETGAASGDV EDQFSAILKH PSTFSDSETM SKLRPILSRL
              490 500 510 520
CARYILLEKR RHLALDPVAN FHLRNGACAH RLNWLGDTST
              530 540 550 560
KGMEESFGLM INYLYSLDHI EMNNQQYLLD GTISVSSKDA
              570 580 590 600
GFQKVLMDSA VGNSQAAGRG VGEEQGGEEG QVVQVNGSSF
RLLEIVTA
```

A sequence for Mortierella elongata AG-77 malonyl-CoA decarboxylase with protein ID 81334 is shown below as SEQ ID NO: 15.

```
                10 20 30 40
RYILEKKCRH LAMDSVANFH LRNGACAHRL NWLDDTSPKG
                50
MEEFFGIVTE SRRSLAD
```

A sequence for Mortierella elongata AG-77 acyl carrier protein with protein ID 127963 is shown below as SEQ ID NO: 16.

```
                10 20 30 40
MFRALVRPAS TIYRQAAIKA TPATVARMPM GLTFARTYAS
                50 60 70 80
AGLARSDVEK RVLDILAGFN KVDSNKISLN ANFNNDLGLD
                90 100 110 120
SLDTVEVVMA IEEEFSIEIP DKDADEIKSA AQAVEYITKR
DDAH
```

Another sequence for Mortierella elongata AG-77 acyl carrier protein is shown below as SEQ ID NO: 17 (Uniprot A0A197JHD1).

```
    1 MFRAIRPAAL YRSAALYKTA PAVVARNAMA LNFARTYASA
  41 GLARSDVEKR VLDILAGFNK IDANKIALKA NFNADLGLDS
  81 LDTVEVVMAI EEEFSIEIPD KDADEIKSAE QAVEYISKRE
121 DAH
```

A sequence for Nannochloropsis gaditana acyl carrier protein is shown below as SEQ ID NO: 18 (Uniprot W7TK08).

```
                10 20 30 40
MRVLAFLALL AAPAFAFVPR MPAPVRARAG LTLRFSGEYS
                50 60 70 80
EKVRAIVLEN MGDDAKVQDY LKANGDDTAE FAAMGFDSLD
                90 100 110 120
LVEFSMAVQK EFDLPDLNEE DFANLKTIKD VVTMVEANKK
```

A sequence for Nannochloropsis gaditana malonyl-ACP transacylaseis shown below as SEQ ID NO: 19 (Uniprot S5VRZ9).

```
                10 20 30 40
MMSKSLIMLG LLSPTAFAFV PKLSTNVLSR AISSHARKNL
                50 60 70 80
VKASAVDYKT AFMFPGQGAQ YVGMGAQVSE EVPAAKALFE
                90 100 110 120
KASEILGYDL LDRAMNGPKD LLDSTAVSQP AIFVASAAAV
              130 140 150 160
EKLRATEGED AANAATVAMG LSLGEYSALC YAGAFSFEDG
              170 180 190 200
VRLTKARGEA MQAAADLVDT TMVSVIGLEA DKVNELCAAA
              210 220 230 240
SSKSGEKIQI ANYLCPGNYA VSGSLKAAQV LEEIAKPEFG
              250 260 270 280
ARMTVRLAVA GAFHTEYMAP ALEKLKEVLA KTEFKTPRIP
              290 300 310 320
VISNVDGKPH SDPEEIKAIL AKQVTSPVQW ETTMNDLVKG
              330 340 350
GLETGYELGP GKVCAGILKR IDRKAKMVNI EA
```

A sequence for Mortierella elongata AG-77 fatty acid synthase is shown below as SEQ ID NO:20 (Uniprot A0A197K6H1).

```
                10 20 30 40
MESISQFIPN KLPQDLFIDF ATAFGVRAAP YVDPLEDALT
                50 60 70 80
AQMEKFFPAL VHHYRAFLTA VESPLAAQLP LMNPFHVVLI
                90 100 110 120
VIAYLVTVFV GMQIMKNFNR FEVKTFSLFH NFCLVSISAY
              130 140 150 160
MCGGILYEAY QSKYGLFENL ADHTSTGFPM AKMIWLFYFS
              170 180 190 200
KIMEFVDTMI MVLKKNNRQI SFLHVYHHSS IFAIWWLVTF
              210 220 230 240
VAPNGEAYFS AALNSFIHVI MYGYYFLSAL GFKQVSFIKF
              250 260 270 280
YITRSQMTQF CMMSVQSSWD MFAMKVMGRP GYPFFITALL
              290 300 310
WFYMWTMLGL FYNFYRKNAK LAKQAKADAA KEKSKKLQ
```

Another sequence for Mortierella elongata AG-77 fatty acid synthase is shown below as SEQ ID NO:21 (Uniprot A0A197K854).

```
                10 20 30 40
MAAAFLDQVN FSLDQPFGIK LDNYFAKGYE LVTGKSIDSF
                50 60 70 80
VFQEGVTPLS TQYEVAMWTV TYFIVIFGGR QIMKSQEAFK
                90 100 110 120
LKPLFILHNF LLTIASGALL LLFIENLVPI LARNGLFYAI
              130 140 150 160
CDQGAWTQRL ELLYYLNYLV KYWELADTVF LVLKKKPLEF
              170 180 190 200
LHYFHHSMTM ILCFVQLGGY TSVSWVPITL NLTVHVLMYY
              210 220 230 240
YYMRSAAGVR IWWKQYLTTL QIVQFVLDLG FIYFCSYTYF
              250 260 270 280
AFTYWPHLPN VGKCAGTEGA ALFGCGLLSS YLLLFINFYR
              290 300 310
LTYNAKAKAA KERGSNVTPK TPKADKKKSK HI
```

Another sequence for Mortierella elongata AG-77 fatty acid synthase is shown below as SEQ ID NO:22 (Uniprot A0A197JPT7).

```
                10 20 30 40
MESAPMPAGV PFPEYYDFFM NWKTPLAIAA TYTVAVTLFN
                50 60 70 80
PKVGKVSRVV AKSANAKPAE KTQSGAAMTA FVFVHNLILC
                90 100 110 120
VYSGITFYNM FPAMIKNFAT HSIFDAYCDT DQSLWNGSLG
              130 140 150 160
YWGYIFYLSK FYEVIDTIII ILKGRRSSLL QTYHHAGAMI
              170 180 190 200
TMWSGINYQA TPIWIFVVFN SFIHTIMYAY YAATSVGLHP
              210 220 230 240
PGKKYLTSMQ ITQFLVGMSI AVSYLFIPGC IRTPGAQMAV
              250 260 270
WINVGYLFPL TYLFVDFAKR TYSKRSAAPA KKTE
```

A sequence for Nannochloropsis gaditana fatty acid synthase is shown below as SEQ ID NO:23 (Uniprot W7TQY4).

```
                10 20 30 40 50
MGNQNSVYFG APPVRKKAPQ HADIQEAWRQ IASKVARDKG FEHGRKRKVA
                60 70 80 90 100
IIGSGVAGLG AAYHLLTCAA PGEEVELVVY EASGTPGGHA HTELVREEDG
              110 120 130 140 150
KIIACDTGFM VENHQNYPNL VELFAELGVD DENTNMSFAV SMDEGKVEWC
              160 170 180 190 200
SESVKTLAGP VYRAMIKDMI RFNRTASNLL LAEPEDPRRA WTLAEFLEKE
              210 220 230 240 250
KYGPEFTNYY IVPMCAALWS SSAADVLAAS AYALLTFMDN HCMLQIENRP
              260 270 280 290 300
QWKTVAQRSQ TYVQKIVALL GERLRLNAPV KKVVVHGKGK VEVTDASYHA
              310 320 330 340 350
ETEDEAIFAC HPDQSLALLE GEARVRLAPY LEAFKYAPNA CYLHSDPRIM
              360 370 380 390 400
PRKKEAWGSW NYIGTSAGML GPGREKPVFV TYWLNQLQNL ETETPYFVSL
              410 420 430 440 450
NPLFPPDRAL THKILRESHP QFTPATEAAQ RRMTEVQGQD GLWFCGAWMG
              460 470 480 490 500
HGFHEDGLRS GLEVATALSG QKAAWMPPEA EAPVYPMVKA HMNARSTWER
              510 520 530 540 550
CQDLLGQLAC VPIRNFLASS IQEGCLVLRL PGTGDKLWFG DRTAGRKETV
              560 570 580 590 600
VLRVQSWWFF VRVALEYDLG LARAYMAGEF EVEGTGWNSD GLTRLFLLFI
              610 620 630 640 650
RNRDAPSGGK RFAVSALLTS WIGYGLNFLR YRLSMDNSLA GSRQNISAHY
              660 670 680 690 700
DIGNDLYTLM LDKSLMMYSS AIYHLELTPS SLTASAEATS SDLVPAGNGN
              710 720 730 740 750
GVVVKSSFPP SSYSMAFKGS LEDAQLRKVD TLIRTCRVER KHILLDIGFG
              760 770 780 790 800
WGGIAIRAAE TIGCKVVGIT LSKEQKALAE EKVRAKGLEH LIHFELVDYR
VFARR
```

A sequence for a Mortierella elongata AG-77 FabD protein is shown below as SEQ ID NO:24 (Uniprot A0A197K6C6).

```
                10 20 30 40 50
MGRDLYESYP IVRQIIDEAD AILSSMPSSS SSSSPQEEGY LKRVMFEGPQ
                60 70 80 90 100
EELTRIENAQ PAILITSIAL IRVIETEHGL DIKESCRFAL GHSLGEYSAL
              110 120 130 140 150
VATRALSIPD AVRIVRIRGD AMAMAVTDKK GMTAMSALVV RASKIDELVK
              160 170 180 190 200
AMHEIQTELS STVEIAEIAN INSSFQVVIS GTVKGVDHAS KTIQFRKIAA
              210 220 230 240 250
KAVDIPVEAP FHCSLMEPAA RVMKDALADI SFKQPIIPIV SNVQAQPIES
              260 270 280 290 300
SNDIPSLIVQ QVIDIVQWRQ SIVNIHSQQQ QYDISEYICI GPGKVICNIL
              310 320
RKEYPLDTIR SVSTVEDIQQ WKL
```

A sequence for Saccharomyces cerevisiae malonyl CoA-acyl carrier protein transacylase is shown below as SEQ ID NO:25 (Uniprot Q12283).

```
                10 20 30 40 50
MKLLTFPGQG TSISISILKA IIRNKSREFQ TILSQNGKES NDLLQYIFQN
                60 70 80 90 100
PSSPGSIAVC SNLFYQLYQI LSNPSDPQDQ APKNMTKIDS PDKKDNEQCY
              110 120 130 140 150
LLGHSLGELT CLSVNSLFSL KDLFDIANFR NKLMVTSTEK YLVAHNINRS
              160 170 180 190 200
NKFEMWALSS PRATDLPQEV QKLLNSPNLL SSSQNTISVA NANSVKQCVV
              210 220 230 240 250
TGLVDDLESL RTELNLRFPR LRITELTNPY NIPFHNSTVL RPVQEPLYDY
              260 270 280 290 300
IWDILKKNGT HTLMELNHPI IANLDGNISY YIHHALDRE7 KCSSRTVQFT
              310 320 330 340 350
MCYDTINSGT PVEIDKSICF GPGNVIYNLI RRNCPQVDTI EYTSLATIDA
              360
YHKAAEENKD
```

A sequence for Nannochloropsis gaditana malonyl CoA-acyl carrier protein is shown below as SEQ ID NO:110 (Uniprot S5VRZ9).

```
                10 20 30 40 50
MMSKSLIMLG LLSPTAFAFV PKLSTNVLSR AISSHARKNL VKASAVDYKT
                60 70 80 90 100
AFMFPGQGAQ YVGMGAQVSE EVPAAKAIFE KASEILGYDL LDRAMNGPKD
              110 120 130 140 150
LLDSTAVSQP AIFVASAAAV EKLRATEGED AANAATVAMG LSLGEYSALC
              160 170 180 190 200
YAGAFSFEDG VRLTKARGEA MQAAADLVDT TMVSVIGLEA DKVNELCAAA
              210 220 230 240 250
SSKSGEKIQI ANYLCPGNYA VSGSLKAAQV LEEIAKPEFG ARMTVRLAVA
              260 270 280 290 300
GAFHTEYMAP ALEKLKEVLA KTEFKTPRIP VISNVDGKPH SDPEEIKAIL
              310 320 330 340 350
AKQVISPVQW ETTMNDLVKG GLETGYELGP GKVCAGILKR IDRKAKMVNI
EA
```

A sequence for a Pseudomonas aeruginosa beta-ketoacyl-[acyl-carrier-protein]synthase protein is shown below as SEQ ID NO:111 (NCBI accession no. Q9HU15.1).

```
    1 MSRLPVIVGF GGYNAAGRSS FHHGFRRMVI ESMDPQARQE
  41 TIAGLAVMMK LVKAEGGRYL AEDGTPLSPE DIERRYAERI
  81 FASTLVRRIE PQYLDPDAVH WHKVLELSPA EGQALTFKAS
121 PKQLPEPLPA NWSIAPAEDG EVLVSIHERC EFKVDSYRAL
161 TVKSAGQLPT GFEPGELYNS RFHPRGLQMS VVAATDAIRS
201 TGIDWKTIVD NVQPDEIAVF SGSIMSQLDD NGFGGIMQSR
241 LKGHRVSAKQ LPLGFNSMPT DFINAYVLGS VGMTGSITGA
281 CATFLYNLQK GIDVITSGQA RVVIVGNSEA PILPECIEGY
321 SAMGALATEE GLRLIEGRDD VDFRRASRPF GENCGFTLAE
361 SSQYVVLMDD ELAIRIGADI HGAVTDVFIN ADGFKKSISA
401 PGPGNYLTVA KAVASAVQIV GLDTVRHASF VHAHGSSTPA
441 NRVTESEILD RVASAFGIDG WPVTAVKAYV GHSLATASAD
481 QIISALGTEK YGILPGIKTI DKVADDVHQQ RISISNRDMR
521 QDKPLEVCFI NSKGFGGNNA SGVVLSPRIA EKMLRKRHGQ
561 AAFAAYVEKR EQTRAAARAY DQRALQGDLE IIYNFGQDLI
601 DEHAIEVSAE QVIVPGESQP LVYKKDARFS DMLD
```

A sequence for a Mortierella elongata AG-77 3-oxoacyl-[acyl-carrier-protein]synthase protein is shown below as SEQ ID NO:26 (Uniprot A0A197JR20).

```
                10 20 30 40 50
MSLNARRVVV TGLGLVTPLG IGVQQSWSKL IAGECGVVSL KDLPSPIPGI
                60 70 80 90 100
PGFDTLPSQV GAIVKRTGGK ELGGFDSTEW LDRGDEKRMA VFTQYAIAAA
              110 120 130 140 150
RMAIKDANWE TTTEEEKERT GVCLGSGIGS LDDMATTALS FAESGYRKMS
              160 170 180 190 200
PMFVPKILIN MAAGHLTMKY GFKGPNHAVS TACTTGAHSL GDAMRFIQYG
              210 220 230 240 250
DADVMVAGGS EACIHPLAVA GFAKAKSLAT KYNDSPSEAS RPFDKNRDGF
              260 270 280 290 300
VIGEGAGVVV LEEYEHAKKR GAHIYAELRG YGLSGDAHHM TAPPENGTGA
              310 320 330 340 350
AMAMRRALKA ARLTPADIGY VNAHATSTHQ GDIAENRAIK SVFDGHHDTI
              360 370 380 390 400
AVSSTKGAVG HLLGAAGAVE AIFATLAVKN NILPPTLNLH EHDDSGEFTL
              410 420 430
NYVPLKAQEK VLKAAITNSF GEGGINASLC FAKVDTK
```

A sequence for a Nannochloropsis gaditana 3-oxoacyl-[acyl-carrier-protein]synthase protein is shown below as SEQ ID NO:27 (Uniprot accession no. W7TRD5).

```
                10 20 30 40 50
MRLSTLSVLG PALGCAFLLF DSSLAYLPSY MRSKGQIYM KEKSQRVVVT
                60 70 80 90 100
GLGPISAVGI GKDAFWKALL EGKSGIDRIS GFDPSGLICQ IGAEVKDFDA
              110 120 130 140 150
KPYFKDRKSA VRNDRVTLMG VAASRIAVDD AKLDLSSVEG ERFGVVVGSA
              160 170 180 190 200
FGGLQTLETQ IQTMNEKGPG SVSPFAVPSL LSNLISGVIA LENGAKGPNY
              210 220 230 240 250
VVNSACAAST HALGLAYAHI AHGEADVCLA GGSEAAVTPF GFAGFCSMKA
              260 270 280 290 300
MATKYNDNPS QGSRPFDKDR CGFVMGEGAG MVVLESLEHA QKRGAHIYAE
              310 320 330 340 350
VAGFGQACDA HHITTPHPEG AGLAQAITLA LEDAGMAKED LTYINABGTS
              360 370 380 390 400
TAYNDKFETL AVKKALGEEV AKKMYLSSTK GSTGHTLGAA GGLEAIATVL
              410 420 430 440 450
AIETKTLPPT INYETPDPDC DLNVVPNKPI TLNEITGAAS QSAGFGGHDS
VVVFKPFK
```

A sequence for a Nannochloropsis gaditana (strain CCMP526) 3-oxoacyl-ACP synthase 3 protein is shown below as SEQ ID NO:28 (Uniprot accession no. I2CQW7).

```
                10 20 30 40 50
MSKRSRASSR GLAYIQRLHL LSLSLCLLLS LQCSIRAAAF LVPSSPLPSL
                60 70 80 90 100
PSSHGPSLPS SRPPSSVPKS QALRMATSLT EGSSVDAPAA VPGRSFLRAK
              110 120 130 140 150
PIGVGSAAPE DVITNTDLES IVETSDEWIF TRTGISQRRI LTSGGQIRAL
              160 170 180 190 200
AATAAARAIA SAGLEGKDID LVVLATSSPD DLFGDATSVA AAVGATQAVA
              210 220 230 240 250
FDLTAACSGF LFGVVSASQF LHSGCYRRAL VVGADALSRW VDWEDRNSCI
              260 270 280 290 300
LFGDGAGAVV LEAAEGEEDS GVIGFAMHSD GTGQGDLNLQ FSRDDSQSPP
              310 320 330 340 350
SIREVTPYKG KYNNIAMNGK EVYKFATRKV PTVIEEALAN AGLGVENVDW
              360 370 380 390 400
LLLHQANIRI MDVVADRLGL SKDKILTNLS EYGNTSAGSI PLALDEAVKA
              410 420
AKVKKGDIIA CAGFGAGLSW GSAIIRWQG
```

A sequence for a (3R)-hydroxymiyristoyl-[ACP] dehydratase from a bacterium endosymbiont of Mortierella elongata FMR23-6 is shown below as SEQ ID NO:29 (NCBI GAM51895.1).

```
    1 MLDWRFFTER TCAAVRALGS ERHRHSTRWA LCLSDPFEFA
  41 CGLFALLAAG KQIVLPSNHK PAALLPLAGL YDSV1DDLDG
  81 LLANGAGGPC AKLRIDPRAP LSLVTSGSSG VPKVIQKTLA
121 QFEAEIHTLA TLWGTVMRGV TVVASVPHHH IYGLLFRLLW
161 PLAAGQPFDR MTCVEPADVR ARLAALQNTV LVSSPAQLTR
201 WPSLINLTQL TPPPGLIFSS GGPLPAETAA IYTQAFaAAP
241 IEVYGSTETG GIAWRCQPQA THQNEVSDAW TPMPAIDVRC
281 DTEGALQLRS PHLPDDQWWR MEDAVQIEAD GRFRIRGRLD
321 RIIKLEEKRV SLPELEHVLM RHPWVKQAAV APLNaARMIL
361 GALLTLTEEG IQAWRSAASR RFITQALRRY LAEYFDGVVL
401 PRHWRFCMQL PFDERGKLSV TQLATRFATH PLQPEVLAEW
441 CDDNTALLEL HVPATLIHFS GHFPGLPILP GVVQIDWVVR
481 YAAHYFARCN GFQTLEQIKE LSMVRPGTTL RLALAHDPER
521 ARITFRYYVG ERDYATGRIV YSKSAVV
```

A sequence for a beta-hydroxyacyl-ACP dehydratase (FabA) from Nannochloropsis gaditana is shown below as SEQ ID NO:30 (Uniprot W7TUB8).

```
                10 20 30 40 50
MHLLAALVAL PAMCTAFVVP LPSAPKHAVR MMADGDAAGA EWRGGQAASA
                60 70 80 90 100
VSKDLKILLT NENVASILPH RYPELLVDKV IEMEPGKKNV GIKQITANEP
              110 120 130 140 150
QFIGHFPERP IMPGVLMVEA MAQLSGVLCL QPPVSDGKGL FFFAGIDGVK
              160 170 180 190 200
FRKPVVPGDT LVMEVELVKF MESFGIAKLK GKAYVDGDVA VEIKEMTFAL
SK
```

A sequence for a 3-hydroxyacyl-CoA dehydrogenase (FabA) from Nannochloropsis gaditana (strain CCMP526) is shown below as SEQ ID NO:31 (Uniprot K8YU30).

```
                10 20 30 40 50
MADGDAAGAE WRGGQAASAV SKDLKILLIN ENVASILPHR YPELLVDKVI
                60 70 80 90 100
EMEPGKKAVG IKQITANEPQ FIGHFPERPI MPGVLMVEAM AQLSGVLCLQ
              110 120 130 140 150
PPVSDGKGLF FFAGIDGVKF RKPVVPGDTL VMEVELVKFM ESFGIAKIKG
              160 170
KAYVDGDVAV EIKEMTFALS K
```

A sequence for a 3-oxoacyl-(Acyl-carrier-protein) reductase from Nannochloropsis gaditana is shown below as SEQ ID NO:32 (Uniprot W7U8F0).

```
                10 20 30 40 50
MASHHLTTQE HARRKVAVVT GAAGTLuESI TGMLLSEGYV VAALDIRAEG
                60 70 80 90 100
LSAFKATLDK KSDQYHAFAV DISSASAVEE VCRTILTRLG AVSVLINNAG
              110 120 130 140 150
LLSNHKCVQT SLTEWHRVMH VNVDGAFLLS QQLLPCMRSM HFGRIVNITS
              160 170 180 190 200
MAAKTGGVTA GTAYAVSKGA LASLIFSLAR ETAGDGITVN GVAPAYVKIP
              210 220 230 240 250
MVMQQLREEQ RVQVLNSIPV GRFCEPEEVA HTVRFLISPL AGFITGEIID
QNGGYHMD
```

A sequence for a 3-oxoacyl-ACP reductase (FabG) from a bacterium endosymbiont of Mortierella elongata FMR23-6 is shown below as SEQ ID NO:33 (NCBI WP_045362092.1).

```
    1 MRRRVLVTGA SRGIGRAIAE QLASDGFALT IHAHSGWTEA
  41 QAVVAGIVAQ GGQAQALRED VRERALCSKI LTEDVAAHGA
  81 YYGIVCNAGV VRDAVFPALS GEDWDTVIDT SLDGFYNVVH
121 PLTMPMVRAK AGGRIITISS VSGMIGNRGQ VNYSAAKAGL
161 IGASKALALE LASRAITVNC VAPGIIATEM INTELREQAS
201 KEVPMKRVGT PSEVAALVSF LMSDAAAYIT RQVIGVNGGI
241 V
```

A sequence for an elongation of fatty acids (ELO) protein from Mortierella elongata AG-77 is shown below as SEQ ID NO:34 (Uniprot A0A197K6H1).

```
                10 20 30 40 50
MESISUIPN KLPQDLFIDF ATAFGVRLAID YVDPLEDALT AQMEKFFPAL
                60 70 80 90 100
VHHYRAFLTA VESPLAAQLP LMNPFHVVLI VIAYLVTVFV GMQIMKNFNR
              110 120 130 140 150
FEVKTFSLFH NFCLVSISAY MCGGILYEAY QSKYGLFENL ADHTSTGFPM
              160 170 180 190 200
AKMIWLFYFS KIMEFVDTMI MVLKKNNRQI SFLHVYHHSS IFAIWWLVTF
              210 220 230 240 250
VAPNGEAYFS AALNSFIHVI MYGYYFLSAL GFKQVSFIKF YITRSQMTQF
              260 270 280 290 300
CMMSVQSSWD MFAMKVMGRP GYPFFITALL WFYMWTMLGL FYNFYRKNAK
              310
LAKQAKADAA KEKSKKLQ
```

Another sequence for an elongation of fatty acids (ELO) protein from Mortierella elongata AG-77 is shown below as SEQ ID NO:35 (Uniprot A0A197K854).

```
                10 20 30 40 50
MAAAFLDQVN FSLDQPFGIK LDNYFAKGYE LVTGKSIDSF VFQEGVTPLS
                60 70 80 90 100
TQYEVAMWTV TYFIVIFGGR QIMKSQEAFK LKPLFILHNF LLTIASGALL
              110 120 130 140 150
LLFIENLVPI LARNGLFYAI CDQGAWTQRL ELLYYLNYLV KYWELADTVF
              160 170 180 190 200
LVLKKKPLEF LHYFHHSMTM ILCFVQLGGY TSVSWVPITL NLTVHVLMYY
              210 220 230 240 250
YYMRSAAGVR IWWKQYLTTL QIVQFVLDLG FIYFCSYTYF AFTYWPHLPN
              260 270 280 290 300
VGKCAGTEGA ALFGCGLLSS YLLLFINFYR LTYNAKAKAA KERGSNVTPK
              310
TPKADKKKSK HI
```

Another sequence for an elongation of fatty acids (ELO) protein from Mortierella elongata AG-77 is shown below as SEQ ID NO:36 (Uniprot A0A197JPT7).

```
                10 20 30 40
MESAPMPAGV PFPEYYDFFM NWKTPLAIAA TYTVAVTLFN
                50 60 70 80
PKVGKVSRVV AKSANAKPAE KTQSGAAMTA FVFVHNLILC
                90 100 110 120
VYSGITFYNM FPAMIKNFAT HSIFDAYCDT DQSLWNGSLG
              130 140 150 160
YWGYIFYLSK FYEVIDTIII ILKGRRSSLL QTYHHAGAMI
              170 180 190 200
TMWSGINYQA TPIWIFVVFN SFIHTIMYAY YAATSVGLHP
              210 220 230 240
PGKKYLTSMQ ITQFLVGMSI AVSYLFIPGC IRTPGAQMAV
              250 260 270
WINVGYLFPL TYLFVDFAKR TYSKRSAAPA KKTE
```

Another sequence for an elongation of fatty acids (ELO) protein from Mortierella elongata AG-77 is shown below as SEQ ID NO:37 (Uniprot A0A197KI55).

```
                10 20 30 40
MGLSKTVGQA SDKNICMIFC KGQPIGQVQP EGILYPEYFD
                50 60 70 80
VLVNWRTPVS VAALYVLMVV LLNPKQGKVS RVVAADSAAK
                90 100 110 120
GDNKKQQELS SSSPAMTALV FVHNAILCVY SAWTFYGMFF
              130 140 150 160
AWKKAFATHT FMEAVCDSDN TFWDSLGYYS YYFYLSKYYE
              170 180 190 200
IVDTIIILLK GRRSSLLQTY HHAGAIFTMY MGFNYRAEPI
              210 220 230 240
WIFTTFNSFI HTIMYAYYAA TSVGLKPPGK KYLTSMQITQ
              250 260 270 280
FWTGTALAFW YEIGSPKGCF TNPGSRFAIW TVLAYVFPLI
              290 300 310
YLFTSFASKM YGNRVKLAAA AKATSQQKKV L
```

A sequence for an elongation of fatty acids (ELO) protein from Nannochloropsis oculata is shown below as SEQ ID NO:38 (Uniprot D2DPY9).

```
                10 20 30 40
MPKLPKISNI FKFLKADPSK IVPYKSIPDK VPFTQLFQHY
                50 60 70 80
PVLDPLYTQY EKNFYASTYV KFAQDTWPVL PLALCGMYAL
                90 100 110 120
MIIVGTKVMV SRPKHEWKTA LACWNLMLSI FSFCGMIRTV
              130 140 150 160
PHLLHNVATL PFKDTICRHP AETYGEGACG MWVMLFIFSK
              170 180 190 200
VPELVDTVFI VFRKSKLQFL HWYHHITVLL FCWHSYAVTS
              210 220 230 240
STGLYFVAMN YSVHAIMYAY YYLTAINAWP KWIPPSIITV
              250 260 270 280
AQISQMIVGV GICASSFYFL YTDPEHCQVK RQNVYAGALM
              290 300 310 320
YGSYLYLFCD FFVRRFLRGG KPRLGEEKSA VLTMAKKIKA
M
```

Another sequence for an elongation of fatty acids (ELO) protein from Nannochloropsis oculata is shown below as SEQ ID NO:39 (Uniprot E7DDK1).

```
10 20 30 40
MSFLIRTPAD QIKPYFSEAA QTHYTQLFQH FPILERAYFP
50 60 70 80
FEKNFRAEPF VDFAKATWPL LPLALCTAYA LMIVIGTRVM
                90 100 110 120
KNREKFDWRG PLAYWNLTLS LFSFCGMLRT VPHLLNNITT
              130 140 150 160
LSFRDTVCTS AAKSYGEGVS GLWVMLFIFS KIPELVDTVF
              170
TVFRKSKLQF LHW
```

A sequence for a delta-9 fatty acid desaturase protein from Nannochloropsis oceanica is shown below as SEQ ID NO:40 (Uniprot A0A1S7C7S1).

```
                10 20 30 40
MVFQLARDSV SALVYHFKEG NLNWPMIIYL VLVHLAGYIG
                50 60 70 80
LTTILACKWQ TLLEAFILWP ITGLGITAGV HRLWAHRSYN
                90 100 110 120
ATLPYRILLM LFNSIANQGS IYHWSRDHRV HHKYSETDAD
              130 140 150 160
PHNATRGFFF AHMGWLIVKK HPKVVEGGKQ LDFSDLAADP
              170 180 190 200
VVRFQRDWDP WFAQFMCFVM PALVASRFWG EAFWNAFWVA
              210 220 230 240
GALRYMLVLH FTWMVNSAAH LYGDHPYDPT MWPAENPLVS
              250 260 270 280
VVAIGEGWHN WHHRYPYDYA ASEFGISQQF NPTKAFIDFF
              290 300 310 320
AAIGMVTNRK RATGAWAKLK ESRARDAANG KSMKDFKGRG
              330 340 350
SGSDYGTTNT NYAVSNKTVV TDKGAQQPGW EESNHPKYN
```

A sequence for a fatty acid hydroxylase protein from Nannochloropsis gaditana is shown below as SEQ ID NO:41 (Uniprot W7UAP1).

```
                10 20 30 40
MAAYFQVFRN SKIGIVLTLS LIFTTAMASP SAYFPEKLSL
                50 60 70 80
LLKTLSGSDR LVNPHCIDNP FCAFNDWVNA FLFRDAVKAD
                90 100 110 120
VMARLGPAGA HYFLTYVRDL VAGSVLYYLT AGLWHTYIYQ
              130 140 150 160
WHGDYFFTQQ GFEKPSAATI KDQIQLAQAS MFLYAALPVL
              170 180 190 200
AEWLVESGWT QCYYYVEEIG GWPYYLAFTL LYLAMVEVGV
              210 220 230 240
YWMHRTLHEN KVLYKYIHGL HHKYNKPSTL SPWASVAFNP
              250 260 270 280
IDGILQASPY VICLFLVPCH YLTHVAMVFF TAVWATNIHD
              290 300 310 320
AMDGNTEPVM GSKYHTVHHT HYHYNFGQFF IFADWMFGTL
              330 340 350
RIPEPRAAKA VLSPGVVPSS GVRTTGKSGR GKMD
```

A sequence for an omega-6 fatty acid desaturase delta-12 protein from Nannochloropsis gaditana (strain CCMP526) is shown below as SEQ ID NO:42 (Uniprot K8YR13).

```
                10 20 30 40
MGRGGEKTVT PPSKTFHAHG HSLTASDLSR ADAASTISSS
                50 60 70 80
VRPSKSLEAM PTEELRKKAL QYGHDASADR ASLLQILAPY
                90 100 110 120
GDILLRIDAP PSLPLTPPPF TLADIKAAVP RHCFERSLTT
              130 140 150 160
SFFHLACDLV LVALLGYLAT LIGHPDVPTM SRYLLWPLYW
              170 180 190 200
YAQGSVLTGV WVIAHECGHQ SFSPYERVNN LVGWVLHSAL
              210 220 230 240
LVPYHSWRIS HGKHHNNTGS CENDEVFAPP IKEDLMDEIL
              250 260 270 280
LHSPLANLAQ IIIMLTVGWM PGYLLMNATG PRKYKGKNNS
              290 300 310 320
HFDPNSALFS PKDRLDIIWS DIGFFLALAG VVAWCTQYGF
              330 340 350 360
STVGKYYLLP YMVVNYHLVL ITYLQHTDVF IPHFRGAEWS
              370 380 390 400
WFRGALCTVD RSFGWLLDHT FHHISDTHVC HHIFSKMPFY
              410 420 430 440
HAQEASEHIK KALGPYYLKD DTPIWKALWR SYTLCKYVDT
              450
DKNAVFYKHR AS
```

A sequence for an omega-6 fatty acid desaturase delta-12 protein from Nannochloropsis gaditana (strain CCMP526) is shown below as SEQ ID NO:43 (Uniprot K8Z8R1).

```
                10 20 30 40
MSRYLLWPLY WYAQGSVLTG VWVIAHECGH QSFSPYERVN
                50 60 70 80
NLVGWVLHSA LLVPYHSWRI SHGKHHNNTG SCENDEVFAP
                90 100 110 120
PIKEDLMDEI LLHSPLANLA QIIIMLTVGW MPGYLLMNAT
              130 140 150 160
GPRKYKGKNN SHFDPNSALF SPKDRLDIIW SDIGFFLAIA
              170 180 190 200
GVVWACTQYG FSTVGKYYLL PYMVVNYHLV LITYLQHTDV
              210 220 230 240
FIPHFRGAEW SWFRGALCTV DRSFGWLLDH TFHHISDTHV
              250 260 270 280
CHHIFSKMPF YHAQEASEHI KKALGPYYLK DDTPIWKALW
              290 300 310 320
RSYTLCKTAE EEEDDEWGVV PKPTEQLYLG NRKARELIGG
              330
AYADVNLAVK VAHDDTK
```

A sequence for a delta 5 fatty acid desaturase protein from Nannochloropsis gaditana (strain CCMP526) is shown below as SEQ ID NO:44 (Uniprot K8YSX2).

```
                10 20 30 40
MGSTEPVLST AAVPATEPAG KSYTWQEVAE HNTEKSLWVT
                50 60 70 80
VRGKVYDISS WVDNHPGGKE ILLLAAGRDI TYAFDSYHPF
                90 100 110 120
TEKPTQVLNK FEIGRVTSYE FPQYKADTRG FYKALCTRVN
              130 140 150 160
DYFVAHKLNP KDPIPGIWRM CLVALVALAS FVVCNGYVGV
              170 180 190 200
EGTWAGTTWA RLVAAVVFGI CQALPLLHVM HDSSHLAFGN
              210 220 230 240
TERWWQVGGR LAMDFFAGAN MTSWHNQHVI GHHIYTNVFL
              250 260 270 280
ADPDLPDKAA GDPRRLVQKQ AWQAMYKWQH LYLPPLYGIL
              290 300 310 320
GIKFRVQDIM ETFGSGTNGP VRVNPLSFFQ WAEMIFTKMF
              330 340 350 360
WAGWRIAFPL LSPSFHTGWA AFSALFLVSE FMTGYFLAFN
              370 380 390 400
FQVSHVSSEC DYPLGEAPRE GEDGNIVDEW AVSQIKSSVD
              410 420 430 440
YAHNNPVTTF LCGALNYQVT HHLFPTVSQY HYPAIAPIIQ
              450 460 470 480
DVCREFNVDY KVLPDPVTAF HAHIAHLKTL GERGEAAEVH
MG
```

A sequence for a fatty acid desaturase protein from Nannochloropsis gaditana (strain CCMP526) is shown below as SEQ ID NO:45 (Uniprot K8Z7K3).

```
                10 20 30 40
MSGSQGRPER VGEGHPRDAR REEKCGSADN GLRDGRAERA
                50 60 70 80
KEEGRGAYPD AMNEVACVFL YPTLPRITSS SPVTVPPGLQ
                90 100 110 120
VMAAVVLRHA PFPLLLFLTY TLSGSCNHFL TLIMHEVAHN
              130 140 150 160
LAFKRLFANR VFSIIVNLPL GIPAAMWVWE GGPEGGVQAP
TSG
```

A sequence for a delta-9 acyl-CoA desaturase (FADS9) protein from Mortierella elongata AG-77 is shown below as SEQ ID NO:46 (Uniprot A0A197K9U9).

```
                10 20 30 40
MATPLPPTFV VPATLTETRR DPLKHQELPP LFPEKVNILN
                50 60 70 80
IWKYLDYKHV VGLGVTPLIA LYGLLTTEIQ RKTLIWSIIY
                90 100 110 120
YYATGLGITA GYHRLWAHRS YNAGPAMSFV LALLGAGAVE
              130 140 150 160
GSIKWWSRGH RAHHRWTDTE KDPYSAHRGL FFSHLGWMLI
              170 180 190 200
KRPGWKIGHA DVDDLNKNKL VQWQHKNYLA LIFLMGVVFP
              210 220 230 240
TVVAGLGWGD WRGGYFYAAI LRLVFVHHAT FCVNSLAHWL
              250 260 270 280
GEGPFDDRHS PRDHFITAFM TLGEGYHNFH HQFPQDYRNA
              290 300 310 320
IRFYQYDPTK WVIATCAFLG LASHLKTFPE NEVRKGQLQM
              330 340 350 360
IEKRVLEKKT KLQWGTPIAD LPVMSFEDYR HACKNDNKKW
              370 380 390 400
ILLEGVVYDV ADFMSEHPGG EKYIKMGIGK DMTAAFNGGL
              410 420 430 440
YDHSNAARNL LSLMRVAVVE FGGEVEAQKK NPSAPIYGDD
HAKAA
```

A sequence for an acyl-CoA desaturase (FAD) protein from Mortierella alpina is shown below as SEQ ID NO:47 (Uniprot 094747).

```
                10 20 30 40
MATPLPPSFV VPATQTETRR DPLQHEELPP LFPEKITIYN
                50 60 70 80
IWRYLDYKHV VGLGLTPLIA LYGLLTTEIQ TKTLIWSIIY
                90 100 110 120
YYATGLGITA GYHRIWAHRA YNAGPAMSFV LALLGAGAVE
              130 140 150 160
GSIKWWSRGH RAHHRWTDTE KDPYSAHRGL FFSHIGWMLI
              170 180 190 200
KRPGWKIGHA DVDDLNKSKL VQWQHKNYLP LVLIMGVVFP
              210 220 230 240
TLVAGLGWGD WRGGYFYAAI LRLVFVHHAT FCVNSLAHWL
              250 260 270 280
GDGPFDDRHS PRDHFITAFM TLGEGYHNFH HQFPQDYRNA
              290 300 310 320
IRFYQYDPTK WVIAICAFFG LASHLKTFPE NEVRKGQLQM
              330 340 350 360
IEKKVLEKKT KLQWGTPIAD LPVLSFEDYQ HACKNDNKKW
              370 380 390 400
ILLEGVVYDV ADFMSEHPGG EKYIKMGVGK DMTAAFNGGM
              410 420 430 440
YDHSNAARNL LSLMRVAVVE YGGEVEAQKK NPSMPIYGTD
HAKAE
```

A sequence for an acyl-CoA desaturase (FAD) protein from Mortierella elongata AG-77 is shown below as SEQ ID NO:48 (Uniprot A0A197JWT1).

```
                10 20 30 40
MATPLPPTFV VPATQTETRR LPLEHDELPP LFPEKLTITN
                50 60 70 80
IWKYLDYKHV LGLGLTPLIA LYGLLTTEIQ TKTLIWSIVY
                90 100 110 120
YYATGLGITA GYHRLWAHRA YSAGPAMSFA LALLGAGAVE
              130 140 150 160
GSIKWWSRGH RAHHRWTDTE KDPYSAHRGL FFSHIGWMLI
              170 180 190 200
KRPGWKIGHA DVDDLNKNKL VQWQHKHYLP LVLFMGVIFP
              210 220 230 240
TIVAGLGWGD WRGGYFYAAI LRLVFVHHAT FCVNSLAHWL
              250 260 270 280
GEGPFDDRHS PRDHFITAFM TLGEGYHNFH HQFPQDYRNA
              290 300 310 320
IRFYQYDPTK WVIAICAFFG LASHLKTFPE NEVRKGQLQM
              330 340 350 360
IEKKVLEKKT KLQWGTPIAD LPVLSFEDYQ HACKNDGKKW
              370 380 390 400
ILLEGVVYDV AEFMNEHPGG EKYIKMGVGK DMTAAFNGGM
              410 420 430 440
YDHSNAARNL LSLMRVAIVE FGGEVEAQKK NPSVPIYGDD
HHSKSE
```

A sequence for a delta-6 acyl-CoA desaturase (FAD) protein from Mortierella elongata AG-77 is shown below as SEQ ID NO:49 (Uniprot A0A197JJR0).

```
                10 20 30 40
MAATPSVRTF TRSEILNAEA LNEGKKDAEA PFLMIIDNKV
                50 60 70 80
YDVREFVPEH PGGSVILTHV GKDGTDVFDT FHPEAAWETL
                90 100 110 120
ANFYVGDIAE HDRAIKGDDF AAEVRKLRSL FQSLGYYDSS
              130 140 150 160
KAYYAFKVSF NLCLWALSTF IVAKWGQTST LATIASASIL
              170 180 190 200
GLFWQQCGWL AHDFLHHQVF QDRFWGDLFG AFLGGVCQGF
              210 220 230 240
SSSWWKDKHN THHAAPNVHG EDPDIDTHPL LTWSEHALEM
              250 260 270 280
FSDVPDEELT RMWSRFMVLN QTWFYFPILS FARLSWCLQS
              290 300 310 320
ILFVLPNGQA HKPSGARVPI SLVEQLSLAM HWTWYFATMF
              330 340 350 360
LFIKDPVNMI VYFLVSQAVC GNLLALVFSL NHNGMPVISK
              370 380 390 400
EEAVDMDFFT KQIITGRDVH PGLFANWFTG GLNYQIEHHL
              410 420 430 440
FPSMPRHNFS KIQPAVESLC KKYGVRYHTT GMVDGTAEVF
              450
ARLNEVSRAA SKMGKST
```

A sequence for a delta-5 acyl-CoA desaturase (FAD) protein from Mortierella elongata AG-77 is shown below as SEQ ID NO:50 (Uniprot A0A197KDG7).

```
                10 20 30 40
MGAEKEFTWE ELAKHNIAGD LYVAVRGNVY DVTKFLSRHP
                50 60 70 80
GGVDTLLLGA GRDVTPVFDM YHAFGTGDAI MKKYYVGKLV
                90 100 110 120
SNELPIFPEP SGFHKVVKSR VEGYFKDSGK DPKNRPEIWG
              130 140 150 160
RYFLIFAALF LSYYAQFPVP FVVERTWLQV IFAVIMGFAC
              170 180 190 200
AQIGLNPLHD ASHFSTTHNP TVWKILGATH DFFNGASYLV
              210 220 230 240
WMYQHMLGHH PYTNIAGADP DVSTAERDVR RIKPSQKWFW
              250 260 270 280
NHINQHMFVP FLYGLLAFKV RIQDVNILYF VGTNDAIRVN
              290 300 310 320
PISLWHTVMF WGGKIFFFWY RIYVPLQVLP LKKVLILFTI
              330 340 350 360
ADMISSYWLA LTFQANHVVE EVEWPLPDEN GIIQKDWAAM
              370 380 390 400
QVETTQDYAH ESYIWTSITG SLNIQAVHHL FPNVSQHYYP
              410 420 430 440
EILSIIRDAC TEYKVPYLVK DTFWQAFSSH LEHMRVLGLR
PKEE
```

A sequence for a delta-12 acyl-CoA desaturase (FAD) protein from Mortierella elongata AG-77 is shown below as SEQ ID NO:51 (Uniprot A0A197K3I9).

```
                10 20 30 40
MAPPNTIDAG LTHRHVVNPT AAPVKAAYER NYELPEFTIK
                50 60 70 80
EIRECIPAHC FERSGFRGLC HVAIDLTWAS LLFLAATQID
                90 100 110 120
KFENPLIRYL AWPVYWVMQG IVCTGIWVLA HECGHQSFST
              130 140 150 160
SKTLNNTVGW ILHSFLLVPY HSWRISHSKH HKATGHMTKD
              170 180 190 200
QVFVPKTRTQ VGLPAKKENV VEEDEAVHLD EEAPIVTLFW
              210 220 230 240
MLVQFTFGWP AYLAVNASGQ DYGQWTSHFH TWSPIFEARN
              250 260 270 280
FTDVILSDLG VLVTLGALIY ASLQTSLLAV TKYYIVPYLF
              290 300 310 320
VNFWLVLITF LQHTDPKLPH YRENVWNFQR GALCTVDRSF
              330 340 350 360
GKFLDHMFHG IVHTHVAHHL FSQMPFYHAE EATACLKKLL
              370 380 390
GKHYIYDDTP IVLATWRSFR ECRFVEDEGD VVFFKK
```

A sequence for a delta-6 acyl-CoA desaturase (FADS6) protein from Mortierella alpina is shown below as SEQ ID NO:52 (Uniprot Q9UVY3).

```
                10 20 30 40
MAAAPSVRTF TRAEILNAEA LNEGKKDAEA PFLMIIDNKV
                50 60 70 80
YDVREFVPDH PGGSVILTHV GKDGTDVFDT FHPEAAWETL
                90 100 110 120
ANFYVGDIDE SDRAIKNDDF AAEVRKLRTL FQSLGYYDSS
              130 140 150 160
KAYYAFKVSF NLCIWGLSTF IVAKWGQTST LANVLSAALL
              170 180 190 200
GLFWQQCGWL AHDFLHHQVF QDRFWGDLFG AFLGGVCQGF
              210 220 230 240
SSSWWKDKHN THHAAPNVHG EDPDIDTHPL LTWSEHALEM
              250 260 270 280
FSDVPDEELT RMWSRFMVLN QTWFYFPILS FARLSWCLQS
              290 300 310 320
IMFVLPNGQA HKPSGARVPI SLVEQLSLAM HWTWYLATMF
              330 340 350 360
LFIKDPVNMI VYFLVSQAVC GNLLAIVFSL NHNGMPVISK
              370 380 390 400
EEAVDMDFFT KQIITGRDVH PGLFANWFTG GLNYQIEHHL
              410 420 430 440
FPSMPRHNFS KIQPAVETLC KKYGVRYHTT GMIEGTAEVF
              450
SRLNEVSKAA SKMGKAQ
```

A sequence for a delta-6 acyl-CoA desaturase (FADS6) protein from Mortierella alpina is shown below as SEQ ID NO:53 (Uniprot AMRI59).

```
                10 20 30 40
MAAAPSVRTF TRAEILNAEA LNEGKKDAEA PFLMIIDNKV
                50 60 70 80
YDVREFVPDH PGGSVILTHV GKDGTDVFDT FHPEAAWETL
                90 100 110 120
ANFYVGDIDE SDRAIKNDDF AAEVRKLRTL FQSLGYYDSS
              130 140 150 160
KAYYAFKVSF NLCIWGLSTF IVAKWGQTST LANVLSAALL
              170 180 190 200
GLFWQQCGWL AHDFLHHQVF QDRFWGDLFG AFLGGVCQGF
              210 220 230 240
SSSWWKDKHN THHAAPNVHG EDPDIDTHPL LTWSEHALEM
              250 260 270 280
FSDVPDEELT RMWSRFMVLN QTWFYFPILS FARLSWCLQS
              290 300 310 320
IMFVLPNGQA HKPSGARVPI SLVEQLSLAM HWTWYLATMF
              330 340 350 360
LFIKDPVNMI VYFLVSQAVC GNLLAIVFSL NHNGMPVISK
              370 380 390 400
EEAVDMDFFT KQIITGRDVH PGLFADWFTG GLNYQIEHHL
              410 420 430 440
FPSMPRHNFS KIQPAVETLC KKYGVRYHTT GMIEGTAEVF
              450
SRLNEVSKAA SKMGKAQ
```

A sequence for acyl-CoA desaturase (FAD) protein from Mortierella verticillata is shown below as SEQ ID NO:54 (NCBI KFH-69129.1).

| | |
|---|---|
| 1 | MVATRTFTRS EILNAEALNE GKKNADAPFL MIIDNKVYDV |
| 41 | REFVPDHPGG SVILTHVGKD GTDVFDTFHP EAAWETLANF |
| 81 | YVGDIAENDR AIKNDDFAAE VRKLRTLFQS LGYYDSSKAY |
| 121 | YAFKVSFNLC LWALSTFIVA KWGQTSTLAN VLSASILGLF |
| 161 | WQQCGWLAHD FLHHQVFQDR FWGDLFGAFL GGVCQGFSSS |
| 201 | WWKDKHNTHH AAPNVEGEDP DIDTHPLLTW SEHALEMFSD |
| 241 | VPDEELTKMW SRFMVLNQTW FYFPILSFAR LSWCLQSIMF |
| 281 | VMPNGQAHKP SGARVPISLV EQLSLAMHWT WYFATMFLFI |
| 321 | KDPVNIMVYF LVSQAVCGNL LALVFSLNHN GMPVISKEEA |
| 361 | VDMDFFTKQI ITGRDVHPGL FANWFTGGLN YQIEHHLFPS |
| 401 | MPRHNFSKIQ PAVASLCKKY NVRYHTTGMV DGTAEVFARL |
| 441 | NEVSRAASKM GKSA |

A sequence for a delta-6 acyl-CoA desaturase (FAD) protein from Mortierella alpina is shown below as SEQ ID NO:55 (NCBI ADE06661.1).

| | |
|---|---|
| 1 | MAAAPSVRTF TRAEILNAEA LNEGKKDAEA PFLMIIDNKV |
| 41 | YDVREFVPDH PGGSVILTHV GKDGTDVFDT FHPEAAWETL |
| 81 | ANFYVGDIHE SDRDIKNDDF AAEVRKLRTL FQSLGYYDSS |
| 121 | KAYYAFKVSF NLCIWGLSTF VVAKWGQTST LANVVSAALL |
| 161 | GLFWQQCGWL AHDFLHHQVF QDRFWGDLFG AFLGGVCQGF |
| 201 | SSSWWKDKHN THHAAPNVHG EDPDIDTHPL LTWSEHALEM |
| 241 | FSDVPDEELT RMWSRFMVLN QTWFYFPILS FARLSWCLQS |
| 281 | ILFVMPNGQA HKPSGARVPI SLVEQLSLAM HWTWYLATMF |
| 321 | LFVKDPINMF VYFLVSQAVC GNLLALVFSL NHNGMPVISK |
| 361 | EEAVDMDFFT KQIITGRDVH PGLFANWFTG GLNYQIEHHL |
| 401 | FPSMPRHNFS KIQPAVETLC KKYNVRYHTT GMIEGTAEVF |
| 441 | SRLNEVSRAA SKMGKAQ |

A sequence for an acyl-coenzyme A thioesterase protein from Mortierella elongata AG-77 is shown below as SEQ ID NO:56 (Uniprot A0A197JUG8).

```
                10 20 30 40
MSDSHLTVDP TSTTPHPDAD GTTNNTIIET MLDLEEIDKD
                50 60 70 80
LYRSKKLWVP MGARGVFGGN VVGQALVAAT NTVSTDYSVH
                90 100 110 120
SLHSYFLLPG DHTTPILYHV ERVRDGKSYC TRTVTAKQRG
              130 140 150 160
KNIFVCTASY QVPRPGAPSH QYPMPNVPHH STLPSQEELI
              170 180 190 200
HAMIDNTKLP ENLKDFLRLR LDEPVALEFK DTKRHTFKEL
              210 220 230 240
MNPEVRTDQS FWIRCKGQLG DALALHQCVV AYGSDHNLLN
              250 260 270 280
TVPLAHGSSW FSRRSGLSPK ITMMASLDHS MWFHCPFRAD
              290 300 310 320
EWLLYVCETP RSGCDRGLTF GRIYKEDGTL AISVAQEGVV
              330
RLQPKTPTPA ATVETPKL
```

A sequence for an acyl-coenzyme A thioesterase protein from Lobosporangium transversale is shown below as SEQ ID NO:57 (Uniprot A0A1Y2G902).

```
                10 20 30 40
MSSVSEPGST LNLAPTPDGS SNNTIIETML DLEEIDKDLY
                50 60 70 80
RSKKLWLPLG ARGVFGGNVV GQALVAATNT VSDLYSVHSL
                90 100 110 120
HSYFLLPGDP TIPILYHVDR LRDGHSYCTR TVTATQRGKN
              130 140 150 160
IFVCTASFQV PRPNAPSHQY PMPNVPHHST LPSQEDLIRA
              170 180 190 200
MIDSPKIPEN LVEFLKQRLD EPVALDFKDT RRHTLKDLMN
              210 220 230 240
PPVRTEQTFW IKCKGGLGDA LALHQCVVAY GSDHNLLNTV
              250 260 270 280
PLAHGSTWLS RRSSSPSIVM MASLDHSMWF HCPFRADEWM
              290 300 310 320
LYVCETPRSG CDRGLTFGRI YKEDGTLAVS VAQEGVVRLR
              330
SKAPSSATVD QPKL
```

A sequence for an acyl-coenzyme A thioesterase protein from bacterium endosymbiont of Mortierella elongata FMR23-6 is shown below as SEQ ID NO:58 (NCBI WP_045362096.1).

| | |
|---|---|
| 1 | MMAKQITQTV LTATVGIEVP FHDIDSMNIC WHGHYVKYFE |
| 41 | IARSALLRSF EYDAMRLSNY LWPVVECRLK YLRPARYGQL |
| 81 | LDVSAKLVEY ESRLKIGYLI TDRESGAQLT KGYTIQVAVD |
| 121 | AQTQALQFVL PRELLDKLEP MLSAVC |

Another sequence for an acyl-coenzyme A thioesterase protein from bacterium endosymbiont of Mortierella elongata FMR23-6 is shown below as SEQ ID NO:59 (NCBI WP_045363294.1).

| | |
|---|---|
| 1 | MHSLSHLPHD KTLALRAVPQ PSNANMHGDV FGGWIMAQVD |
| 41 | IAGSIPATRR AHGRVVTVAV NSLVFKQPVF VGDLLSFYAD |
| 81 | IAKVGNTSVA VSVEVYAQRL NFAEQIFKVA EATLTYVATD |
| 121 | NDRRPRALPA EG |

A sequence for an acyl-coenzyme A thioesterase 13 protein from Nannochloropsis gaditana is shown below as SEQ ID NO:60) (Uniprot W7TZE5).

```
                10 20 30 40
MSLKTISPHD YRSKMTRQER TSRQVLELLH AVSKSAFSGV
                50 60 70 80
LLRRDIEPNA TELQNVKALK IGPGPQVRLR LRVPSHLCDN
                90 100 110 120
YNNNHRLLDA GAVTAWFDEV SSWAFVSADG RHRPGVSVSL
              130 140 150 160
NTTVLSWVPV GTEVEIQSHC KKIGETLGFA DMMLLDVATG
              170 180 190 200
KELAHGRHVK FLKMGTAWTV AMHAWAFPLT YLMASAVLLP
              210 220 230 240
SVRQRTQKSS SFPPEMAPSP DLPRTEPGSA VNINRLLALD
              250 260 270 280
NFHVYEPAGA ASPPLAFPAS VPLTMEASAS FRVIPQVCNS
              290 300 310 320
FGSLHGGAAA ILAERAALAL YHQAARWAGE RSQHALPRVR
              330 340 350 360
SLSIDYMSPC KKNTELLLLV RGMRVERGAG EGDKHSPSRS
              370 380 390 400
LFPPLDVAPH PQGNLIPMSY QVLFTRKKDG RYLTQCHVLL
              410 420
DSQGDAWHHQ RQSRGEGNRA RL
```

A sequence for a thioesterase superfamily member 2 protein from Nannochloropsis gaditana (strain CCMP526) is shown below as SEQ ID NO:61 (Uniprot K8Z9R6).

```
                10 20 30 40
MSLKTISPHG YRSKMTRQEQ TSRQVLELLH AVSKSAFSGV
                50 60 70 80
LLRRDIEPNA TELQNVKALK IGPGPRVRLR LRVPSHLCDN
                90 100 110 120
YDNNHCLLDA GAVTAWFDEV SSWAFVSADG RHRPGVSVSL
              130 140 150 160
NTTVLSWVPV GTEVEIQSHC KKIGETLGFA DMMLLDVATG
              170 180 190 200
KELAHGRHVK FLKMGTAWTV AMHAWAFPLT YLMASAVLLP
              210 220 230 240
SVRQRTQKSS SFPPEMAPSP DLPRTEPGSA ASVLSMVGPP
              250 260 270
QFWLSALLLP CITKPLGGPE RGASTLCRVF VL
```

A sequence for an acyl-CoA synthetase from Mortierella elongata FMR23-6 is shown below as SEQ ID NO:62 (NCBI GAM51895.1).

| | |
|---|---|
| 1 | MLDWRFFTER TCAAVRALGS ERHRHSTRWA LCLSDPFEFA |
| 41 | CGLFALLAAG KQIVLPSNHK PAALLPLAGL YDSVLDDLDG |
| 81 | LLANGAGGPC AKLRIDPRAP LSLVTSGSSG VPKVIQKTLA |
| 121 | QFEAEIHTLA TLWGTVMRGV TVVASVPHHH IYGLLFRLLW |
| 161 | PLAAGQPFDR MTCVEPADVR ARLAALQNTV LVSSPAQLTR |
| 201 | WPSLINLTQL TPPPGLIFSS GGPLPAETAA IYTQAFGAAP |
| 241 | IEVYGSTETG GIAWRCQPQA THQNEVSDAW TPMPAIDVRC |
| 281 | DTEGALQLRS PHLPDDQWWR MEDAVQIEAD GRFRLRGRLD |
| 321 | RIIKLEEKRV SLPELEHVLM RHPWVKQAAV APLNGARMTL |
| 361 | GALLTLTEEG IQAWRSAASR RFITQALRRY LAEYFDGVVL |
| 401 | PRHWRFCMQL PFDERGKLSV TQLATRFATH PLQPEVLAEW |
| 441 | CDDNTALLEL HVPATLIHFS GHFPGLPILP GVVQIDWVVR |
| 481 | YAAHYFARCN GFQTLEQIKF LSMVRPGTTL RLALAHDPER |
| 521 | ARITFRYYVG ERDYATGRIV YSKSAVV |

A sequence for an acyl-CoA synthetase from Mortierella elongata AG-77 is shown below as SEQ ID NO:63 (Uniprot A0A197JCK7).

```
                10 20 30 40
MPDLAWSLPV ARWSAWNAET SAALDMGLKV ANDCAPVGQP
                50 60 70 80
VRVIFASRHG ESRRTTELLK AQAQDPMQPL SPNAFSLSVL
                90 100 110 120
NAAAGVFSMM RGDHSNATAL AAGSETLGYA LLEAFAQYAS
              130 140 150 160
DPQAPVLVIY ADEPPDPIYA SVDDTDAPSG ALALWIADDA
              170 180 190 200
PGVLECRLLI DALNLEDLTL ADIGDDTPLF DTDGIGLDSI
              210 220 230 240
DALEIGIALR KKYQLQIETT DSRMREHFRS LLLDALAGVS
              250 260 270 280
QRPTLFRMTT PLHLLFSNDC VATRPVCIDG DHILDWRFFT
              290 300 310 320
ERTCAAVRAL GSERHRRSAR WALCLSDPFE FACGLFALLA
              330 340 350 360
AGKQIVLPSN HKPAALLPLA GLYDSVLDDL DSLFANGAGG
              370 380 390 400
PCAKLRIDPR APLSLVTSGS SGVPKVIHKT LAQFEAEIHT
              410 420 430 440
LATLWGTVMR DVTVVASVPH HHIYGLLFRL LWPLAAGQPF
              450 460 470 480
DRMTCVEPAD VRARLAALQN TVLVSSPAQL TRWPSLINLA
              490 500 510 520
QLTPPPGLIF SSGGPLPTET AAIYAQAFGA APIEVYGSTE
              530 540 550 560
TGGIAWRCQP QAMHQNEVSD AWTPMPAIDV RCDTDGALQL
              570 580 590 600
RSPHLPDDQW WRMEDAVQIK VDGRFRLRGR LDRIIKLEEK
              610 620 630 640
RVSLPELEHV LMRHPWVKQA AVAPLNGARM TLGALLTLTE
              650 660 670 680
EGIQAWRSAA SRRFITQALR RYLAEYFDGV VLPRHWRFCM
              690 700 710 720
QLPFDERGKL SVTQLAARFA THPLQPEVLA EWCDGNTALL
              730 740 750 760
ELHVPATLSH FSGHFPGLPI LPGVVQIDWV VRYAAHYFAR
              770 780 790 800
CNGFQTLEQI KFLSMVRPGT TLRLALAHDP ERARITFRYY
              810
VGERDYATGR IVYSKSAVV
```

A sequence for an acyl-CoA synthetase from a bacterium endosymbiont of Mortierella elongata FMR23-6 is shown below as SEQ ID NO:64 (NCBI WP 045365524.1).

| | |
|---|---|
| 1 | MTTPLHLLFS HDCVATRPVC IDGDHMLDWR FFTERTCAAV |
| 41 | RALGSERHRH STRWALCLSD PFEFACGLFA LLAAGKQIVL |
| 81 | PSNHKPAALL PLAGLYDSVL DDLDGLLANG AGGPCAKLRI |
| 121 | DPRAPLSLVT SGSSGVPKVI QKTLAQFEAE IHTLATLWGT |
| 161 | VMRGVTVVAS VPHHHIYGLL FRLLWPLAAG QPFDRMTCVE |
| 201 | PADVRARLAA LQNTVLVSSP AQLTRWPSLI NLTQLTPPPG |
| 241 | LIFSSGGPLP AETAAIYTQA FGAAPIEVYG STETGGIAWR |
| 281 | CQPQATHQNE VSDAWTPMPA IDVRCDTEGA LQLRSPHLPD |
| 321 | DQWWRMEDAV QIEADGRFRL RGRLDRIIKL EEKRVSLPEL |
| 361 | EHVLMRHPWV KQAAVAPLNG ARMTLGALLT LTEEGIQAWR |
| 401 | SAASRRFITQ ALRRYLAEYF DGVVLPRHWR FCMQLPFDER |
| 441 | GKLSVTQLAT RFATHPLQPE VLAEWCDDNT ALLELHVPAT |
| 481 | LIHFSGHFPG LPILPGVVQI DWVVRYAAHY FARCNGFQTL |
| 521 | EQIKFLSMVR PGTTLRLALA HDPERARITF RYYVGERDYA |
| 561 | TGRIVYSKSA VV |

A sequence for an acyl-CoA synthetase from Neurospora crassa is shown below as SEQ ID NO:65 (NCBI EAA28332.1).

| | |
|---|---|
| 1 | MANTGPGNVP LHFIQKPPFT VEDPNAQPIP GETIPRRHPK |
| 41 | AKNGLATRPA PGVNTTLDLL TRTVELYGDE RAIGSRKLIK |
| 81 | LHKDIKKVPK VVDGETVMVD KEWQCFELTP YSYITYGEYF |
| 121 | TIVKQIGAGL RKLGLEPKDK LHIFATTSPQ WLGMSHAASS |
| 161 | QSLTIVTAYD TLGESGVQHS LVQSKASAMF TDPHLLKTAT |
| 201 | NPLKEATSVK VVIYNNHTTQ PVSQDKIDAF KAEHPDLTVL |
| 241 | SFEELRALGE ENPVPLTPPN PDDTYCIMYT SGSTGPPKGV |
| 281 | PVSHAGFVAA VAGLYAVMEE SVTHRDRVLA YLPLAHIFEL |
| 321 | VLENLGVFVG GTLGYSNART LSDTSMRNCP GDMRAFKPTI |
| 361 | MVGVPQVWET VKKGIEGKVN SAGALTKALF WGAYNIKSFL |
| 401 | VSNNLPGKTI FDDLVFGQVR TMTGGELRFI VNGASGIAAS |
| 441 | TQHFMSMVVA PMLNGYGLTE TCGNGALGSP MQWTSNAIGA |
| 481 | MPAAVEMKLV SLPELNYHTD TVPPQGEILF RGACVIKEYY |
| 521 | ENPEETAKAI TPDGWFKSGD IGEIDANGHL RVIDRVKNLV |
| 561 | KLQGGEYIAL EKLEAVYRGA VFVHNIMVHG DNSAPRPIAV |
| 601 | VVPNEKALAE KAEELGLGAE APGEMHRNRK LRDAVLKELQ |
| 641 | SVGRRAGLSG METVAGVVLV DDEWTPANGF VTATQKINRR |
| 681 | AVKERYSKEI SDCLDGK |

A sequence for a long-chain acyl-CoA synthetase from Nannochloropsis gaditana (strain CCMP526) is shown below as SEQ ID NO:66 (Uniprot I2CP03).

```
                10 20 30 40
MDRYKWRTLP DVFETVASLA PEAVAVEDMV HTPTAKMTYG
                50 60 70 80
ELNRQIGALA AFFQHEGLKP GQCVSVFAEN SHRWLIADQA
                90 100 110 120
ILKAGACNAV RGVKAPVDEL QYIYQNSESV ASVVESVEQI
              130 140 150 160
EALMRTNGGL TGRYGPPRFI LVLFPGERSG QEIRELANLP
              170 180 190 200
PPTQVLTFDE ALSASLARPL TFRPVPKDVR SVATLVYTSG
              210 220 230 240
TTNKPKGVVL RHSNLLHQVN YNSFTDSPSK EPAYNPVLGD
              250 260 270 280
VLVSVLPCWH IFERTAEYWM FSKGIHVVYS NVKNFKADLA
              290 300 310 320
KHQPQFIVAV PRLLETIYRG VLQKFATEKG AKKKIIEFFT
              330 340 350 360
RVGSAWVKAW RVARGLVLRS RAPNPIERLL ALVLALVLSP
              370 380 390 400
LAAVGDKLVW SKVRAGLGGR IKVLVAGGSS MPLVLEDFFE
              410 420 430 440
LLRTPVIVGY GMTETSPVIT NRVAEKNLAG SVGRTARDTE
              450 460 470 480
VKIVDPESGA RLPEGQPGLV LMRGPQMMAG YKSNAEASKA
              490 500 510 520
VLDQEGFLDT GDLGRIHPLT KHLIITGRAK DTIVLSNGEN
              530 540 550 560
VEPQPIEDVV CANSALVDQV MCVGQDEKVL GMLVVPNVRA
              570 580 590 600
LARAGLVDRG LAERVAELLG GQVLTNGIAG SRAELEEVEA
              610 620 630 640
SLREKKEVKK ALLADIARAM GKSFRETERV GAVEVVLEPF
              650 660 670
NMANGFLTQT LKVKRNVVSG HYAQEIEQMY R
```

A sequence for an acyl-CoA synthetase from Nannochloropsis gaditana (strain CCMP526) is shown below as SEQ ID NO:67 (Uniprot K8YP55).

```
                10 20 30 40
MHGRSKKLGN ILEELGVKKG DRVATLAMNT YRHMELYFAV
                50 60 70 80
SGAGAVLHTL NPRLFAETLT WIVHHAQDSV LFFDPCFASL
                90 100 110 120
VERLLPHCPS VKHWICLVDE ERMPVLPSLS PSSPFLSLHN
              130 140 150 160
YEALLREGKE DYVWPILEET AASSLCYTSG TTGIPYTAAM
              170 180 190 200
VGCKLVLPGS ALDGASLYEL MKEEGVTLAA GVPTVWLPVL
              210 220 230 240
HHLDQDPGQG LPKLRRLVIG GAACPPSMLR AFKERHGIEG
              250 260 270 280
KHLALPTEDQ HNVLSTQGRT IYGVDLRIVA PSPPPYLPSS
              290 300 310 320
SSSYSPPYPP RWSEVPWDGV SPGELCARGH WVATDYFSPT
              330 340 350 360
QAPEEGERDG GVRAGHQESF YTDDDGERWF LTGDVATICP
              370 380 390 400
DGYIKITDRS KDVIKSGGEW ISSIELENIA TNHPEVALAA
              410 420 430 440
VIAMPHRKWD ERPLLIVVLK DSAALSLHYS TTSSSPSTSS
              450 460 470 480
DTDRAIRLTK EALLDHFKGK VAKWWVPDDV IFVDSLPQGP
              490
TGKILKTELR QRFSRRP
```

A sequence for a long chain acyl-CoA synthetase from Nannochloropsis gaditana is shown below as SEQ ID NO:68 (Uniprot W7TGG5).

```
                10 20 30 40
MPKYTTTVAS GEVDLRIEKE GPGSWAPKTV FQVFEETVKK
                50 60 70 80
YGDSPALHYK KVPHGGSLAT TEWSSYTWRE YYDLTLEFCK
                90 100 110 120
SLLSLGFPAH GAINLIGFNS PEWLIANCGA IAAGGVGVGI
              130 140 150 160
YTSNGVDACK YITEHSEAEV VVVENAKQLE KYLKIAKELP
              170 180 190 200
RLKALVIYSG TAEGYKCDVP IYSWKDFMAL GSGVKDEAVR
              210 220 230 240
ARIEAQRPGH CCTLIYTSGT TGPPKAVMIS HDNLTWTVKN
              250 260 270 280
FVASLPFTLT CEDRSVSYLP LSHVAAQMLD IHCPIATGAK
              290 300 310 320
IYFAQPDALR GSLPVTLKDV CPTYFFGVPR VWEKIYEKMQ
              330 340 350 360
EVARSTTGVK RALAQWAKAK GLEKNRRQQY GCGGGAPVGF
              370 380 390 400
GCAHALVLSK VKAALGLHQT KMCITSAAPI AVEILEYFAS
              410 420 430 440
LDIPVLELFG QSECTGPHTS NFSYAWKIGS IGRDIPGVKT
              450 460 470 480
KQHANMSEFC MYGRHIMMGY MKMEDKTQEA VDNEGWLHSG
              490 500 510 520
DVAQVDADGF WSITGRIKEL IITAGGENIP PVLIENEIMS
              530 540 550 560
ALPAVANCMV VGDKKKFLTV LLTMKAKLDD QGNPTKELNK
              570 580 590 600
EALDIGKEIG SNASTTEQVA SDPHWKKYFD EGLKKANSTA
              610 620 630 640
TSNAQFVQKW SVLPLDFSEK GGELTPTLKL KRSVVAEKYA
DVIADMYKA
```

A sequence for a long chain acyl-CoA synthetase from Nannochloropsis gaditana is shown below as SEQ ID NO:69 (Uniprot S5PTC7).

```
                10 20 30 40
MPKYTTTVAS GEVDLRIEKE GPGSWAPKTV FQVFEETVKK
                50 60 70 80
YGDSPALHYK KVPHGGSLAT TEWSSYTWRE YYDLTLKFCK
                90 100 110 120
SLLSLGFPAH GAINLIGFNS PEWLIANCGA IAAGGVGVGI
              130 140 150 160
YTSNGVDACK YITEHSEAEV VVVENAKQLE KYLKIAKELP
              170 180 190 200
RLKALVIYSG TAEGYKCDVP IYSWKDFMAL GSGVKDEAVR
              210 220 230 240
ARIEAQRPGH CCTLITTSGT TGPPKAVMIS HDNLTWTVKN
              250 260 270 280
FVASLPFTLT CEDRSVSYLP LSHVAAQMLD IHCPIATGAK
              290 300 310 320
IYFAQPDALR GSLPVTLKDV CPTYFFGVPR VWEKIYEKMQ
              330 340 350 360
EVARSTTGVK RALAQWAKAK GLEKNRRQQY GCGGGAPVGF
              370 380 390 400
GCAHALVLSK VKAALGLHQT KMCITSAAPI AVEILEYFAS
              410 420 430 440
LDIPVLELFG QSECTGPHTS NFSYAWKIGS IGRDIPGVKT
              450 460 470 480
KQHANMSEFC MYGRHIMMGY MKMEDKTQEA VDNEGWLHSG
              490 500 510 520
DVAQVDADGF WSITGRIKEL IITAGGENIP PVLIENEIMS
              530 540 550 560
ALPAVANCMV VGDKKKFLTV LLTMKAKLDD QGNPTKELNK
              570 580 590 600
EALDIGKEIG SNASTTEQVA SDPHWKKYFD EGLKKANSTA
              610 620 630 640
TSNAQFVQKW SVLPLDFSEK GGELTPTLKL KRSVVAEKYA
DVIADMYKA
```

A sequence for an alcohol dehydrogenase from Mortierella elongata AG-77 is shown below as SEQ ID NO:70 (Uniprot A0A197K9R3).

```
                10 20 30 40
MSASNAKVED TTTTFTGWAS TGSLPLKKFS YHPRPLGPKD
                50 60 70 80
IEIEITHCGI CGSDVSTVTG GFGPLSTPCI AGHEIVGTVV
                90 100 110 120
KAGPTVFTRS ATLSVLVALL IPAVTGGFAD RLRVSSEYAY
              130 140 150 160
KIPSEIPPAE AAPPLCAGIT TYTPLKHFGA GPGKRVGVMG
              170 180 190 200
IGGLGHLAIQ WAAALKADEV VAISTSDNKR EEAKKLGATK
              210 220 230 240
FVNSRNEEER KAARHSMDIL LLTSNDKNTD WGELIDYVAS
              250 260 270 280
HGTLVLLALP EIPTIAVPPS SLLMRHVSIA GSLTGGREIT
              290 300 320 330
QEMLEFAAKH NVHPWITTMP MSDANTAVKL WLETIWCDVA
              340
ESVVAIVVAV AGEPVMPARK
```

Another sequence for an alcohol dehydrogenase from Mortierella elongata AG-77 is shown below as SEQ ID NO:71 (Uniprot A0A197JDD8).

```
                10 20 30 40
MTGGRTIKAA LYEGVNPSAP LLKVIDLPAP VANNGDAVVK
                50 60 70 80
ILATRVVSYA KEVLDGTRPY PNLLPMVPGP GGVGIIQSVA
                90 100 110 120
PGAIHIKPGQ MVFIDPTVRS RDHPVSPEAM LQGLVAFGSG
              130 140 150 160
QELQKVWNNG SWAEEMLVPL ENLTVIPESI QAKFNPAELT
              170 180 190 200
SISNYAVPLG GLYPNLRPGQ TVVITGSTGM FGSSAVAVAL
              210 220 230 240
ALGARRVIAS GRNKKQLDEF VRLYGPRVVP VVVTGDVAQD
              250 260 270 280
TQAFLKAAGE GFDIDVTFDI LPPQATFGAV QSSILALRNG
              290 300 310 320
GTAVLMGGLN SSAEIPYPAI MNKGLTIKGH FMYDRSGPTT
              330 340 350 360
IIGLADAGLL DLHHRQEPKF FKLSEINDAV EWSAAHPGAF
DATLVLP
```

Another sequence for an alcohol dehydrogenase from Mortierella elongata AG-77 is shown below as SEQ ID NO:72 (Uniprot A0A197JLB4).

```
                10 20 30 40
MKAALYEGVN HSAPLLKVTD LPVPIATNGD AVVKILASRV
                50 60 70 80
VSYAKDVLDG TRPFPNLLPM VPGTGGVGII QSVAPGAIHI
                90 100 110 120
KPGQMVFINS AVRSRDHPVT PEGMVQGLLA FGRSKELQRA
              130 140 150 160
EEMLVPLENL TVIPESVQAK FDPAELTSIS NYAVSFGGLY
              170 180 190 200
PNLRPGQTVV ITGSTGVFGS SAVAVALALG ARCVIASGRN
              210 220 230 240
KKQLDEFATL YGPRVVPVVT TGDVAKDTAA FVKAAGEGFD
              250 260 270 280
IDVSFDILPP QAGFGAVKSS ILALRAGGTA LLMGGVNSSV
              290 300 310 320
EIPYSVIMNK GLTIKGVFMS DRAGPTTIIG LAEAGLLDLH
              330 340 350
HRQEPKIFKL DEINDAVEWS SNHSSAFDAT IVIP
```

A sequence for an alcohol dehydrogenase from Nannochloropsis gaditana (strain CCMP526) is shown below as SEQ ID NO:73 (Uniprot I2CR67).

```
                10 20 30 40
MPVIGLGTWK APKGEVKKAV LAALKQGYRH LDCACDYGNE
                50 60 70 80
EEVGAAIKEA MEAGVVTRKD LFVTSKLWNT FHAREHVEVA
                90 100 110 120
IQKSLKDLGL DYLDLYLIHF PISMKYVPIE ELYPPEWLNP
              130 140 150 160
TSKKIEFVDV PVSETWAGME GVCRKGLARN IGVSNFCAQT
              170 180 190 200
LMDLLKYAEI KPAVNQIELH PYLTQDSLVA FCQEKGIVLT
              210 220 230 240
AFSPLGASSY IELGMDRGEG VGVLNNPVVQ AIAREHSRTP
              250 260 270 280
AQVCLRWAVQ RGYTAIPKST HESRLQENLH VFDFTLSAED
              290 300 310
MVKISRLNRH LRYNDPGEFC KGMGLPNGYP IYA
```

Another sequence for an alcohol dehydrogenase from Nannochloropsis gaditana is shown below as SEQ ID NO:74 (Uniprot W7TDK1).

```
                10 20 30 40
MTDPSASTTA AAQLPGRMLA GVADHHGDRF DMREIPVTPP
                50 60 70 80
GVGQALVKVV TSGVCHTDVH AVDGDWPAPT KLPLVPGHEG
                90 100 110 120
AGVVVAVGPG VSSTVVSLGD RVGIPWLHSS CGSCEFCLSG
              130 140 150 160
RENLCPLQDN TGYSVDGCFA QYVLAPAAHL AKIPDEVSFE
              170 180 190 200
QAAPILCAGV TTYSAIKATE ARPGQFLTVI GAAGGLGHLA
              210 220 230 240
VQFGVALGLR VMALDRGADK LKFCTDTLGA EAAFEAMDPG
              250 260 270 280
VVDQVIATTK GGSHGVLCLA PSIGAFKSAV SLCRRGGTIV
              290 300 310 320
MVGLPKGDLP LNIFDIVIRG ITVRGSIVGT RKDLDEALDF
              330 340 350 360
AARGKVKCHT EMHGFGELNQ VFDQLRSGKV MGRLVLSVDG
M
```

Another sequence for an alcohol dehydrogenase from Nannochloropsis gaditana is shown below as SEQ ID NO:75 (Uniprot W7TYB6).

```
                10 20 30 40
MGKRQVSYFA FSTSPVSGKP AAIPPSLIGI STLNALRDAE
                50 60 70 80
KVADAVKHAV SSVVKYVDCS SDSQNEKQIG NALSAFDRSS
                90 100 110 120
FYVGSKLSCC DAAPEDVTEA CKRSITELGV SYLDNYMMHW
              130 140 150 160
PVQLKSDSKP VSLDDGDTYE LVQDGDMDCI MATYEAMERL
              170 180 190 200
VDQGLVRSLG VSNMGIRTLS ELLSRCRIRP TVLEVEMHLY
              210 220 230 240
LAQPKLLEFC REENIHVVAN SPPGKMRNRH PNDPSLLDDP
              250 260 270 280
VLLRIAEEAV RAAQVLLRRG IQRGRSITRK TPSQSLMDEN
              290 300 310 320
KDLLDWCLSR DHMSRLDALD KGSRFPSVLP SMCDLDRDSE
              330 340 350 360
NYAGAGHPVS QPHRTPCTMD KNGGFRNRFE RPGKYLKTDI
              370 380 390 400
LVQRGALSDL ARLGKSIIPE ESHGSANYLI TDSVVDALYG
              410 420 430 440
DTVLNGLKSA GLDMTKIVVP AVSMDESGEP STEPNKNGAI
              450 460 470 480
FNACVDRVLG NGISKHSCII SLGGGVINNL CGVIAATLYR
              490 500 510 520
GIKLVHFTTT TMGMLDAAID FKQAFNHSCG KNLVGAYYPA
              530 540 550 560
DLIVMDPECL KTLSNRHMLN GVAEALKHGL TQSWELTSAI
              570 580 590 600
VEPLRGDSAR LGDSKYLETL CKETIEIKVP TLTHYKESDF
              610 620 630 640
NEMVPQYGHA VAHAVEHLSW EEGQVPLLHG EAVAIGMCVT
              650 660 670 680
AELGHLLGLC DKSVVDHHYD LVGTTGLPCN VPDTMKVNDI
              690 700 710 720
LHVMTYDKHF MSKPCMGFCK EIGVMAKNKD GSYAFSVEME
              730
PVREALQLNM SK
```

A sequence for a glycerol kinase from Mortierella elongata AG-77 is shown below as SEQ ID NO:76 (Uniprot A0A197JVE6).

```
                10 20 30 40
MPSFIGAIDN GTTSSRFLIF DEKGNLVIGH QLEYRQIFPH
                50 60 70 80
PGWVEHDPMD ILGSVTACIE GALRKFELQG NDVKNLRGIG
                90 100 110 120
ITNQRETAVV WDRTTGKPLH NAIVWSDTRT QDVVTKLCES
              130 140 150 160
SDKGTDALKD ICGLPLTTYF SAVKLKWLLE NSSEVKEAHE
              170 180 190 200
NGNLMFGTVD SWLIYNLTGG KEGGVHVTDV TNASRTMLMD
              210 220 230 240
IKTLQWSEEA LKFFGINADI LPEIKPSSTL FGKVQHPALE
              250 260 270 280
QLQDVPIAGC LGDQHAALVG QHCFQVGEAK NTYGTGCFML
              290 300 310 320
FNTGSKITPS NNGLLTTVGY QFEGEPAAYA LEGSIAVAGS
              330 340 350 360
AVKWLRDNMG IIRSAEEIND LAAQVDSNGG VVFVTAFSGL
              370 380 390 400
FAPYWRPDVR GSIVGISQHT TKHHLARATL EATCFQTRAI
              410 420 430 440
LDAMNADSGH PLATLRVDGG LSNSDLCMQL QSNILGLEVA
              450 460 470 480
RPQMRESTAL GAATAAGVHL GIGIWKGGFK AFAERARESK
              490 500 510 520
EVLQIFTPKI NDEEREKEYA LWQKAIDTTI GVKSKTTGKR
EP
```

A sequence for a glucose kinase from Nannochloropsis gaditana is shown below as SEQ ID NO:77 (Uniprot W7U0M7).

```
                10 20 30 40
MTSSYINSYV GAIDQGTSST KFIIYNHSGQ QVGLHQLEHA
                50 60 70 80
QIYPQPGWVE HDPMEIWANT VTCIRRAMES ANVDAELLEA
                90 100 110 120
VGITNQREST LIWNKKTGVP YYNVIVWNDA RTRGICEDLK
              130 140 150 160
TAGRRGIDRF REKTGLPIAT YFSASKILWL LDNVPGLRDD
              170 180 190 200
AEKGEAIFGT LDSWLIYKLT DGQVHSGPCV AYPGGLSPSS
              210 220
LSSALRPPAS PPSQAPSLSP DP
```

A sequence for a diacylglycerol kinase from Nannochloropsis gaditana is shown below as SEQ ID NO:78 (Uniprot W7UAL1).

```
                10 20 30 40
MDEELNVLSP FLVKAEVLLV LVVVLVASVV WLFWEIVSFM
                50 60 70 80
MDRGKEETNP DWWEVLRNCQ HRRLIIPPYC VQEVPELGTF
                90 100 110 120
SRLTTATTNA MKNMSGVIQR TSHLISGGSG KSAAAIKKGA
              130 140 150 160
RQDLPSTQQE GDENMKGYTV DGNARGVKLR RRGSKQSIVG
              170 180 190 200
LSNHGTSAGG KPALQPTANP TPLTLSENGA NPDASAASDA
              210 220 230 240
RPKPHRLDLN GEEGNMVPCN GSLSSRAGDG KRVVGMSGLA
              250 260 270 280
STSAAAGSDA SSANVKSMEI SPADTPCRGR IRFLPHQRER
              290 300 310 320
QQIENEEKSH EGKPTRSGLP LRALDSQPPL TPYALPDAEG
              330 340 350 360
VLASSAQSSR HAPDAIAATP RLSSSHAANG EPITTPAQPV
              370 380 390 400
RLPSMEHAHS GTGVALSGGS SGVAGRGFIF SPLPEDCTPL
              410 420 430 440
LAFVNSRSGV SQGAYLIHQL RRLLNPIQVI DIANEDPARA
              450 460 470 480
LRLYLELPRL RVLVCGGDGT AKWIMNVLED LNPECWPPIA
              490 500 510 520
ILPLGTGNDM ARVLGWGGGY NNQSIVEFLA QVQRAHVVVV
              530 540 550 560
DRWEMKLTPA GKGSSRAKTV TFNNYFGIGV DAQAALKFHH
              570 580 590 600
LREQKPQLFF SRLVNKLWYG MLGAQDLFRR TCVSLPERLK
              610 620 630 640
IVADGKELTL PAHVQGVIEL NIESYGGGVK LWNVEEDDES
              650 660 670 680
AGNGLFDASS SSCSSEEGDR SEDESRRQRR RRRRRERQRR
              690 700 710 720
QQSQAEEEAH RQREQQEKPS SMALTSSSMQ DGLMEVVAIN
              730 740 750 760
GVVHLGQLQV GLSKAVKICQ CREAVITTTR DLPMQVDGEP
              770 780 790 800
WPQAKSTIKI TRKKDPAYLL RRTMDSGGAV VGEVVELLES
              810 820 830 840
AVKDGVISLP QKKSLLTELS RRVEMKRKVF EQELSQNDGV
              850 860
PSFSKGFDVS RLRLAADSNS KDCVLM
```

A sequence for glycerol-3-phosphate dehydrogenase from Mortierella elongata AG-77 is shown below as SEQ ID NO:79 (Uniprot A0A197JEE6).

```
                10 20 30 40
MWRRIPATGA RHSTSFRTKA VYATAGATTL ALSGYYYNLK
                50 60 70 80
QQQRALDDSF EYPPQSSMIY LEPQQAARDP TRPHAFWAPP
                90 100 110 120
SREDMIRMLQ EGPGSIVKEK TAAAAAAAAA AAAGTTPGSK
              130 140 150 160
PVVAVAATME DDKDSDVFDL LIIGGGATGA GCAVDAATRG
              170 180 190 200
LKVAMVERDD FSSGTSSRST KLVHGGVRYL EKAVRELDIE
              210 220 230 240
QYKLVKEAIN ERANFLKVAP YLSYQLPIML PIYKWWQVPY
              250 260 270 280
YWAGSKAYDL LAGHQGMESS YFLSRGKALE AFPMLKNDKL
              290 300 310 320
VGAMVYYDGQ HNDSRMNVAL GLTAVQYGAV IANHVEVIEL
              330 340 350 360
HKDENRRLCG ARVRDAMTGK EFNVKAKGVI NATGPFTDGI
              370 380 390 400
RQLDDPSIQS IVSPSAGVHI ILPNYYSPGN MGLLDPATSD
              410 420 430 440
GRVIFFLPWQ GNTIAGTTDS ATKVTPNPMA TEEEINWILG
              450 460 470 480
EVKNYLNPDV KVRRGDVLAA WSGIRPLVRD PAAKSTEGLV
              490 500 510 520
RNHMINVSPS GLLTIAGGKW TTYRAMAAET IDEAIKEFGL
              530 540 550 560
TPARGCSTER VKLIGSHGYS NTMFIRLIQQ FGLETEIAQH
              570 580 590 600
LANSYGDRAW AVASLAQSTG KRWPVFGRRV SNQYPYIEAE
              610 620 630 640
VRYAVRREYA CTAVDVLARR LRLAFLNVHA ALDALPRVVE
              650 660 670 680
IMAEELKWDA ARQAKETEDA KAFLTTMGLP VSPIAYPTNV
              690 700 710 720
PEAVVGHPVV DGEKVQPTSF WGRMSGKSAS GAIVTDSFYS
              730 740 750 760
RAQFNPEELA EFHKVFGALD HDGDGHIDGH DLEEVLIHLD
              770 780 790 800
VQVEPQVLKS IIEEVDLDNS GTIEFNEFLE VMGGLKEHAS
              810 820 830
RTAFSKIIVE VESKRNVDYG IKAKTTDRSG GGA
```

Another sequence for glycerol-3-phosphate dehydrogenase from Mortierella elongata AG-77 is shown below as SEQ ID NO: 80 (Uniprot A0A197JIF5).

```
                10 20 30 40
MTERVALIGS GNWGSAVAKI IGRNVRKFDH FDNKVKMWVF
                50 60 70 80
EEKVNGQNLT EIINTKHENV KYLPGIQLPS NIVACPDLLE
                90 100 110 120
TCRDATMLVF VVPHQFVTSI CKQLKGRIPA NCKAISLIKG
              130 140 150 160
IDVNADGFRL ITDMIQESLG VPTCVLSGAN IANEVAEEKF
              170 180 190 200
CETTIGYRNR ADGELFRDIF HTPSFRVNIV PDVVGVELCG
              210 220 230 240
ALKNIVAIGG GLVDGLKLGD NTKAAIIRIG LYEMRKFSKM
              250 260 270 280
FYADVKDETF FESCGVADLI TTCAGGRNRK VAEAHVTTGK
              290 300 310 320
SFDQLEQEML NGQKLQGTST AQDMYNILSK KNLCHEFPLM
              330 340
TTIYKICYEG LPPIRIVEDI
```

Another sequence for glycerol-3-phosphate dehydrogenase from Mortierella elongata AG-77 is shown below as SEQ ID NO: 81 (Uniprot A0A197KEB5).

```
                10 20 30 40
MLITECISLF HRGSAVAKIV GGNVQKYDHI QNEVKMWVFE
                50 60 70 80
EQVDGQNLTE IINAKHENVK YLPGIKLPEN IVACPDLIKT
                90 100 110 120
CEDATMLVFV VPHQFVASVC RQLKGKISPK CKAISLIKGV
              130 140 150 160
DVEENDNGFR LITDMIQDSL GIRACMLSGA NIATEVAEER
              170 180 190 200
FCETTIGYRN KADGELFKEI FNTPTFRVNI VEDVVGVELC
              210 220 230 240
GALKNIIAIG GGLVDGLKLG DNTKAAIIRI GLYEMRKFAK
              250 260 270 280
MFYADVKDET FFESCGVADL VTTCAGGRNR KVAEAHVTTG
              290 300 310 320
KSFDQLEKEM LGGQKLQGTS TAKDMYGILS KKGLCKEFPL
              330 340
MTTIYRICYE DLPPIRIVED I
```

A sequence for glycerol-3-phosphate dehydrogenase from Nannochloropsis gaditana is shown below as SEQ ID NO:82 (Uniprot W7U0Y7).

```
                10 20 30 40
MATLHISNLT LTIYNHGIFV LMSAALSFLL IVWRFSLAEA
                50 60 70 80
GRSHHFEGPS SNPVKPHSIT IVGSGNFGSA IARLLGRNVL
                90 100 110 120
RSPKHFRSEV RMWVFEEELD DGRKLSDVIN ADHENVKYLP
              130 140 150 160
GIQLPINVRA VPDLSDAVRN ASIVVFVLPH QFLPGLLPRI
              170 180 190 200
SSCLHRGAMA VSLVKGLDFD DEGPVLITDM IREGLGEDVS
              210 220 230 240
EVCVLMGANV ADEMARDEFC EATLGCPDPE GAGAVLQQLF
              250 260 270 280
DCPTFRVEVT PDPIGVELCG ALKNVVALAA GFCDGLDWGG
              290 300 310 320
NTKAAIIRRG LEEMRLFCKL LHPSVRDMTF FESCGVADLI
              330 340 350 360
TTCYGGRNRK CAETFARAGG TMAWDEIEKE ELGGQHLQGP
              370 380 390 400
QTTSKLHKVL EQKKWLSRFP LFRSVYQIAY QGRPPATLVQ
DL
```

Another sequence for glycerol-3-phosphate dehydrogenase from Nannochloropsis gaditana is shown below as SEQ ID NO:83 (Uniprot W7TAY6).

```
                10 20 30 40
MSPTFRRRHS NAPFKLQIFM VKFLAVVALL GCCCLHGVAS
                50 60 70 80
GTPPHAAFVP RASTKSLGNR LAKAPQARRE QTIMQLSARR
                90 100 110 120
SRSMRPLPYP VRFAVLGGGS FGLALASVLG KKSIPVTILV
              130 140 150 160
RKEEVAEHIN LHHRHPTYLS DIALAPSIRA TVQPEEALRD
              170 180 190 200
ASFIIHAVPV QYSRKFLEDI APHVPKNTPI ISTSKGIETG
              210 220 230 240
TLCMMQDILL ETLGPNRETA YLSGPSFARE IALGLVTAVV
              250 260 270 280
AASESEALAN EICDIMGCNY FRVFTSTDVV GVEVGGAVKN
              290 300 310 320
VIAIAAGMCE GLGLGTNAMA ALVTRGCNEM QRLALSLGAR
              330 340 350 360
PSTLTGLSGV GDTFGTCFGP LSRNRNLGVR LGKGERLENI
              370 380 390 400
LGSSTEVAEG HATAFSLVQL IEKTNRAYRR ELEFPIIYGV
              410 420
KEILEGKRTP AEGLRDLMAM PVRVEMWNL
```

Another sequence for glycerol-3-phosphate dehydrogenase from Nannochloropsis gaditana is shown below as SEQ ID NO:84 (Uniprot W7TIR6).

```
                10 20 30 40
MSLQPHLALL GMAGSLVVAD RLRSGPGRKS RAKDSHRHLP
                50 60 70 80
PTSRSANCEA SGGKRELSPV EQLEDMRTTP IKCRDGTLVY
                90 100 110 120
PYSLPTRDAQ LNRLKKEKFD VLVIGGGCVG SGVALDAQIR
              130 140 150 160
GLKTAMVEAN DFSAGTSGRS TKLIHGGIRY LETAFWKLDY
              170 180 190 200
GSFALVQEAL EERAHMLNAA PYMNSPLPIM IPIYKWWEVP
              210 220 230 240
YFWAGAKAYD LVASRQKSVP SSHYMDVDEA LFQFPMLRGK
              250 260 270 280
GLKGAIIYYD GQMNDTRMGL TIALTAAQEG AAIANRVEVV
              290 300 310 320
SLLKDPGTGQ VNGARVQDRL TGVEWDIAAK VVVNATGVFA
              330 340 350 360
DKIRKFDDPK AVELIEPAAG VHVMFPAHFS PAKMGLIVPK
              370 380 390 400
TTDGRVLFFL PWEGCTLAGT TDSHSDITMH PQPTAQEVNF
              410 420 430 440
IMQETNRYLT TNVAAKDLIA AWSGLRPLVK DPEKIKEGTA
              450 460 470 480
ALSRNHVIEV SETGKLITIT GGKWTTYRRM AEDTVDRILQ
              490 500 510 520
EHAGLLANGD VSPQASTWNR KLLGADRAGI VCAQKFNQIG
              530 540 550 560
ITLRNDYELP EDVSAHLVKS YGTRALQVAE WVRAGYLDTK
              570 580 590 600
PGKAKRLHSR YPFLEAEVIF AVDQEYALKP MDILARRTRL
              610 620 630 640
AFLDTEAARA AVPRVVKLMG DLLGWSWRQR TMEKAEALAF
              650
LETMNVEKTA LLKK
```

A sequence for a GPAT acyltransferase from Mortierella elongata AG-77 is shown below as SEQ ID NO:85 (Uniprot A0A197K296).

```
                10 20 30 40
MASKNSKTGP DNAGASTGPA LELKPLKNVM PIVPAQQVDS
                50 60 70 80
SSCPPSGETS PLLENAPNGK LATQSGGPDN DESGVENITK
                90 100 110 120
KHAGRIREDP VGFVVQTAAF YQGTGWRSYS NYVGTRIFYE
              130 140 150 160
GFSASFKDRI LASQKVVELV KSMANKQLEV LIKQRQDAHE
              170 180 190 200
AEKVANAGKK NFKPKVWPMR PEDVEVRRKT LEAELTAVAK
              210 220 230 240
TNIDKLVCDM NSMKFIRFFA FLINNILVRM YHQGIHIKES
              250 260 270 280
EFLELRRVAE YCAEKKYSMV ILPCHKSHID YLVISYIFFR
              290 300 310 320
MGLALPHIAA GDNLDMPVVG KALKGAGAFF IRRSWADDQL
              330 340 350 360
YTSIVQEYVQ ELLEGGYNIE CFIEGTRSRT GKLLPPKLGV
              370 380 390 400
LKIIMDAMLS NRVQDCYIVP ISIGYDKVIE TETYINELLG
              410 420 430 440
IPKEKESLWG VITNSRLLQL KMGRIDVRFA KPYSLREFMN
              450 460 470 480
HEIDRREIIN EQEMTSNAAK SQLLKALGYK VLADINSVSV
              490 500 510 520
VMPTALVGTV ILTLRGRGVG RNELIRRVDW LKREILSKGG
              530 540 550 560
RVANFSGMET GEVVDRALGV LKDLVALQKN LLEPVFYAVK
              570 580 590 600
RFELSFYRNQ LIHLFIHEAI VAVTMYTRIK IGGAKSTQQI
              610 620 630 640
SQTELLNEVT FLSRLLKTDF IYNPGDIQSN LENTLEYLKK
              650 660 670 680
SNVIEINSEG FVGLSDVERG IGRENYDFYC FLLWPFVETY
              690 700 710 720
WLAAVSLYTL IPTAKEITEQ ANAGGDQLHW VEERVFVEKT
              730 740 750 760
QMFGKTLYYQ GDLSYFESVN METLKNGFNR LCDYGILMIK
              770 780 790 800
KPTGPKERTK VALHPDFMPS RGSDGHVIAS GALWDMVEHI
              810 820 830 840
GTFRREGKNR RDNATVSSRV LRFAEVVANS PAPVKVPMPS
              850
PAPKQGNGAP KL
```

A sequence for glycero-3-phosphate acyltransferase from a bacterium endosymbiont of Mortierella elongata AG-77 is shown below as SEQ ID NO:86 (NCBI GAM53307.1).

| | |
|---|---|
| 1 | MTYLFIAALA YGIGSISFAV VVSAAMRLQD PRSYGSKNPG |
| 41 | ATNVLRSGNT LAAVLTLIGD ALKGWLAVWL TAQFVHSFGS |
| 81 | QYEVGNEAIG LAALAVFLGH LWPIFFHFKG GKGVATAAGV |
| 121 | LFAIHPILGL ATAASWLIIA FFFRYSSLAA LVAAIFAPLY |
| 161 | EILMFGFDSN SIAVLAMSLL LISRHRSNIQ NLFAGKEGRL |
| 201 | GQKSKDKSL |

A sequence for a 1-acyl-sn-glycerol-3-phosphate acyltransferase from Mortierella elongata AG-77 is shown below as SEQ ID NO:87 (Uniprot A0A197KCL2).

```
                10 20 30 40
MSIVTYLQAA IGIPLEYFLV LPKILAVLPK KAQFLAKCII
                50 60 70 80
VLLATLIMSV AGCFISIACA LVNKRYIINY VVSRFFGILA
                90 100 110 120
AGPCGVTYKV VGEEKLENYP AIVVCNHQSS MDMMVLGRVF
              130 140 150 160
PKHCVVMAKK ELLYFPFLGV FMKLSNAIFI DRKNHKKAIE
              170 180 190 200
STTQAVADMK KHNSGIWIFP EGTRSRLDKA DLLAFKKGAF
              210 220 230 240
HLAIQAQLPI LPIISEGYSH IYDSSKRSFP GGELEIRVLD
              250 260 270 280
PIPTTGLTAD DVNDLMEKTR DLMIKHLKEM DRSSSTVISP
              290 300
AATVGKTTAT APQDEASVKK RRTLKD
```

Another sequence for a 1-acyl-sn-glycerol-3-phosphate acyltransferase from Mortierella elongata AG-77 is shown below as SEQ ID NO:88 (Uniprot A0A197K8I3).

```
                10 20 30 40
MSSESTIPWC IITTPVFILA LPRLLAVLPQ KIQPVTKCCI
                50 60 70 80
VLIATFIMSI VGCFVAIVFA LLRRRHEINF VVARIFSFIA
                90 100 110 120
SYPCGVTFKV VGEEHLEKYP AIVVCNHQSS MDMMILGRVF
              130 140 150 160
PKHCVVMAKK ELQYFPFLGI FMTLSNAIFI DRKNHKKAIE
              170 180 190 200
STTQAVTDMK KHNSGIWIFP EGTRSRLETA DLLPFKKGAF
              210 220 230 240
HIAIQSQQPV MPIVAAGYSN IYDSANRSFP GGELEIRVLE
              250 260 270 280
PISTIGMTAD DVNELMERTR AVMLKNLKEM DHSVKSSSNS
              290 300
NGSSTAVAEG KTDEGLTQR RPVKE
```

A sequence for glycerol-3-phosphate acyltransferase from Nannochloropsis gaditana (strain CCMP526) is shown below, as SEQ ID NO:89 (Uniprot K8ZBC7).

```
                10 20 30 40
MVISFIFSWM LQILACIFIC PFLPSCKERL LLLGWIFRSV
                50 60 70 80
SSLVIRLNPY WHLRVLGPRP TRPPSKTLIM CNHLSNADAF
                90 100
FLSSALLPWE TKYIAKASLF Q
```

A sequence for 1-acylglycerol-3-phosphate O-acyltransferase from Nannochloropsis gaditana (strain CCMP526) is shown below as SEQ ID NO:90 Uniprot K8YRH4).

```
                10 20 30 40
MRSNKSCKTC PNRIHVGIAI LFPLLLSAFC FCHFLMLPPA
                50 60 70 80
IALLIMPYAP VRRVLRLWEA TIAAYWLSFG AWLLENFGGV
                90 100 110 120
KLIISGDTFT KKDNVLIICN HRTRLDWMWL WSWAAYFDVL
              130 140 150 160
SSYRVILKDS LRCFPWWGWG MSLCLFPFIR RGQKHRSTDL
              170 180 190
AHLKRNCRYL IQLKVPNSLI IFPEGTDLSP SNQERDRNY
```

A sequence for 1-acyl-sn-glycerol-3-phosphate acyltransferase from Nannochloropsis gaditana is shown below as SEQ ID NO:91 (Uniprot W7U0D6).

```
                10 20 30 40
MTSTASLACG ACTAAVLVCL TTGDGVATRH IDANVGNRRT
                50 60 70 80
SAFLPVMPPM GTPVTGRIRS HPLEAHKMYY VCQGGTRLSQ
                90 100 110 120
RRHERLGTRT AVMVVKTDVE ISDKRDVDPE VGSSSKSTDH
              130 140 150 160
TGVSRFGSAM PKSAEGVGPP PAPQDNFKHK SLAGVPTDYG
              170 180 190 200
PYLTIKGFKI NAFGFFFCFM AILWAIPWAV FLVVYKALLE
              210 220 230 240
FVDKLDPCRY NVDRSSSLWG WLTSLSTDSL PEMTGLENIP
              250 260 270 280
DGPAVFVANH ASWMDVPYSA QLPVRAKYLA KADLTKVPIL
              290 300 310 320
GNAMSMAQHV LVDRDDKRSQ MEALRSALLI LKTGTPLFVF
              330 340 350 360
PEGTRGPGGK MQAFKMGAFK VATKAGVPIV PVSIAGTHIM
              370 380 390 400
MPKEVIMPQC AGRGITAIHV HPAIPSTDRT DQELSDLAFK
              410 420
IINDALPNEQ QCESTSKETG GA
```

A sequence for phosphatidic acid phosphatase from Nannochloropsis gaditana is shown below as SEQ ID NO:92 (Uniprot W7U311).

```
                10 20 30 40
MSSHMPVCRG DPEAGVVPAG GTVGNEEMAG RENGGSGMYR
                50 60 70 80
LAEDVDGNGR DEGCQWVPPA LRTSLERYRW LEIILLSVIV
                90 100 110 120
ILAKEGFGSG VKNHRQYIPL VTQVLPGGAV VVLGNATAFS
              130 140 150 160
YPVRFREGTL ECPPVTLEFC ATSPESALAD PCCEFMTTGA
              170 180 190 200
KPFQTVSHDD LIWITVGLPL ILLVLRHLLL KWYLCSVPAS
              210 220 230 240
SADPMFSSED KSALRPLSGL PFGYSATFCL RDVLIGLFFS
              250 260 270 280
LALTRATTNS LKMLTSQPRP NHFALRLFAS LSPDSSAAIH
              290 300 310 320
YAESAWKAWP SGHSSMSMAS GAFLSLVLLR DLRQFAGPLQ
              330 340 350 360
RQLRACLVIL ALGPVYLAMF VAGTRVHDYF HTTADAVTGS
              370 380 390
ALGLLWAVLA FYQVVPAGGL EVRANTPLKY L
```

A sequence for a diacylglycerol kinase from Mortierella elongata AG-77 is shown below as SEQ ID NO:93 (Uniprot A0A197JW38).

```
                10 20 30 40
MASFPFVLQA HQGNHQVELV YNGQQLEFDG LSLDEPKQSS
                50 60 70 80
SCLPCGPSSA FAGGHRIIKT VEILNIDIEH EDSLVLSVAS
                90 100 110 120
AKNGPTKESV LERLVFQVRD KANAVQWQSN VLSHVYKDIK
              130 140 150 160
KGRHFKVLVN PFGGQGHAKK LWETIAEPIF KAAGCTYDLT
              170 180 190 200
YTTHRYHAKE IARDLNIRLF DAVVSVSGDG VLHEVINGLM
              210 220 230 240
ERPDAIAAHK LPIGAIPGGS GNALSYSLLG EDHGSHVTNA
              250 260 270 280
VLGIIKGRAM PWDLCSVTQG QNRYFSFVLQ SFGLVADVDL
              290 300 310 320
GTEDMRWMGE ARFTVAAVGK LLSQQTYPCE ISYIPVETNV
              330 340 350 360
DKIRAEYNYR RQQSVVWADQ THDELDQSHP TIVDRFGGVN
              370 380 390 400
AQLNKSDGWV TDSEDVITAV GAKLPWISKG MLLNPASTPN
              410 420 430 440
DGLIDLIVFP KGTGRMNGIQ IMLGTETGEH IYHDKVRYMK
              450 460 470 480
VKAFRLTPKN ESGFISMDGE HTPYSPYQVE AHPGLISVLS
              490
IEGRYARSMR E
```

Another sequence for a diacylglycerol kinase from Mortierella elongata AG-77 is shown below as SEQ ID NO: 94 (Uniprot A0A197K901).

```
                10 20 30 40
MDEKKIGFIV NRRGGGGKGG KTWDKLEPAV TTRLASAKWK
                50 60 70 80
VEYTQHSGHA SDLAREFVNE GYNIIVAVGG DGTISQVVNG
                90 100 110 120
YMLADGNSKG CAVGIISSGT GGDFVRTTKT PKDPLEALEL
              130 140 150 160
ILSTESTLVD VGHVSATKPN SPSVTNEQYF INICSVGISG
              170 180 190 200
SIIKRVESSS IAKYISGSLV YWLYTYLTGL VYRPPPVKYT
              210 220 230 240
LTGGSAGADD GKEKHMGLYI MAVANGRYLG GNMHIAPKAQ
              250 260 270 280
ISDGQFDVVC LHDLTLIDAF FKASPALKSG NLMNLPAHQA
              290 300 310 320
FTQRNTKVSI SPVNAKDHIY VEADGEVAGV LPARWEIIPQ
              330
GCRMILPLVQ GSTQSV
```

Another sequence for a diacylglycerol kinase from Mortierella elongata AG-77 is shown below as SEQ ID NO:95 (Uniprot A0A197 KB11).

```
                10 20 30 40
MGIIPTSDKF TVLVVINTHS GRKQGLEAWE NTVKPALNAA
                50 60 70 80
NKPFRLIESN SQGHVVSYFV DNIKPIITDL AQSLSTVTQG
                90 100 110 120
AGDDETIVYP TSAKLQIIVL GGDGTVHEIV NGILKGVEGT
              130 140 150 160
GFVTDAFRPE VEFSVIPTGT GNAISTSLGV TSVQNAVDRF
              170 180 190 200
IAGKTVPLHL MSVATQTSQL YTVVVNSYGL HCATVYDSEE
              210 220 230 240
FRHLGNDRFR QAAMKNVENL KQYEGKLSFF GPIQRYNRIS
              250 260 270 280
ASLVDTETDN NIAQADSKSS AVATLTLPGP FTYLLISKQA
              290 300 310 320
SLEPGFTPTP FAKTSDDWMD VLAVQNVGQA EIMQMTGSTA
              330 340 350 360
TGTHVNQDHV DYIKAKTIEL ETPTQGRLCI DGEFLTIEAG
              370 380
PEGKVRFEVN SDPNIQIFHI FA
```

Another sequence for a diacylglycerol kinase from Mortierella elongata AG-77 is shown below as SEQ ID NO:96 (Uniprot A0A197K5S8.

```
                10 20 30 40 50
MSPNQFQAKA SFAGHQRVSD ARLSLGTHEL TIHAPKGSDN NITTIQVPYS
                60 70 80 90 100
CIYGYETSTD KATGENYKNK VIVHYVAFSG PDLRNPSAAK RTTAQLLFER
              100 120 130 140 150
TEDADRFIQT ARDIGALPTP RRILLLVNPN GGVGKAKRIS DTVVKPMIQH
              160 170 180 190 200
SGLIVKEQYT EYGRHAVDIA SKVNLDEVDS LVVVSGDGVL HEVINGLLSR
              210 220 230 240 250
PDWDRARKTS IGIVPAGSGN AIAASLGIVS UVATITVIR GETSKLDIFS
              260 270 280 990 300
LSQLNRPKIY SMISFSWGMM ADADIESDSY RWIGPIREDV AGFIRMIRIR
              310 320 330 340 350
RYPGKVYVLP PKHQQNPSTT EQQLTPPQSP SHKREPESQF QHLLDSNIKE
              360 370 380 390 400
PPKPWSLIPN MPFYSMILLI NCPNVGETIF FTDTIRFNDG IMRLWYSAET
              410 420 430 440 450
RFWKIIMPFI FDQQNGKMVE RDLMKDLECG GILIIPGVEG KPDDPSTHKV
              460 470 480 490 500
IEPDWVTSSA AKAQNIYQNP GLFDVDGEVM PTARTLIEIH PSLMNILVPE
              510 520
WLYHKDDDNT TARAHEVAVI QAIKAQQKL
```

A sequence for diacylglycerol kinase from Nannochloropsis gaditana is shown below as SEQ ID NO:97 (Uniprot W7UAL1).

```
                10 20 30 40 50
MDEELNVLSP FLVKAEVLLV LVVVLVASVV WLFWEIVSFM MDRGKEETNP
                60 70 80 90 100
DWWEVIRNCQ HRRLIIPPYC VQEVPELGTF SRLTTATTNA MKNMSGVIQR
              110 120 130 140 150
TSHLISGGSG KSAAAIKKGA RQDLPSTQQE GDENMKGYTV DGNARGVKIR
              160 170 180 190 200
RRGSKQSIVG LSNHGTSAGG KPALQPTANP TPLTLSENGA NPDASAASDA
              210 220 230 240 250
RPKPHRLDLN GEEGNMVPCN GSLSSRAGDG KRVVGMSGLA STSAAAGSDA
              260 270 280 290 300
SSANVKSMEI SPADTPCRGR IRFLPHQRER QQIENHEKSH EGKPTRSGLP
              310 320 330 340 350
LRALDSQPPL TPYALPDAEG VLASSAQSSR HAPDAIAATP RLSSSHAANG
              360 370 380 390 400
EPITTPAQPV RLPSMEHAHS GTGVALSGGS SGVAGRGFIF SPLPEDCTPL
              410 420 430 440 450
LAFVNSRSGV SQGAYLIHQL RRLLNPIQVI DLANEDPARA LRLYLELPRL
              460 470 480 490 500
RVLVCGGDGT AKWIMNVLED LNPECWPPIA ILPLGTGNDM ARVLGWGGGY
              510 520 530 540 550
NNQSIVEFLA QVQRAHVVVV DRWEMKLTPA GKGSSRAKTV TFNNYEGIGV
              560 570 580 590 600
DAQAALKEHH LREQKPQLFF SRLVNKLWYG MLGAQDLERR TCVSLPERLK
              610 620 630 640 650
IVADGKELTL PAHVQGVIEL NIESYGGGVK LWNVEEDDES AGNGLFDASS
              660 670 680 690 700
SSCSSEEGDR SEDESRRQRR RRRRRERQRR QQSQAEEEAH RQREQQEKPS
              710 720 730 740 750
SMALTSSSMQ DGLMEVVAIN GVVHLGQLQV GLSKAVKICQ CREAVITTIR
              760 770 780 790 800
DLPMQYDGEP WPQAKSTIKI TRKKDPAYLL RRTMDSGGAV VGEVVELLES
              810 820 830 840 850
AVKDGVISLP QKKSLLTELS RRVEMKRKVF EQELSQNDGV PSFSKGFDVS
              860
RLRLAADSNS KDCVLM
```

Another sequence for diacylglycerol kinase from Nannochloropsis gaditana is shown below as SEQ ID NO:98 (Uniprot W7TXY0).

```
                10 20 30 40 50
MKLIQYFGTA LCVVILSCVT NIIPGGRIAL GRPFSRLFGG SSRNLRAEVE
                60 70 80 90 100
AAVPHFIVPE DRVEYPTPKL AALKSKLKEI GHHKAMGHPH QHQGLDGRRR
              110 120 130 140 150
VSLHPSHRPA PSSLGAAEDK EQEEEGGEEE EEGQEGVIAP PAWKPGHMNP
              160 170 180 190 200
RDSSSDMGKA TKGKPGTPSA FLPLGV2PPS LFPPSARPIR RSPWSLLFRR
              210 220 230 240 250
GLPRPRRKRP IGINRIKTLP PSVTPLIAIV NSKSGGRQGK NLFKRLRAAL
              260 270 280 290 300
SRAQVFDIQK VDLKEALSLY CHLPNSCTLL VCGGDGTASR VFEVVDGMEW
              310 320 330 340 350
KHGPPKIAIV PLGIGNDIAR VLDWNLGHDW SGGYFPWSND AADANLLSVF
              360 370 380 390 400
SDLTRAMERK MDRWELRMTE AVPSSDRERQ PVKYMLGYLG IGVDGKVAID
              410 420 430 440 450
FHKLRDRAPY LFLSPTLNKF YYALMGLRDF FVRSCKNLPD KVELWCDGKP
              460 470 480
IVLPPQTESF IVININSHAG GVELWPEYLM GGGMEG
```

Another sequence for diacylglycerol kinase from Nannochloropsis gaditana is shown below as SEQ ID NO:99 (Uniprot W7TP09).

```
                10 20 30 40 50
MKLIQYFGTA LCVVILSCVT NIIPGGRIAL GRPFSRLFGG SSRNLRAEVE
                60 70 80 90 100
AAVPHFIVPE DRVEYPTPKL AALKSKLKEI GHHKAMGHPH QHQGLDGRRR
              110 120 130 140 150
VSLHPSHRPA PSSLGAAEDK EQEEEGGEEE EEGQEGVIAP PAWKPGHMNP
              160 170 180 190 200
RDSSSDMGKA TKGKPGTPSA FLPLGVPPPS LFPPSARPIR RSPWSLLFRR
              210 220 230 240 250
GLPRPRRKRP IGINRIKTLP PSVTPLIAIV NSKSGGRQGK NLFKRLRAAL
              260 270 280 290 300
SRAQVFDIQK VDLKEALSLY CHLPNSCTLL VCGGDGTASR VFEVVDGMEW
              310 320 330 340 350
KHGPPKIAIV PLGTGNDIAR VLDWNLGHDW SGGYFPWSND AADANLLSVF
              360 370 380 390 400
SDLTRAMERK MDRWELRMTE AVPSSDRERQ PVKYMLGYLG IGVDGKVAID
              410 420 430 440 450
FHKLRDRAPY LFLSPTLNKF YYALMGLRDF FVRSCKNLPD KVELWCDGKP
              460 470 480 490 500
IVLPPQTESF IVININSHAG GVELWPEYLM GGGMEGAFKP SRFDDGYLEV
              510 520 530 540 550
VAISGVLHLG RaRVGLDRPL RLAQAKEVRa RTKSFLPGQV DGEPWRIPRC
              560 570 580 590 600
ELTLRHNGQA PVLQHVSKEL LQYNEWLVGQ GKLDAAGKDQ LLQAFKRRLQ
VSQ
```

A sequence for a diacylglycerol O-acyltransferase 2A (DGAT2A) from Mortierella ramanniana is shown below as SEQ ID NO: 100 (Uniprot Q96UY2).

```
                10 20 30 40 50
MASKDQHLQQ KVKHTLEAIP SPRYAPLRVP LRRRLQTLAV LLWCSMMSIC
                60 70 80 90 100
MFIFFFLCSI PVLLWFPIIL YLTWILVWDK APENGGRPIR WLRNAAWWKL
              110 120 130 140 150
FAGYFPAHVI KEADLDPSKN YIFGYHPHGI ISMGSFCTFS TNATGFDDLF
              160 170 180 190 200
PGIRPSLLTL TSNFNIPLYR DYLMACGLCS VSKTSCQNIL TKGGPGRSIA
              210 220 230 240 250
IVVGGASESL NARPGVMDLV LKRRFGFIKI AVQTGASLVP TISFGENELY
              260 270 280 990 300
EQIESNENSK LHRWQKKIQH ALGFTMPLFH GRGVFNYDFG LLPHRHPIYT
              310 320 330 340 350
IVGKPIPVPS IKYGQTKDEI IRELHDSYMH AVQDLYDRYK DIYAKDRVKE
LEFVE
```

A sequence for a diacylglycerol O-acyltransferase 2B (DGAT2B) from Mortierella ramanniana is shown below as SEQ ID NO: 101 (Uniprot Q96UY1).

```
                10 20 30 40 50
MEQYQYTALL DHIPKVHWAP LRGIPLKRRL QTSAIVTWLA LLPICLIIYL
                60 70 80 90 100
YLFTIPLLWP ILIMYTIWLF FDKAPENGGR RISLVRKLPL WKHFANYFPV
              110 120 130 140 150
TLIKEGDLDP KGNYIMSYHP HGIISMAAFA NFATEATGFS EQYPGIVPSL
              160 170 180 190 200
LTLASNFRLP LYRDFMMSLG MCSVSRHSCE AILRSGPGRS IVIVTGGASE
              210 220 230 240 250
SLSARPGIND LTLKKRLGFI RLAIRNGASL VPIFSFGEND IYEQYDNKKG
              260 270 280 990 300
SLIWRYQKWF QKITGFTVPL AHARGIFNYN AGFIPFRHPI VTVVGKPIAV
              310 320 330 340
PLLAEGETEP SEEQMHQVQA QYIESLQAIY DKYKDIYAKD RIKDMTMIA
```

A sequence for an O-acyltransferase from Mortierella elongata AG-77 is shown below as SEQ ID NO:102 (Uniprot A0A197K574).

```
                10 20 30 40 50
MSQGDAITTS HSDGTEKRHD STTNILSDVP PQTEDVKSSS SKKKRSTYRH
                60 70 80 90 100
TFPVHTKTLP SPLSKEAPPE SYRGFVNLGM LLLFGNNIRL IIENYQKYGF
              110 120 130 140 150
LLSIPGSNVS KQDWILAGIT HAILPLHVIV AYQLEQWASR KAKGFRKRLA
              160 170 180 190 200
DQKENPTIKD DEDKKAVPAG DKVRGGKKDK KNLTLEEQIK ENRKTVGWLH
              210 220 230 240 250
FANVSLILGW PSFMSYFVIF HPFLAMGCLM TSLILFLKMV SFALVNQDLR
              260 270 280 290 300
YAYIQDTPAT EQSSPHLTKV HNDTITTTNT TSDGATTITT LTTITTVVKT
              310 320 330 340 350
ITVKKDAEKH GGAYQYEVHY PQNITPGNIG YFYLAPTLCY QPSYPRSTVF
              360 370 380 390 400
RPSFFFKRVL EIVTCLGMMY FLIEQYATPT LQNSVRAFDE LAFGRLLERV
              410 420 430 440 450
LKLSTTSVII WILMETIFFH AFFNALAEVL YFGDRRFYLS WWNATSVGMI
              460 470 480 490 500
WKTWNSPVYT FFKRHVYLPM ITSGHSAITA SVVIFTISAL LHEVLIGIPT
              510 520 530 540 550
KMIYGYAFAG MFFQIPLIAL TAPLEKWRGT GSGLGNMIFW VSFTILGQPA
              560
CALLYYYHWT KRSMNA
```

A sequence for a diacylglycerol acyltransferase from Mortierella alpina is shown below as SEQ ID NO:103 (Uniprot A0A1S6XXG5).

```
                10 20 30 40 50
MPLFAPLRMP IQRRMQTGAV LLWISGIIYT LGIFVFLCTF KVLRPLIIIY
                60 70 80 90 100
LLWAFMLDRG PQRGARAVQW YRNWVGWKHF AQYFPMTLVK EGELDPSKNY
              110 120 130 140 150
IFGYHPHGII SLGAFCTFGT EGLHFSKRFP GIKPQLLTLH ANFQIPLYRE
              160 170 180 190 200
MVMAHGCASV SRASCEHILR SGEGCSVVIV VGGAQESLST QPGTLNLTLK
              210 220 230 240 250
KRLGFCKLAL VNGASLVPTL AFGENELYEV YTAKPKSLMY KIQQFAKRTM
              260 270 280 290 300
GFIMPVENGR GVFNYEFGLL PRRKPVYIVV GKPIHVDKVE NPTVEQMQKL
              310 320 330
QSIYIDEVLN IWERYKDKYA AGRIQELCII E
```

A sequence for a type two diacylglycerol acyltransferase from Nannochloropsis oceanica is shown below as SEQ ID NO:104 (Uniprot A0A1S6KM83).

```
                10 20 30 40 50
MYPIKLCFLF ILTIPPYAHV RTRTPHRRGT TSKMAKANFP PSARYVNMIQ
                60 70 80 90 100
VYATGAHNMP DEDRLKVMNG LSKPLTEAKP GDLGFGDVES MTFCEEFVAI
              110 120 130 140 150
MFLLIIVGSM LWIPIAVLGF ALYVRSAMAW VVMLIVFFTL SLHPVPRIHD
              160 170 180 190 200
MVHSPLNHFI FKYFSLKMAS DAPLDSAGRY IFVAPPHGVL PMGNLMTVHA
              210 220 230 240 250
MKACGGLEFR GLTTDVALRL PLFRHYLGAa GTIAATRHVA KQYLDKGWSI
              260 270 280 290 300
GISSGGVAEI FEVNNKDEVV LMKERKGFVK LALRTGTPLV ACYIFGNTKL
              310 320 330 340 350
LSAWYDDGGV LEGLSRYLKC GVIPLWGRFG LPLMHRHPVL GAMAKPIVVP
              360 370 380 390
KVEGEPTQEM IDEYHSLFCQ TLVDLFDRYK TLYGWPDKKL LIK
```

A sequence for a diacylglycerol acyltransferase from Nannochloropsis gaditana (strain CCMP526) is shown below as SEQ ID NO:105 (Uniprot I2CPZ8).

```
                10 20 30 40 50
MGHVGKLDLL KALGELLRLA IPSTFVWLIT FYVYFHCTLN LFAEITRFGD
                60 70 80 90 100
RLFFKDWWNC TSFSRXWRTW NLPVHQFLVR HVYFPLLRAG ASKMTANVTV
              110 120 130 140 150
FAVSAFFHEL LISIPCHVVR LWAFLAMMGQ IPLIYITDHL DKTLFKETQA
              160 170
GNYMFWLIFC IFGQPMAVLL YYADFSARS
```

A sequence for a diacylglycerol acyltransferase 2 from Nannochloropsis gaditana (strain CCMP526) is shown below as SEQ ID NO:106 (Uniprot K8YXL9).

```
                10 20 30 40 50
MVCPLRSLVR DYRKTQGLVT SPHRSHGPDM SFKCKPSQKP NKQFWRYASF
                60 70 80 90 100
LAFIATFLLV PSTTSWASAL HRACFMAYVM TYLDTSYRDG SRAWPWFQRL
              110 120 130 140 150
PVWRLYCRYI KGQVITTVPL DPHRQYIFAA HPHGIATWNH FLTMTDGCRF
              160 170 180 190 200
LSRIYPRPRL DLGATVLFFI PLVKEVLLWV GCVDAGAATA NAILERGFSS
              210 220 230 240 250
LIYVGGEKEQ ILTERGRDLV VVIPRKGFCK LALRYDCPIV PAYAFGENDL
              260 270
YRTFNYFKGL QLWVERHAGR VVPRNRSEH
```

A sequence for a type 2 diacylglycerol acyltransferase (DGTT5) from Nannochloropsis oceanica is shown below as SEQ ID NO:107 (Uniprot A0A1S6KMA4).

```
                10 20 30 40 50
MTPQADITSK TTPNLKTAAS SPSKTSPAPS VQYKAANGKV ITVAMAEQDD
                60 70 80 90 100
GNMGIFRECF AMVTMGIIMS WYYIVVILSL LCLVGICIFP AWRAVAATVF
              110 120 130 140 150
VLMWSAALLP LDYQGWDAFC NSFIFRLWRD YFHYEYVLEE MIDPNKRYLF
              160 170 180 190 200
AEMPHGIFPW GEVISISITK QLFPGSRVGS IGASVIFLLP GLRHFFAWIG
              210 220 230 240 250
CRPASPENIK KIFEDGQDCA VTVGGVAEMF LVGGDKERLY LKKHKGFVRE
              260 270 280 290 300
AMKNGADLVP VFCFGNSKLF NVVGESSRVS MGLMKRLSRR IKASVLIFYG
              310 320 330 340 350
RLFLPIPIRH PLLFVVGKPL PVVHKAEPTK EEIAATHALF CEKVEELYYK
              360
YRPEWETRPL SIE
```

A sequence for a lecithin:cholesterol acyltransferase from Mortierella elongata AG-77 is shown below as SEQ ID NO:108 (Uniprot A0A197JIB8).

```
                10 20 30 40 50
MDKQQPDIVT MIPGIVSTGL ESWSTTNNSC SQKYFRKRMW GITTMFKAVL
                60 70 80 90 100
LDKDCWITNL RLDPETGVDP EGVRLRAAQG LEAADYFVQG YWVWAPIIKN
              110 120 130 140 150
LAAIGYDNNN MYLASYDWRL SFANLENRDN YFSRLKSNLE LSLKMTGEKS
              160 170 180 190 200
VLVAHSMGSN VMFYFFKWVE SDKGGKGGPN WVNDHVHTFV NIAGPMLGVP
              210 220 230 240 250
KTLAAVISGE VRDTAQLGVV SAYVLEKFFS RRERADLFRS WGGLSSMIPK
              260 270 280 290 300
GGNRIWGTIH GAPDDGTHDE EETVRNEKIA KSEETPGATT KRKHGEQSPT
              310 320 330 340 350
FGAMLAFAEG SNMENHGMDE SMGLLSKMAG NAYNTMLAKN YTVGASVTQK
              360 370 380 390 400
QMDKITKDPA SWTNPLEATL PYAPKMKIYC LYGVGKSTER SYTYNRVSDL
              410 420 430 440 450
APQIEDQRPG NVSDETGQVP NIYIDTTVHD DKLGISYGVH QGDGDGIVPL
              460 470 480 490 500
MSTGYMCVDG WSKKLYNPAG LKVITREFTH QSSLSPVDIR GGKRTADHVD
              510 520 530 540
ILGNYQVTKD LLAaVAGRDG DGLEEQIYSK IKEYSAKVDL
```

A sequence for a diacylglycerol acyltransferase (DGAT23) from Nannochloropsis oceanica strain IMET1 is shown below as SEQ ID NO:112 (Uniprot A0A290G0P3).

```
                10 20 30 40 50
MAHLFRRRSK GEGNSTSSRC LSLSEGNKAM LILSSEIEPP ASATSKAATS
                60 70 80 90 100
GIKEIGDPSL PTVALLSLPS ISKADKNSAT AAVAAGTLED AAAGALTAPF
              110 120 130 140 150
ADRSVKKQYG QDGDGAQCKE AEGGRKRSGS VGNLLLSSMT SFSKGTSLSF
              160 170 180 190 200
LTGEDKTPSP PETGPAGIDF STPAHPTMQF VDFIITFLLV HYIQVFYSLV
              210 220 230 240 250
FLFIYLVKHG HRWPYFLAAI YAPSYF1PLQ RLGGWPFKGF MRRPFWRCVQ
              260 270 280 290 300
RTLALQVERE VELSPDEQYI FGWHPHGILL LSRFAIYGGL WEKLFPGIHF
              310 320 330 340 350
KTLAASPLFW IPPIREVSIL LGGVDAGRAS AARALTDGYS VSLYPGGSKE
              360 370 380 390 400
IYTTDPYTPE TTLVLKIRKG FIRMAIRYGC ALVPVYTFGE KYAYHRIGQA
              410 420 430 440 450
TGFARWLLAV LKVPFLIFWG RWGTFMPLKE TQVSVVVGTP LRVPKIEGEP
              460 470 480
SPEVVE WLH KYCDEVQALF RRHKHKYAKP EEFVAIS
```

A sequence for a type two diacylglycerol acyltransferase (DGTT2) from Nannochloropsis oceanica is shown below as SEQ ID NO:109 (Uniprot A0A1S6KMB4).

```
                10 20 30 40 50
MAHLFRRRSK GEGNSTSSRC LSLSEGNKAM LILSSEIEPP ASATSKAATS
                60 70 80 90 100
GIKEIGDPSL PTVALLSLPS ISKADTNSAT AAVAAGTLED AAAGALTAPF
              110 120 130 140 150
ADRSVKKQYG QDGDGAQCKE AEGGRKRSGS VGNLLLSSMT SFSKGTSLSF
              160 170 180 190 200
LTGEDKTPSP PETGPAGIDF STPAHPTMQF VDFIITFLLV HYIQVFYSLV
              210 220 230 240 250
FLFIYLVKHG HRWPYFLAAI YAPSYFIPLQ RLGGWPFKGF MRRPFWRCVQ
              260 270 280 290 300
RTLALQVERE VELSPDEQYI FGWHPEVSIL LGGGSKEIYT TDPYTPETTL
              310 320 330 340 350
VLKIRKGFIR MAIRYGCALV PVYTFGEKYA YHRLGQATGF ARWLLAVLKV
              360 370
PFLIFWGRHK HKYAKPEEFV AIS
```

The following Examples illustrate some of the experimental work involved in the development of the invention.

### Example 12: Myco-Filtering to Harvest Algae

This Example illustrates methods for harvesting microalgae (e.g. N. oceanica) by micro-filtration with M. elongata.

### Materials and Methods for Growing Myco-Filters

To utilize the flocculation/interaction between the microalgae (N. oceanica) and fungi (M. elongata) for harvesting algae, a fungal-filter system was developed that utilizes the attraction of algae to Mortierella mycelium. The M. elongata was grown into a filter to collect algae from the culture by filtration. The filtration is based on the affinity/physical cell wall-cell wall attraction between the microalgae and fungi instead of regular filters that isolate microalgae by pore size exclusion. One advantage of the fungal filter is that it won't get clogged like other regular filters, even when the mycelium is saturated by trapped microalgae and the algal culture can still pass through the filter. This lends itself to continuous-flow filtration systems, but also work for batch processing. Following incubation in regular growth medium, Mortierella fungi form dense biofilms along culture surfaces. The mycelium is indeterminant in growth form, which means that they can grow into the size and shape of the incubation container chosen.

Taking advantage of this feature, Mortierella fungi were inoculated and incubated in standard size disposable petri dishes (i.e. 60×15, 100×15 mm) that are common and widely used in research and industry. They grow in half strength potato dextrose broth medium into a standard size of fungal-filters within 2 to 5 days incubation (depending on how much materials are inoculated and the incubation temperature, ideally room temperature 20-25° C. for most strains). Mycelia can also be grown on a silicon, mesh or large pored fabric membrane to easy harvesting of fungal-algal aggregates for down-stream processing. Stand size fungal filters are then ready for use and they can be stacked together for the filtration of microalgae.

### Example 13: Myco-Filtering to Harvest Blue-Green Algae

This Example illustrates methods for harvesting blue-green algae (also called cyanobacteria) by micro-filtration with Mortierella elongata.

### Methods

Filamentous cyanobacteria of genus Anabaena were cultured in BG-11 medium. Mortierella elongata membranes were added into the algae culture and the coculture was incubated for two days.

### Results

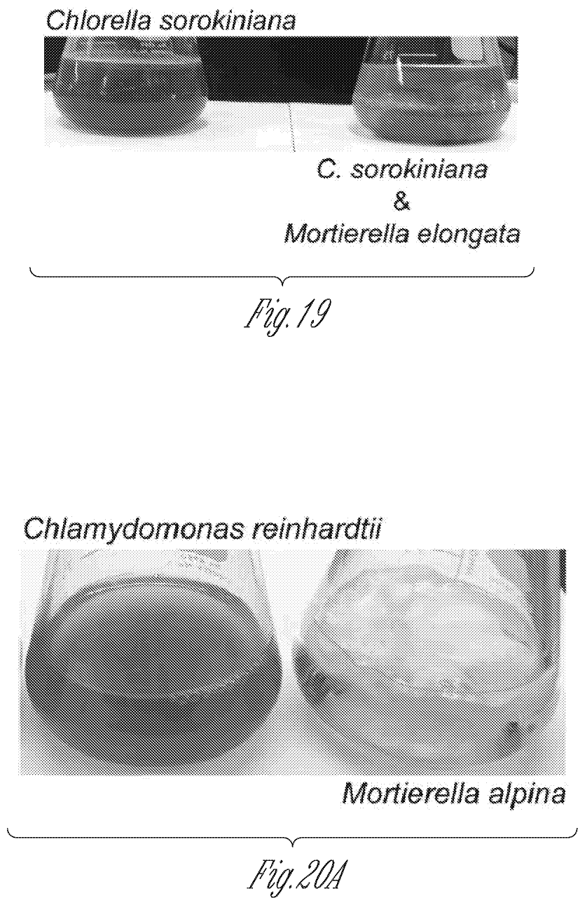

FIG. 18A illustrates that cultures of Anabaena variabilis, Anabaena cylindrica, and Anabaena sp. PCC 7120 form a substantially uniform suspension when cultured in BG-11 medium. However, after co-culture with Mortierella elongata membranes, these Anabaena species flocculate into clumps (FIG. 18B) that are readily harvested.

### Example 14: Myco-Filtering Chlorella sorokiniana with Mortierella

This Example illustrates methods for harvesting green freshwater microalgae (e.g., Chlorella sorokiniana) by Mortierella alpina.

### Methods

Chlorella sorokiniana algae were cultured in BG-11/TAP medium. Mortierella alpina were added into the algae culture and cocultured overnight.

### Results

As shown in FIG. 19, Chlorella sorokiniana algae readily flocculate with Mortierella.

Chlorella has fast growth rate and high biomass enriched in proteins and oils. For example, each Chlorella can divide into four new cells every 17 to 24 hours. Such a fast growth rate facilitates production of useful products made by the Chlorella.

### Example 15: Myco-Filtering Chlamydomonas with Different Mortierella Species

This Example illustrates methods for harvesting green algae (e.g., Chlamydomonas reinhardtii) by micro-filtration with different Mortierella species.

### Methods

Chlamydomonas reinhardtii algae were cultured in TAP medium. Mortierella alpina were added into this culture of algae and the mixture was cocultured overnight.

### Results

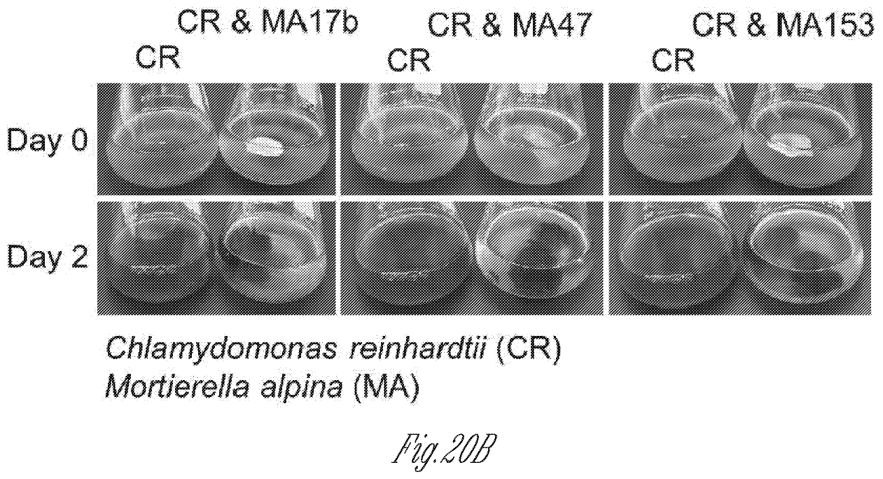

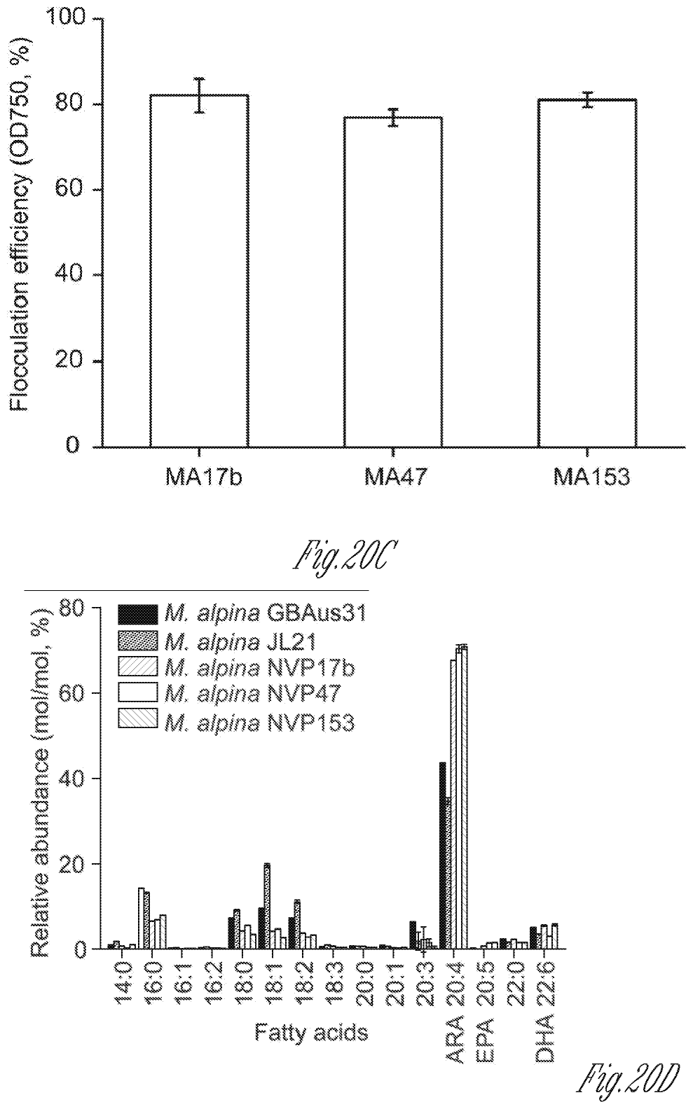

FIG. 20A (left) shows Chlamydomonas reinhardtii algae alone in culture. FIG. 20A (right) shows Chlamydomonas reinhardtii algae after co-culture with Mortierella alpina. As illustrated, Chlamydomonas reinhardtii algae form a uniform, dispersed suspension when cultured without Mortierella (FIG. 20A left). However, after co-culture with Mortierella alpina the Chlamydomonas reinhardtii algae clump up or flocculate with the Mortierella alpina (FIG. 20A right), which facilitates harvesting of the algae/fungal flocculate. FIG. 20B shows that Chlamydomonas reinhardtii algae clump up or flocculate with different strains of Mortierella alpina, including Mortierella alpina NVP17b, Mortierella alpina NVP47, and Mortierella alpina NVP153. FIG. 20C graphically illustrates the flocculation efficiency of different strains of Mortierella alpina, including Mortierella alpina NVP17b, Mortierella alpina NVP47, and Mortierella alpina NVP153, when mixed with Chlamydomonas reinhardtii algae.

FIG. 20D graphically illustrates that various Mortierella alpina strains are enriched in poly-unsaturated fatty acids such as ARA, EPA, and DHA. Hence, co-cultures of algae with Mortierella alpina form commercially useful sources of such oils.

### REFERENCES

1. R. F. Service, Algae's second try. Science. 333, 1238-1239 (2011).
2. N. Okamoto, I. Inouye, A secondary symbiosis in progress? Science. 310, 287 (2005).
3. A. F. Little, M. J. H. van Oppen, B. L. Willis, Flexibility in algal endosymbioses shapes growth in reef corals. Science. 304, 1492-1494 (2004).
4. E. Tisserant et al., Genome of an arbuscular mycorrhizal fungus provides insight into the oldest plant symbiosis. Proc. Nal. Acad. Sci. U.S.A. 110, 20117-20122 (2013).
5. E. F. Y. Hom, A. W. Murray, Plant-fungal ecology. Niche engineering demonstrates a latent capacity for fungal-algal mutualism. Science. 345, 94-98 (2014).
6. J. Simon et al., Self-supporting artificial system of the green alga Chlamydomonas reinhardtii and the ascomycetous fungus Alternaria infectoria. Symbiosis, 1-11 (2016).
7. G. Bonito et al., Isolating a functionally relevant guild of fungi from the root microbiome of Populus. Fungal Ecol. 22, 35-42 (2016).
8. K. Brenner, L. You, F. H. Arnold, Engineering microbial consortia: a new frontier in synthetic biology. Trends Biotechnol. 26, 483-489 (2008).
9. D. Mollenhauer, R. Mollenhauer, M. Kluge, Studies on initiation and development of the partner association in Geosiphon pyriforme (Kütz.) v. Wettstein, a unique endocytobiotic system of a fungus (Glomales) and the cyanobacterium Nostoc punctiforme (Kütz.) Hariot. Protoplasma. 193, 3-9 (1996).
10. P. Bonfante, A. Genre, Mechanisms underlying beneficial plant-fungus interactions in mycorrhizal symbiosis. Nat. Commun. 1, 48 (2010).
11. P. M. Delaux et al., Algal ancestor of land plants was preadapted for symbiosis. Proc. Natl. Acad. Sci. U.S.A. 112, 13390-13395 (2015).
12. K. J. Field et al., Functional analysis of liverworts in dual symbiosis with Glomeromycota and Mucoromycotina fungi under a simulated Palaeozoic CO₂ decline. ISME J. 10, 1514-1526 (2015).
13. J. W. Spatafora et al., A phylum-level phylogenetic classification of zygomycete fungi based on genome-scale data. Mycologia. Resubmitted. Dataset DOI: 10.5281/zenodo.46700 TreeBase: TB2:S18957
14. D. Redecker, R. Kodner, L. E. Graham, Glomalean fungi from the Ordovician. Science. 289, 1920-1921 (2000).
15. S. Wodniok et al., Origin of land plants: do conjugating green algae hold the key? BMC Evol. Biol. 11, 104 (2011).
16. K. J. Field, S. Pressel, J. G. Duckett, W. R. Rimington, M. I. Bidartondo, Symbiotic options for the conquest of land. Trends Ecol. Evol. 30, 477-486 (2015).
17. P. R. Atsatt, Are vascular plants “inside-out” lichens? Ecology. 69, 17-23 (1988).
18. A. Vieler et al., Genome, functional gene annotation, and nuclear transformation of the heterokont oleaginous alga Nannochloropsis oceanica CCMP1779. PLoS Genet. 8, e1003064 (2012).
19. L. P. Partida-Martinez, C. Hertweck, A gene cluster encoding rhizoxin Biosynthesis in Burkholderia rhizoxina, the bacterial endosymbiont of the fungus Rhizopus microsporus. Chembiochem. 8, 41-45 (2007).
20. H. L. Chen, S. S. Li, R Huang, H. J. Tsai, Conditional production of a functional fish growth hormone in the transgenic line of Nannochloropsis oculata (Eustigmatophyceae). J. Phycol. 44, 768-776 (2008).
21. A. D. Velichkov, A simple procedure for dissolving fungal cell wall preparations for the analysis of neutral sugars. World J. Microbiol. Biotechnol. 8, 527-528 (1992).
22. M. J. Scholz et al., Ultrastructure and composition of the Nannochloropsis gaditana cell wall. Eukaryot. Cell. 13, 1450-1464 (2014).
23. C. H. Tsai et al., The protein compromised hydrolysis of triacylglycerols 7 (CHT7) acts as a repressor of cellular quiescence in Chlamydomonas. Proc. Natl. Acad. Sci. U.S.A. 111, 15833-15838 (2014).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements of the invention are intended to describe and summarize various embodiments of the invention according to the foregoing description in the specification.

### Statements:

1. A consortium comprising at least one viable fungus and at least one viable algae linked to or within hyphae of the fungus, wherein the fungus, algae, or both have been modified to express a heterologous (exogenous) lipid synthesizing enzyme.
2. The consortium of statement 1, wherein algae is a diatom (bacillariophyte), green algae (chlorophyte), blue-green algae (cyanophyte), golden-brown algae (chrysophyte), haptophyte, or a combination thereof.
3. The consortium of statement 1 or 2, wherein algae is a species of Amphipleura, Amphora, Anabaena, Aquamortierella, Chaetoceros, Charophyceae, Chlorodendrophyceae, Chlorella, Chlorokybophyceae, Chlorophyceae, Coleochaetophyceae, Cyclotella, Cymbella, Dissophora, Embryophytes, Endogaceae, Fragilaria, Gamsiella, Hantzschia, Klebsormidiophyceae, Lobosporangium, Mamiellophyceae, Mesostigmatophyceae, Modicella, Mortierella, Mucor, Navicula, Nephroselmidophyceae, Nitzschia, Palmophyllales, Prasinococcales, Prasinophytes, Pedinophyceae, Phaeodactylum, Pyramimonadales, Pycnoccaceae, Pythium, Phytophthora, Phytopythium, Rhizopus, Thalassiosira, Trebouxiophyceae, Ulvophyceae, Zygnematophyceae, or the algae is a combination of species.
4. The consortium of statement 1, 2, or 3, wherein algae is of genera Ankistrodesmus, Boekelovia, Botryococcus, Chlorella, Chlorococcum, Dunaliella, Isochrysis, Monoraphidium, Nannochloropsis, Oocystis, Oscillatoria, Pleurochrysis, Scenedesmus, Synechococcus, Tetraselmis, or a combination thereof.
5. The consortium of statement 1-3, or 4, wherein algae is Emiliania huxleyi, Gephyrocapsa oceanica, Isochrysis galbana, Isochrysis sp. T-Iso, Isochrysis sp. C-Iso, Nannochloropsis oceanica, or a combination thereof
6. The consortium of statement 1-4, or 5, wherein algae is a photosynthetic algae.
7. The consortium of statement 1-5, or 6, wherein algae may not, in some cases, be Nostoc punctiforme.
8. The consortium of statement 1-6, or 7, wherein algae is Nannochloropsis oceanica CCMP1779.
9. The consortium of statement 1-7 or 8, wherein the fungus is Aspergillus, Blakeslea, Botrytis, Candida, Cercospora, Cryptococcus, Cunninghamella, Fusarium (Gibberella), Kluyveromyces, Lipomyces, Morchella, Mortierella, Mucor, Neurospora, Penicillium, Phycomyces, Pichia (Hansenula), Puccinia, Pythium, Rhodosporidium, Rhodotorula, Saccharomyces, Sclerotium, Trichoderma, Trichosporon, Xanthophyllomyces (Phqffia), Yarrowia, or a combination thereof.
10. The consortium of statement 1-8 or 9, wherein the fungus is Mortierella elongata, Mortierella elongata AG77, Mortierella gamsii, Mortierella gamsii GBAus22, Umbelopsis sp., Umbelopsis PMI120, Lecythophora sp., Lecythophora PMI546, Leptodontidium sp., Leptodontidium PMI413, Lachnum sp., Lachnum PMI789, Morchella sp., Saccharomyces cerevisiae, Atractiella sp., Atractiella PMI152, Clavulina, Clavulina PMI390, Grifola frondosa, Grifola frondosa GMNB41, Flagelloscypha sp., Flagelloscypha PMI1526, or a combination thereof.
11. The consortium of statement 1-9 or 10, wherein the fungus is Aspergillus terreus, Aspergillus nidulans, Aspergillus niger, Atractiella PMI152, Blakeslea trispora, Botrytis cinerea, Candida japonica, Candida pulcherrima, Candida revkaufi, Candida tropicalis, Candida utilis, Cercospora nicotianae, Clavulina PMI390, Cryptococcus curvatus, Cunninghamella echinulata, Cunninghamella elegans, Flagelloscypha PMI526, Fusarium fujikuroi (Gibberella zeae), Grifola frondosa GMNB41, Kluyveromyces lactis, Lecythophora PMI546, Leptodontidium PMI413, Lachnum PMI789, Lipomyces starkeyi, Lipomyces lipoferus, Mortierella alpina, Mortierella elongata AG77, Mortierella gamsii GBAus22, Mortierella ramanniana, Mortierella isabellina, Mortierella vinacea, Mucor circinelloides, Neurospora crassa, Phycomyces blakesleanus, Pichia pastoris, Puccinia distincta, Pythium irregulare, Rhodosporidium toruloides, Rhodotorula glutinis, Rhodotorula graminis, Rhodotorula mucilaginosa, Rhodotorula pinicola, Rhodotorula gracilis, Saccharomyces cerevisiae, Sclerotium rolfsii, Trichoderma reesei, Trichosporon cutaneum, Trichosporon pullans, Umbelopsis PMI120, Xanthophyllomyces dendrorhous (Phqffia rhodozyma). Yarrowia lipolytica, or a combination thereof.
12. The consortium of statement 1-10 or 11, wherein the fungus is not Geosiphon pyriformis.
13. The consortium of statement 1-11 or 12, wherein the fungus has more than one algae cell within the fungus hyphae.
14. The consortium of statement 1-12 or 13, wherein the fungus has more than two algae cells within the fungus hyphae.
15. The consortium of statement 1-13 or 14, wherein the fungus has more than five, or more than ten, or more than twenty, or more than twenty five, or more than thirty, or more than forty, or more than fifty, or more than one hundred algae cells within the fungus hyphae.
16. The consortium of statement 1-14 or 15, wherein the fungus has less than 10,000 algae cells within the fungus hyphae, or less than 5000 algae cells within the fungus hyphae, or less than 2000 algae cells within the fungus hyphae, or less than 1000 algae cells within the fungus hyphae.
17. The consortium of statement 1-15 or 16, wherein the algae photosynthetically synthesizes sugars.
18. The consortium of statement 1-16 or 17, wherein the algae has a degraded or missing outer cell wall.
19. The consortium of statement 1-17 or 18, wherein the algae has cell wall extensions.
20. The consortium of statement 1-18 or 19, wherein the algae has cell wall is associated with, bound to, or linked to hyphae of the fungus.
21. The consortium of statement 1-19 or 20, wherein the algae or the fungus comprises at least one heterologous expression cassette or expression vector that includes a promoter operably linked to nucleic acid segment encoding a lipid synthetic enzyme.
22. The consortium of statement 21, wherein the lipid synthesizing enzyme is acetyl-CoA carboxylase, malonyl-CoA decarboxylase, acyl carrier protein, fatty acid synthase, malonyl-CoA:ACP malonyltransferase, 3-oxoacyl-ACP synthase, KASI/II, 3-hydroxydecanoyl-ACP dehydratase, 3-hydroxydecanoyl-ACP dehydratase, 3-ketoacyl-ACP reductase, acyl-CoA elongase, fatty acid desaturase, acyl-CoA thioesterase, acyl-CoA synthetase, aldehyde dehydrogenase, alcohol dehydrogenase, glycerol kinase, glycerol-3-phosphate dehydrogenase, glycero-3-phosphate acyltransferase, 1-sn-acyl-glycero-3-phosphate acyltransferase, phosphatidic acid phosphatase, lipin-like phosphatidate phosphatase, diacylglycerol kinase, diacylglycerol acyltransferase, phospholipid diacylglycerol acyltransferase, or any combination thereof.
23. The consortium of statement 21 or 22, wherein the algae or the fungus comprises two or more heterologous expression cassettes or expression vectors, each cassette or vector having a promoter operably linked to nucleic acid segment encoding a lipid synthetic enzyme.
24. A method comprising incubating at least one fungus and at least one algae cell until at least one algae cell is incorporated into hyphae of the fungus, to thereby form a consortium of the at least one fungus and the at least one algae cell, wherein the at least one fungus or at least one algae has been modified to express a heterologous lipid synthesizing enzyme.
25. The method of statement 24, wherein at least one fungus and at least one algae cell are incubated together for one or more days, one or more weeks, one or months, one or more years, or indefinitely.
26. The method of statement 24 or 25 wherein at least one fungus and at least one algae cell are incubated at a fungus tissue and algae cell density sufficient for the fungus and the algae come into contact.
27. The method of statement 24, 25, or 26, wherein algae is added to the fungus at a density of about 1×10⁴ algae cells/mL to 1×10⁹ algae cells/mL, or at a density of about 1×10⁵ algae cells/mL to 1×10⁸ algae cells/mL, or at a density of about 1×10⁶ algae cells/mL to 1×10⁸ algae, or at a density of about 1-3×10⁷ cells/mL.
28. The method of statement 24-26 or 27, wherein more fungus tissue by mass than algae cells by mass is incubated together.
29. The method of statement 24-27 or 28, wherein the fungus and the algae cells are incubated at a ratio of from about 10:1 by mass fungal tissue to algal cells, to about 1:1 by mass fungal tissue to algal cells; or from about 5:1 by mass of fungal tissue to algal cells to about 1:1 by mass fungal tissue to algal cells; or at a ratio of about 3:1 by mass fungal tissue to algal cells.
30. The method of statement 24-28 or 29, wherein more algae cells by mass than fungal tissue by mass is incubated.
31. The method of statement 24-29 or 30, wherein the fungus and the algae cells are incubated at a ratio of from about 10:1 by mass algal cells to fungal tissue mass to about 1:1 by mass algal cells to fungal tissue mass, or at a ratio of from about 5:1 by mass algal cells to fungal tissue mass to about 1:1 by mass algal cells to fungal tissue mass.
32. The method of statement 24-30 or 31, wherein one or more fungal species and one or more algae species are incubated in a culture medium that contains some carbohydrate or some sugar.
33. The method of statement 32, wherein the some comprises dextrose, sucrose, glucose, fructose or a combination thereof.
34. The method of statement 32 or 33, wherein the carbohydrate or sugar is present in an amount of about 1 g/liter to about 20 g/liter, or of about 3 g/liter to about 18 g/liter, or of about 5 g/liter to about 15 g/liter.
35. The method of statement 24-33 or 34, wherein one or more fungal species and one or more algae species is incubated in a liquid media, in a semi-solid media, or on a solid media.
36. The method of statement 24-34 or 35, wherein the consortium of the at least one fungus and the at least one algae cell is incubated in a minimal medium.
37. The method of statement 24-35 or 36, wherein the consortium comprising the at least one fungus and the at least one algae cell is incubated or maintained in a minimal medium containing no added carbohydrate or sugar.
38. The method of statement 24-36 or 37, wherein the consortium comprising the at least one fungus and the at least one algae cell grows in a minimal medium containing no added carbohydrate or sugar.
39. The method of statement 24-37 or 38, wherein the one or more fungal species and one or more algae species are incubated in a culture medium that contains sodium bicarbonate.
40. The method of statement 24-38 or 39, wherein the one or more fungal species and one or more algae species are incubated in a culture medium that contains ammonium salts.
41. The method of statement 24-39 or 40, wherein the consortium synthesizes one or more lipid, carbohydrate, or protein.
42. The method of statement 24-40 or 41, wherein the consortium comprises a lipid content greater than 40%, 50%, 60%, 70%, 80%, or 90% by weight of the consortium.
43. The method of statement 24-41 or 42, wherein after incubating the algae has a degraded or missing outer cell wall.
44. The method of statement 24-42 or 43, wherein after incubating the algae has cell wall extensions.
45. The method of statement 24-43 or 44, wherein after incubating the algae has a cell wall associated with, bound to, or linked to hyphae of the fungus.
46. The method of statement 24-44 or 45, wherein the algae or the fungus comprises at least one heterologous expression cassette or expression vector that includes a promoter operably linked to nucleic acid segment encoding a lipid synthetic enzyme.
47. The method of statement 26, wherein the lipid synthesizing enzyme is acetyl-CoA carboxylase, malonyl-CoA decarboxylase, acyl carrier protein, fatty acid synthase, malonyl-CoA:ACP malonyltransferase, 3-oxoacyl-ACP synthase, KASI/II, 3-hydroxydecanoyl-ACP dehydratase, 3-hydroxydecanoyl-ACP dehydratase, 3-ketoacyl-ACP reductase, acyl-CoA elongase, fatty acid desaturase, acyl-CoA thioesterase, acyl-CoA synthetase, aldehyde dehydrogenase, alcohol dehydrogenase, glycerol kinase, glycerol-3-phosphate dehydrogenase, glycero-3-phosphate acyltransferase, 1-sn-acyl-glycero-3-phosphate acyltransferase, phosphatidic acid phosphatase, lipin-like phosphatidate phosphatase, diacylglycerol kinase, diacylglycerol acyltransferase, phospholipid diacylglycerol acyltransferase, or any combination thereof.
48. The method of statement 46 or 47, wherein the algae or the fungus comprises two or more heterologous expression cassettes or expression vectors, each cassette or vector having a promoter operably linked to nucleic acid segment encoding a lipid synthetic enzyme.
49. A consortium comprising Mortierella elongata AG77 and Nannochloropsis oceanica CCMP1779 within hyphae of the Mortierella elongata AG77.
50. The consortium of statement 49, wherein the Mortierella elongata AG77, the Nannochloropsis oceanica CCMP1779, or both are modified to express a heterologous lipid synthesizing enzyme.
51. The consortium of statement 49 or 50, wherein the Mortierella elongata AG77, the Nannochloropsis oceanica CCMP1779, or both comprises at least one heterologous expression cassette or expression vector that includes a promoter operably linked to nucleic acid segment encoding a lipid synthetic enzyme.
52. The consortium of statement 49, 50 or 51, wherein the lipid synthesizing enzyme is acetyl-CoA carboxylase, malonyl-CoA decarboxylase, acyl carrier protein, fatty acid synthase, malonyl-CoA:ACP malonyltransferase, 3-oxoacyl-ACP synthase, KASI/II, 3-hydroxydecanoyl-ACP dehydratase, 3-hydroxydecanoyl-ACP dehydratase, 3-ketoacyl-ACP reductase, acyl-CoA elongase, fatty acid desaturase, acyl-CoA thioesterase, acyl-CoA synthetase, aldehyde dehydrogenase, alcohol dehydrogenase, glycerol kinase, glycerol-3-phosphate dehydrogenase, glycero-3-phosphate acyltransferase, 1-sn-acyl-glycero-3-phosphate acyltransferase, phosphatidic acid phosphatase, lipin-like phosphatidate phosphatase, diacylglycerol kinase, diacylglycerol acyltransferase, phospholipid diacylglycerol acyltransferase, or any combination thereof.
53. The consortium of statement 51 or 52, wherein the Mortierella elongata AG77, the Nannochloropsis oceanica CCMP1779, or both comprises two or more heterologous expression cassettes or expression vectors, each cassette or vector having a promoter operably linked to nucleic acid segment encoding a lipid synthetic enzyme.
54. A method of generating a consortium between Mortierella elongata AG77 and Nannochloropsis oceanica CCMP1779, comprising incubating the Mortierella elongata AG77 with Nannochloropsis oceanica CCMP1779 until the Nannochloropsis oceanica CCMP1779 are incorporated within hyphae of the Mortierella elongata AG77.
55. The method of statement 54, wherein the Mortierella elongata AG77, the Nannochloropsis oceanica CCMP1779, or both are modified to express a heterologous lipid synthesizing enzyme.
56. The method of statement 55, wherein the lipid synthetic enzyme is one or more acetyl-CoA carboxylase, malonyl-CoA decarboxylase, acyl carrier protein, fatty acid synthase, malonyl-CoA:ACP malonyltransferase, 3-oxoacyl-ACP synthase, KASI/II, 3-hydroxydecanoyl-ACP dehydratase, 3-hydroxydecanoyl-ACP dehydratase, 3-ketoacyl-ACP reductase, acyl-CoA elongase, fatty acid desaturase, acyl-CoA thioesterase, acyl-CoA synthetase, aldehyde dehydrogenase, alcohol dehydrogenase, glycerol kinase, glycerol-3-phosphate dehydrogenase, glycero-3-phosphate acyltransferase, 1-sn-acyl-glycero-3-phosphate acyltransferase, phosphatidic acid phosphatase, lipin-like phosphatidate phosphatase, diacylglycerol kinase, diacylglycerol acyltransferase, phospholipid diacylglycerol acyltransferase, or any combination thereof.
57. A consortium comprising at least one viable fungus and at least one viable photosynthetically active alga within hyphae of the fungus, wherein the fungus, alga, or both have been modified to express at least one of the following lipid synthetic enzymes: acetyl-CoA carboxylase, malonyl-CoA decarboxylase, acyl carrier protein, fatty acid synthase, malonyl-CoA:ACP malonyltransferase, 3-oxoacyl-ACP synthase, KASI/II, 3-hydroxydecanoyl-ACP dehydratase, 3-hydroxydecanoyl-ACP dehydratase, 3-ketoacyl-ACP reductase, acyl-CoA elongase, fatty acid desaturase, acyl-CoA thioesterase, acyl-CoA synthetase, aldehyde dehydrogenase, alcohol dehydrogenase, glycerol kinase, glycerol-3-phosphate dehydrogenase, glycero-3-phosphate acyltransferase, 1-sn-acyl-glycero-3-phosphate acyltransferase, phosphatidic acid phosphatase, lipin-like phosphatidate phosphatase, diacylglycerol kinase, diacylglycerol acyltransferase, phospholipid diacylglycerol acyltransferase, or any combination thereof.
58. The consortium of statement 57, wherein alga is a diatom (bacillariophyte), green algae (chlorophyte), blue-green algae (cyanophyte), golden-brown algae (chrysophyte), haptophyte, or a combination thereof.
59. The consortium of statement 57 or 58, wherein alga is a species of Amphipleura, Amphora, Anabaena, Ankistrodesmus, Aquamortierella, Boekelovia, Botryococcus, Chaetoceros, Charophyceae, Chlorella, Chlorococcum, Chlorodendrophyceae, Chlorokybophyceae, Chlorophyceae, Coleochaetophyceae, Cyclotella, Cymbella, Dissophora, Dunaliella, Embryophytes, Endogaceae, Fragilaria, Gamsiella, Hantzschia, Isochrysis, Klebsormidiophyceae, Lobosporangium, Mamiellophyceae, Mesostigmatophyceae, Modicella, Monoraphidium, Mortierella, Mucor, Nannochloropsis, Navicula, Nephroselmidophyceae, Nitzschia, Oocystis, Oscillatoria, Palmophyllales, Pleurochrysis, Prasinococcales, Prasinophytes, Pedinophyceae, Phaeodactylum, Pyramimonadales, Pycnoccaceae, Pythium, Phytophthora, Phytopythium, Rhizopus, Scenedesmus, Synechococcus, Tetraselmis, Thalassiosira, Trebouxiophyceae, Ulvophyceae, Zygnematophyceae, or the algae is a combination of species.
60. The consortium of statement 57, 58 or 59, wherein alga is Emiliania huxleyi, Gephyrocapsa oceanica, Isochrysis galbana, Isochrysis sp. T-Iso, Isochrysis sp. C-Iso, Nannochloropsis oceanica, or a combination thereof
61. The consortium of statement 57-59 or 60 wherein algae is Nannochloropsis oceanica CCMP1779.
62. The consortium of statement 57-60 or 61, wherein the fungus is a species of Aspergillus, Atractiella, Blakeslea, Botrytis, Candida, Cercospora, Clavulina, Cryptococcus, Cunninghamella, Flagelloscypha, Fusarium (Gibberella), Grifola, Kluyveromyces, Lachnum, Lecythophora, Leptodontidium, Lipomyces, Morchella, Mortierella, Mucor, Neurospora, Penicillium, Phycomyces, Pichia (Hansenula), Puccinia, Pythium, Rhodosporidium, Rhodotorula, Saccharomyces, Sclerotium, Trichoderma, Trichosporon, Umbelopsis, Xanthophyllomyces (Phqffia), Yarrowia, or a combination thereof.
63. The consortium of statement 57-61 or 62, wherein the fungus is Atractiella PMI152, Clavulina PMI390, Flagelloscypha PMI526, Grifola frondosa, Grifola frondosa GMNB41, Lecythophora PMI546, Leptodontidium PMI413, Lachnum PMI789, Mortierella elongata, Mortierella elongata AG77, Mortierella gamsii, Mortierella gamsii GBAus22, Saccharomyces cerevisiae, Umbelopsis PMI120, or a combination thereof.
64. The consortium of statement 57-62 or 63, wherein the fungus has more than one algae cell within the fungus hyphae.
65. The consortium of statement 57-63 or 64, wherein the alga synthesizes sugars.
66. A method comprising incubating at least one fungus and at least one alga cell in a culture medium until at least one alga cell is incorporated into hyphae of the fungus, to thereby form a consortium of the at least one fungus and the at least one alga cell, wherein the fungus, alga, or both have been modified to express at least one of the following lipid synthetic enzymes: acetyl-CoA carboxylase, malonyl-CoA decarboxylase, acyl carrier protein, fatty acid synthase, malonyl-CoA:ACP malonyltransferase, 3-oxoacyl-ACP synthase, KASI/II, 3-hydroxydecanoyl-ACP dehydratase, 3-hydroxydecanoyl-ACP dehydratase, 3-ketoacyl-ACP reductase, acyl-CoA elongase, fatty acid desaturase, acyl-CoA thioesterase, acyl-CoA synthetase, aldehyde dehydrogenase, alcohol dehydrogenase, glycerol kinase, glycerol-3-phosphate dehydrogenase, glycero-3-phosphate acyltransferase, 1-sn-acyl-glycero-3-phosphate acyltransferase, phosphatidic acid phosphatase, lipin-like phosphatidate phosphatase, diacylglycerol kinase, diacylglycerol acyltransferase, phospholipid diacylglycerol acyltransferase, or any combination thereof.
67. The method of statement 66, wherein at least one fungus and at least one alga cell are incubated together for one or more days, one or more weeks, one or months, one or more years, or indefinitely.
68. The method of statement 66 or 67, wherein at least one fungus and at least one alga cell are incubated at a fungus cell or fungus tissue, and an algae cell density sufficient for the fungus and the alga come into contact.
69. The method of statement 66, 67 or 68, wherein more fungi cells or fungus tissue by mass than algal cells by mass is incubated together.
70. The method of statement 66-68 or 69, wherein more algae cells by number than fungal cells or fungus tissue pieces by number is incubated.
71. The method of statement 66-69 or 70, wherein the fungus and the algae cells are incubated at a ratio of from about 10:1 by mass algal cells to fungal tissue mass to about 1:1 by mass algal cells to fungal tissue mass.
72. The method of statement 66-70 or 71, wherein one or more fungal species and one or more algal species are incubated in a culture medium that contains some carbohydrate or some sugar.
73. The method of statement 72, wherein the carbohydrate or sugar is present in an amount of about 1 g/liter to about 20 g/liter.
74. The method of statement 66-72 or 73, wherein the consortium of the at least one fungus and the at least one alga cell is incubated in a minimal medium.
75. The method of statement 66-73 or 74, comprising incubating a Mortierella elongata AG77 fungus with one or more Nannochloropsis oceanica CCMP1779 cell until the Nannochloropsis oceanica CCMP1779 are incorporated within hyphae of the Mortierella elongata AG77.
76. The method of statement 66-74 or 75, wherein prior to or during the incubating, at least one fungus or at least one alga cell, or a combination thereof are incubated in a culture medium that that is sparged with carbon dioxide and that does not contain added bicarbonate salts.
77. The method of statement 66-75 or 76, wherein prior to or during the incubating, at least one fungus or at least one alga cell, or a combination thereof are incubated in a culture medium that contains ammonium salts.
78. The method of statement 66-76 or 77, further comprising incubating the consortium for a time and under conditions for the consortium to produce lipid, carbohydrate, protein, or a combination thereof.
79. The method of statement 66-77 or 78, further comprising harvesting the alga by collecting the consortium from the culture medium.
80. The method of statement 66-78 79, wherein the consortium comprises a lipid content greater than 40% by weight of the consortium.
81. A method comprising incubating fungi within a culture medium in a container or on a solid surface to form a fungal-filter and contacting a culture of algae with the fungal-filter.
82. The method of statement 81, wherein the fungi are incubated in half strength potato dextrose broth medium.
83. The method of statement 81 or 82, wherein the fungi are incubated for about 2 to 5 days at 20-25° C.
84. The method of statement 81, 82, or 83, wherein the container or the solid surface is a petri dish, a silicon membrane, mesh, or large pored fabric membrane.
85. The method of statement 81-83 or 84, wherein two or more fungal-filters are stacked together and the culture of algae is contacted with the stacked fungal-filters.
86. The method of statement 81-84 or 85, wherein the algae are microalgae, green algae, or blue-green algae.
87. The method of statement 81-85 or 86, wherein the algae are Nannochloropsis oceanica.
88. The method of statement 81-86 or 87, wherein the algae are genetically modified.
89. The method of statement 81-86 or 87, wherein the algae comprise a heterologous expression cassette comprising a promoter operably linked to a nucleic acid segment encoding a protein with at least 90% sequence identity to any of SEQ ID NO:7-112.
90. The method of statement 81-88 or 89, wherein the fungi are oil-producing fungi.
91. The method of statement 81-89 or 90, wherein the fungi are Mortierella elongata or Mortierella alpina.
92. The method of statement 89-90 or 91, wherein the fungi comprise a heterologous expression cassette comprising a promoter operably linked to a nucleic acid segment encoding a protein with at least 90% sequence identity to any of SEQ ID NO:7-112.
93. The method of statement 81-91 or 92, wherein the algae are strained, pumped, or passed through the fungal-filter.
94. The method of statement 81-92 or 93, further comprising harvesting the fungal-filter, which comprises algal cells.
95. The method of statement 81-93 or 94, further comprising harvesting the fungal-filter, which comprises algal cells, and extracting oil, protein, or carbohydrate therefrom.
96. The method of statement 81-94 or 95, further comprising harvesting harvesting the fungal-filter, which comprises algal cells, and isolating a product made by the fungi or the algae.

The specific compositions and methods described herein are representative, exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “an algae” or “a fungus” or “a cell” includes a plurality of such algae, fungi, or cells, and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

What is claimed:

1. A method comprising contacting a fungal-filter comprising fungal mycelia with a culture of algae to generate an aggregate of algae bound to the fungal-filter hyphae to thereby capture the algae from the culture.

2. The method of claim 1 wherein the fungal-filter is in a container, on a solid surface, or on a solid surface within the container.

3. The method of claim 1 wherein the fungal-filter is pre-made and stored as a dry or moist filter.

4. The method of claim 1 wherein the fungal mycelia are in solution and the fungal-filter is formed in situ after the fungal mycelia are contacted with the algae.

5. The method of claim 1, the fungal mycelia comprises fungal cells.

6. The method of claim 4, the fungal mycelia comprises fungal cells incubated in half strength potato dextrose broth medium.

7. The method of claim 6, wherein the fungal mycelia or fungal cells are incubated for about 2 to 5 days at 20-25° C.

8. The method of claim 2, wherein the container or the solid surface is a petri dish, a silicon membrane, a mesh, or a large pored fabric membrane.

9. The method of claim 3, wherein two or more fungal-filters are stacked together and the culture of algae is contacted with the stacked fungal-filters.

10. The method of claim 1, wherein the contacting comprises passing the culture of the algae through the fungal-filter.

11. The method of claim 1, wherein the algae and the fungal-filter form a flocculate that is collected.

12. The method of claim 1, wherein the fungal mycelia comprise Mortierella mycelia.

13. The method of claim 1, wherein the Mortierella are Mortierella elongata or Mortierella alpina.

14. The method of claim 1, wherein the algae are microalgae, green algae, or blue-green algae.

15. The method of claim 1, wherein the algae are Nannochloropsis oceanica.

16. The method of claim 1, further comprising harvesting the aggregate of algae bound to the fungal-filter hyphae.

17. The method of claim 1, further comprising harvesting the aggregate of algae bound to the fungal-filter hyphae, and separating the algae from the fungal-filter hyphae.

18. The method of claim 17, wherein the algae are separated from the fungal-filter hyphae by one or more of digestion of the fungal-filter, addition of salt, addition of detergent, vortexing, re-suspension of the algae, or a combination thereof.

19. The method of claim 1, further comprising harvesting the aggregate of algae bound to the fungal-filter hyphae and extracting oil, protein, or carbohydrate therefrom.

20. The method of claim 1, wherein the algae is modified to express a selected product, the fungal filter comprises fungal cells modified to express a product, or the algae and the fungal cells are separately modified to express one or more products.

21. The method of claim 20, wherein the product is one or more enzymes that can contribute to synthesizing one or more oils, carbohydrates, vitamins, proteins, or polymers.

22. A method comprising inoculating fungal cells into a dish comprising culture medium, and incubating the fungal cells in the culture medium, for a time and under conditions sufficient to form a fungal filter.

23. The method of claim 22, wherein the dish is a petri dish.

24. The method of claim 22, wherein the dish further comprises a paper, silicon, mesh or fabric membrane for harvesting the fungal filter.

25. The method of claim 22, wherein the culture medium is half strength potato dextrose broth medium.

26. The method of claim 22, wherein the conditions comprise room temperature ranging from 20 to 25° C.


SEQUENCE LISTING (112 sequences)

SEQ ID NO: 1 - DNA, 19 bp, N. oceanica
agaggagccatggtaggac

SEQ ID NO: 2 - DNA, 17 bp, N. oceanica
tcgttccacgcgctggg

SEQ ID NO: 3 - DNA, 19 bp, M. elongata
cttgccacccttgccatcg

SEQ ID NO: 4 - DNA, 21 bp, M. elongata
aacgtcgtcgttatcggacac

SEQ ID NO: 5 - DNA, 19 bp, M. elongata
tcacgwcctcccatggcgt

SEQ ID NO: 6 - DNA, 20 bp, M. elongata
aaggagggtcgtcttcgtgg

SEQ ID NO: 7 - protein, 2226 aa, Mortierella elongata
MTSNVQSFIGGNALDKAPAGAVHDFVSQHGGHSVITKILIANNGIAAVKEIRSVRKWAYETFGDERAIQFTVMATPEDLKVNAEYIRMADQYVEVPGGSNNNNYANVDLIVDIAERTGVHAVWAGWGHASENPKLPESLRDSPQKIIFIGPPGSAMRSLGDKISSTIVAQSADVPTMGWSGTGITETEMDPNGFVTVPEDAYQAACVTDAEDGLKKAHAIGFPIMIKASEGGGGKGIRKVEDPEKFAQAFHQVLGEVPGSPVFIMKLAGNARHLEVQLLADQYGHAISLFGRDCSVQRRHQKIIEEAPVTIAKPDTFEAMEKAAVRLAKLVGYVSAGTVEYLYSHATDTYFFLELNPRLQVEHPTTEIVSGVNLPAAQLQIAMGLPLNRIKDIRVLYGLQPSGTSEIDFEFAQQVSFETQRKPAPKGHVIAVRITAENPDAGFKPSSGMMHDLNFRSSTNVWGYFSVSSAGGLHEFADSQFGHIFAYGQDRGQSRKNMVVALKELSIRGDFRTTVEYLIRLLETQEFEENTINTGWLDSLISNNLTAERPETMLAVMCGAVNRAHTISENCLKEYKKSLEKGQIPSKDVLRSVNQLDFIYDGVRYNFTATRSGPNSYTMYLNGSMISISVRPLTDGGLLVLLDGKAHTTYSLEEVQATRLMVDGKTCLLEKENDPTQLRSPSPGKLVRFLVESGDHVKASQAYAEIEVMKMYMPLIATEDGIVQFIKQPGTTLDAGDIIGILSLDDPSRVKHAKPFEGQLPPMGQPTIHGAKPHQRYRELRLILDNAMDGYDNQALVQPTLKEIFEVLQTPELPYLEFNEVFAALSGRIPPKLEISLHQEVDQSMKNHEHFPARTLQALIDAHCRANFSKPADVSSFLASVAPLTTIIQEYQTGLKTHSWTFIAHYLTKYHEVESLFDDSAREEETILAIRDQYKDDVEKVINIALSHSRVTAKNNLVLSLLDQIKPTSSGGALDKFFSPILKKLAELNGRLTSKVSLKARELLIHVQLPSFEERQAQMEKILRSSVTEEIYGGDHEARMPNYDNLKELVDTTYTVFDVLPNFFYHESAHVRLAAFEVYCRRAYHAYEILDINYHMEHNPLLITWKFLLNTPNKSSEGGPNRVASVSDMSYLINKADPEPVRTGGILAVRDIKELEGRFQSVLDFFPTVKSNKHLAHVQATSVHNNVLNVVLKSESIHPNDDDYWLNLLSPIVKGQSEHLRSHGIRRMTFLIFRQGNYPSYFTFRERNNYAEDQTIRHIEPAMAYRLELSRLSNFDIKPCFIDNRQVHVYYAVGKENVSDCRFFVCALVRPGRLRSSVRTADYLISETDRLLNDILDALEIVGATYKQSDCNHLFINFIPTFQLDATEVESALKGFIDRHGKRLWRLRVTGAEIRFNVQSKNDAADPIPLRFIISNVSGYVLNVDTYREIQTDKGAIFKSVGPSGPFHLLPVNQPYPTKEWLQPRRYKAHLMGTTYVYDFGELFRQAVRAQWNHAVKVNPSLKAPNQVLEMRELVLDEKQQLQQVVREAGSNNCGMVAWIFTLRTPEYPEGRQIIVIANDITYNIGSFGPEEDLVFYKASELARKLGIPRVYLSANSGARIGLASEVIGLFNSCWNDASNPSKGFKYIYLTDAGLKQLEAQEERSGKKSVLTETVVEDGETRHKITDVIGAVDGLGVENLRGSGLIAGETSRAYDDIFTITLVTCRSVGIGAYLVRLGQRTIQNEGQPIILTGAPALNKLLGRDVYTSNLQLGGTQIMYKNGVSHLTAQNDYEGIGKIVNWLSYIPERKNAPVPITVSNDTWDRDIDYLPPKGAVYDPRWLIGGKDAEEEGAAFQTGFFDKGSFTETLTGWARTVVVGRARLGGVPMGVIAVETRSVEHIIPADPANGDSVEQVLMEAGNVWYPNSAYKTAQAINDFNKGEQLPLMIFANWRGFSGGQRDMYNEILKYGSFIVDALSSYKQPVFVYVVPNGELRGGAWVVVDPTINENMMEMYADKRSRAGVLEPEGIVEIKFRKAQLLATMERLDDKYRDLKAQYEKPDLAGADREAIKTKLTEREQELLPVYQQLAIQFADLHDTAGRMKAKGTIRESLDWTNARRYFYWRVRRRLAEEYIRRRMTIASKTQTRDDQTATLKAWFGRDTVHASEAELTQIWEHEDRVVLEWFEGQSRKVDALIQELTAAGTAEEVVRMYTSDRAGVVEGFDRILQSLSDQEKQDILAKFATMTV

SEQ ID NO: 8 - protein, 2137 aa, Nannochloropsis oculate
MATTIPSSNRRAMRAGAALVAVSSILVLLMGPVAEAWRVPGFGQGRSSGVTKPVHAPGFLGRFSTPSSLGPSSASCPTISAVGPLSAATMAPPALSPEAQKKKDAVAAYVKSRGGNLAIRKVLIANNGMAATKSILSMRQWAYMELGDDRAIEFVVMATPEDLNANAEFIRLADRFVEVPGGSNKNNYANVDLIVQMAQREGVDAVWPGWGHASENPRLPNTLKQLGIKFIGPTGPVMSVLGDKIAANILAQTAKVPSIPWSGDGLTAELTAEGTIPDETFQKAMVRTSEEALAAANRIGYPVMLKASEGGGGKGIRMSNNDKELETNFIQVQNEVPGSPMFMMQLCTQARHIEVQIVGDEHGNAAALNGRDCSTQRRFQKIFEEGPPTIVPPEVFKQMELAAQRLTQSIGYIGAGTVEYLFNAATGKYFFLELNPRLQVEHPVTEGLSLVNLPATQLQIAMGIPLNRIPDIRRFYGKDDPYGDSPIDFFNDDYAELPSHVIAARITAENPDEGFKPTSGRIERVKFQSTANVWGYFSVGANGGIHEYADSQFGHLFAKGKSREDARKSLVLALKEIEVRGDIRTTVEYLVQLLETEAFKENTIDTSWLDGLIREKSVRVELNPHDVALSAAIARAFARSVDEERKFVENLSKGQVSIQGIRSINSFPMEITYKDYKYSFHCTRVGPDKLRLAINDQILETKVRQQPDGSLIAEFGGTTHTIYALEEPLGLRMVLDGVTVLLPTVYDPSELRTDVTGKIVRYLQEDGTEIQAGQPYVEVEAMKMIMPLKATESGTVAHRLSPGSIITAGDLLANVQLKDPSKVKKITPFKGALELVGSDDEPGVTGFQAVLKTMNMVLDGYDYEVEFLAQNLVTSAQDGKELLDAATALVTKYLAVEEQFAGKVLDEAMVGLVKANKDSLPTVLALATAHRELPRRNKMVSALIRQLQALVERSSNDLSLDTLIALLDRASRLPGKEYGEVAISSAQALLALRAPPFSTRQDELRTTLLNTKDNDALARSATLTAGVDLLTAMFTDPDANVRKNAIEVYIRRIYRAHRILSLTVEEVDGVMIANWSFKFADTPDEESPLRRGFFTVFPSLEAYTAGSEKFSKVLKTALAGQEAYSQPTNVFHVAVAQLPESQQPEVIANIEGILAENKDLLTECRVRMVNVLFVQGAKNPRYFTFTAVKDFKEDPLRRDMRPTFPQLLELSRLAANYELQRLPSIGRNTQVYLGSERAPVGTKKRGPGNQVLFVRGISHSEQTQTPMGAERVLLMAMDELDYALLDERVGGSASSRLFLNLLVPIDSDPKTLAGEWSKIMDRLLAKYATRLLKLGVDEIEIKVRVAAGSGSAITPVRLMASSMTGEFLRTDAFLEYPDPVTGITKQFCSVTSEDQVCLLNPYPASNSIQTRRASARRIGSTYAYDFLGVMEVSLIQKWDKHLKELTSVYTSRVDDKMPEQLFQADELVLEDGVLKPTQRLVGLNDVGMVAWHATMKTPEYPEGRELVIIANDVTFQSGSFGVKEDDFFRAASEYARVRGLPRIYLSSNSGARIGLVDDLKGKFRIAWNDPANPSLGFKYLYLTPEEYEGLKPGTVNANLVLSEEGEKRWALQDIIGQVHGIGVENLRGSGMIAGETSRAYDETFTLSYVTGRSVGIGAYLVRLGQRTIQMVNGPLILTGYSALNKLLGREVYTSQDQLGGPQIMAPNGVSHLVVDNDKEGISSIIDWLSFVPKDKFSSVPIIDLPTDSPERDVEFQPTKTPYDPRHMLAGTVGPDGAFVPGFFDRGSFIETLGGWGKSVVTGRAKLGGIPMGIISVETRLVEQRIPADPANPESRESLLPQAGQVWYPDSAFKTAQAIEDFNRGENLPLMIFANWRGFSGGTRDMYGEILKFGAKIVDALRTYRHPVFVYIPPNGELRGGAWVVIDPTINEEMMEMYADKDSRGGILEPPGICEVKFRAADQISAMHRLDPVIQALDGELQNAKTEADAIKLKQQLKEREEALLPLYMQVAHEFADLHDRAGRMKAKGVIRDVVTWKRSRSYFYWRARRRVAEDGLVRAMQKADASLSVQDGREKLEALATSGVYGDDKAFVAWVTESGSKIEEQLVSVKHAAVKASLASLLEELSPEERKKVLSGL

SEQ ID NO: 9 - protein, 2139 aa, Nannochloropsis gaditana
MASFPPSNRRATPARVMVVIFSSVLILLAGPVGDAWRMPSIAPGQSTGVAKTSRWAGFLGNFARRSPSISTSPSLPPSLPASSLGPLSAATMAPPSTLSPAAQKKKDAVAAYVKSRGGNLGIRKVLIANNGMAATKSILSIRQWAYMELGDDKAIEFVVMATPEDLNANAEFIRLADRFVEVPGGSNKNNYANVDLIVQVAEREGVDAVWPGWGHASENPRLPNTLKEMGIKFIGPTGPVMSVLGDKIAANILAQTAKVPSIPWSGDGLTAELTAEGTIPDETFQKAMVRTAEEALAAANRIGYPVMLKASEGGGGKGIRMSNNDEELKNNFVQVSNEVPGSPMFMMQLCTQARHIEVQIVGDEHGNAAALNGRDCSTQRRFQKIFEEGPPTIVPPEVFKQMELAAQRLTQSIGYIGAGTVEYLFNAATGKYFFLELNPRLQVEHPVTEGLSLVNLPATQLQIAMGIPLNRIPDIRRFYGKEDPYGDSPIEFFEDDYADLASHVIAARITAENPDEGFKPTSGRIERVKFQSTANVWGYFSVGANGGIHEFADSQFGHLFAKGKTREDARKSLVLALKEIEVRGDIRTTVEYLVQLLETDAFKENTIDTSWLDGLIREKSVRVELAPHEVALSAAIARAFARSQEEEKKFVENLGKGQVSIQSIRSINSFPMEITYKDSKYSFLCSRIGPDKLRLTINGQVLETKVRQQPDGSLIAEFGGTTHTIYALEEPLGLRMVLDGVTVLLPTVYDPSELRTDVTGKVVRYLQDDGAEIQAGQPYVEVEAMKMIMPLKASESGTVTHRLSPGSIITAGDLLANIQLKDPSKVKKIIPFKDTLELAGSGEEPGTTEIESVLKTMNLVLDGFDYEVEFLAQNLVTSVRDGKELLDAAVALVSKYLAVEEQFAGKALDEAMVALVKANKESLGTVLQLATAHRELPRRNKMVSALIRQLQALVERPGTSELALGPLIDLLERTSHLPGKEYGEVAISSAQALLALKAPPFNIRKDELRATLMQTQDNDALARSATLTAGVDLLTAMFTDPDVTVRKNAIEVYIRRIYRAHRILSLSVEEVDGVMVARWSFKFADTPDEESPLRYGFFTVFPSLEAYTEGTEKFSKVLKSSLGGKEVYSEPTNVFHVAVAQLPESDQPEVIANIEAILAEKKELLTECQVRMVNVLFVKGASNPRYYTFTAAENFKEDPLRRDMRPTFPQLLELSRLAANYELQRLPSIGRNTQVYLGTERAAAGVKKRGGSQVLFVRGISHSEQTQTPLGAERVLLMAMDELDYALLDPRVGGSASSRLFLNLLVPITTDPEALAGEWNQVMDRLLAKYATRLLKLGVDEIEIKVRVTADGNTITPVRLMATSMTGEFLRTDAFLEYPDPVNGITKQFCSITREDQICLLNPYPASNSIQTRRASARRIGSTYAYDFLGVMEVSLIQKWDKHLKELSSVYPSRVDDKMPEQLFTAHELVLEDDELQPTQRLVGLNDIGMIAWHATMKTPEYPEGRELVIIANDVTFQSGSFGVKEDEFFRAASEYARVRGLPRIYLSSNSGARIGLVDDLKGKFRIAWNDPANPSLGFKYLYLPPEEYEALKPGTVNANLVETEEGEKRWALQDIVGQVHGIGVENLRGSGMIAGETSRAYDETFTLSYVTGRSVGIGAYLVRLGQRTIQMVNGPLILTGYSALNKLLGREVYTSQDQLGGPQIMAPNGVSHLVVGNDKEGVSSIIDWLSFVPKDKFSAPPILDLPTDSPERDVEFLPTKTPYDPRHMLAGTVGPDGAFVPGFFDRGSFIETLGGWGKSVVTGRAKLGGIPMGVISVETRLVEQRVPADPANPDSRESILPQAGQVWYPDSAFKTAQAMEDFNRGENLPLIIFANWRGFSGGTRDMFGEILKFGAKIVDALRTYRHPVFVYIPPNGELRGGAWVVIDPTINEEMMEMYADKDSRGGILEPPGICEVKFRNADQVSAMHRLDPVIQALDGELQNAKTEQDAAKLTQQLKEREEALLPLYTQVAHEFADLHDRAGRMKAKGVIRDVVTWKRSRSYFFWRARRRIAEDGLIREMQRVDPTLSVQQGREKVSALASPAVYEDDKAFVAWVEEGGEAIAKELEKIKQAAVKASLASLLEGLSAEERKQVLAGL

SEQ ID NO: 10 - protein, 288 aa, Streptococcus salivarius
MGLFDRKEKYIRINPNRSVRNGVDHQVPEVPDELFAKCPGCKQAIYQKDLGQAKICPNCSYTFRISAKERLDLTVDEGSFQELFTGIKTENPLNFPGYMEKLAATKEKTGLDEAVVTGFASIKGQKTALAIMDSNFIMASMGTVVGEKITKLFEHAIEEKLPVVIFTASGGARMQEGIMSLMQMAKISAAVKRHSNAGLLYLTVLTDPTTGGVTASFAMEGDIILAEPQTLIGFAGRRVIENTVRETLPDDFQKAEFLQEHGFVDAIVKRTELADTIATLLSFHGGVQ

SEQ ID NO: 11 - protein, 253 aa, Collimonas fungivorans
MYRTDLESNIHVCPKCDHHMRIRARERLDALLDAGGRYEIGQETLPIDTLKFKDSKKYPDRLKAAMDATGETDALIVLGGSIMTLPVVVAAFEFEFMGGSMGSVVGERFVRGAQVALEQKVPFICITATGGARMQEGLLSLMQMAKTTSMLTKLSEKKLPFISVLTDPTMGGVSASFAFMGDVVIAEPKALIGFAGPRVIENTVREKLPEGFQRAEFLVTKGAVDMIVDRRKMREEIARLLALLQDQPVESIA

SEQ ID NO: 12 - protein, 308 aa, Marinobacter sp.
MSNWLDKIMPSKIRSESKQRTGVPEGLWKKCPKCGAFLYKPELDKNLDVCPKCQHHLRITARRRLDVFLDADGRQEIAADLEPWDRLKFKDSKRYKDRLSQNQKTTGEKDALVAMRGACLDIPLVAVAFEFNFLGGSMGQVVGEKFVQAANVCLEERIPLVCFSASGGARMQEAILSLMQMSKTAAVLERMKQEGIPYISVMTDPVFGGVSASLAMLGDLNIAEPYALIGFAGPRVIEQTVREKLPEGFQRSEFLLEHGAIDMILHRHQMRERIAAVLAKFTDLDQPATEAPIEFEVSERPETDVPAE

SEQ ID NO: 13 - protein, 471 aa, Helicosporidium ex
MTILAWIKDKKNKAILNTPEYSSQSSLSWCFTHKEAASNKAVSFINLSKRRALWTRCEKCGMIQFMRFFKENANLCLSCSYHHIMTSDERIALLVEKGTWYPLNETISPKDPIKFTDTQSYAQRIQSTQEKLGMQDAVQTGTGLINGIPFAIGIMDFRFMGGSMGSVVGEKLTRLIEYATKQGLFLLIVSASGGARMQEGIYSLMQMAKISAALNVYQNEANLLYISLCTSPTTGGVTASFAMLGDIIFSEPEAIIGFAGRRVIQQTLQQELPEDFQTSESLLHHGLIDAIVPRCFLVNAISEVASIFAYAPSKYKKLGNISHYHENTLSWATEEILRRNCINNKKVEYRTIEKIYQTTLYKESFFRLNKLLSKLKSEINFTNKMKKQNNAFNTSSVYANYYDVMLCNYNIGTHSLNLLFNEESEFCKYFPFNMDHMKKENRIKYNFITENSNDFIRKKTINDFSIMLIGD

SEQ ID NO: 14 - protein, 608 aa, Mortierella elongata
MSRRLIISHLSKPSSRVWSSSSSSSSFYSPAFSTSTTVRSPFHIATLQRHRTMASISNGGSNNNNNNSASSSSNAAGSGTLQALRANVVEQYWNDIAAHFREPGFSTFDKERTRRAADRDPEFMRKLLLAVITDRPGQGDILPSVIAKSSCDFFSSLDRNGKTEFLRLLARDFGVLQEDVVKAAEQYQDYAHKEPESKALLRAEQLLRHAIVPGHSKFFDRVSRLPGGLKFLIDMRQDLLSIIQANKGDVYLSSLNESLKEKLQAWFVGFLDLERLTWQSPAVLLEKITQYEAVHKFKDVQDLKRRVGPGRRVFALMNKSLPAEPLVFVQVALVERLSDNVQDILNDPSPGHANPAETVKCAIFYSITTQQPYLQWLSGIELGNFLIKRVVRSLKVEFPQIETFSTLSPIPGFRKWIGQCQNLGQKLLLPQEESIVSQLGQETGAASGDVEDQFSAILKHPSTFSDSETMSKLRPILSRLCARYILLEKRRHLALDPVANFHLRNGACAHRLNWLGDTSTKGMEESFGLMINYLYSLDHIEMNNQQYLLDGTISVSSKDAGFQKVLMDSAVGNSQAAGRGVGEEQGGEEGQVVQVNGSSFRLLEIVTA

SEQ ID NO: 15 - protein, 57 aa, Mortierella elongata
RYILEKKCRHLAMDSVANFHLRNGACAHRLNWLDDTSPKGMEEFFGIVTESRRSLAD

SEQ ID NO: 16 - protein, 124 aa, Mortierella elongata
MFRALVRPASTIYRQAAIKATPATVARMPMGLTFARTYASAGLARSDVEKRVLDILAGFNKVDSNKISLNANFNNDLGLDSLDTVEVVMAIEEEFSIEIPDKDADEIKSAAQAVEYITKRDDAH

SEQ ID NO: 17 - protein, 123 aa, Mortierella elongata
MFRAIRPAALYRSAALYKTAPAVVARNAMALNFARTYASAGLARSDVEKRVLDILAGFNKIDANKIALKANFNADLGLDSLDTVEVVMAIEEEFSIEIPDKDADEIKSAEQAVEYISKREDAH

SEQ ID NO: 18 - protein, 120 aa, Nannochloropsis gaditana
MRVLAFLALLAAPAFAFVPRMPAPVRARAGLTLRFSGEYSEKVRAIVLENMGDDAKVQDYLKANGDDTAEFAAMGFDSLDLVEFSMAVQKEFDLPDLNEEDFANLKTIKDVVTMVEANKK

SEQ ID NO: 19 - protein, 352 aa, Nannochloropsis gaditana
MMSKSLIMLGLLSPTAFAFVPKLSTNVLSRAISSHARKNLVKASAVDYKTAFMFPGQGAQYVGMGAQVSEEVPAAKALFEKASEILGYDLLDRAMNGPKDLLDSTAVSQPAIFVASAAAVEKLRATEGEDAANAATVAMGLSLGEYSALCYAGAFSFEDGVRLTKARGEAMQAAADLVDTTMVSVIGLEADKVNELCAAASSKSGEKIQIANYLCPGNYAVSGSLKAAQVLEEIAKPEFGARMTVRLAVAGAFHTEYMAPALEKLKEVLAKTEFKTPRIPVISNVDGKPHSDPEEIKAILAKQVTSPVQWETTMNDLVKGGLETGYELGPGKVCAGILKRIDRKAKMVNIEA

SEQ ID NO: 20 - protein, 318 aa, Mortierella elongata
MESISQFIPNKLPQDLFIDFATAFGVRAAPYVDPLEDALTAQMEKFFPALVHHYRAFLTAVESPLAAQLPLMNPFHVVLIVIAYLVTVFVGMQIMKNFNRFEVKTFSLFHNFCLVSISAYMCGGILYEAYQSKYGLFENLADHTSTGFPMAKMIWLFYFSKIMEFVDTMIMVLKKNNRQISFLHVYHHSSIFAIWWLVTFVAPNGEAYFSAALNSFIHVIMYGYYFLSALGFKQVSFIKFYITRSQMTQFCMMSVQSSWDMFAMKVMGRPGYPFFITALLWFYMWTMLGLFYNFYRKNAKLAKQAKADAAKEKSKKLQ

SEQ ID NO: 21 - protein, 312 aa, Mortierella elongata
MAAAFLDQVNFSLDQPFGIKLDNYFAKGYELVTGKSIDSFVFQEGVTPLSTQYEVAMWTVTYFIVIFGGRQIMKSQEAFKLKPLFILHNFLLTIASGALLLLFIENLVPILARNGLFYAICDQGAWTQRLELLYYLNYLVKYWELADTVFLVLKKKPLEFLHYFHHSMTMILCFVQLGGYTSVSWVPITLNLTVHVLMYYYYMRSAAGVRIWWKQYLTTLQIVQFVLDLGFIYFCSYTYFAFTYWPHLPNVGKCAGTEGAALFGCGLLSSYLLLFINFYRLTYNAKAKAAKERGSNVTPKTPKADKKKSKHI

SEQ ID NO: 22 - protein, 274 aa, Mortierella elongata
MESAPMPAGVPFPEYYDFFMNWKTPLAIAATYTVAVTLFNPKVGKVSRVVAKSANAKPAEKTQSGAAMTAFVFVHNLILCVYSGITFYNMFPAMIKNFATHSIFDAYCDTDQSLWNGSLGYWGYIFYLSKFYEVIDTIIIILKGRRSSLLQTYHHAGAMITMWSGINYQATPIWIFVVFNSFIHTIMYAYYAATSVGLHPPGKKYLTSMQITQFLVGMSIAVSYLFIPGCIRTPGAQMAVWINVGYLFPLTYLFVDFAKRTYSKRSAAPAKKTE

SEQ ID NO: 23 - protein, 805 aa, Nannochloropsis gaditana
MGNQNSVYFGAPPVRKKAPQHADIQEAWRQIASKVARDKGFEHGRKRKVAIIGSGVAGLGAAYHLLTCAAPGEEVELVVYEASGTPGGHAHTELVREEDGKIIACDTGFMVFNHQNYPNLVELFAELGVDDENTNMSFAVSMDEGKVEWCSESVKTLAGPVYRAMLKDMLRFNRTASNLLLAEPEDPRRAWTLAEFLEKEKYGPEFTNYYIVPMCAALWSSSAADVLAASAYALLTFMDNHCMLQLFNRPQWKTVAQRSQTYVQKIVALLGERLRLNAPVKKVVVHGKGKVEVTDASYHAETFDEAIFACHPDQSLALLEGEARVRLAPYLEAFKYAPNACYLHSDPRLMPRKKEAWGSWNYIGTSAGMLGPGREKPVFVTYWLNQLQNLETETPYFVSLNPLFPPDRALTHKILRESHPQFTPATEAAQRRMTEVQGQDGLWFCGAWMGHGFHEDGLRSGLEVATALSGQKAAWMPPEAEAPVYPMVKAHMNARSTWERCQDLLGQLACVPIRNFLASSIQEGCLVLRLPGTGDKLWFGDRTAGRKETVVLRVQSWWFFVRVALEYDLGLARAYMAGEFEVEGTGWNSDGLTRLFLLFIRNRDAPSGGKRFAVSALLTSWIGYGLNFLRYRLSMDNSLAGSRQNISAHYDIGNDLYTLMLDKSLMMYSSAIYHLELTPSSLTASAEATSSDLVPAGNGNGVVVKSSFPPSSYSMAFKGSLEDAQLRKVDTLIRTCRVERKHTLLDIGFGWGGIAIRAAETIGCKVVGITLSKEQKALAEEKVRAKGLEHLIHFELVDYRVFARR

SEQ ID NO: 24 - protein, 323 aa, Mortierella elongata
MGRDLYESYPIVRQTIDEADAILSSMPSSSSSSSPQEEGYLKRVMFEGPQEELTRTENAQPAILTTSIALLRVLETEHGLDLKESCRFALGHSLGEYSALVATRALSLPDAVRLVRIRGDAMAMAVTDKKGMTAMSALVVRASKLDELVKAMHEIQTELSSTVEIAEIANINSSFQVVISGTVKGVDHASKTLQFRKIAAKAVDLPVSAPFHCSLMEPAARVMKDALADISFKQPIIPIVSNVQAQPIESSNDIPSLLVQQVTDTVQWRQSLVNLHSQQQQYDISEYICIGPGKVICNLLRKEYPLDTIRSVSTVEDIQQWKL

SEQ ID NO: 25 - protein, 360 aa, Saccharomyces cerevisiae
MKLLTFPGQGTSISISILKAIIRNKSREFQTILSQNGKESNDLLQYIFQNPSSPGSIAVCSNLFYQLYQILSNPSDPQDQAPKNMTKIDSPDKKDNEQCYLLGHSLGELTCLSVNSLFSLKDLFDIANFRNKLMVTSTEKYLVAHNINRSNKFEMWALSSPRATDLPQEVQKLLNSPNLLSSSQNTISVANANSVKQCVVTGLVDDLESLRTELNLRFPRLRITELTNPYNIPFHNSTVLRPVQEPLYDYIWDILKKNGTHTLMELNHPIIANLDGNISYYIHHALDRFVKCSSRTVQFTMCYDTINSGTPVEIDKSICFGPGNVIYNLIRRNCPQVDTIEYTSLATIDAYHKAAEENKD

SEQ ID NO: 26 - protein, 437 aa, Mortierella elongata
MSLNARRVVVTGLGLVTPLGIGVQQSWSKLIAGECGVVSLKDLPSPTPGLPGFDTLPSQVGAIVKRTGGKELGGFDSTEWLDRGDEKRMAVFTQYAIAAARMAIKDANWETTTEEEKERTGVCLGSGIGSLDDMATTALSFAESGYRKMSPMFVPKILINMAAGHLTMKYGFKGPNHAVSTACTTGAHSLGDAMRFIQYGDADVMVAGGSEACIHPLAVAGFAKAKSLATKYNDSPSEASRPFDKNRDGFVIGEGAGVVVLEEYEHAKKRGAHIYAELRGYGLSGDAHHMTAPPENGTGAAMAMRRALKAARLTPADIGYVNAHATSTHQGDIAENRAIKSVFDGHHDTIAVSSTKGAVGHLLGAAGAVEAIFAILAVKNNILPPTLNLHEHDDSGEFTLNYVPLKAQEKVLKAAITNSFGFGGTNASLCFAKVDTK

SEQ ID NO: 27 - protein, 458 aa, Nannochloropsis gaditana
MRLSTLSVLGPALGCAFLLFDSSLAYLPSYMRGSKGQIYMKEKSQRVVVTGLGPISAVGIGKDAFWKALLEGKSGIDRISGFDPSGLTCQIGAEVKDFDAKPYFKDRKSAVRNDRVTLMGVAASRIAVDDAKLDLSSVEGERFGVVVGSAFGGLQTLETQIQTMNEKGPGSVSPFAVPSLLSNLISGVIALENGAKGPNYVVNSACAASTHALGLAYAHIAHGEADVCLAGGSEAAVTPFGFAGFCSMKAMATKYNDNPSQGSRPFDKDRCGFVMGEGAGMVVLESLEHAQKRGAHIYAEVAGFGQACDAHHITTPHPEGAGLAQAITLALEDAGMAKEDLTYINAHGTSTAYNDKFETLAVKKALGEEVAKKMYLSSTKGSTGHTLGAAGGLEAIATVLAIETKTLPPTINYETPDPDCDLNVVPNKPITLNEITGAASQSAGFGGHDSVVVFKPFK

SEQ ID NO: 28 - protein, 429 aa, Nannochloropsis gaditana
MSKRSRASSRGLAYIQRLHLLSLSLCLLLSLQCSIRAAAFLVPSSPLPSLPSSHGPSLPSSRPPSSVPKSQALRMATSLTEGSSVDAPAAVPGRSFLRAKPIGVGSAAPEDVITNTDLESIVETSDEWIFTRTGISQRRILTSGGQIRALAATAAARALASAGLEGKDIDLVVLATSSPDDLFGDATSVAAAVGATQAVAFDLTAACSGFLFGVVSASQFLHSGCYRRALVVGADALSRWVDWEDRNSCILFGDGAGAVVLEAAEGEEDSGVLGFAMHSDGTGQGDLNLQFSRDDSQSPPSIREVTPYKGKYNNIAMNGKEVYKFATRKVPTVIEEALANAGLGVENVDWLLLHQANIRIMDVVADRLGLSKDKILTNLSEYGNTSAGSIPLALDEAVKAAKVKKGDIIACAGFGAGLSWGSAIIRWQG

SEQ ID NO: 29 - protein, 547 aa, Mortierella elongata
MLDWRFFTERTCAAVRALGSERHRHSTRWALCLSDPFEFACGLFALLAAGKQIVLPSNHKPAALLPLAGLYDSVLDDLDGLLANGAGGPCAKLRIDPRAPLSLVTSGSSGVPKVIQKTLAQFEAEIHTLATLWGTVMRGVTVVASVPHHHIYGLLFRLLWPLAAGQPFDRMTCVEPADVRARLAALQNTVLVSSPAQLTRWPSLINLTQLTPPPGLIFSSGGPLPAETAAIYTQAFGAAPIEVYGSTETGGIAWRCQPQATHQNEVSDAWTPMPAIDVRCDTEGALQLRSPHLPDDQWWRMEDAVQIEADGRFRLRGRLDRIIKLEEKRVSLPELEHVLMRHPWVKQAAVAPLNGARMTLGALLTLTEEGIQAWRSAASRRFITQALRRYLAEYFDGVVLPRHWRFCMQLPFDERGKLSVTQLATRFATHPLQPEVLAEWCDDNTALLELHVPATLIHFSGHFPGLPILPGVVQIDWVVRYAAHYFARCNGFQTLEQIKFLSMVRPGTTLRLALAHDPERARITFRYYVGERDYATGRIVYSKSAVV

SEQ ID NO: 30 - protein, 202 aa, Nannochloropsis gaditana
MHLLAALVALPAMCTAFVVPLPSAPKHAVRMMADGDAAGAEWRGGQAASAVSKDLKTLLTNENVASILPHRYPFLLVDKVIEMEPGKKAVGIKQITANEPQFTGHFPERPIMPGVLMVEAMAQLSGVLCLQPPVSDGKGLFFFAGIDGVKFRKPVVPGDTLVMEVELVKFMESFGIAKLKGKAYVDGDVAVEIKEMTFALSK

SEQ ID NO: 31 - protein, 171 aa, Nannochloropsis gaditana
MADGDAAGAEWRGGQAASAVSKDLKTLLTNENVASILPHRYPFLLVDKVIEMEPGKKAVGIKQITANEPQFTGHFPERPIMPGVLMVEAMAQLSGVLCLQPPVSDGKGLFFFAGIDGVKFRKPVVPGDTLVMEVELVKFMESFGIAKLKGKAYVDGDVAVEIKEMTFALSK

SEQ ID NO: 32 - protein, 258 aa, Nannochloropsis gaditana
MASHHLTTQEHARRKVAVVTGAAGTLGESITGMLLSEGYVVAALDIRAEGLSAFKATLDKKSDQYHAFAVDISSASAVEEVCRTILTRLGAVSVLINNAGLLSNHKCVQTSLTEWHRVMHVNVDGAFLLSQQLLPCMRSMHFGRIVNITSMAAKTGGVTAGTAYAVSKGALASLTFSLARETAGDGITVNGVAPAYVKTPMVMQQLREEQRVQVLNSIPVGRFCEPEEVAHTVRFLISPLAGFITGEIIDQNGGYHMD

SEQ ID NO: 33 - protein, 241 aa, Mortierella elongata
MRRRVLVTGASRGIGRAIAEQLASDGFALTIHAHSGWTEAQAVVAGIVAQGGQAQALRFDVRERALCSKILTEDVAAHGAYYGIVCNAGVVRDAVFPALSGEDWDTVIDTSLDGFYNVVHPLTMPMVRAKAGGRIITISSVSGMIGNRGQVNYSAAKAGLIGASKALALELASRAITVNCVAPGIIATEMINTELREQASKEVPMKRVGTPSEVAALVSFLMSDAAAYITRQVIGVNGGIV

SEQ ID NO: 34 - protein, 318 aa, Mortierella elongata; identical to SEQ ID NO: 20

SEQ ID NO: 35 - protein, 312 aa, Mortierella elongata; identical to SEQ ID NO: 21

SEQ ID NO: 36 - protein, 274 aa, Mortierella elongata; identical to SEQ ID NO: 22

SEQ ID NO: 37 - protein, 311 aa, Mortierella elongata
MGLSKTVGQASDKNICMIFCKGQPIGQVQPEGILYPEYFDVLVNWRTPVSVAALYVLMVVLLNPKQGKVSRVVAADSAAKGDNKKQQELSSSSPAMTALVFVHNAILCVYSAWTFYGMFFAWKKAFATHTFMEAVCDSDNTFWDSLGYYSYYFYLSKYYEIVDTIIILLKGRRSSLLQTYHHAGAIFTMYMGFNYRAHPIWIFTTFNSFIHTIMYAYYAATSVGLKPPGKKYLTSMQITQFWTGTALAFWYEIGSPKGCFTNPGSRFAIWTVLAYVFPLIYLFTSFASKMYGNRVKAAAAAKATSQQKKVL

SEQ ID NO: 38 - protein, 321 aa, Nannochloropsis oculata
MPKLPKISNIFKFLKADPSKIVPYKSIPDKVPFTQLFQHYPVLDPLYTQYEKNFYASTYVKFAQDTWPVLPLALCGMYALMIIVGTKVMVSRPKHEWKTALACWNLMLSIFSFCGMIRTVPHLLHNVATLPFKDTICRHPAETYGEGACGMWVMLFIFSKVPELVDTVFIVFRKSKLQFLHWYHHITVLLFCWHSYAVTSSTGLYFVAMNYSVHAIMYAYYYLTAINAWPKWIPPSIITVAQISQMIVGVGICASSFYFLYTDPEHCQVKRQNVYAGALMYGSYLYLFCDFFVRRFLRGGKPRLGEEKSAVLTMAKKIKAM

SEQ ID NO: 39 - protein, 173 aa, Nannochloropsis oculata
MSFLIRTPADQIKPYFSEAAQTHYTQLFQHFPILERAYFPFEKNFRAEPFVDFAKATWPLLPLALCTAYALMIVIGTRVMKNREKFDWRGPLAYWNLTLSLFSFCGMLRTVPHLLNNITTLSFRDTVCTSAAKSYGEGVSGLWVMLFIFSKIPELVDTVFIVFRKSKLQFLHW

SEQ ID NO: 40 - protein, 359 aa, Nannochloropsis oceanica
MVFQLARDSVSALVYHFKEGNLNWPMIIYLVLVHLAGYIGLTTILACKWQTLLEAFILWPITGLGITAGVHRLWAHRSYNATLPYRILLMLFNSIANQGSIYHWSRDHRVHHKYSETDADPHNATRGFFFAHMGWLIVKKHPKVVEGGKQLDFSDLAADPVVRFQRDWDPWFAQFMCFVMPALVASRFWGEAFWNAFWVAGALRYMLVLHFTWMVNSAAHLYGDHPYDPTMWPAENPLVSVVAIGEGWHNWHHRYPYDYAASEFGISQQFNPTKAFIDFFAAIGMVTNRKRATGAWAKLKESRARDAANGKSMKDFKGRGSGSDYGTTNTNYAVSNKTVVTDKGAQQPGWEESNHPKYN

SEQ ID NO: 41 - protein, 354 aa, Nannochloropsis gaditana
MAAYFQVFRNSKIGIVLTLSLIFTTAMASPSAYFPEKLSLLLKTLSGSDRLVNPHCIDNPFCAFNDWVNAFLFRDAVKADVMARLGPAGAHYFLTYVRDLVAGSVLYYLTAGLWHTYIYQWHGDYFFTQQGFEKPSAATIKDQIQLAQASMFLYAALPVLAEWLVESGWTQCYYYVEEIGGWPYYLAFTLLYLAMVEVGVYWMHRTLHENKVLYKYIHGLHHKYNKPSTLSPWASVAFNPIDGILQASPYVICLFLVPCHYLTHVAMVFFTAVWATNIHDAMDGNTEPVMGSKYHTVHHTHYHYNFGQFFIFADWMFGTLRIPEPRAAKAVLSPGVVPSSGVRTTGKSGRGKMD

SEQ ID NO: 42 - protein, 452 aa, Nannochloropsis gaditana
MGRGGEKTVTPPSKTFHAHGHSLTASDLSRADAASTISSSVRPSKSLEAMPTEELRKKALQYGHDASADRASLLQILAPYGDILLRTDAPPSLPLTPPPFTLADIKAAVPRHCFERSLTTSFFHLACDLVLVALLGYLATLIGHPDVPTMSRYLLWPLYWYAQGSVLTGVWVIAHECGHQSFSPYERVNNLVGWVLHSALLVPYHSWRISHGKHHNNTGSCENDEVFAPPIKEDLMDEILLHSPLANLAQIIIMLTVGWMPGYLLMNATGPRKYKGKNNSHFDPNSALFSPKDRLDIIWSDIGFFLALAGVVWACTQYGFSTVGKYYLLPYMVVNYHLVLITYLQHTDVFIPHFRGAEWSWFRGALCTVDRSFGWLLDHTFHHISDTHVCHHIFSKMPFYHAQEASEHIKKALGPYYLKDDTPIWKALWRSYTLCKYVDTDKNAVFYKHRAS

SEQ ID NO: 43 - protein, 337 aa, Nannochloropsis gaditana
MSRYLLWPLYWYAQGSVLTGVWVIAHECGHQSFSPYERVNNLVGWVLHSALLVPYHSWRISHGKHHNNTGSCENDEVFAPPIKEDLMDEILLHSPLANLAQIIIMLTVGWMPGYLLMNATGPRKYKGKNNSHFDPNSALFSPKDRLDIIWSDIGFFLALAGVVWACTQYGFSTVGKYYLLPYMVVNYHLVLITYLQHTDVFIPHFRGAEWSWFRGALCTVDRSFGWLLDHTFHHISDTHVCHHIFSKMPFYHAQEASEHIKKALGPYYLKDDTPIWKALWRSYTLCKTAEEEEDDEWGVVPKPTEQLYLGNRKARELIGGAYADVNLAVKVAHDDTK

SEQ ID NO: 44 - protein, 482 aa, Nannochloropsis gaditana
MGSTEPVLSTAAVPATEPAGKSYTWQEVAEHNTEKSLWVTVRGKVYDISSWVDNHPGGKEILLLAAGRDITYAFDSYHPFTEKPTQVLNKFEIGRVTSYEFPQYKADTRGFYKALCTRVNDYFVAHKLNPKDPIPGIWRMCLVALVALASFVVCNGYVGVEGTWAGTTWARLVAAVVFGICQALPLLHVMHDSSHLAFGNTERWWQVGGRLAMDFFAGANMTSWHNQHVIGHHIYTNVFLADPDLPDKAAGDPRRLVQKQAWQAMYKWQHLYLPPLYGILGIKFRVQDIMETFGSGTNGPVRVNPLSFFQWAEMIFTKMFWAGWRIAFPLLSPSFHTGWAAFSALFLVSEFMTGYFLAFNFQVSHVSSECDYPLGEAPREGEDGNIVDEWAVSQIKSSVDYAHNNPVTTFLCGALNYQVTHHLFPTVSQYHYPAIAPIIQDVCREFNVDYKVLPDFVTAFHAHIAHLKTLGERGEAAEVHMG

SEQ ID NO: 45 - protein, 163 aa, Nannochloropsis gaditana
MSGSQGRPERVGEGHPRDARREEKCGSADNGLRDGRAERAKEEGRGAYPDAMNEVACVFLYPTLPRITSSSPVTVPPGLQVMAAVVLRHAPFPLLLFLTYTLSGSCNHFLTLIMHEVAHNLAFKRLFANRVFSIIVNLPLGIPAAMWVWEGGPEGGVQAPTSG

SEQ ID NO: 46 - protein, 445 aa, Mortierella elongata
MATPLPPTFVVPATLTETRRDPLKHQELPPLFPEKVNILNIWKYLDYKHVVGLGVTPLIALYGLLTTEIQRKTLIWSIIYYYATGLGITAGYHRLWAHRSYNAGPAMSFVLALLGAGAVEGSIKWWSRGHRAHHRWTDTEKDPYSAHRGLFFSHLGWMLIKRPGWKIGHADVDDLNKNKLVQWQHKNYLALIFLMGVVFPTVVAGLGWGDWRGGYFYAAILRLVFVHHATFCVNSLAHWLGEGPFDDRHSPRDHFITAFMTLGEGYHNFHHQFPQDYRNAIRFYQYDPTKWVIATCAFLGLASHLKTFPENEVRKGQLQMIEKRVLEKKTKLQWGTPIADLPVMSFEDYRHACKNDNKKWILLEGVVYDVADFMSEHPGGEKYIKMGIGKDMTAAFNGGLYDHSNAARNLLSLMRVAVVEFGGEVEAQKKNPSAPIYGDDHAKAA

SEQ ID NO: 47 - protein, 445 aa, Mortierella alpina
MATPLPPSFVVPATQTETRRDPLQHEELPPLFPEKITIYNIWRYLDYKHVVGLGLTPLIALYGLLTTEIQTKTLIWSIIYYYATGLGITAGYHRLWAHRAYNAGPAMSFVLALLGAGAVEGSIKWWSRGHRAHHRWTDTEKDPYSAHRGLFFSHIGWMLIKRPGWKIGHADVDDLNKSKLVQWQHKNYLPLVLIMGVVFPTLVAGLGWGDWRGGYFYAAILRLVFVHHATFCVNSLAHWLGDGPFDDRHSPRDHFITAFVTLGEGYHNFHHQFPQDYRNAIRFYQYDPTKWVIALCAFFGLASHLKTFPENEVRKGQLQMIEKRVLEKKTKLQWGTPIADLPILSFEDYQHACKNDNKKWILLEGVVYDVADFMSEHPGGEKYIKMGVGKDMTAAFNGGMYDHSNAARNLLSLMRVAVVEYGGEVEAQKKNPSMPIYGTDHAKAE

SEQ ID NO: 48 - protein, 446 aa, Mortierella elongata
MATPLPPTFVVPATQTETRRLPLEHDELPPLFPEKLTITNIWKYLDYKHVLGLGLTPLIALYGLLTTEIQTKTLIWSIVYYYATGLGITAGYHRLWAHRAYSAGPAMSFALALLGAGAVEGSIKWWSRGHRAHHRWTDTEKDPYSAHRGLFFSHIGWMLIKRPGWKIGHADVDDLNKNKLVQWQHKHYLPLVLFMGVIFPTIVAGLGWGDWRGGYFYAAILRLVFVHHATFCVNSLAHWLGEGPFDDRHSPRDHFITAFMTLGEGYHNFHHQFPQDYRNAIRFYQYDPTKWVIAICAFFGLASHLKTFPENEVRKGQLQMIEKKVLEKKTKLQWGTPIADLPVLSFEDYQHACKNDGKKWILLEGVVYDVAEFMNEHPGGEKYIKMGVGKDMTAAFNGGMYDHSNAARNLLSLMRVAIVEFGGEVEAQKKNPSVPIYGDDHHSKSE

SEQ ID NO: 49 - protein, 457 aa, Mortierella elongata
MAATPSVRTFTRSEILNAEALNEGKKDAEAPFLMIIDNKVYDVREFVPEHPGGSVILTHVGKDGTDVFDTFHPEAAWETLANFYVGDIAEHDRAIKGDDFAAEVRKLRSLFQSLGYYDSSKAYYAFKVSFNLCLWALSTFIVAKWGQTSTLATIASASILGLFWQQCGWLAHDFLHHQVFQDRFWGDLFGAFLGGVCQGFSSSWWKDKHNTHHAAPNVHGEDPDIDTHPLLTWSEHALEMFSDVPDEELTRMWSRFMVLNQTWFYFPILSFARLSWCLQSILFVLPNGQAHKPSGARVPISLVEQLSLAMHWTWYFATMFLFIKDPVNMIVYFLVSQAVCGNLLALVFSLNHNGMPVISKEEAVDMDFFTKQIITGRDVHPGLFANWFTGGLNYQIEHHLFPSMPRHNFSKIQPAVESLCKKYGVRYHTTGMVDGTAEVFARLNEVSRAASKMGKST

SEQ ID NO: 50 - protein, 444 aa, Mortierella elongata
MGAEKEFTWEELAKHNIAGDLYVAVRGNVYDVTKFLSRHPGGVDTLLLGAGRDVTPVFDMYHAFGTGDAIMKKYYVGKLVSNELPIFPEPSGFHKVVKSRVEGYFKDSGKDPKNRPEIWGRYFLIFAALFLSYYAQFFVPFVVERTWLQVIFAVIMGFACAQIGLNPLHDASHFSTTHNPTVWKILGATHDFFNGASYLVWMYQHMLGHHPYTNIAGADPDVSTAERDVRRIKPSQKWFWNHINQHMFVPFLYGLLAFKVRIQDVNILYFVGTNDAIRVNPISLWHTVMFWGGKIFFFWYRIYVPLQVLPLKKVLILFTIADMISSYWLALTFQANHVVEEVEWPLPDENGIIQKDWAAMQVETTQDYAHESYIWTSITGSLNYQAVHHLFPNVSQHYYPEILSIIRDACTEYKVPYLVKDTFWQAFSSHLEHMRVLGLRPKEE

SEQ ID NO: 51 - protein, 396 aa, Mortierella elongata
MAPPNTIDAGLTHRHVVNPTAAPVKAAYERNYELPEFTIKEIRECIPAHCFERSGFRGLCHVAIDLTWASLLFLAATQIDKFENPLIRYLAWPVYWVMQGIVCTGIWVLAHECGHQSFSTSKTLNNTVGWILHSFLLVPYHSWRISHSKHHKATGHMTKDQVFVPKTRTQVGLPAKKENVVEEDEAVHLDEEAPIVTLFWMLVQFTFGWPAYLAVNASGQDYGQWTSHFHTWSPIFEARNFTDVILSDLGVLVTLGALIYASLQTSLLAVTKYYIVPYLFVNFWLVLITFLQHTDPKLPHYRENVWNFQRGALCTVDRSFGKFLDHMFHGIVHTHVAHHLFSQMPFYHAEEATACLKKLLGKHYIYDDTPIVLATWRSFRECRFVEDEGDVVFFKK

SEQ ID NO: 52 - protein, 457 aa, Mortierella alpina
MAAAPSVRTFTRAEILNAEALNEGKKDAEAPFLMIIDNKVYDVREFVPDHPGGSVILTHVGKDGTDVFDTFHPEAAWETLANFYVGDIDESDRAIKNDDFAAEVRKLRTLFQSLGYYDSSKAYYAFKVSFNLCIWGLSTFIVAKWGQTSTLANVLSAALLGLFWQQCGWLAHDFLHHQVFQDRFWGDLFGAFLGGVCQGFSSSWWKDKHNTHHAAPNVHGEDPDIDTHPLLTWSEHALEMFSDVPDEELTRMWSRFMVLNQTWFYFPILSFARLSWCLQSIMFVLPNGQAHKPSGARVPISLVEQLSLAMHWTWYLATMFLFIKDPVNMIVYFLVSQAVCGNLLAIVFSLNHNGMPVISKEEAVDMDFFTKQIITGRDVHPGLFANWFTGGLNYQIEHHLFPSMPRHNFSKIQPAVETLCKKYGVRYHTTGMIEGTAEVFSRLNEVSKAASKMGKAQ

SEQ ID NO: 53 - protein, 457 aa, Mortierella alpina
MAAAPSVRTFTRAEILNAEALNEGKKDAEAPFLMIIDNKVYDVREFVPDHPGGSVILTHVGKDGTDVFDTFHPEAAWETLANFYVGDIDESDRAIKNDDFAAEVRKLRTLFQSLGYYDSSKAYYAFKVSFNLCIWGLSTFIVAKWGQTSTLANVLSAALLGLFWQQCGWLAHDFLHHQVFQDRFWGDLFGAFLGGVCQGFSSSWWKDKHNTHHAAPNVHGEDPDIDTHPLLTWSEHALEMFSDVPDEELTRMWSRFMVLNQTWFYFPILSFARLSWCLQSIMFVLPNGQAHKPSGARVPISLVEQLSLAMHWTWYLATMFLFIKDPVNMIVYFLVSQAVCGNLLAIVFSLNHNGMPVISKEEAVDMDFFTKQIITGRDVHPGLFADWFTGGLNYQIEHHLFPSMPRHNFSKIQPAVETLCKKYGVRYHTTGMIEGTAEVFSRLNEVSKAASKMGKAQ

SEQ ID NO: 54 - protein, 454 aa, Mortierella verticillata
MVATRTFTRSEILNAEALNEGKKNADAPFLMIIDNKVYDVREFVPDHPGGSVILTHVGKDGTDVFDTFHPEAAWETLANFYVGDIAENDRAIKNDDFAAEVRKLRTLFQSLGYYDSSKAYYAFKVSFNLCLWALSTFIVAKWGQTSTLANVLSASILGLFWQQCGWLAHDFLHHQVFQDRFWGDLFGAFLGGVCQGFSSSWWKDKHNTHHAAPNVHGEDPDIDTHPLLTWSEHALEMFSDVPDEELTKMWSRFMVLNQTWFYFPILSFARLSWCLQSIMFVMPNGQAHKPSGARVPISLVEQLSLAMHWTWYFATMFLFIKDPVNIMVYFLVSQAVCGNLLALVFSLNHNGMPVISKEEAVDMDFFTKQIITGRDVHPGLFANWFTGGLNYQIEHHLFPSMPRHNFSKIQPAVASLCKKYNVRYHTTGMVDGTAEVFARLNEVSRAASKMGKSA

SEQ ID NO: 55 - protein, 457 aa, Mortierella alpina
MAAAPSVRTFTRAEILNAEALNEGKKDAEAPFLMIIDNKVYDVREFVPDHPGGSVILTHVGKDGTDVFDTFHPEAAWETLANFYVGDIHESDRDIKNDDFAAEVRKLRTLFQSLGYYDSSKAYYAFKVSFNLCIWGLSTFVVAKWGQTSTLANVVSAALLGLFWQQCGWLAHDFLHHQVFQDRFWGDLFGAFLGGVCQGFSSSWWKDKHNTHHAAPNVHGEDPDIDTHPLLTWSEHALEMFSDVPDEELTRMWSRFMVLNQTWFYFPILSFARLSWCLQSILFVMPNGQAHKPSGARVPISLVEQLSLAMHWTWYLATMFLFVKDPINMFVYFLVSQAVCGNLLALVFSLNHNGMPVISKEEAVDMDFFTKQIITGRDVHPGLFANWFTGGLNYQIEHHLFPSMPRHNFSKIQPAVETLCKKYNVRYHTTGMIEGTAEVFSRLNEVSRAASKMGKAQ

SEQ ID NO: 56 - protein, 338 aa, Mortierella elongata
MSDSHLTVDPTSTTPHPDADGTTNNTIIETMLDLEEIDKDLYRSKKLWVPMGARGVFGGNVVGQALVAATNTVSTDYSVHSLHSYFLLPGDHTTPILYHVERVRDGKSYCTRTVTAKQRGKNIFVCTASYQVPRPGAPSHQYPMPNVPHHSTLPSQEELIHAMIDNPKLPENLKDFLRLRLDEPVALEFKDTKRHTFKELMNPEVRTDQSFWIRCKGQLGDALALHQCVVAYGSDHNLLNTVPLAHGSSWFSRRSGLSPKITMMASLDHSMWFHCPFRADEWLLYVCETPRSGCDRGLTFGRIYKEDGTLAISVAQEGVVRLQPKTPTPAATVETPKL

SEQ ID NO: 57 - protein, 334 aa, Lobosporangium transversale
MSSVSEPGSTLNLAPTPDGSSNNTIIETMLDLEEIDKDLYRSKKLWLPLGARGVFGGNVVGQALVAATNTVSDLYSVHSLHSYFLLPGDPTIPILYHVDRLRDGHSYCTRTVTATQRGKNIFVCTASFQVPRPNAPSHQYPMPNVPHHSTLPSQEDLIRAMIDSPKIPENLVEFLKQRLDEPVALDFKDTRRHTLKDLMNPPVRTEQTFWIKCKGGLGDALALHQCVVAYGSDHNLLNTVPLAHGSTWLSRRSSSPSIVMMASLDHSMWFHCPFRADEWMLYVCETPRSGCDRGLTFGRIYKEDGTLAVSVAQEGVVRLRSKAPSSATVDQPKL

SEQ ID NO: 58 - protein, 146 aa, Mortierella elongata
MMAKQITQTVLTATVGIEVPFHDIDSMNICWHGHYVKYFEIARSALLRSFEYDAMRLSNYLWPVVECRLKYLRPARYGQLLDVSAKLVEYESRLKIGYLITDRESGAQLTKGYTIQVAVDAQTQALQFVLPRELLDKLEPMLSAVC

SEQ ID NO: 59 - protein, 132 aa, Mortierella elongata
MHSLSHLPHDKTLALRAVPQPSNANMHGDVFGGWIMAQVDIAGSIPATRRAHGRVVTVAVNSLVFKQPVFVGDLLSFYADIAKVGNTSVAVSVEVYAQRLNFAEQIFKVAEATLTYVATDNDRRPRALPAEG

SEQ ID NO: 60 - protein, 422 aa, Nannochloropsis gaditana
MSLKTISPHDYRSKMTRQERTSRQVLELLHAVSKSAFSGVLLRRDIEPNATELQNVKALKIGPGPQVRLRLRVPSHLCDNYNNNHRLLDAGAVTAWFDEVSSWAFVSADGRHRPGVSVSLNTTVLSWVPVGTEVEIQSHCKKIGETLGFADMMLLDVATGKELAHGRHVKFLKMGTAWTVAMHAWAFPLTYLMASAVLLPSVRQRTQKSSSFPPEMAPSPDLPRTEPGSAVNINRLLALDNFHVYEPAGAASPPLAFPASVPLTMEASASFRVIPQVCNSFGSLHGGAAAILAERAALALYHQAARWAGERSQHALPRVRSLSIDYMSPCKKNTELLLLVRGMRVERGAGEGDKHSPSRSLFPPLDVAPHPQGNLIPMSYQVLFTRKKDGRYLTQCHVLLDSQGDAWHHQRQSRGEGNRARL

SEQ ID NO: 61 - protein, 272 aa, Nannochloropsis gaditana
MSLKTISPHGYRSKMTRQEQTSRQVLELLHAVSKSAFSGVLLRRDIEPNATELQNVKALKIGPGPRVRLRLRVPSHLCDNYDNNHCLLDAGAVTAWFDEVSSWAFVSADGRHRPGVSVSLNTTVLSWVPVGTEVEIQSHCKKIGETLGFADMMLLDVATGKELAHGRHVKFLKMGTAWTVAMHAWAFPLTYLMASAVLLPSVRQRTQKSSSFPPEMAPSPDLPRTEPGSAASVLSMVGPPQFWLSALLLPCITKPLGGPERGASTLCRVFVL

SEQ ID NO: 62 - protein, 547 aa, Mortierella elongata; identical to SEQ ID NO: 29

SEQ ID NO: 63 - protein, 819 aa, Mortierella elongata
MPDLAWSLPVARWSAWNAETSAALDMGLKVANDCAPVGQPVRVIFASRHGESRRTTELLKAQAQDPMQPLSPNAFSLSVLNAAAGVFSMMRGDHSNATALAAGSETLGYALLEAFAQYASDPQAPVLVIYADEPPDPIYASVDDTDAPSGALALWIADDAPGVLECRLLIDALNLEDLTLADIGDDTPLFDTDGIGLDSIDALEIGIALRKKYQLQIETTDSRMREHFRSLLLDALAGVSQRPTLFRMTTPLHLLFSNDCVATRPVCIDGDHILDWRFFTERTCAAVRALGSERHRRSARWALCLSDPFEFACGLFALLAAGKQIVLPSNHKPAALLPLAGLYDSVLDDLDSLFANGAGGPCAKLRIDPRAPLSLVTSGSSGVPKVIHKTLAQFEAEIHTLATLWGTVMRDVTVVASVPHHHIYGLLFRLLWPLAAGQPFDRMTCVEPADVRARLAALQNTVLVSSPAQLTRWPSLINLAQLTPPPGLIFSSGGPLPTETAAIYAQAFGAAPIEVYGSTETGGIAWRCQPQAMHQNEVSDAWTPMPAIDVRCDTDGALQLRSPHLPDDQWWRMEDAVQIKVDGRFRLRGRLDRIIKLEEKRVSLPELEHVLMRHPWVKQAAVAPLNGARMTLGALLTLTEEGIQAWRSAASRRFITQALRRYLAEYFDGVVLPRHWRFCMQLPFDERGKLSVTQLAARFATHPLQPEVLAEWCDGNTALLELHVPATLSHFSGHFPGLPILPGVVQIDWVVRYAAHYFARCNGFQTLEQIKFLSMVRPGTTLRLALAHDPERARITFRYYVGERDYATGRIVYSKSAVV

SEQ ID NO: 64 - protein, 572 aa, Mortierella elongata
MTTPLHLLFSHDCVATRPVCIDGDHMLDWRFFTERTCAAVRALGSERHRHSTRWALCLSDPFEFACGLFALLAAGKQIVLPSNHKPAALLPLAGLYDSVLDDLDGLLANGAGGPCAKLRIDPRAPLSLVTSGSSGVPKVIQKTLAQFEAEIHTLATLWGTVMRGVTVVASVPHHHIYGLLFRLLWPLAAGQPFDRMTCVEPADVRARLAALQNTVLVSSPAQLTRWPSLINLTQLTPPPGLIFSSGGPLPAETAAIYTQAFGAAPIEVYGSTETGGIAWRCQPQATHQNEVSDAWTPMPAIDVRCDTEGALQLRSPHLPDDQWWRMEDAVQIEADGRFRLRGRLDRIIKLEEKRVSLPELEHVLMRHPWVKQAAVAPLNGARMTLGALLTLTEEGIQAWRSAASRRFITQALRRYLAEYFDGVVLPRHWRFCMQLPFDERGKLSVTQLATRFATHPLQPEVLAEWCDDNTALLELHVPATLIHFSGHFPGLPILPGVVQIDWVVRYAAHYFARCNGFQTLEQIKFLSMVRPGTTLRLALAHDPERARITFRYYVGERDYATGRIVYSKSAVV

SEQ ID NO: 65 - protein, 697 aa, Neurospora crassa
MANTGPGNVPLHFIQKPPFTVEDPNAQPIPGETIPRRHPKAKNGLATRPAPGVNTTLDLLTRTVELYGDERAIGSRKLIKLHKDIKKVPKVVDGETVMVDKEWQCFELTPYSYITYGEYFTIVKQIGAGLRKLGLEPKDKLHIFATTSPQWLGMSHAASSQSLTIVTAYDTLGESGVQHSLVQSKASAMFTDPHLLKTATNPLKEATSVKVVIYNNHTTQPVSQDKIDAFKAEHPDLTVLSFEELRALGEENPVPLTPPNPDDTYCIMYTSGSTGPPKGVPVSHAGFVAAVAGLYAVMEESVTHRDRVLAYLPLAHIFELVLENLGVFVGGTLGYSNARTLSDTSMRNCPGDMRAFKPTIMVGVPQVWETVKKGIEGKVNSAGALTKALFWGAYNIKSFLVSNNLPGKTIFDDLVFGQVRTMTGGELRFIVNGASGIAASTQHFMSMVVAPMLNGYGLTETCGNGALGSPMQWTSNAIGAMPAAVEMKLVSLPELNYHTDTVPPQGEILFRGACVIKEYYENPEETAKAITPDGWFKSGDIGEIDANGHLRVIDRVKNLVKLQGGEYIALEKLEAVYRGAVFVHNIMVHGDNSAPRPIAVVVPNEKALAEKAEELGLGAEAPGEMHRNRKLRDAVLKELQSVGRRAGLSGMETVAGVVLVDDEWTPANGFVTATQKINRRAVKERYSKEISDCLDGK

SEQ ID NO: 66 - protein, 671 aa, Nannochloropsis gaditana
MDRYKWRTLPDVFETVASLAPEAVAVEDMVHTPTAKMTYGELNRQIGALAAFFQHEGLKPGQCVSVFAENSHRWLIADQAILKAGACNAVRGVKAPVDELQYIYQNSESVASVVESVEQIEALMRTNGGLTGRYGPPRFILVLFPGERSGQEIRELANLPPPTQVLTFDEALSASLARPLTFRPVPKDVRSVATLVYTSGTTNKPKGVVLRHSNLLHQVNYNSFTDSPSKEPAYNPVLGDVLVSVLPCWHIFERTAEYWMFSKGIHVVYSNVKNFKADLAKHQPQFIVAVPRLLETIYRGVLQKFATEKGAKKKIIEFFTRVGSAWVKAWRVARGLVLRSRAPNPIERLLALVLALVLSPLAAVGDKLVWSKVRAGLGGRIKVLVAGGSSMPLVLEDFFELLRTPVIVGYGMTETSPVITNRVAEKNLAGSVGRTARDTEVKIVDPESGARLPEGQPGLVLMRGPQMMAGYKSNAEASKAVLDQEGFLDTGDLGRIHPLTKHLIITGRAKDTIVLSNGENVEPQPIEDVVCANSALVDQVMCVGQDEKVLGMLVVPNVRALARAGLVDRGLAERVAELLGGQVLTNGIAGSRAELEEVEASLREKKEVKKALLADIARAMGKSFRETERVGAVEVVLEPFNMANGFLTQTLKVKRNVVSGHYAQEIEQMYR

SEQ ID NO: 67 - protein, 497 aa, Nannochloropsis gaditana
MHGRSKKLGNILEELGVKKGDRVATLAMNTYRHMELYFAVSGAGAVLHTLNPRLFAETLTWIVHHAQDSVLFFDPCFASLVERLLPHCPSVKHWICLVDEERMPVLPSLSPSSPFLSLHNYEALLREGKEDYVWPILEETAASSLCYTSGTTGIPYTAAMVGCKLVLPGSALDGASLYELMKEEGVTLAAGVPTVWLPVLHHLDQDPGQGLPKLRRLVIGGAACPPSMLRAFKERHGIEGKHLALPTEDQHNVLSTQGRTIYGVDLRIVAPSPPPYLPSSSSSYSPPYPPRWSEVPWDGVSPGELCARGHWVATDYFSPTQAPEEGERDGGVRAGHQESFYTDDDGERWFLTGDVATICPDGYIKITDRSKDVIKSGGEWISSIELENIATNHPEVALAAVIAMPHRKWDERPLLIVVLKDSAALSLHYSTTSSSPSTSSDTDRAIRLTKEALLDHFKGKVAKWWVPDDVIFVDSLPQGPTGKILKTELRQRFSRRP

SEQ ID NO: 68 - protein, 649 aa, Nannochloropsis gaditana
MPKYTTTVASGEVDLRIEKEGPGSWAPKTVFQVFEETVKKYGDSPALHYKKVPHGGSLATTEWSSYTWREYYDLTLEFCKSLLSLGFPAHGAINLIGFNSPEWLIANCGAIAAGGVGVGIYTSNGVDACKYITEHSEAEVVVVENAKQLEKYLKIAKELPRLKALVIYSGTAEGYKCDVPIYSWKDFMALGSGVKDEAVRARIEAQRPGHCCTLIYTSGTTGPPKAVMISHDNLTWTVKNFVASLPFTLTCEDRSVSYLPLSHVAAQMLDIHCPIATGAKIYFAQPDALRGSLPVTLKDVCPTYFFGVPRVWEKIYEKMQEVARSTTGVKRALAQWAKAKGLEKNRRQQYGCGGGAPVGFGCAHALVLSKVKAALGLHQTKMCITSAAPIAVEILEYFASLDIPVLELFGQSECTGPHTSNFSYAWKIGSIGRDIPGVKTKQHANMSEFCMYGRHIMMGYMKMEDKTQEAVDNEGWLHSGDVAQVDADGFWSITGRIKELIITAGGENIPPVLIENEIMSALPAVANCMVVGDKKKFLTVLLTMKAKLDDQGNPTKELNKEALDIGKEIGSNASTTEQVASDPHWKKYFDEGLKKANSTATSNAQFVQKWSVLPLDFSEKGGELTPTLKLKRSVVAEKYADVIADMYKA

SEQ ID NO: 69 - protein, 649 aa, Nannochloropsis gaditana
MPKYTTTVASGEVDLRIEKEGPGSWAPKTVFQVFEETVKKYGDSPALHYKKVPHGGSLATTEWSSYTWREYYDLTLKFCKSLLSLGFPAHGAINLIGFNSPEWLIANCGAIAAGGVGVGIYTSNGVDACKYITEHSEAEVVVVENAKQLEKYLKIAKELPRLKALVIYSGTAEGYKCDVPIYSWKDFMALGSGVKDEAVRARIEAQRPGHCCTLIYTSGTTGPPKAVMISHDNLTWTVKNFVASLPFTLTCEDRSVSYLPLSHVAAQMLDIHCPIATGAKIYFAQPDALRGSLPVTLKDVCPTYFFGVPRVWEKIYEKMQEVARSTTGVKRALAQWAKAKGLEKNRRQQYGCGGGAPVGFGCAHALVLSKVKAALGLHQTKMCITSAAPIAVEILEYFASLDIPVLELFGQSECTGPHTSNFSYAWKIGSIGRDIPGVKTKQHANMSEFCMYGRHIMMGYMKMEDKTQEAVDNEGWLHSGDVAQVDADGFWSITGRIKELIITAGGENIPPVLIENEIMSALPAVANCMVVGDKKKFLTVLLTMKAKLDDQGNPTKELNKEALDIGKEIGSNASTTEQVASDPHWKKYFDEGLKKANSTATSNAQFVQKWSVLPLDFSEKGGELTPTLKLKRSVVAEKYADVIADMYKA

SEQ ID NO: 70 - protein, 340 aa, Mortierella elongata
MSASNAKVEDTTTTFTGWASTGSLPLKKFSYHPRPLGPKDIEIEITHCGICGSDVSTVTGGFGPLSTPCIAGHEIVGTVVKAGPTVFTRSATLSVLVALLIPAVTGGFADRLRVSSEYAYKIPSEIPPAEAAPPLCAGITTYTPLKHFGAGPGKRVGVMGIGGLGHLAIQWAAALKADEVVAISTSDNKREEAKKLGATKFVNSRNEEERKAARHSMDILLLTSNDKNTDWGELIDYVASHGTLVLLALPEIPTIAVPPSSLLMRHVSIAGSLTGGREITQEMLEFAAKHNVHPWITTMPMSDANTAVKLWLETIWCDVAESVVAIVVAVAGEPVMPARK

SEQ ID NO: 71 - protein, 367 aa, Mortierella elongata
MTGGRTIKAALYEGVNPSAPLLKVIDLPAPVANNGDAVVKILATRVVSYAKEVLDGTRPYPNLLPMVPGPGGVGIIQSVAPGAIHIKPGQMVFIDPTVRSRDHPVSPEAMLQGLVAFGSGQELQKVWNNGSWAEEMLVPLENLTVIPESIQAKFNPAELTSISNYAVPLGGLYPNLRPGQTVVITGSTGMFGSSAVAVALALGARRVIASGRNKKQLDEFVRLYGPRVVPVVVTGDVAQDTQAFLKAAGEGFDIDVTFDILPPQATFGAVQSSILALRNGGTAVLMGGLNSSAEIPYPAIMNKGLTIKGHFMYDRSGPTTIIGLADAGLLDLHHRQEPKFFKLSEINDAVEWSAAHPGAFDATLVLP

SEQ ID NO: 72 - protein, 354 aa, Mortierella elongata
MKAALYEGVNHSAPLLKVTDLPVPIATNGDAVVKILASRVVSYAKDVLDGTRPFPNLLPMVPGTGGVGIIQSVAPGAIHIKPGQMVFINSAVRSRDHPVTPEGMVQGLLAFGRSKELQRAEEMLVPLENLTVIPESVQAKFDPAELTSISNYAVSFGGLYPNLRPGQTVVITGSTGVFGSSAVAVALALGARCVIASGRNKKQLDEFATLYGPRVVPVVTTGDVAKDTAAFVKAAGEGFDIDVSFDILPPQAGFGAVKSSILALRAGGTALLMGGVNSSVEIPYSVIMNKGLTIKGVFMSDRAGPTTIIGLAEAGLLDLHHRQEPKIFKLDEINDAVEWSSNHSSAFDATIVIP

SEQ ID NO: 73 - protein, 313 aa, Nannochloropsis gaditana
MPVIGLGTWKAPKGEVKKAVLAALKQGYRHLDCACDYGNEEEVGAAIKEAMEAGVVTRKDLFVTSKLWNTFHAREHVEVAIQKSLKDLGLDYLDLYLIHFPISMKYVPIEELYPPEWLNPTSKKIEFVDVPVSETWAGMEGVCRKGLARNIGVSNFCAQTLMDLLKYAEIKPAVNQIELHPYLTQDSLVAFCQEKGIVLTAFSPLGASSYIELGMDRGEGVGVLNNPVVQAIAREHSRTPAQVCLRWAVQRGYTAIPKSTHESRLQENLHVFDFTLSAEDMVKISRLNRHLRYNDPGEFCKGMGLPNGYPIYA

SEQ ID NO: 74 - protein, 361 aa, Nannochloropsis gaditana
MTDPSASTTAAAQLPGRMLAGVADHHGDRFDMREIPVTPPGVGQALVKVVTSGVCHTDVHAVDGDWPAPTKLPLVPGHEGAGVVVAVGPGVSSTVVSLGDRVGIPWLHSSCGSCEFCLSGRENLCPLQDNTGYSVDGCFAQYVLAPAAHLAKIPDEVSFEQAAPILCAGVTTYSAIKATEARPGQFLTVIGAAGGLGHLAVQFGVALGLRVMALDRGADKLKFCTDTLGAEAAFEAMDPGVVDQVIATTKGGSHGVLCLAPSIGAFKSAVSLCRRGGTIVMVGLPKGDLPLNIFDIVIRGITVRGSIVGTRKDLDEALDFAARGKVKCHTEMHGFGELNQVFDQLRSGKVMGRLVLSVDGM

SEQ ID NO: 75 - protein, 732 aa, Nannochloropsis gaditana
MGKRQVSYFAFSTSPVSGKPAAIPPSLIGISTLNALRDAEKVADAVKHAVSSVVKYVDCSSDSQNEKQIGNALSAFDRSSFYVGSKLSCCDAAPEDVTEACKRSITELGVSYLDNYMMHWPVQLKSDSKPVSLDDGDTYELVQDGDMDCIMATYEAMERLVDQGLVRSLGVSNMGIRTLSELLSRCRIRPTVLEVEMHLYLAQPKLLEFCREENIHVVANSPPGKMRNRHPNDPSLLDDPVLLRIAEEAVRAAQVLLRRGIQRGRSITRKTPSQSLMDENKDLLDWCLSRDHMSRLDALDKGSRFPSVLPSMCDLDRDSENYAGAGHPVSQPHRTPCTMDKNGGFRNRFERPGKYLKTDILVQRGALSDLARLGKSIIPEESHGSANYLITDSVVDALYGDTVLNGLKSAGLDMTKIVVPAVSMDESGEPSTEPNKNGAIFNACVDRVLGNGISKHSCIISLGGGVINNLCGVIAATLYRGIKLVHFTTTTMGMLDAAIDFKQAFNHSCGKNLVGAYYPADLIVMDPECLKTLSNRHMLNGVAEALKHGLTQSWELTSAIVEPLRGDSARLGDSKYLETLCKETIEIKVPTLTHYKESDFNEMVPQYGHAVAHAVEHLSWEEGQVPLLHGEAVAIGMCVTAELGHLLGLCDKSVVDHHYDLVGTTGLPCNVPDTMKVNDILHVMTYDKHFMSKPCMGFCKEIGVMAKNKDGSYAFSVEMEPVREALQLNMSK

SEQ ID NO: 76 - protein, 522 aa, Mortierella elongata
MPSFIGAIDNGTTSSRFLIFDEKGNLVIGHQLEYRQIFPHPGWVEHDPMDILGSVTACIEGALRKFELQGNDVKNLRGIGITNQRETAVVWDRTTGKPLHNAIVWSDTRTQDVVTKLCESSDKGTDALKDICGLPLTTYFSAVKLKWLLENSSEVKEAHENGNLMFGTVDSWLIYNLTGGKEGGVHVTDVTNASRTMLMDIKTLQWSEEALKFFGINADILPEIKPSSTLFGKVQHPALEQLQDVPIAGCLGDQHAALVGQHCFQVGEAKNTYGTGCFMLFNTGSKITPSNNGLLTTVGYQFEGEPAAYALEGSIAVAGSAVKWLRDNMGIIRSAEEINDLAAQVDSNGGVVFVTAFSGLFAPYWRPDVRGSIVGISQHTTKHHLARATLEATCFQTRAILDAMNADSGHPLATLRVDGGLSNSDLCMQLQSNILGLEVARPQMRESTALGAATAAGVHLGIGIWKGGFKAFAERARESKEVLQIFTPKINDEEREKEYALWQKAIDTTIGVKSKTTGKREP

SEQ ID NO: 77 - protein, 222 aa, Nannochloropsis gaditana
MTSSYINSYVGAIDQGTSSTKFIIYNHSGQQVGLHQLEHAQIYPQPGWVEHDPMEIWANTVTCIRRAMESANVDAELLEAVGITNQRESTLIWNKKTGVPYYNVIVWNDARTRGICEDLKTAGRRGIDRFREKTGLPIATYFSASKILWLLDNVPGLRDDAEKGEAIFGTLDSWLIYKLTDGQVHSGPCVAYPGGLSPSSLSSALRPPASPPSQAPSLSPDP

SEQ ID NO: 78 - protein, 866 aa, Nannochloropsis gaditana
MDEELNVLSPFLVKAEVLLVLVVVLVASVVWLFWEIVSFMMDRGKEETNPDWWEVLRNCQHRRLIIPPYCVQEVPELGTFSRLTTATTNAMKNMSGVIQRTSHLISGGSGKSAAAIKKGARQDLPSTQQEGDENMKGYTVDGNARGVKLRRRGSKQSIVGLSNHGTSAGGKPALQPTANPTPLTLSENGANPDASAASDARPKPHRLDLNGEEGNMVPCNGSLSSRAGDGKRVVGMSGLASTSAAAGSDASSANVKSMEISPADTPCRGRIRFLPHQRERQQIENHEKSHEGKPTRSGLPLRALDSQPPLTPYALPDAEGVLASSAQSSRHAPDAIAATPRLSSSHAANGEPITTPAQPVRLPSMEHAHSGTGVALSGGSSGVAGRGFIFSPLPEDCTPLLAFVNSRSGVSQGAYLIHQLRRLLNPIQVIDLANEDPARALRLYLELPRLRVLVCGGDGTAKWIMNVLEDLNPECWPPIAILPLGTGNDMARVLGWGGGYNNQSIVEFLAQVQRAHVVVVDRWEMKLTPAGKGSSRAKTVTFNNYFGIGVDAQAALKFHHLREQKPQLFFSRLVNKLWYGMLGAQDLFRRTCVSLPERLKIVADGKELTLPAHVQGVIFLNIESYGGGVKLWNVEEDDESAGNGLFDASSSSCSSEEGDRSEDESRRQRRRRRRRERQRRQQSQAEEEAHRQREQQEKPSSMALTSSSMQDGLMEVVAINGVVHLGQLQVGLSKAVKICQCREAVITTTRDLPMQVDGEPWPQAKSTIKITRKKDPAYLLRRTMDSGGAVVGEVVELLESAVKDGVISLPQKKSLLTELSRRVEMKRKVFEQELSQNDGVPSFSKGFDVSRLRLAADSNSKDCVLM

SEQ ID NO: 79 - protein, 833 aa, Mortierella elongata
MWRRIPATGARHSTSFRTKAVYATAGATTLALSGYYYNLKQQQRALDDSFEYPPQSSMIYLEPQQAARDPTRPHAFWAPPSREDMIRMLQEGPGSIVKEKTAAAAAAAAAAAAGTTPGSKPVVAVAATMEDDKDSDVFDLLIIGGGATGAGCAVDAATRGLKVAMVERDDFSSGTSSRSTKLVHGGVRYLEKAVRELDIEQYKLVKEALNERANFLKVAPYLSYQLPIMLPIYKWWQVPYYWAGSKAYDLLAGHQGMESSYFLSRGKALEAFPMLKNDKLVGAMVYYDGQHNDSRMNVALGLTAVQYGAVIANHVEVIELHKDENRRLCGARVRDAMTGKEFNVKAKGVINATGPFTDGIRQLDDPSIQSIVSPSAGVHIILPNYYSPGNMGLLDPATSDGRVIFFLPWQGNTIAGTTDSATKVTPNPMATEEEINWILGEVKNYLNPDVKVRRGDVLAAWSGIRPLVRDPAAKSTEGLVRNHMINVSPSGLLTIAGGKWTTYRAMAAETIDEAIKEFGLTPARGCSTERVKLIGSHGYSNTMFIRLIQQFGLETEIAQHLANSYGDRAWAVASLAQSTGKRWPVFGRRVSNQYPYIEAEVRYAVRREYACTAVDVLARRLRLAFLNVHAALDALPRVVEIMAEELKWDAARQAKETEDAKAFLTTMGLPVSPIAYPTNVPEAVVGHPVVDGEKVQPTSFWGRMSGKSASGAIVTDSFYSRAQFNPEELAEFHKVFGALDHDGDGHIDGHDLEEVLIHLDVQVEPQVLKSIIEEVDLDNSGTIEFNEFLEVMGGLKEHASRTAFSKIIVEVESKRNVDYGIKAKTTDRSGGGA

SEQ ID NO: 80 - protein, 340 aa, Mortierella elongata
MTERVALIGSGNWGSAVAKIIGRNVRKFDHFDNKVKMWVFEEKVNGQNLTEIINTKHENVKYLPGIQLPSNIVACPDLLETCRDATMLVFVVPHQFVTSICKQLKGRIPANCKAISLIKGIDVNADGFRLITDMIQESLGVPTCVLSGANIANEVAEEKFCETTIGYRNRADGELFRDIFHTPSFRVNIVPDVVGVELCGALKNIVAIGGGLVDGLKLGDNTKAAIIRIGLYEMRKFSKMFYADVKDETFFESCGVADLITTCAGGRNRKVAEAHVTTGKSFDQLEQEMLNGQKLQGTSTAQDMYNILSKKNLCHEFPLMTTIYKICYEGLPPIRIVEDI

SEQ ID NO: 81 - protein, 341 aa, Mortierella elongata
MLITECISLFHRGSAVAKIVGGNVQKYDHIQNEVKMWVFEEQVDGQNLTEIINAKHENVKYLPGIKLPENIVACPDLIKTCEDATMLVFVVPHQFVASVCRQLKGKISPKCKAISLIKGVDVEENDNGFRLITDMIQDSLGIRACMLSGANIATEVAEERFCETTIGYRNKADGELFKEIFNTPTFRVNIVEDVVGVELCGALKNIIAIGGGLVDGLKLGDNTKAAIIRIGLYEMRKFAKMFYADVKDETFFESCGVADLVTTCAGGRNRKVAEAHVTTGKSFDQLEKEMLGGQKLQGTSTAKDMYGILSKKGLCKEFPLMTTIYRICYEDLPPIRIVEDI

SEQ ID NO: 82 - protein, 402 aa, Nannochloropsis gaditana
MATLHISNLTLTIYNHGIFVLMSAALSFLLIVWRFSLAEAGRSHHFEGPSSNPVKPHSITIVGSGNFGSAIARLLGRNVLRSPKHFRSEVRMWVFEEELDDGRKLSDVINADHENVKYLPGIQLPTNVRAVPDLSDAVRNASIVVFVLPHQFLPGLLPRISSCLHRGAMAVSLVKGLDFDDEGPVLITDMIREGLGEDVSEVCVLMGANVADEMARDEFCEATLGCPDPEGAGAVLQQLFDCPTFRVEVTPDPIGVELCGALKNVVALAAGFCDGLDWGGNTKAAIIRRGLEEMRLFCKLLHPSVRDMTFFESCGVADLITTCYGGRNRKCAETFARAGGTMAWDEIEKEELGGQHLQGPQTTSKLHKVLEQKKWLSRFPLFRSVYQIAYQGRPPATLVQDL

SEQ ID NO: 83 - protein, 429 aa, Nannochloropsis gaditana
MSPTFRRRHSNAPFKLQIFMVKFLAVVALLGCCCLHGVASGTPPHAAFVPRASTKSLGNRLAKAPQARREQTIMQLSARRSRSMRPLPYPVRFAVLGGGSFGLALASVLGKKSIPVTILVRKEEVAEHINLHHRHPTYLSDIALAPSIRATVQPEEALRDASFIIHAVPVQYSRKFLEDIAPHVPKNTPIISTSKGIETGTLCMMQDILLETLGPNRETAYLSGPSFAREIALGLVTAVVAASESEALANEICDIMGCNYFRVFTSTDVVGVEVGGAVKNVIAIAAGMCEGLGLGTNAMAALVTRGCNEMQRLALSLGARPSTLTGLSGVGDTFGTCFGPLSRNRNLGVRLGKGERLENILGSSTEVAEGHATAFSLVQLIEKTNRAYRRELEFPIIYGVKEILEGKRTPAEGLRDLMAMPVRVEMWNL

SEQ ID NO: 84 - protein, 654 aa, Nannochloropsis gaditana
MSLQPHLALLGMAGSLVVADRLRSGPGRKSRAKDSHRHLPPTSRSANCEASGGKRELSPVEQLEDMRTTPIKCRDGTLVYPYSLPTRDAQLNRLKKEKFDVLVIGGGCVGSGVALDAQIRGLKTAMVEANDFSAGTSGRSTKLIHGGIRYLETAFWKLDYGSFALVQEALEERAHMLNAAPYMNSPLPIMIPIYKWWEVPYFWAGAKAYDLVASRQKSVPSSHYMDVDEALFQFPMLRGKGLKGAIIYYDGQMNDTRMGLTIALTAAQEGAAIANRVEVVSLLKDPGTGQVNGARVQDRLTGVEWDIAAKVVVNATGVFADKIRKFDDPKAVELIEPAAGVHVMFPAHFSPAKMGLIVPKTTDGRVLFFLPWEGCTLAGTTDSHSDITMHPQPTAQEVNFIMQETNRYLTTNVAAKDLIAAWSGLRPLVKDPEKIKEGTAALSRNHVIEVSETGKLITITGGKWTTYRRMAEDTVDRILQEHAGLLANGDVSPQASTWNRKLLGADRAGIVCAQKFNQIGITLRNDYELPEDVSAHLVKSYGTRALQVAEWVRAGYLDTKPGKAKRLHSRYPFLEAEVIFAVDQEYALKPMDILARRTRLAFLDTEAARAAVPRVVKLMGDLLGWSWRQRTMEKAEALAFLETMNVEKTALLKK

SEQ ID NO: 85 - protein, 852 aa, Mortierella elongata
MASKNSKTGPDNAGASTGPALELKPLKNVMPIVPAQQVDSSSCPPSGETSPLLENAPNGKLATQSGGPDNDESGVENITKKHAGRIREDPVGFVVQTAAFYQGTGWRSYSNYVGTRIFYEGFSASFKDRILASQKVVELVKSMANKQLEVLIKQRQDAHEAEKVANAGKKNFKPKVWPMRPEDVEVRRKTLEAELTAVAKTNIDKLVCDMNSMKFIRFFAFLINNILVRMYHQGIHIKESEFLELRRVAEYCAEKKYSMVILPCHKSHIDYLVISYIFFRMGLALPHIAAGDNLDMPVVGKALKGAGAFFIRRSWADDQLYTSIVQEYVQELLEGGYNIECFIEGTRSRTGKLLPPKLGVLKIIMDAMLSNRVQDCYIVPISIGYDKVIETETYINELLGIPKEKESLWGVITNSRLLQLKMGRIDVRFAKPYSLREFMNHEIDRREIINEQEMTSNAAKSQLLKALGYKVLADINSVSVVMPTALVGTVILTLRGRGVGRNELIRRVDWLKREILSKGGRVANFSGMETGEVVDRALGVLKDLVALQKNLLEPVFYAVKRFELSFYRNQLIHLFIHEAIVAVTMYTRIKIGGAKSTQQISQTELLNEVTFLSRLLKTDFIYNPGDIQSNLENTLEYLKKSNVIEINSEGFVGLSDVERGIGRENYDFYCFLLWPFVETYWLAAVSLYTLIPTAKEITEQANAGGDQLHWVEERVFVEKTQMFGKTLYYQGDLSYFESVNMETLKNGFNRLCDYGILMIKKPTGPKERTKVALHPDFMPSRGSDGHVIASGALWDMVEHIGTFRREGKNRRDNATVSSRVLRFAEVVANSPAPVKVPMPSPAPKQGNGAPKL

SEQ ID NO: 86 - protein, 209 aa, Mortierella elongata
MTYLFIAALAYGIGSISFAVVVSAAMRLQDPRSYGSKNPGATNVLRSGNTLAAVLTLIGDALKGWLAVWLTAQFVHSFGSQYEVGNEAIGLAALAVFLGHLWPIFFHFKGGKGVATAAGVLFAIHPILGLATAASWLIIAFFFRYSSLAALVAAIFAPLYEILMFGFDSNSIAVLAMSLLLISRHRSNIQNLFAGKEGRLGQKSKDKSL

SEQ ID NO: 87 - protein, 306 aa, Mortierella elongata
MSIVTYLQAAIGIPLFYFLVLPKILAVLPKKAQFLAKCIIVLLATLIMSVAGCFISIACALVNKRYIINYVVSRFFGILAAGPCGVTYKVVGEEKLENYPAIVVCNHQSSMDMMVLGRVFPKHCVVMAKKELLYFPFLGVFMKLSNAIFIDRKNHKKAIESTTQAVADMKKHNSGIWIFPEGTRSRLDKADLLAFKKGAFHLAIQAQLPILPIISEGYSHIYDSSKRSFPGGELEIRVLDPIPTTGLTADDVNDLMEKTRDLMLKHLKEMDRSSSTVTSPAATVGKTTATAPQDEASVKKRRTLKD

SEQ ID NO: 88 - protein, 304 aa, Mortierella elongata
MSSESTIPWCIITTPVFILALPRLLAVLPQKIQFVTKCCIVLIATFIMSIVGCFVAIVFALLRRRHEINFVVARIFSFIASYPCGVTFKVVGEEHLEKYPAIVVCNHQSSMDMMILGRVFPKHCVVMAKKELQYFPFLGIFMTLSNAIFIDRKNHKKAIESTTQAVTDMKKHNSGIWIFPEGTRSRLETADLLPFKKGAFHLAIQSQQPVMPIVAAGYSNIYDSANRSFPGGELEIRVLEPISTIGMTADDVNELMERTRAVMLKNLKEMDHSVKSSSNSNGSSTAVAEGKTDEGLTQRRPVKE

SEQ ID NO: 89 - protein, 101 aa, Nannochloropsis gaditana
MVISFIFSWMLQILACIFICPFLPSCKERLLLLGWIFRSVSSLVIRLNPYWHLRVLGPRPTRPPSKTLIMCNHLSNADAFFLSSALLPWETKYIAKASLFQ

SEQ ID NO: 90 - protein, 199 aa, Nannochloropsis gaditana
MRSNKSCKTCPNRIHVGIAILFPLLLSAFCFCHFLMLPPAIALLIMPYAPVRRVLRLWEATIAAYWLSFGAWLLENFGGVKLIISGDTFTKKDNVLIICNHRTRLDWMWLWSWAAYFDVLSSYRVILKDSLRCFPWWGWGMSLCLFPFIRRGQKHRSTDLAHLKRNCRYLIQLKVPNSLIIFPEGTDLSPSNQERDRNY

SEQ ID NO: 91 - protein, 422 aa, Nannochloropsis gaditana
MTSTASLACGACTAAVLVCLTTGDGVATRHIDANVGNRRTSAFLPVMPPMGTPVTGRIRSHPLEAHKMYYVCQGGTRLSQRRHERLGTRTAVMVVKTDVEISDKRDVDPEVGSSSKSTDHTGVSRFGSAMPKSAEGVGPPPAPQDNFKHKSLAGVPTDYGPYLTIKGFKINAFGFFFCFMAILWAIPWAVFLVVYKALLEFVDKLDPCRYNVDRSSSLWGWLTSLSTDSLPEMTGLENIPDGPAVFVANHASWMDVPYSAQLPVRAKYLAKADLTKVPILGNAMSMAQHVLVDRDDKRSQMEALRSALLILKTGTPLFVFPEGTRGPGGKMQAFKMGAFKVATKAGVPIVPVSIAGTHIMMPKEVIMPQCAGRGITAIHVHPAIPSTDRTDQELSDLAFKIINDALPNEQQCESTSKETGGA

SEQ ID NO: 92 - protein, 391 aa, Nannochloropsis gaditana
MSSHMPVCRGDPEAGVVPAGGTVGNEEMAGRENGGSGMYRLAEDVDGNGRDEGCQWVPPALRTSLERYRWLEIILLSVIVILAKEGFGSGVKNHRQYIPLVTQVLPGGAVVVLGNATAFSYPVRFREGTLECPPVTLEFCATSPESALADPCCEFMTTGAKPFQTVSHDDLIWITVGLPLILLVLRHLLLKWYLCSVPASSADPMFSSEDKSALRPLSGLPFGYSATFCLRDVLIGLFFSLALTRATTNSLKMLTSQPRPNHFALRLFASLSPDSSAAIHYAESAWKAWPSGHSSMSMASGAFLSLVLLRDLRQFAGPLQRQLRACLVILALGPVYLAMFVAGTRVHDYFHTTADAVTGSALGLLWAVLAFYQVVPAGGLEVRANPPLKYL

SEQ ID NO: 93 - protein, 491 aa, Mortierella elongata
MASFPFVLQAHQGNHQVELVYNGQQLEFDGLSLDEPKQSSSCLPCGPSSAFAGGHRIIKTVEILNIDIEHEDSLVLSVASAKNGPTKESVLERLVFQVRDKANAVQWQSNVLSHVYKDIKKGRHFKVLVNPFGGQGHAKKLWETIAEPIFKAAGCTYDLTYTTHRYHAKEIARDLNIRLFDAVVSVSGDGVLHEVINGLMERPDAIAAHKLPIGAIPGGSGNALSYSLLGEDHGSHVTNAVLGIIKGRAMPVDLCSVTQGQNRYFSFVLQSFGLVADVDLGTEDMRWMGEARFTVAAVGKLLSQQTYPCEISYIPVETNVDKIRAEYNYRRQQSVVWADQTHDELDQSHPTIVDRFGGVNAQLNKSDGWVTDSEDVITAVGAKLPWISKGMLLNPASTPNDGLIDLIVFPKGTGRMNGIQIMLGTETGEHIYHDKVRYMKVKAFRLTPKNESGFISMDGEHTPYSPYQVEAHPGLISVLSIEGRYARSMRE

SEQ ID NO: 94 - protein, 336 aa, Mortierella elongata
MDEKKIGFIVNRRGGGGKGGKTWDKLEPAVTTRLASAKWKVEYTQHSGHASDLAREFVNEGYNIIVAVGGDGTISQVVNGYMLADGNSKGCAVGIISSGTGGDFVRTTKTPKDPLEALELILSTESTLVDVGHVSATKPNSPSVTNEQYFINICSVGISGSIIKRVESSSIAKYISGSLVYWLYTYLTGLVYRPPPVKYTLTGGSAGADDGKEKHMGLYIMAVANGRYLGGNMHIAPKAQISDGQFDVVCLHDLTLTDAFFKASPALKSGNLMNLPAHQAFTQRNTKVSISPVNAKDHIYVEADGEVAGVLPARWEIIPQGCRMILPLVQGSTQSV

SEQ ID NO: 95 - protein, 382 aa, Mortierella elongata
MGIIPTSDKFPVLVVLNPHSGRKQGLEAWENTVKPALNAANKPFRLIESNSQGHVVSYFVDNIKPIITDLAQSLSTVTQGAGDDETIVYPTSAKLQIIVLGGDGTVHEIVNGILKGVEGTGFVTDAFRPEVEFSVIPTGTGNAISTSLGVTSVQNAVDRFIAGKTVPLHLMSVATQTSQLYTVVVNSYGLHCATVYDSEEFRHLGNDRFRQAAMKNVENLKQYEGKLSFFGPIQRYNRISASLVDTETDNNIAQADSKSSAVATLTLPGPFTYLLISKQASLEPGFTPTPFAKTSDDWMDVLAVQNVGQAEIMQMFGSTATGTHVNQDHVDYIKAKTIELETPTQGRLCIDGEFLTIEAGPEGKVRFEVNSDPNIQIFHIFA

SEQ ID NO: 96 - protein, 529 aa, Mortierella elongata
MSPNQFQAKASFAGHQRVSDARLSLGTHELTIHAPKGSDNNTTTIQVPYSCIYGYETSTDKATGENYKNKVIVHYVAFSGPDLRNPSAAKRTTAQLLFERTEDADRFIQTARDLGALPTPRRILLLVNPNGGVGKAKRISDTVVKPMLQHSGLTVKEQYTEYGRHAVDIASKVNLDEVDSLVVVSGDGVLHEVINGLLSRPDWDRARKTSIGIVPAGSGNAIAASLGIVSQFVATLTVIRGETSKLDIFSLSQLNRPKIYSMLSFSWGMMADADIESDSYRWLGPLRFDVAGFIRMIRLRRYPGKVYVLPPKHQQNPSTTEQQLTPPQSPSHKREPESQFQHLLDSNIKEPPKPWSLIPNMPFYSMLLLLNCPNVGETIFFTDTIRFNDGIMRLWYSAETRFWKILMPFIFDQQNGKMVERDLMKDLECGGILIIPGVEGKPDDPSTHKVIEPDWVTSSAAKAQNIYQNPGLFDVDGEVMPTARTLIEIHPSLMNILVPEWLYHKDDDNTTARAHEVAVIQAIKAQQKL

SEQ ID NO: 97 - protein, 866 aa, Nannochloropsis gaditana; identical to SEQ ID NO: 78

SEQ ID NO: 98 - protein, 486 aa, Nannochloropsis gaditana
MKLIQYFGTALCVVILSCVTNIIPGGRIALGRPFSRLFGGSSRNLRAEVEAAVPHFIVPEDRVEYPTPKLAALKSKLKEIGHHKAMGHPHQHQGLDGRRRVSLHPSHRPAPSSLGAAEDKEQEEEGGEEEEEGQEGVIAPPAWKPGHMNPRDSSSDMGKATKGKPGTPSAFLPLGVPPPSLFPPSARPIRRSPWSLLFRRGLPRPRRKRPIGINRIKTLPPSVTPLIAIVNSKSGGRQGKNLFKRLRAALSRAQVFDIQKVDLKEALSLYCHLPNSCTLLVCGGDGTASRVFEVVDGMEWKHGPPKIAIVPLGTGNDIARVLDWNLGHDWSGGYFPWSNDAADANLLSVFSDLTRAMERKMDRWELRMTEAVPSSDRHRQPVKYMLGYLGIGVDGKVALDFHKLRDRAPYLFLSPTLNKFYYALMGLRDFFVRSCKNLPDKVELWCDGKPIVLPPQTESFIVLNINSHAGGVELWPEYLMGGGMEG

SEQ ID NO: 99 - protein, 603 aa, Nannochloropsis gaditana
MKLIQYFGTALCVVILSCVTNIIPGGRIALGRPFSRLFGGSSRNLRAEVEAAVPHFIVPEDRVEYPTPKLAALKSKLKEIGHHKAMGHPHQHQGLDGRRRVSLHPSHRPAPSSLGAAEDKEQEEEGGEEEEEGQEGVIAPPAWKPGHMNPRDSSSDMGKATKGKPGTPSAFLPLGVPPPSLFPPSARPIRRSPWSLLFRRGLPRPRRKRPIGINRIKTLPPSVTPLIAIVNSKSGGRQGKNLFKRLRAALSRAQVFDIQKVDLKEALSLYCHLPNSCTLLVCGGDGTASRVFEVVDGMEWKHGPPKIAIVPLGTGNDIARVLDWNLGHDWSGGYFPWSNDAADANLLSVFSDLTRAMERKMDRWELRMTEAVPSSDRHRQPVKYMLGYLGIGVDGKVALDFHKLRDRAPYLFLSPTLNKFYYALMGLRDFFVRSCKNLPDKVELWCDGKPIVLPPQTESFIVLNINSHAGGVELWPEYLMGGGMEGAFKPSRFDDGYLEVVAISGVLHLGRIRVGLDRPLRLAQAKEVRIRTKSFLPGQVDGEPWRLPRCELTLRHNGQAPVLQHVSKELLQYNEWLVGQGKLDAAGKDQLLQAFKRRLQVSQ

SEQ ID NO: 100 - protein, 355 aa, Mortierella ramanniana
MASKDQHLQQKVKHTLEAIPSPRYAPLRVPLRRRLQTLAVLLWCSMMSICMFIFFFLCSIPVLLWFPIILYLTWILVWDKAPENGGRPIRWLRNAAWWKLFAGYFPAHVIKEADLDPSKNYIFGYHPHGIISMGSFCTFSTNATGFDDLFPGIRPSLLTLTSNFNIPLYRDYLMACGLCSVSKTSCQNILTKGGPGRSIAIVVGGASESLNARPGVMDLVLKRRFGFIKIAVQTGASLVPTISFGENELYEQIESNENSKLHRWQKKIQHALGFTMPLFHGRGVFNYDFGLLPHRHPIYTIVGKPIPVPSIKYGQTKDEIIRELHDSYMHAVQDLYDRYKDIYAKDRVKELEFVE

SEQ ID NO: 101 - protein, 349 aa, Mortierella ramanniana
MEQVQVTALLDHIPKVHWAPLRGIPLKRRLQTSAIVTWLALLPICLIIYLYLFTIPLLWPILIMYTIWLFFDKAPENGGRRISLVRKLPLWKHFANYFPVTLIKEGDLDPKGNYIMSYHPHGIISMAAFANFATEATGFSEQYPGIVPSLLTLASNFRLPLYRDFMMSLGMCSVSRHSCEAILRSGPGRSIVIVTGGASESLSARPGTNDLTLKKRLGFIRLAIRNGASLVPIFSFGENDIYEQYDNKKGSLIWRYQKWFQKITGFTVPLAHARGIFNYNAGFIPFRHPIVTVVGKPIAVPLLAEGETEPSEEQMHQVQAQYIESLQAIYDKYKDIYAKDRIKDMTMIA

SEQ ID NO: 102 - protein, 566 aa, Mortierella elongata
MSQGDAITTSHSDGTEKRHDSTTNILSDVPPQTEDVKSSSSKKKRSTYRHTFPVHTKTLPSPLSKEAPPESYRGFVNLGMLLLFGNNIRLIIENYQKYGFLLSIPGSNVSKQDWILAGLTHAILPLHVIVAYQLEQWASRKAKGFRKRLADQKENPTTKDDEDKKAVPAGDKVRGGKKDKKNLTLEEQIKENRKTVGWLHFANVSLILGWPSFMSYFVIFHPFLAMGCLMTSLILFLKMVSFALVNQDLRYAYIQDTPATEQSSPHLTKVHNDTITTTNTTSDGATTTTTLTTTTTVVKTITVKKDAEKHGGAYQYEVHYPQNITPGNIGYFYLAPTLCYQPSYPRSTVFRPSFFFKRVLEIVTCLGMMYFLIEQYATPTLQNSVRAFDELAFGRLLERVLKLSTTSVIIWLLMFYTFFHAFFNALAEVLYFGDRRFYLSWWNATSVGMYWKTWNSPVYTFFKRHVYLPMITSGHSALTASVVIFTISALLHEVLIGIPTKMIYGYAFAGMFFQIPLIALTAPLEKWRGTGSGLGNMIFWVSFTILGQPACALLYYYHWTKRSMNA

SEQ ID NO: 103 - protein, 331 aa, Mortierella alpina
MPLFAPLRMPIQRRMQTGAVLLWISGIIYTLGIFVFLCTFKVLRPLIIIYLLWAFMLDRGPQRGARAVQWYRNWVGWKHFAQYFPMTLVKEGELDPSKNYIFGYHPHGIISLGAFCTFGTEGLHFSKRFPGIKPQLLTLHANFQIPLYREMVMAHGCASVSRASCEHILRSGEGCSVVIVVGGAQESLSTQPGTLNLTLKKRLGFCKLALVNGASLVPTLAFGENELYEVYTAKPKSLMYKIQQFAKRTMGFTMPVFNGRGVFNYEFGLLPRRKPVYIVVGKPIHVDKVENPTVEQMQKLQSIYIDEVLNIWERYKDKYAAGRTQELCIIE

SEQ ID NO: 104 - protein, 393 aa, Nannochloropsis oceanica
MYPIKLCFLFILTIPPYAHVRTRTPHRRGTTSKMAKANFPPSARYVNMTQVYATGAHNMPDEDRLKVMNGLSKPLTEAKPGDLGFGDVESMTFCEEFVAIMFLLIIVGSMLWIPIAVLGFALYVRSAMAWVVMLIVFFTLSLHPVPRIHDMVHSPLNHFIFKYFSLKMASDAPLDSAGRYIFVAPPHGVLPMGNLMTVHAMKACGGLEFRGLTTDVALRLPLFRHYLGAIGTIAATRHVAKQYLDKGWSIGISSGGVAEIFEVNNKDEVVLMKERKGFVKLALRTGTPLVACYIFGNTKLLSAWYDDGGVLEGLSRYLKCGVLPLWGRFGLPLMHRHPVLGAMAKPIVVPKVEGEPTQEMIDEYHSLFCQTLVDLFDRYKTLYGWPDKKLLIK

SEQ ID NO: 105 - protein, 179 aa, Nannochloropsis gaditana
MGHVGKLDLLKALGELLRLAIPSTFVWLITFYVYFHCTLNLFAEITRFGDRLFFKDWWNCTSFSRYWRTWNLPVHQFLVRHVYFPLLRAGASKMTANVTVFAVSAFFHELLISIPCHVVRLWAFLAMMGQIPLIYITDHLDKTLFKETQAGNYMFWLIFCIFGQPMAVLLYYADFSARS

SEQ ID NO: 106 - protein, 279 aa, Nannochloropsis gaditana
MVCPLRSLVRDYRKTQGLVTSPHRSHGPDMSFKCKPSQKPNKQFWRYASFLAFIATFLLVPSTTSWASALHRACFMAYVMTYLDTSYRDGSRAWPWFQRLPVWRLYCRYIKGQVITTVPLDPHRQYIFAAHPHGIATWNHFLTMTDGCRFLSRIYPRPRLDLGATVLFFIPLVKEVLLWVGCVDAGAATANAILERGFSSLIYVGGEKEQILTERGRDLVVVLPRKGFCKLALRYDCPIVPAYAFGENDLYRTFNYFKGLQLWVERHAGRVVPRNRSEH

SEQ ID NO: 107 - protein, 363 aa, Nannochloropsis oceanica
MTPQADITSKTTPNLKTAASSPSKTSPAPSVQYKAANGKVITVAMAEQDDGNMGIFRECFAMVTMGIIMSWYYIVVILSLLCLVGICIFPAWRAVAATVFVLMWSAALLPLDYQGWDAFCNSFIFRLWRDYFHYEYVLEEMIDPNKRYLFAEMPHGIFPWGEVISISITKQLFPGSRVGSIGASVIFLLPGLRHFFAWIGCRPASPENIKKIFEDGQDCAVTVGGVAEMFLVGGDKERLYLKKHKGFVREAMKNGADLVPVFCFGNSKLFNVVGESSRVSMGLMKRLSRRIKASVLIFYGRLFLPIPIRHPLLFVVGKPLPVVHKAEPTKEEIAATHALFCEKVEELYYKYRPEWETRPLSIE

SEQ ID NO: 108 - protein, 540 aa, Mortierella elongata
MDKQQPDIVTMIPGIVSTGLESWSTTNNSCSQKYFRKRMWGTTTMFKAVLLDKDCWITNLRLDPETGVDPEGVRLRAAQGLEAADYFVQGYWVWAPIIKNLAAIGYDNNNMYLASYDWRLSFANLENRDNYFSRLKSNLELSLKMTGEKSVLVAHSMGSNVMFYFFKWVESDKGGKGGPNWVNDHVHTFVNIAGPMLGVPKTLAAVLSGEVRDTAQLGVVSAYVLEKFFSRRERADLFRSWGGLSSMIPKGGNRIWGTIHGAPDDGTHDEEETVRNEKIAKSEETPGATTKRKHGEQSPTFGAMLAFAEGSNMENHGMDESMGLLSKMAGNAYNTMLAKNYTVGASVTQKQMDKTTKDPASWTNPLEATLPYAPKMKIYCLYGVGKSTERSYTYNRVSDLAPQIFDQRPGNVSDETGQVPNIYIDTTVHDDKLGISYGVHQGDGDGTVPLMSTGYMCVDGWSKKLYNPAGLKVITREFTHQSSLSPVDIRGGKRTADHVDILGNYQVTKDLLAIVAGRDGDGLEEQIYSKIKEYSAKVDL

SEQ ID NO: 109 - protein, 373 aa, Nannochloropsis oceanica
MAHLFRRRSKGEGNSTSSRCLSLSEGNKAMLILSSEIEPPASATSKAATSGIKEIGDPSLPTVALLSLPSISKADTNSATAAVAAGTLEDAAAGALTAPFADRSVKKQYGQDGDGAQCKEAEGGRKRSGSVGNLLLSSMTSFSKGTSLSFLTGEDKTPSPPETGPAGIDFSTPAHPTMQFVDFIITFLLVHYIQVFYSLVFLFIYLVKHGHRWPYFLAAIYAPSYFIPLQRLGGWPFKGFMRRPFWRCVQRTLALQVEREVELSPDEQYIFGWHPEVSILLGGGSKEIYTTDPYTPETTLVLKIRKGFIRMALRYGCALVPVYTFGEKYAYHRLGQATGFARWLLAVLKVPFLIFWGRHKHKYAKPEEFVAIS

SEQ ID NO: 110 - protein, 352 aa, Nannochloropsis gaditana; identical to SEQ ID NO: 19

SEQ ID NO: 111 - protein, 634 aa, Pseudomonas aeruginosa
MSRLPVIVGFGGYNAAGRSSFHHGFRRMVIESMDPQARQETLAGLAVMMKLVKAEGGRYLAEDGTPLSPEDIERRYAERIFASTLVRRIEPQYLDPDAVHWHKVLELSPAEGQALTFKASPKQLPEPLPANWSIAPAEDGEVLVSIHERCEFKVDSYRALTVKSAGQLPTGFEPGELYNSRFHPRGLQMSVVAATDAIRSTGIDWKTIVDNVQPDEIAVFSGSIMSQLDDNGFGGLMQSRLKGHRVSAKQLPLGFNSMPTDFINAYVLGSVGMTGSITGACATFLYNLQKGIDVITSGQARVVIVGNSEAPILPECIEGYSAMGALATEEGLRLIEGRDDVDFRRASRPFGENCGFTLAESSQYVVLMDDELALRLGADIHGAVTDVFINADGFKKSISAPGPGNYLTVAKAVASAVQIVGLDTVRHASFVHAHGSSTPANRVTESEILDRVASAFGIDGWPVTAVKAYVGHSLATASADQLISALGTFKYGILPGIKTIDKVADDVHQQRLSISNRDMRQDKPLEVCFINSKGFGGNNASGVVLSPRIAEKMLRKRHGQAAFAAYVEKREQTRAAARAYDQRALQGDLEIIYNFGQDLIDEHAIEVSAEQVTVPGFSQPLVYKKDARFSDMLD

SEQ ID NO: 112 - protein, 487 aa, Nannochloropsis oceanica
MAHLFRRRSKGEGNSTSSRCLSLSEGNKAMLILSSEIEPPASATSKAATSGIKEIGDPSLPTVALLSLPSISKADKNSATAAVAAGTLEDAAAGALTAPFADRSVKKQYGQDGDGAQCKEAEGGRKRSGSVGNLLLSSMTSFSKGTSLSFLTGEDKTPSPPETGPAGIDFSTPAHPTMQFVDFIITFLLVHYIQVFYSLVFLFIYLVKHGHRWPYFLAAIYAPSYFIPLQRLGGWPFKGFMRRPFWRCVQRTLALQVEREVELSPDEQYIFGWHPHGILLLSRFAIYGGLWEKLFPGIHFKTLAASPLFWIPPIREVSILLGGVDAGRASAARALTDGYSVSLYPGGSKEIYTTDPYTPETTLVLKIRKGFIRMALRYGCALVPVYTFGEKYAYHRLGQATGFARWLLAVLKVPFLIFWGRWGTFMPLKETQVSVVVGTPLRVPKIEGEPSPEVVEEWLHKYCDEVQALFRRHKHKYAKPEEFVAIS